(19) United States
(12) Patent Application Publication
(10) Pub. No.: US 2016/0235069 A1
(43) Pub. Date: Aug. 18, 2016

(54) N-ACRYLIMINO HETEROCYCLIC COMPOUNDS

(71) Applicant: BASF SE, Ludwigshafen (DE)

(72) Inventors: Nina Gertrud BANDUR, Ludwigshafen (DE); Martin John MCLAUGHLIN, Bad Duerkheim (DE); Matthias POHLMAN, Freinsheim (DE); Jochen DIETZ, Karlsruhe (DE); Wolfgang VON DEYN, Neustadt (DE)

(21) Appl. No.: 15/023,176

(22) PCT Filed: Sep. 19, 2014
(86) PCT No.: PCT/EP2014/070000

§ 371 Date: Mar. 18, 2016

Related U.S. Application Data
(60) Provisional application No. 61/879,690, filed on Sep. 19, 2013.

(51) Int. Cl. A01N 55/00; A01N 43/40; A01N 43/78; A01N 43/56; C07F 7/18; C07D 417/06; C07D 401/06; C07D 405/06

(52) U.S. Cl. CPC A01N 55/00; C07D 401/06; A01N 43/40; C07D 405/06; A01N 43/56; C07F 7/18; C07D 417/06; A01N 43/78

(57) ABSTRACT

The present invention relates to N-acrylimino compound of formula (I): wherein X is O or S, in particular O; Het is a 5- or 6-membered carbon-bound or nitrogen-bound heterocyclic ring, W¹-W²-W³-W⁴ represents a carbon chain group connected to N and C═N, and thus forming a saturated, unsaturated, or partially unsaturated 5 or 6 membered nitrogen containing heterocycle, wherein W¹, W², W³ and W⁴ each individually represent CR^{v}R^{w}, R¹, R² may be hydrogen, halogen, etc. R³ is selected from the group consisting of hydrogen, halogen, CN, C₁-C₆-alkyl, etc. R⁴ is selected from the group consisting of hydrogen, halogen, CN, C₁-C₁₀-alkyl, etc. R⁵ is selected from the group consisting of hydrogen CN, C₁-C₁₀-alkyl, C₂-C₁₀-alkenyl C₃-C₈-cycloalkyl, S(O)ₙNR^{9a}R^{9b}, O—NR^{9a}R^{9b}, NR^{9a}R^{9b}, NR^{9a} NR^{9a}R^{9b}, NR^{9a}C(═O)R^{7a}, NR^{9a}C(═S)R^{7a}, NR^{9a}C(═O)NR^{9a}R^{9b}, NR^{9a}C(═S)NR^{9a}R^{9b}, C(═O)OR8, C(═O)NR^{9a}R^{9b}, C(═S)NR^{9a}R^{9b}, C(═O)R^{7a}, C(═S)R^{7a}, a moitey Q-phenyl, where the phenyl ring is optionally substituted with 1, 2, 3, 4 or 5 identical or different substituents R¹⁰, and a moiety Q-Het^{#} where Het^{#} represents a 3- to 10-membered saturated, partly saturated or unsaturated aromatic heterocyclic ring. The invention also relates to the use of the N-acrylimino heterocyclic compounds, their stereoisomers, their tautomers and their salts, for combating invertebrate pests. Furthermore the invention relates also to methods of combating invertebrate pests, which comprises applying such compounds.

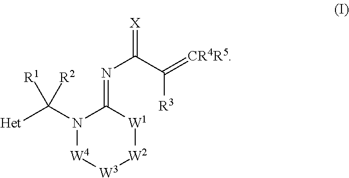

The present invention relates to N-acrylimino heterocyclic compounds, including their stereoisomers, tautomers and salts, and to compositions comprising such compounds. The invention also relates to the use of the N-acrylimino heterocyclic compounds, their stereoisomers, their tautomers and their salts, for combating invertebrate pests. Furthermore the invention relates also to methods of combating invertebrate pests, which comprises applying such compounds.

### BACKGROUND OF INVENTION

Invertebrate pests, such as insects, acaridae and nematode pests destroy growing and harvested crops and attack wooden dwelling and commercial structures, causing large economic loss to the food supply and to property. While a large number of pesticidal agents are known, due to the ability of target pests to develop resistance to said agents, there is an ongoing need for new agents for combating animal pests. In particular, animal pests such as insects and acaridae are difficult to be effectively controlled.

EP 259738 discloses compounds of the formula A, which have insecticidal activity:

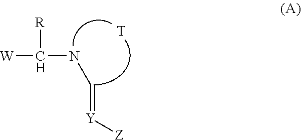

where W is a substituted pyridyl radical or a 5- or 6-membered heterocyclic radical, R is hydrogen or alkyl, T together with the atoms to which it is bound forms a 5- or 6-membered heterocyclic ring, Y is inter alia a nitrogen atom and Z is an electron withdrawing group selected from nitro and cyano.

Pesticidal compounds, which are similar to those of EP 259738, are known from EP 639569, where the electron withdrawing moiety Z is an electron withdrawing group such as alkoxcarbonyl, arylcarbonyl, heterocyclic carbonyl, C₁-C₄-alkylsulfonyl, sulfamoyl or C₁-C₄-acyl.

US 2013/0150414 describe, inter alia, pesticidal compounds of the formula B

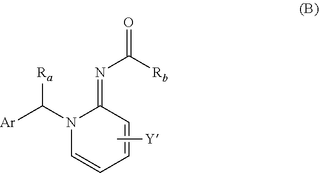

wherein Ar is an aryl or 5- or 6-membered heterocyclic group, Rₐ is hydrogen or alkyl, Y′ is hydrogen, halogen, a hydroxyl group, an alkyl group or an alkoxy group and Rb is an alkyl group substituted with halogen or an alkoxy group, optionally substituted with halogen.

Pesticidal compounds, which are similar to those of US 2013/0150414, are known from WO 2013/129688.

WO2013/129692 relates to compounds of formula C which have an insecticidal activity

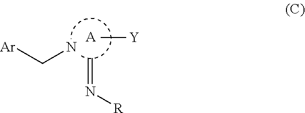

wherein Ar is an aryl, a 5- to 6-membered heterocycle, or a 4- to 10-membered heterocy-cloalkyl group, A represents a heterocycle having a 5- to 10-membered unsaturated bond including one or more nitrogen atoms, and has an imino group substituted with an R group at a position adjacent to the nitrogen atom present on the cycle, Y is a hydrogen, a halogen, a hydroxyl group, an alkyl group or an alkoxy group, and R is inter alia an imino or a carbonyl or a thiocarbonyl or a sulphur or a phosphorous bound group.

The pesticidal activity of the compounds is not satisfactory. It is therefore an object of the present invention to provide compounds having a good pesticidal activity, especially against difficult to control insects and acarid pests.

### SUMMARY OF INVENTION

It has been found that these objects are solved by N-substituted acryl-imino compounds of the general formula (I) described below, by their stereoisomers, their tautomers and their salts. Therefore, the present invention relates to N-acrylimino compound of formula (I):

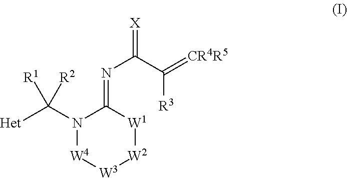

wherein X is O or S, in particular O;
Het is a 5- or 6-membered carbon-bound or nitrogen-bound heterocyclic ring, comprising 1, 2, 3, 4 or 5 carbon atoms and 1, 2 or 3 heteroatoms as ring members, which are independently selected from sulfur, oxygen or nitrogen, wherein the carbon, sulfur and nitrogen ring members can independently be partly or fully oxidized, and wherein each ring is optionally substituted by k identical or different substituents R⁶, wherein k is an integer selected from 0, 1, 2, 3 or 4;
W¹-W²-W³-W⁴ represents a carbon chain group connected to N and C═N, and thus forming a saturated, unsaturated, or partially unsaturated 5- or 6-membered nitrogen containing heterocycle, wherein W¹, W², W³ and W⁴ each individually represent CR^{v}R^{w}, wherein
   each R^{w} independently from each other, is selected from the group consisting hydrogen, halogen, cyano, azido, nitro, SCN, SF₅, C₁-C₁₀-alkyl, C₃-C₈-cycloalkyl, C₂-C₁₀-alkenyl, C₂-C₁₀-alkynyl, and wherein the carbon atoms of the aforementioned aliphatic and cycloaliphatic radicals may be unsubstituted or may be partly or fully halogenated and/or may optionally be substituted with 1, 2 or 3 identical or different radicals R⁷, OR⁸, NR^{9a}R^{9b}, S(O)ₙR⁸, S(O)ₙNR^{9a}R^{9b}, C(═O)R^{7a}, C(═O)NR^{9a}R^{9b}, C(═O)OR⁸, C(═S)R^{7a}, C(═S)NR^{9a}R^{9b}, C(═S)OR⁸, C(═S)SR⁸, C(═NR¹⁷)R^{7a}, C(═NR¹⁷)NR^{9a}R^{9b} and Si(R¹¹)₂R¹²,
   each R^{v} independently from each other, is selected from the group consisting of hydrogen, halogen, cyano, C₁-C₁₀-alkyl, C₃-C₈-cycloalkyl, C₂-C₁₀-alkenyl and C₂-C₁₀-alkynyl, wherein the carbon atoms of the aforementioned aliphatic and cycloaliphatic radicals may be unsubstituted or may be partly or fully halogenated or may optionally be further substituted with 1, 2 or 3 identical or different radicals R⁷; or
   R^{v} and R^{w}, which are present in one of the groups CR^{v}R^{w}, may together form ═O, ═CR¹³R¹⁴, ═S, ═NR¹⁷, ═NOR¹⁶, ═NNR^{9a}R^{9b},
      or
      two R^{v} of adjacent groups CR^{v}R^{w} may form both together and together with the existing bond a double bond between the adjacent carbon atoms;
   and wherein one of W² or W³ may optionally represent a single or a double bond between the adjacent carbon atoms;
R¹, R² are independently from each other selected from the group consisting of hydrogen, halogen, CN, SCN, nitro, C₁-C₆-alkyl, C₃-C₆-cycloalkyl, wherein each of the two aforementioned radicals are unsubstituted, partly or completely halogenated or may carry any combination of 1, 2 or 3 radicals R⁷,
   Si(R¹¹)₂R¹², OR⁸, OSO₂R⁸, S(O)ₙR⁸, S(O)ₙNR^{9a}R^{9b}, NR^{9a}R^{9b}, C(═O)NR^{9a}R^{9b}, C(═S)NR^{9a}R^{9b}, C(═O)OR⁸, C(═O)R^{7a}, C(═S)R^{7a},
   phenyl, benzyl, where the phenyl ring in the last two radicals is unsubstituted or optionally substituted with 1, 2, 3, 4 or 5 identical or different substituents R¹⁰,
   a 3-, 4-, 5-, 6- or 7-membered saturated, partly saturated or unsaturated aromatic heterocyclic ring comprising 1, 2 or 3 identical or different heteroatoms as ring members, which are selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized,
   or
   R¹ and R² form, together with the carbon atom, which they attached to, a 3-, 4-, 5- or 6-membered saturated or partly unsaturated carbocyclic or heterocyclic ring, wherein each of the carbon atoms of said cycle are unsubstituted or may carry any combination of 1 or 2 identical or different radicals R⁷,
   or
   R¹ and R² may together be ═O, ═CR¹³R¹⁴, ═S, ═NR¹⁷, ═NOR¹⁶ or ═NNR^{9a}R^{9b};
R³ is selected from the group consisting of hydrogen, halogen, CN, C₁-C₆-alkyl, C₃-C₈-cycloalkyl, C₃-C₈-cycloalkyl-C₁-C₄-alkyl, wherein each of the three last mentioned radicals are unsubstituted, partly or completely halogenated, or may carry any combination of 1, 2 or 3 radicals R⁷,
   OR⁸, S(O)ₙR⁸, S(O)ₙNR^{9a}R^{9b}, NR^{9a}R^{9b}, C(═O)OR⁸, C(═O)NR^{9a}R^{9b}, C(═S)NR^{9a}R^{9b}, C(═O)R^{7a}, C(═S)R^{7a},
   a moiety Q-phenyl, where the phenyl ring is optionally substituted with 1, 2, 3, 4 or 5 identical or different substituents R¹⁰,
   and a moiety Q-Het^{#} where Het^{#} represents a 3-, 4-, 5-, 6-, 7-, 8-, 9- or 10-membered saturated, partly saturated or unsaturated aromatic heterocyclic ring comprising 1, 2, 3 or 4 heteroatoms as ring members, which are identical or different and selected from oxygen, nitrogen and sulfur, where the heterocyclic ring is optionally substituted with 1, 2, 3, 4, 5 or 6 identical or different substituents R¹⁰, and wherein the nitrogen and/or the sulfur atom(s) of the heterocyclic ring may optionally be oxidized,
R⁴ is selected from the group consisting of hydrogen, halogen, CN, C₁-C₁₀-alkyl, C₂-C₁₀-alkenyl, C₃-C₈-cycloalkyl, wherein each of the three last mentioned radicals are unsubstituted, partly or completely halogenated, or may carry any combination of 1, 2 or 3 radicals R⁷,
   S(O)ₙNR^{9a}R^{9b}, NR^{9a}R^{9b}, C(═O)OR⁸, C(═O)NR^{9a}R^{9b}, C(═S)NR^{9a}R^{9b}, C(═O)R^{7a}, C(═S)R^{7a},
   a moiety Q-phenyl, where the phenyl ring is optionally substituted with 1, 2, 3, 4 or 5 identical or different substituents R¹⁰,
   and a moiety Q-Het^{#} where Het^{#} represents a 3-, 4-, 5-, 6-, 7-, 8-, 9- or 10-membered saturated, partly saturated or unsaturated aromatic heterocyclic ring comprising 1, 2, 3 or 4 heteroatoms as ring members, which are identical or different and selected from oxygen, nitrogen and sulfur, where the heterocyclic ring is optionally substituted with 1, 2, 3, 4, 5 or 6 identical or different substituents R¹⁰, and wherein the nitrogen and/or the sulfur atom(s) of the heterocyclic ring may optionally be oxidized;
R⁵ is selected from the group consisting of hydrogen CN, C₁-C₁₀-alkyl, C₂-C₁₀-alkenyl, C₃-C₈-cycloalkyl, wherein each of the three last mentioned radicals are unsubstituted, partly or completely halogenated, or may carry any combination of 1, 2 or 3 radicals R⁷,
   S(O)ₙNR^{9a}R^{9b}, O—NR^{9a}R^{9b}, NR^{9a}R^{9b}, NR^{9a}NR^{9a}R^{9b}, NR^{9a}C(═O)R^{7a}, NR^{9a}C(═S)R^{7a}, NR^{9a}C(═O)NR^{9a}R^{9b}, NR^{9a}C(═S)NR^{9a}R^{9b}, C(═O)OR⁸, C(═O)NR^{9a}R^{9b}, C(═S)NR^{9a}R^{9b}, C(═O)R^{7a}, C(═S)R^{7a},
   a moiety Q-phenyl, where the phenyl ring is optionally substituted with 1, 2, 3, 4 or 5 identical or different substituents R¹⁰,
   and a moiety Q-Het^{#} where Het^{#} represents a 3-, 4-, 5-, 6-, 7-, 8-, 9- or 10-membered saturated, partly saturated or unsaturated aromatic heterocyclic ring comprising 1, 2, 3 or 4 heteroatoms as ring members, which are identical or different and selected from oxygen, nitrogen and sulfur, where the heterocyclic ring is optionally substituted with 1, 2, 3, 4, 5 or 6 identical or different substituents R¹⁰, and wherein the nitrogen and/or the sulfur atom(s) of the heterocyclic ring may optionally be oxidized, or
R⁴ and R⁵ together form a moiety selected from Alk, S(O)ₙ-Alk, NR^{9c}-Alk, S(O)ₙ-Alk′-S(O)ₙ, O-Alk′-O, O-Alk′-NR^{9d}, S(O)ₙ-Alk′NR^{9d}, O-Alk′-S and NR^{9c}-Alk′-NR^{9d}, where
   Alk represents a saturated or unsaturated 2-, 3-, 4- or 5-membered carbon chain group, which is unsubstituted or carries 1, 2, 3 or 4 radicals R⁷ or a fused phenylene ring, where the fused phenyl ring is unsubstituted or substituted with 1, 2, 3 or 4 radicals R¹⁰;
   Alk′ is CH₂, where one or both hydrogen atoms may optionally be replaced by R⁷ or represents a saturated or unsaturated 2-, 3- or 4-membered carbon chain group, which is unsubstituted or carries 1, 2, 3 or 4 radicals R⁷ or a fused phenylene ring, where the fused phenyl ring is unsubstituted or substituted with 1, 2, 3 or 4 radicals R¹⁰;
      provided that R⁵ is different from hydrogen and C₁-C₆-alkyl, if R³ is hydrogen;
      where, independently of their occurrence,
n is 0, 1 or 2;
Q is a single bond, C₁-C₄-alkylene, a moiety NR^{9a}, or NR^{9a}—C₁-C₄-alkylene, where the heteroatom in the last 2 moieties is bound to the carbon atom of CR⁴R⁵;
R⁶ is selected from the group consisting of halogen, cyano, azido, nitro, SCN, SF₅, C₁-C₁₀-alkyl, C₃-C₈-cycloalkyl, C₂-C₁₀-alkenyl, C₂-C₁₀-alkynyl, and wherein the carbon atoms of the aforementioned aliphatic and cyclo-aliphatic radicals may optionally be further substituted independently from one another with 1, 2 or 3 radicals R⁷, OR⁸, NR^{17a}R^{17b}, S(O)ₙR⁸, S(O)ₙNR^{17a}R^{17b}, C(═O)R^{7a}, C(═O)NR^{17a}R^{17b}, C(═O)OR⁸, C(═S)R^{7a}, C(═S)NR^{17a}R^{17b}, C(═S)OR⁸, C(═S)SR⁸, C(═NR¹⁷)R^{7a}, C(═NR¹⁷)NR^{17a}R^{17b}, Si(R¹¹)₂R¹²;
   phenyl, optionally substituted with 1, 2, 3, 4 or 5 identical or different substituents R¹⁰,
   a 3-, 4-, 5-, 6- or 7-membered saturated, partly saturated or unsaturated aromatic heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized,
   or two of R⁶ present on one ring carbon may together form ═O, ═CR¹³R¹⁴, ═S, ═NR¹⁷, ═NOR¹⁶, ═NNR^{9a}R^{9b},
   or two R⁶, which are bound to adjacent ring atoms, together may form a linear C₂-C₇ alkylene chain, thus forming, together with the ring atoms to which they are bound, a 4-, 5-, 6-, 7-, 8- or 9-membered ring, where 1 or 2 CH₂ moieties of the alkylene chain may be replaced by 1 or 2 heteroatom moieties selected from O, S and NR^{17c} and/or 1 or 2 of the CH₂ groups of the alkylene chain may be replaced by a group C═O, C═S and/or C═NR¹⁷; and where the alkylene chain is unsubstituted or may be substituted with 1, 2, 3, 4, 5 or 6 radicals selected from the group consisting of halogen, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted with 1, 2, 3, 4 or 5 radicals R¹⁰, and a 3-, 4-, 5-, 6- or 7-membered saturated, partially unsaturated or aro-uratic heterocyclic ring containing 1, 2 or 3 heteroatoms or heteroatom groups selected from N, O, S, NO, SO and SO₂, as ring members, where the heterocyclic ring may be substituted with 1, 2, 3, 4 or 5 radicals R¹⁰;
R⁷ independently of its occurrence, is selected from the group consisting of cyano, azido, nitro, —SCN, SF₅, C₁-C₆-alkyl, C₁-C₆-haloalkyl, C₃-C₈-cycloalkyl, where the C₃-C₈-cycloalkyl ring in the last group is optionally substituted with 1, 2, 3 or 4 C₁-C₄-radicals, C₃-C₈-halocycloalkyl, C₂-C₆-alkenyl, C₂-C₆-haloalkenyl, C₂-C₆-alkynyl, C₂-C₆-haloalkynyl, Si(R¹¹)₂R¹², OR⁸, OSO₂R⁸, S(O)ₙR⁸, S(O)ₙNR^{17a}R^{17b}, NR^{17a}R^{17b}, C(═O)NR^{17a}R^{17b}, C(═S)NR^{17a}R^{17b}, C(═O)OR⁸, C(═O)R¹⁵, C(═S)R¹⁵, C(═NR¹⁷)R¹⁵, phenyl, phenyl-C₁-C₄-alkyl, where the phenyl ring in the last two groups is optionally substituted with 1, 2, 3, 4 or 5 identical or different substituents R¹⁰, and a 3-, 4-, 5-, 6- or 7-membered saturated, partly saturated or unsaturated aromatic heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized,
   or two R⁷ present on one carbon atom may together form ═O, ═CR¹³R¹⁴, ═S, ═NR¹⁷, ═NOR¹⁶, ═NNR^{9a}R^{9b},
   or two R⁷ may form a 3-, 4-, 5-, 6-, 7- or 8-membered saturated or partly unsaturated carbocyclic or heterocyclic ring together with the carbon atoms to which the two R⁷ are bonded, where the heterocyclic ring comprises 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰;
R^{7a} independently of its occurrence, is selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆ haloalkynyl,
   phenyl, optionally substituted with 1, 2, 3, 4 or 5 identical or different substituents R¹⁰,
   benzyl, where the phenyl ring in the last radical is unsubstituted or substituted by 1, 2, or 3 identical or different substituents selected from the group consisting of halogen, CN, C₁-C₄-alkyl, C₁-C₄-haloalkyl, C₁-C₄-alkoxy and C₁-C₄-haloalkoxy, and a 3-, 4-, 5-, 6- or 7-membered saturated, partly saturated or unsaturated aromatic heterocyclic ring comprising 1, 2 or 3 heteroatoms as ring members, which are identical or different and selected from oxygen, nitrogen and sulfur, where the heterocy-R¹⁰, and wherein the nitrogen and/or the sulfur atom(s) of the heterocyclic ring may optionally be oxidized;
R⁸ independently of its occurrence, is selected from the group consisting of hydrogen, C₁-C₆-alkyl, C₁-C₄-alkoxy-C₁-C₄-alkyl, C₁-C₆-haloalkyl, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl, C(═O)R¹⁵, C(═O)NR^{17a}R^{17b}, C(═S)NR^{17a}R^{17b}, C(═O)OR¹⁶, phenyl, phenyl-C₁-C-₄-alkyl, where the phenyl ring in the last two mentioned radicals is unsubstituted or substituted with 1, 2, 3, 4 or 5 identical or different substituents R¹⁰, and
   a 3-, 4-, 5-, 6- or 7-membered saturated, partly saturated or unsaturated aromatic heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized,
R⁹ independently of its occurrence, is selected from the group consisting of hydrogen, C₁-C₆-alkyl, C₁-C₆-haloalkyl, C₁-C₆-alkoxy, C₁-C₆-haloalkoxy, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl,
   phenyl, optionally substituted with 1, 2, 3, 4 or 5 identical or different substituents R¹⁰;
   benzyl, where the phenyl ring in the last radical is unsubstituted or substituted by 1, 2, or 3 identical or different substituents selected from the group consisting of halogen, CN, C₁-C₄-alkyl, C₁-C₄-haloalkyl, C₁-C₄-alkoxy and C₁-C₄-haloalkoxy,
   a 3-, 4-, 5-, 6- or 7-membered saturated, partly saturated or unsaturated aromatic C-bound heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized,
R^{9a}, R^{9b} are each independently from one another selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₃-C₈-cycloalkyl-C₁-C₄-alkyl, C₂-C₆-alkenyl, C₂-C₆-haloalkenyl, C₂-C₆-alkynyl, C₂-C₆ haloalkynyl,
   S(O)ₙR¹⁶, —S(O)ₙNR^{17a}R^{17b}, C(═O)R¹⁵, C(═O)OR¹⁶, C(═O)NR^{17a}R^{17b}, C(═S)R¹⁵,
   C(═S)SR¹⁶, C(═S)NR^{17a}R^{17b}, C(═NR¹⁷)R¹⁵;
   phenyl, benzyl, 1-phenethyl or 2-phenethyl, where the phenyl ring in the last four mentioned radicals is unsubstituted or may be substituted with 1, 2, 3, 4 or 5 identical or different substituents R¹⁰; and
   a 3-, 4-, 5-, 6- or 7-membered saturated, partly saturated or unsaturated aromatic C-bound heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized, or,
R^{9a} and R^{9b} are together a C₂-C₇, alkylene chain and form a 3-, 4-, 5-, 6-, 7- or 8-membered saturated, partly saturated or unsaturated aromatic ring together with the nitrogen atom they are bonded to, wherein the alkylene chain may contain one or two heteroatoms, which are, independently of each other, selected from oxygen, sulfur and nitrogen, and where the alkylene chain may optionally be substituted with 1, 2, 3 or 4 radicals selected from halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl, optionally substituted with 1, 2, 3, 4 or 5 identical or different substituents R¹⁰, and a 3-, 4-, 5-, 6- or 7-membered saturated, partly saturated or unsaturated aromatic C-bound heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized, or
R^{9a} and R^{9b} together may form ═CR¹³R¹⁴, ═NR¹⁷, ═NOR¹⁶, ═NNR^{17a}R^{17b} moiety;
R^{9c}, R^{9d} are each independently from one another selected from the group consisting of hydrogen, C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl,
   S(O)ₙR¹⁶, —S(O)ₙNR^{17a}R^{17b}, C(═O)R¹⁵, C(═O)OR¹⁶, C(═O)NR^{17a}R^{17b}, C(═S)R¹⁵, C(═S)SR¹⁶, C(═S)NR^{17a}R^{17b}, C(═NR¹⁷)R¹⁵;
   phenyl, benzyl, where the phenyl ring in the last two mentioned radicals is unsubstituted or may be substituted with 1, 2, 3, 4 or 5 identical or different substituents R¹⁰; and
   a 3-, 4-, 5-, 6- or 7-membered saturated, partly saturated or unsaturated aromatic C-bound heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized;
R¹⁰ independently of its occurrence, is selected from the group consisting of halogen, cyano, azido, nitro, SCN, SF₅, C₁-C₁₀-alkyl, C₃-C₈-cycloalkyl, C₂-C₁₀-alkenyl, C₂-C₁₀-alkynyl, wherein the carbon atoms of the aforementioned aliphatic and cycloaliphatic radicals may optionally be substituted with 1, 2, 3, 4 or 5 identical or different radi-cats R¹⁰a,
   Si(R¹¹)₂R¹², OR¹⁶, OS(O)ₙR¹⁶, —S(O)ₙR¹⁶, S(O)ₙNR^{17a}R^{17b}, NR^{17a}R^{17b}, C(═O)R¹⁵, C(═S)R¹⁵, C(═O)OR¹⁶, —C(═NR¹⁷)R¹⁵, C(═O)NR^{17a}R^{17b}, C(═S)NR^{17a}R^{17b},
   phenyl, optionally substituted with 1, 2, 3, 4 or 5 identical or different radicals selected from OH, halogen, cyano, nitro, C₁-C₆-alkyl, C₁-C₆-haloalkyl, C₁-C₆-alkoxy and C₁-C₆-haloalkoxy, and
   a 3-, 4-, 5-, 6- or 7-membered saturated, partly saturated or unsaturated aromatic heterocyclic ring comprising 1, 2 or 3 heteroatoms as ring members, which are identical or different and selected from oxygen, nitrogen and sulfur, where the heterocyclic ring is unsubstituted or may be substituted with 1, 2, 3, 4 or 5 substituents selected independently from one another from halogen, cyano, NO₂, C₁-C₆-alkyl, C₁-C₆-haloalkyl, C₁-C₆-alkoxy and C₁-C₆-haloalkoxy, and wherein the nitrogen and/or the sulfur atom(s) of the heterocyclic ring may optionally be oxidized;
   or
   two R¹⁰ present together on one carbon ring atom of a saturated or partly unsaturated heterocyclic radical may form ═O, ═CR¹³R¹⁴, ═S, ═NR¹⁷, ═NOR¹⁶, ═NNR^{17a}R^{17b};
   or,
   two R¹⁰ on adjacent carbon ring atoms may also be a bivalent radical selected from CH₂CH₂CH₂CH₂, CH═CH—CH═CH, N═CH—CH═CH, CH═N—CH═CH, N═CH—N═CH, OCH₂CH₂CH₂, OCH═CHCH₂, CH₂OCH₂CH₂, OCH₂CH₂O, OCH₂OCH₂, CH₂CH₂CH₂, CH═CHCH₂, CH₂CH₂O, CH═CHO, CH₂OCH₂, CH₂C(═O)O, C(═O)OCH₂, O(CH₂)O, SCH₂CH₂CH₂, SCH═CHCH₂, CH₂SCH₂CH₂, SCH₂CH₂S, SCH₂SCH₂, CH₂CH₂S, CH═CHS, CH₂SCH₂, CH₂C(═S)S, C(═S)SCH₂, S(CH₂)S, CH₂CH₂NR¹⁷, CH₂CH═N, CH═CH—NR¹⁷, OCH═N, SCH═N and form together with the carbon atoms to which the two R¹⁰ are bonded to a 5-membered or 6-membered partly saturated or unsaturated, aromatic carbocyclic or heterocyclic ring, wherein the ring may optionally be substituted with one or two substituents selected from ═O, OH, CH₃, OCH₃, halogen, cyano, halomethyl and halomethoxy;
R^{10a} independently of its occurrence, is halogen or has one of the meanings given for R⁷;
R¹¹, R¹² independently of their occurrence, are selected from the group consisting of C₁-C₆-alkyl, C₁-C₆-haloalkyl, C₁-C₆-alkoxy, C₁-C₆-alkoxy-C₁-C₄-alkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₃-C₈-cycloalkyl-C₁-C₄-alkyl, C₃-C₈-halocycloalkyl-C₁-C₄-alkyl, C₁-C₆-haloalkoxy-C₁-C₄-alkyl, phenyl and benzyl, where the phenyl ring in last two radicals are unsubstituted or substituted with 1, 2, 3, 4 or 5 identical or different radicals selected from halogen, OH, cyano, NO₂, C₁-C₆-alkyl, C₁-C₆-haloalkyl, C₁-C₆-alkoxy and C₁-C₆-haloalkoxy;
R¹³, R¹⁴ independently of their occurrence, are selected from the group consisting of hydrogen, halogen, CN, C₁-C₆-alkyl, C₃-C₆-cycloalkyl, C₁-C₄-alkoxy-C₁-C₄-alkyl, phenyl and benzyl;
R¹⁵ independently of its occurrence, is selected from the group consisting of hydrogen, C₁-C₆-alkyl, C₂-C₆-alkenyl, C₂-C₆-alkynyl, C₃-C₈-cycloalkyl, wherein the four last mentioned aliphatic and cycloaliphatic radicals may be unsubstituted, partially or fully halogenated and/or oxygenated and/or may carry 1 or 2 radicals selected from C₁-C₄ alkoxy; C₃-C₈-cycloalkyl-C₁-C₄-alkyl;
   phenyl, benzyl and pyridyl, wherein the last three radicals may be unsubstituted, partially or fully halogenated and/or may carry 1, 2 or 3 substituents selected from C₁-C₆-alkyl, C₁-C₆-haloalkyl, C₁-C₆-alkoxy, C₁-C₆-haloalkoxy, (C₁-C₆-alkoxy)carbonyl, (C₁-C₆-alkyl)amino and di-(C₁-C₆-alkyl)amino;
R₁₆ independently of its occurrence, is selected from the group consisting of hydrogen, C₁-C₆-alkyl, C₂-C₆-alkenyl, C₂-C₆-alkynyl, C₃-C₈-cycloalkyl, C₃-C₈-cycloalkyl-C₁-C₄-alkyl, wherein the five last mentioned aliphatic and cycloaliphatic radicals may be unsubstituted, partially or fully halogenated and/or oxygenated and/or may carry 1 or 2 radicals selected from C₁-C₄-alkoxy,
   phenyl, benzyl and pyridyl, wherein the last three radicals may be unsubstituted, partially or fully halogenated and/or may carry 1, 2 or 3 substituents selected from CN, C₁-C₆-alkyl, C₁-C₆-haloalkyl, C₁-C₆-alkoxy, C₁-C₆-haloalkoxy, (C₁-C₆-alkoxy)carbonyl, (C₁-C₆-alkyl)amino or di-(C₁-C₆-alkyl)amino;
R¹⁷ independently of its occurrence, is selected from the group consisting of hydrogen, C₁-C₆-alkoxy, C₁-C₆-haloalkoxy, C₁-C₆-alkylthio, trimethylsilyl, triethylsilyl, tertbutyl-dimethylsilyl,
   C₁-C₆-alkyl, C₂-C₆-alkenyl, C₂-C₆-alkynyl, C₃-C₈-cycloalkyl, wherein the four last mentioned aliphatic and cycloaliphatic radicals may be unsubstituted, partially or fully halogenated and/or oxygenated and/or may carry 1 or 2 radicals selected from C₁-C₄-alkoxy,
   C₃-C₈-cycloalkyl-C₁-C₄-alkyl;
   phenyl, benzyl, pyridyl, phenoxy, wherein the four last mentioned radicals may be unsubstituted, partially or fully halogenated and/or carry 1, 2 or 3 substituents selected from CN, C₁-C₆-alkyl, C₁-C₆-haloalkyl, C₁-C₆-alkoxy, C₁-C₆-haloalkoxy and (C₁-C₆-alkoxy)carbonyl,
R^{17a}, R^{17b} are each independently from one another selected from the group consisting of hydrogen, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl, C₁-C₆-alkyl, C₂-C₆-alkenyl, C₂-C₆-alkynyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄-alkoxy,
   phenyl, benzyl, pyridyl, phenoxy, wherein the four last mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy and (C₁-C₆-alkoxy)carbonyl,
   or,
   R^{17a} and R^{17b} may together be a C₂-C₆ alkylene chain forming a 3- to 7-membered saturated, partly saturated or unsaturated ring together with the nitrogen atom R^{17a} and R^{17b} are bonded to, wherein the alkylene chain may contain 1 or 2 heteroatoms selected, independently of each other, from oxygen, sulfur and nitrogen, and may optionally be substituted with halogen, C₁-C₄-haloalkyl, C₁-C₄-alkoxy or C₁-C₄-haloalkoxy, and wherein the nitrogen and/or the sulfur atom(s) of the heterocyclic ring may optionally be oxidized;
   or
R^{17a} and R^{17b} together may form ═CR¹³R¹⁴, ═NR¹⁷ or ═NOR¹⁶ moiety;
R^{17c} independently of its occurrence, is selected from the group consisting of hydrogen, CN, C₁-C₆-alkyl, C₂-C₆-alkenyl, C₂-C₆-alkynyl, C₃-C₈-cycloalkyl, C₃-C₈-cycloalkyl-C₁-C₄-alkyl, wherein the five last mentioned aliphatic and cycloaliphatic radicals may be unsubstituted, partially or fully halogenated and/or oxygenated and/or may carry 1 or 2 radicals selected from C₁-C₄-alkoxy,
   phenyl, benzyl and pyridyl, wherein the last three radicals may be unsubstituted, partially or fully halogenated and/or may carry 1, 2 or 3 substituents selected from CN, C₁-C₆-alkyl, C₁-C₆-haloalkyl, C₁-C₆-alkoxy, C₁-C₆-haloalkoxy, (C₁-C₆-alkoxy)carbonyl, (C₁-C₆-alkyl)amino or di-(C₁-C₆-alkyl)amino;
      the stereoisomers, tautomers and the salts thereof.

Moreover, the present invention relates to and includes the following embodiments:
agricultural and veterinary compositions comprising an amount of at least one compound of the formula (I) or a stereoisomer, tautomer or salt thereof;
the use of the compounds of formula (I), the stereoisomers, the tautomers or the salts thereof for combating invertebrate pests;
the use of the compounds of formula (I), the stereoisomers, the tautomers or the salts thereof for protecting growing plants from attack or infestation by invertebrate pests;
the use of the compounds of formula (I), the stereoisomers, the tautomers or the salts, thereof for protecting plant propagation material, especially seeds, from soil insects;
the use of the compounds of formula (I), the stereoisomers, the tautomers or the salts thereof for protecting the seedlings roots and shoots of plants from soil and foliar insects;
a method for combating or controlling invertebrate pests, which method comprises contacting said pest or its food supply, habitat or breeding grounds with a pesticidally effective amount of at least one compound of the formula (I) or a stereoisomer, a tautomer or salt thereof;
a method for protecting growing plants from attack or infestation by invertebrate pests, which method comprises contacting a plant, or soil or water in which the plant is growing, with a pesticidally effective amount of at least one compound of the formula (I) or a stereoisomer, a tautomer or salt thereof, in particular a method protecting crop plants from attack or infestation by animal pests, which comprises contacting the crop plants with a pesticidally effective amount of at least one compound of the formula (I) or stereoisomer, a tautomer or salt thereof;
a method for the protection of plant propagation, especially seeds, from soil insects and of the seedlings' roots and shoots from soil and foliar insects comprising contacting the seeds before sowing and/or after pregermination with at least one compound of the formula (I) or stereoisomer, a tautomer or salt thereof;
seeds comprising a compound of the formula (I) or an enantiomer, diastereomer or salt thereof;
the use of compounds of formula (I), the stereoisomers, the tautomers or the salts, in particular the veterinary acceptable salts, thereof for combating parasites in and on animals, in particular for the use in the treatment of animals infested or infected by parasites, for preventing animals of getting infected or infested by parasites or for protecting animals against infestation or infection by parasites;
a method for treating animals infested or infected by parasites or preventing animals of getting infected or infested by parasites or protecting animals against infestation or infection by parasites which comprises administering or applying to the animals a parasiticidally effective amount of a compound of formula (I) or the stereoisomers and/or salts, in particular veterinary acceptable salts, thereof;
a process for the preparation of a veterinary composition for treating, controlling, preventing or protecting animals against infestation or infection by parasites which comprises formulating a compound of formula (I) or a stereoisomer, tautomer and/or veterinary acceptable salt thereof with a carrier composition suitable for veterinary use;
the use of a compound of formula (I) or the stereoisomers, tautomers and/or veterinary acceptable salt thereof for the preparation of a medicament for treating, controlling, preventing or protecting animals against infestation or infection by parasites.

The present invention also relates to plant propagation materials, in particular as mentioned above to seeds, containing at least one compound of formula (I), a stereoisomer, a tautomer and/or an agriculturally acceptable salt thereof.

### DETAILED DESCRIPTION OF INVENTION

The present invention relates to every possible stereoisomer of the compounds of formula (I), i.e. to single enantiomers, diastereomers and E/Z-isomers as well as to mixtures thereof and also to the salts thereof. The present invention relates to each isomer alone, or mixtures or combinations of the isomers in any proportion to each other. For example, In formula (I), the moieties R³ and R⁵ may be located cis or trans with respect to the double bond between CR³ and CR⁴R⁵. Depending on the substitution pattern, the compounds of the formula (I) may have one or more centers of chirality, in which case they are present as mixtures of enantiomers or diastereomers. One center of chirality is the carbon ring atom carrying radical R¹. The invention provides both the pure enantiomers or diastereomers and their mixtures and the use according to the invention of the pure enantiomers or diastereomers of the compound I or its mixtures. Suitable compounds of the formula (I) also include all possible geometrical stereoisomers (cis/trans isomers) and mixtures thereof.

The present invention also relates to potential tautomers of the compounds of formula (I) and also to the salts of such tautomers. The present invention relates to the tautomer as such as well as to mixtures or combinations of the tautomers in any proportion to each other. The term “tautomers” encompasses isomers, which are derived from the compounds of formula (I) by the shift of an H-atom involving at least one H-atom located at a nitrogen, oxygen or sulphur atom. Examples of tautomeric forms are keto-enol forms, imine-enamine forms, urea-isourea forms, thiourea-isothiourea forms, (thio)amide-(thio)imidate forms etc.

The compounds of the present invention, i.e. the compounds of formula (I), their stereoisomers, their tautomers as well as their salts, in particular their agriculturally acceptable salts and their veterinarily acceptable salts, may be amorphous or may exist in one ore more different crystalline states (polymorphs) or modifications which may have a different macroscopic properties such as stability or show different biological properties such as activities. The present invention includes both amorphous and crystalline compounds of the formula (I), mixtures of different crystalline states or modifications of the respective stereoisomers or tautomers, as well as amorphous or crystalline salts thereof.

Salts of the compounds of the formula (I) are preferably agriculturally salts as well as veterinarily acceptable salts. They can be formed in a customary method, e.g. by reacting the compound with an acid of the anion in question if the compound of formula (I) has a basic functionality or by reacting an acidic compound of formula (I) with a suitable base.

Suitable agriculturally or veterinary useful salts are especially the salts of those cations or anions, in particular the acid addition salts of those acids, whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄+) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammo-nium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethyl-ammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting the compounds of the formulae I with an acid of the corresponding anion, preferably of hydrochloric acid, hydrobromic acid, sulfuric acid, phosphoric acid or nitric acid.

The organic moieties mentioned in the above definitions of the variables are—like the term halogen—collective terms for individual listings of the individual group members. The prefix Cₙ-Cₘ indicates in each case the possible number of carbon atoms in the group.

“Halogen” will be taken to mean fluoro, chloro, bromo and iodo. The term “partially or fully halogenated” will be taken to mean that 1 or more, e.g. 1, 2, 3, 4 or 5 or all of the hydrogen atoms of a given radical have been replaced by a halogen atom, in particular by fluorine or chlorine. For example, partially or fully halogenated alkyl is also termed haloalkyl, partially or fully halogenated cycloalkyl is also termed halocycloalkyl, partially or fully halogenated alkylenyl is also termed haloalkenyl, partially or fully halogenated alkylynyl is also termed haloalkynyl, partially or fully halogenated alkoxy is also termed haloalkoxy, partially or fully halogenated alkylthio is also termed haloalkthio, partially or fully halogenated alkylsulfinyl is also termed haloalkylsulfinyl, partially or fully halogenated alkylsulfonyl is also termed haloalsulfonyl, partially or fully halogenated cycloalkylalkyl is also termed halocycloalkylalkyl.

The term “Cₙ-Cₘ-alkyl” as used herein, and also in Cₙ-Cₘ-alkylamino, di-Cₙ-Cₘ-alkylamino, Cₙ-Cₘ-alkylaminocarbonyl, di-(Cₙ-Cₘ-alkylamino)carbonyl, Cₙ-Cₘ-alkylthio, Cₙ-Cₘ-alkylsulfinyl and Cₙ-Cₘ-alkylsulfonyl, refers to a branched or unbranched saturated hydrocarbon group having n to m, e.g. 1 to 10 carbon atoms (C₁-C₁₀-alkyl), preferably 1 to 6 carbon atoms (C₁-C₆-alkyl), for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, nonyl and decyl and their isomers. C₁-C₄-alkyl means for example methyl, ethyl, propyl, 1-methylethyl, butyl, 1-methylpropyl, 2-methylpropyl or 1,1-dimethylethyl.

The term “Cₙ-Cₘ-haloalkyl” as used herein, and also in Cₙ-Cₘ-haloalkylthio (═C₁-Cₘ-haloalkylsulfenyl), Cₙ-Cₘ-haloalkylsulfinyl and Cₙ-Cₘ-haloalkylsulfonyl, refers to a straight-chain or branched alkyl group having n to m carbon atoms, e.g. 1 to 10 in particular 1 to 6 carbon atoms (as mentioned above), where some or all of the hydrogen atoms in these groups may be replaced by halogen atoms as mentioned above, for example C₁-C₄-haloalkyl, such as chloro-methyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like. The term C₁-C₁₀-haloalkyl in particular comprises C₁-C₂-fluoroalkyl, which is synonym with methyl or ethyl, wherein 1, 2, 3, 4 or 5 hydrogen atoms are substituted by fluorine atoms, such as fluoromethyl, difluoromethyl, trifluoromethyl, 1-fluoroethyl, 2-fluoroethyl, 2,2-difluoroethyl, 2,2,2-trifluoroethyl and pentafluoromethyl. “Halomethyl” is methyl in which 1, 2 or 3 of the hydrogen atoms are replaced by halogen atoms. Examples are bromomethyl, chloromethyl, fluoromethyl, dichloromethyl, trichloromethyl, difluoromethyl, trifluoromethyl, chlorofluoromethyl, dichlorofluoromethyl, chlorodifluoromethyl and the like.

Similarly, “Cₙ-Cₘ-alkoxy”, “Cₙ-Cₘ-alkylthio”, or “Cₙ-Cₘ-alkylsulfenyl”, respectively, “Cₙ-Cₘ-alkylsulfinyl” or “Cₙ-Cₘ-alkylsulfonyl” refer to straight-chain or branched alkyl groups having n to m carbon atoms, e.g. 1 to 10, in particular 1 to 6 or 1 to 4 carbon atoms (as mentioned above) bonded through O, S, S(═O) or S(═O)₂ linkages, respectively, at any bond in the alkyl group. Accordingly, the terms “Cₙ-Cₘ-haloalkoxy”, “Cₙ-Cₘ-haloalkylthio” or “Cₙ-Cₘ-alkylsulfenyl”, respectively, “Cₙ-Cₘ-haloalkylsulfinyl” or “Cₙ-Cₘ-haloalkylsulfonyl”, refer to straight-chain or branched alkyl groups having n to m carbon atoms, e.g. 1 to 10, in particular 1 to 6 or 1 to 4 carbon atoms (as mentioned above) bonded through O, S, S(═O) or S(═O)₂ linkages, respectively, at any bond in the haloalkyl group, where some or all of the hydrogen atoms in these groups may be replaced by halogen atoms as mentioned above.

The term “C₁-Cₘ-alkoxy” is a C₁-Cₘ-alkyl group, as defined above, attached via an oxygen atom. C₁-C₂-Alkoxy is methoxy or ethoxy. C₁-C₄-Alkoxy is, for example, methoxy, ethoxy, n-propoxy, 1-methylethoxy (isopropoxy), butoxy, 1-methylpropoxy (sec-butoxy), 2-methylpropoxy (isobutoxy) or 1,1-dimethylethoxy (tert-butoxy). C₁-C₆-Alkoxy includes the meanings given for C₁-C₄-alkoxy and also includes, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₈-Alkoxy includes the meanings given for C₁-C₆-alkoxy and also includes, for example, heptyloxy, octyloxy, 2-ethylhexyloxy and positional isomers thereof. C₁-C₁₀-Alkoxy includes the meanings given for C₁-C₈-alkoxy and also includes, for example, nonyloxy, decyloxy and positional isomers thereof.

The term “C₁-Cₘ-alkylthio” is a C₁-Cₘ-alkyl group, as defined above, attached via a sulfur atom. C₁-C₂-Alkylthio is methylthio or ethylthio. C₁-C₄-Alkylthio is, for example, methylthio, ethylthio, n-propylthio, 1-methylethylthio (isopropylthio), butylthio, 1-methylpropylthio (sec-butylthio), 2-methylpropylthio (isobutylthio) or 1,1-dimethylethylthio (tert-butylthio). C₁-C₆-Alkylthio includes the meanings given for C₁-C₄-alkylthio and also includes, for example, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio or 1-ethyl-2-methylpropylthio. C₁-C₈-Alkylthio includes the meanings given for C₁-C₆-alkylthio and also includes, for example, heptylthio, octylthio, 2-ethylhexylthio and positional isomers thereof. C₁-C₁₀-Alkylthio includes the meanings given for C₁-C₈-alkylthio and also includes, for example, nonylthio, decylthio and positional isomers thereof.

The term “C₁-Cₘ-alkylsulfinyl” is a C₁-Cₘ-alkyl group, as defined above, attached via a S(═O) group. The term “C₁-Cₘ-alkylsulfonyl” is a C₁-Cₘ-alkyl group, as defined above, attached via a S(═O)₂ group.

The term “C₁-Cₘ-haloalkyloxy” is a C₁-Cₘ-haloalkyl group, as defined above, attached via an oxygen atom. Examples include C₁-C₂-haloalkoxy, such as chloromethoxy, bromomethoxy, dichloromethoxy, trichloromethoxy, fluoromethoxy, difluoromethoxy, trifluoromethoxy, chlorofluo-romethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy and pentafluoro-ethoxy.

The term “C₁-Cₘ-haloalkylthio” is a C₁-Cₘ-haloalkyl group, as defined above, attached via a sulfur atom. Examples include C₁-C₂-haloalkylthio, such as chloromethylthio, bromomethyl-thio, dichloromethylthio, trichloromethylthio, fluoromethylthio, difluoromethylthio, trifluoromethyl-thio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio and pentafluoroethylthio and the like.

Similarly the terms C₁-C₂-fluoroalkoxy and C₁-C₂-fluoroalkylthio refer to C₁-C₂-fluoroalkyl which is bound to the remainder of the molecule via an oxygen atom or a sulfur atom, respectively.

The term “C₁-Cₘ-haloalkylsulfinyl” is a C₁-Cₘ-haloalkyl group, as defined above, attached via a S(═O) group. The term “C₁-Cₘ-haloalkylsulfonyl” is a C₁-Cₘ-haloalkyl group, as defined above, attached via a S(═O)₂ group.

The term “C₂-Cₘ-alkenyl” as used herein denotes a linear or branched ethylenically unsaturated hydrocarbon group having 2 to m, e.g. 2 to 10 or 2 to 6 carbon atoms and a C═C-double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term “C₂-Cₘ-haloalkenyl” as used herein, which is also expressed as “C₂-Cₘ-alkenyl which is partially or fully halogenated”, refers to C₂-Cₘ-alkenyl, where some or all of the hydrogen atoms in these groups are replaced by halogen atoms as mentioned above, in particular fluorine, chlorine and bromine, for example 1-fluoroethenyl, 2-fluoroethenyl, 2,2-difluoroethenyl, 1,2,2-trifluoroethenyl, 1-fluoro-2-propenyl, 2-fluoro-2-propenyl, 3-fluoro-2-propenyl, 1-fluoro-1-propenyl, 1,2-difluoro-1-propenyl, 3,3-difluoropropen-2-yl, 1-chloroethenyl, 2-chloroethenyl, 2,2,-dichloroethenyl, 1-chloro-2-propenyl, and the like.

The term “C₂-Cₘ-alkynyl” as used herein refers to a linear or branched unsaturated hydrocarbon group having 2 to m, e.g. 2 to 10 or 2 to 6 carbon atoms and containing at least one tri-ple bond, such as ethynyl, propynyl, 1-butynyl, 2-butynyl, and the like.

The term “C₂-Cₘ-haloalkynyl” as used herein, which is also expressed as “C₂-Cₘ-alkynyl which is partially or fully halogenated”, refers to C₂-Cₘ-alkynyl, where some or all of the hydrogen atoms in these groups are replaced by halogen atoms as mentioned above, in particular fluorine, chlorine and bromine. Examples of C₂-Cₘ-haloalkynyl include 1-fluoro-2-propenyl, 2-fluoro-2-propenyl, 3-fluoro-2-propenyl, 1-fluoro-2-propynyl and 1,1-difluoro-2-propenyl, and the like.

The term “C₃-Cₘ-cycloalkyl” as used herein refers to a monocyclic or bicyclic or polycyclic 3- to m-membered saturated cycloaliphatic radicals, e.g. cyclopropyl, cyclobutyl, cyclopentyl, cyclohexyl, cycloheptyl, cyclooctyl, cyclodecyl, bicyclo[2.2.1]heptyl, bicyclo[3.1.1]heptyl, bicyclo[2.2.2]octyl and bicyclo[3.2.1]octyl. Preferably, the term cycloalkyl denotes a monocyclic saturated hydrocarbon radical.

The term “C₃-Cₘ-halocycloalkyl” as used herein, which is also expressed as “cycloalkyl which is partially or fully halogenated”, refers to C₃-Cₘ-cycloalkyl as mentioned above, in which some or all of the hydrogen atoms are replaced by halogen atoms as mentioned above, in particular fluorine, chlorine and bromine. Examples of C₃-Cₘ-halocycloalkyl include 1-fluorocyclopropyl, 2-fluorocyclopropyl, 2,2-difluorocyclopropyl, 1-chlorocyclopropyl, 2-chlorocyclopropyl, 2,2-dichlorocyclopropyl, 2,3-difluorocyclopropyl, 1-fluorocycobutyl etc.

The term “C₃-Cₘ-cycloalkyl-C₁-C₄-alkyl” refers to a C₃-Cₘ-cycloalkyl group as defined above, which is bound to the remainder of the molecule via a C₁-C₄-alkyl group, as defined above. Examples for C₃-Cₘ-cycloalkyl-C₁-C₄-alkyl are cyclopropylmethyl, cyclopropylethyl, cy-clopropylpropyl, cyclobutylmethyl, cyclobutylethyl, cyclobutylpropyl, cyclopentylmethyl, cyclo-pentylethyl, cyclopentylpropyl, cyclohexylmethyl, cyclohexylethyl and cyclohexylpropyl.

The term “C₃-Cₘ-halocycloalkyl-C₁-C₄-alkyl” refers to a C₃-Cₘ-halocycloalkyl group as defined above which is bound to the remainder of the molecule via a C₁-C₄-alkyl group, as defined above.

The term “C₁-C₄-alkoxy-C₁-C₄-alkyl” as used herein refers to alkyl having 1 to 4 carbon atoms, e.g. like specific examples mentioned above, wherein one hydrogen atom of the alkyl radical is replaced by an C₁-C₄-alkoxy group. Examples are methoxymethyl, ethoxymethyl, propoxymethyl, isopropoxymethyl, n-butoxymethyl, sec-butoxymethyl, isobutoxymethyl, tert-butoxymethyl, 1-methoxyethyl, 1-ethoxyethyl, 1-propoxyethyl, 1-isopropoxyethyl, 1-n-butoxyethyl, 1-sec-butoxyethyl, 1-isobutoxyethyl, 1-tert-butoxyethyl, 2-methoxyethyl, 2-ethoxyethyl, 2-propoxyethyl, 2-isopropoxyethyl, 2-n-butoxyethyl, 2-sec-butoxyethyl, 2-isobutoxyethyl, 2-tert-butoxyethyl, 1-methoxypropyl, 1-ethoxypropyl, 1-propoxypropyl, 1-isopropoxypropyl, 1-n-butoxypropyl, 1-sec-butoxypropyl, 1-isobutoxypropyl, 1-tert-butoxypropyl, 2-methoxypropyl, 2-ethoxypropyl, 2-propoxypropyl, 2-isopropoxypropyl, 2-n-butoxypropyl, 2-sec-butoxypropyl, 2-isobutoxypropyl, 2-tert-butoxypropyl, 3-methoxypropyl, 3-ethoxypropyl, 3-propoxypropyl, 3-isopropoxypropyl, 3-n-butoxypropyl, 3-sec-butoxypropyl, 3-isobutoxypropyl, 3-tert-butoxypropyl and the like.

The term C₁-C₄-haloalkoxy-C₁-C₄-alkyl is a straight-chain or branched alkyl group having from 1 to 4 carbon atoms, wherein one of the hydrogen atoms is replaced by a C₁-C₄-alkoxy group and wherein at least one, e.g. 1, 2, 3, 4 or all of the remaining hydrogen atoms, either in the alkoxy moiety or in the alkyl moiety or in both, are replaced by halogen atoms. Examples are difluoromethoxymethyl (CHF₂OCH₂), trifluoromethoxymethyl, 1-difluoromethoxyethyl, 1-trifluoromethoxyethyl, 2-difluoromethoxyethyl, 2-trifluoromethoxyethyl, difluoro-methoxymethyl (CH₃OCF₂), 1,1-difluoro-2-methoxyethyl, 2,2-difluoro-2-methoxyethyl and the like.

The term “C₁-Cₘ-alkoxycarbonyl” is a C₁-Cₘ-alkoxy group, as defined above, attached via an carbonyl group atom. C₁-C₂-Alkoxycarbonyl is methoxycarbonyl or ethoxycarbonyl. C₁-C₄-Alkoxy is, for example, methoxycarbonyl, ethoxycarbonyl, n-propoxycarbonyl, 1-methylethoxycarbonyl, butoxycarbonyl, 1-methylpropoxycarbonyl, 2-methylpropoxycarbonyl or 1,1-dimethylethoxycarbonyl. C₁-C₆-Alkoxycarbonyl includes the meanings given for C₁-C₄-alkoxycarbonyl and also includes, for example, pentoxycarbonyl, 1-methylbutoxycarbonyl, 2-methylbutoxycarbonyl, 3-methylbutoxycarbonyl, 1,1-dimethylpropoxycarbonyl, 1,2-dimethylpropoxycarbonyl, 2,2-dimethylpropoxycarbonyl, 1-ethylpropoxycarbonyl, hexoxycar-bonyl, 1-methylpentoxycarbonyl, 2-methylpentoxycarbonyl, 3-methylpentoxycarbonyl, 4-methylpentoxycarbonyl, 1,1-dimethylbutoxycarbonyl, 1,2-dimethylbutoxycarbonyl, 1,3-dimethylbutoxycarbonyl, 2,2-dimethylbutoxycarbonyl, 2,3-dimethylbutoxycarbonyl, 3,3-dimethylbutoxycarbonyl, 1-ethylbutoxy, 2-ethylbutoxycarbonyl, 1,1,2-trimethylpropoxycarbonyl, 1,2,2-trimethylpropoxycarbonyl, 1-ethyl-1-methylpropoxycarbonyl or 1-ethyl-2-methylpropoxycarbonyl.

The term “aryl” as used herein refers to an aromatic hydrocarbon radical such as naphthyl or in particular phenyl.

The term “3- to 6-membered carbocyclic ring” as used herein refers to cyclopropane, cyclobutane, cyclopentane and cyclohexane rings. The term “3- to 7-membered carbocyclic ring” as used herein refers to cyclopropane, cyclobutane, cyclopentane, cyclohexane and cycloheptane rings.

The term “3-, 4-, 5-, 6- or 7-membered saturated, partially unsaturated or aromatic heterocyclic ring containing 1, 2 or 3 heteroatoms” or “containing heteroatom groups”, wherein those heteroatom(s) (group(s)) are selected from N, O, S, NO, SO and SO₂ and are ring members, as used herein refers to monocyclic radicals, the monocyclic radicals being saturated, partially unsaturated or aromatic. The heterocyclic radical may be attached to the remainder of the molecule via a carbon ring member or via a nitrogen ring member.

Examples of 3-, 4-, 5-, 6- or 7-membered saturated heterocyclic rings include: oxiranyl, aziridinyl, azetidinyl, 2 tetrahydrofuranyl, 3-tetrahydrofuranyl, 2-tetrahydrothienyl, 3-tetrahydrothienyl, 2-pyrrolidinyl, 3-pyrrolidinyl, 3 pyrazolidinyl, 4 pyrazolidinyl, 5-pyrazolidinyl, 2 imidazolidinyl, 4 imidazolidinyl, 2-oxazolidinyl, 4-oxazolidinyl, 5 oxazolidinyl, 3-isoxazolidinyl, 4 isoxazolidinyl, 5 isoxazolidinyl, 2 thiazolidinyl, 4-thiazolidinyl, 5-thiazolidinyl, 3 isothiazolidinyl, 4-isothiazolidinyl, 5 isothiazolidinyl, 1,2,4-oxadiazolidin-3-yl, 1,2,4 oxadiazolidin-5-yl, 1,2,4-thiadiazolidin-3-yl, 1,2,4 thiadiazolidin-5-yl, 1,2,4 triazolidin-3-yl, 1,3,4-oxadiazolidin-2-yl, 1,3,4-thiadiazolidin-2-yl, 1,3,4 triazolidin-2-yl, 2-tetrahydropyranyl, 4-tetrahydropyranyl, 1,3-dioxan-5-yl, 1,4-dioxan-2-yl, 2-piperidinyl, 3-piperidinyl, 4-piperidinyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4 hexahydrotriazin-3-yl, 2-morpholinyl, 3-morpholinyl, 2-thiomorpholinyl, 3-thiomorpholinyl, 1-oxothiomorpholin-2-yl, 1-oxothiomorpholin-3-yl, 1,1-dioxothiomorpholin-2-yl, 1,1-dioxothiomorpholin-3-yl, hexahydroazepin-1-, -2-, -3- or -4-yl, hexahydrooxepinyl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl and the like. Examples of 3-, 4-, 5-, 6- or 7-membered partially unsaturated heterocyclic rings include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3 dihydrothien-3-yl, 2,4 dihydrothien-2-yl, 2,4-dihydrothien-3-yl, 2-pyrrolin-2-yl, 2-pyrrolin-3-yl, 3 pyrrolin-2-yl, 3-pyrrolin-3-yl, 2-isoxazolin-3-yl, 3-isoxazolin-3-yl, 4 isoxazolin 3 yl, 2-isoxazolin-4-yl, 3-isoxazolin-4-yl, 4-isoxazolin-4-yl, 2 isoxazolin-5-yl, 3-isoxazolin-5-yl, 4-isoxazolin-5-yl, 2-isothiazolin-3-yl, 3 isothiazolin-3-yl, 4-isothiazolin-3-yl, 2-isothiazolin-4-yl, 3-isothiazolin-4-yl, 4 isothiazolin-4-yl, 2-isothiazolin-5-yl, 3-isothiazolin-5-yl, 4-isothiazolin-5-yl, 2,3 dihydropyrazol-1-yl, 2,3-dihydropyrazol-2-yl, 2,3-dihydropyrazol-3-yl, 2,3 dihydropyrazol-4-yl, 2,3-dihydropyrazol-5-yl, 3,4-dihydropyrazol-1-yl, 3,4 dihydropyrazol-3-yl, 3,4-dihydropyrazol-4-yl, 3,4-dihydropyrazol-5-yl, 4,5 dihydropyrazol-1-yl, 4,5-dihydropyrazol-3-yl, 4,5-dihydropyrazol-4-yl, 4,5 dihydropyrazol-5-yl, 2,3-dihydrooxazol-2-yl, 2,3-dihydrooxazol-3-yl, 2,3 dihydrooxazol-4-yl, 2,3-dihydrooxazol-5-yl, 3,4-dihydrooxazol-2-yl, 3,4 dihydrooxazol-3-yl, 3,4-dihydrooxazol-4-yl, 3,4-dihydrooxazol-5-yl, 3,4 dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4 di- or tetrahydropyridazinyl, 2-di- or tetrahydropyrimidinyl, 4-di- or tetrahydropyrimidinyl, 5 di- or tetrahydropyrimidinyl, di- or tetrahydropyrazinyl, 1,3,5-di- or tetrahydrotriazin-2-yl, 1,2,4-di- or tetrahydrotriazin-3-yl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7 tetrahydro[1H]azepin-1-, -2-, -3-, -4-, -5-, -6- or -7-yl, 2,3,6,7 tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7 tetrahydro[1H]oxepin-2-, -3-, -4-, -5-, -6- or -7-yl, 2,3,6,7 tetrahydro[1H]oxepin-2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl and tetrahydro-1,4-dioxepinyl.

The term “3- to 10-membered (also referred to as 3-, 4-, 5-, 6-, 7-, 8-, 9- or 10-membered) saturated, partly saturated or unsaturated aromatic heterocyclic ring comprising 1, 2, 3 or 4 heteroatoms as ring members, which are identical or different and selected from oxygen, nitrogen and sulfur and wherein the nitrogen and/or the sulfur atom(s) of the heterocyclic ring may optionally be oxidized”, as used herein, refers to a 3-, 4-, 5-, 6- or 7-membered saturated, partially saturated or unsaturated aromatic heteromonocyclic ring as defined above or a 8-, 9- or 10-membered saturated, partially saturated or unsaturated aromatic heterobicyclic ring containing 1, 2, 3 or 4 heteroatoms or heteroatom groups selected from N, O, S, NO, SO and SO₂, as ring members. As a matter of course, the heterocyclic ring contains at least one carbon ring atom. If the ring contains more than one O ring atom, these are not adjacent. The heterocyclic radical may be attached to the remainder of the molecule via a carbon ring member or via a nitrogen ring member.

Examples of 5- or 6-membered aromatic heterocyclic rings, also termed heteroaromatic rings or hetaryl, include: 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4 thiazolyl, 5-thiazolyl, 2-imidazolyl, 4-imidazolyl, 1,3,4-triazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

Examples of 8-, 9- or 10-membered aromatic heterobicyclic rings include hetaryl which has one of the aforementioned 5- or 6-membered heteroaromatic rings and a further aromatic carbocycle or 5- or 6-membered heterocycle fused thereto, for example a fused benzene, thiophene, furane, pyrrole, pyrazole, imidazole, pyridine or pyrimidine ring. These bicyclic hetaryl include for example quinolinyl, isoquinolinyl, cinnolinyl, indolyl, indolizynyl, isoindolyl, indazolyl, benzofuryl, in particular 2-benzofuryl, benzothienyl, in particular 2-benzothienyl, benzo[b]thiazolyl, in particular 2-benzo[b]thiazolyl, benzoxazolyl, in particular 2-benzoxazolyl, benzthiazolyl, in particular 2-benzthiazolyl, benzimidazolyl, in particular 2-benzimidazol, imidazo[1,2-a]pyridine-2-yl, thieno[3,2-b]pyridine-5-yl, imidazo-[2,1-b]-thiazol-6-yl and 1,2,4-triazolo[1,5-a]pyridine-2-yl.

A “C₁-C₄-alkylene” is divalent linear or branched saturated aliphatic chain having 1 to 4 carbon atoms, for example CH₂, CH₂CH₂, CH₂CH₂CH₂, CH(CH₃)CH₂, CH₂CH(CH₃), CH₂CH₂CH₂CH₂, CH(CH₃)CH(CH₃)₂, CH₂C(CH₃)₂ or C(CH₃)₂CH₂.

A “C₂-Cₘ-alkylene” is divalent branched or preferably non-branched or linear saturated aliphatic chain having 2 to m, e.g. 2 to 7 carbon atoms, for example CH₂CH₂, —CH(CH₃)—, CH₂CH₂CH₂, CH(CH₃)CH₂, CH₂CH(CH₃), CH₂CH₂CH₂CH₂, CH₂CH₂CH₂CH₂CH₂, CH₂CH₂CH₂CH₂CH₂CH₂, and CH₂CH₂CH₂CH₂CH₂CH₂CH₂.

Embodiments of the present invention as well preferred compounds of the present invention are outlined in the following paragraphs. The remarks made below concerning preferred embodiments of the variables of the compounds of formula (I), especially with respect to their substituents X, Y, W¹, W², W³, W⁴, Het, R¹, R², R³, R⁴ and R⁵ and their variable k and m are valid both on their own and, in particular, in every possible combination with each other.

When # appears in a formula showing a preferred substructure of a compound of the present invention, it denotes the attachment bond in the remainder molecule.

In particular groups of embodiments, in the compounds of formula (I), the variables R³, R⁴, R⁵, R⁷, R^{7a}, R⁸, R⁹, R¹⁰, R¹⁵, R¹⁶, R¹⁷ and R^{17c} have the following meanings:
R³ is selected from the group consisting of hydrogen, halogen, CN, C₁-C₆-alkyl, C₃-C₈-cycloalkyl, C₃-C₈-cycloalkyl-C₁-C₄-alkyl, wherein each of the three last mentioned radicals are unsubstituted, partly or completely halogenated, or may carry any combination of 1, 2 or 3 radicals R⁷,
   OR⁸, S(O)ₙR⁸, S(O)ₙNR^{9a}R^{9b}, NR^{9a}R^{9b}, C(═O)OR⁸, C(═O)NR^{9a}R^{9b}, C(═S)NR^{9a}R^{9b}, C(═O)R^{7a}, C(═S)R^{7a},
   a moiety Q-phenyl, where the phenyl ring is optionally substituted with 1, 2, 3, 4 or 5 identical or different substituents R¹⁰,
   and a moiety Q-Het^{#} where Het^{#} represents a 3-, 4-, 5-, 6- or 7-membered saturated, partly saturated or unsaturated aromatic heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized,
R⁴ is selected from the group consisting of hydrogen, halogen, CN, C₁-C₆-alkyl, C₃-C₈-cycloalkyl, wherein each of the two last mentioned radicals are unsubstituted, partly or completely halogenated, or may carry any combination of 1, 2 or 3 radicals R⁷, S(O)ₙNR^{9a}R^{9b}, NR^{9a}R^{9b}, C(═O)OR⁸, C(═O)NR^{9a}R^{9b}, C(═S)NR^{9a}R^{9b}, C(═O)R^{7a}, C(═S)R^{7a}, a moiety Q-phenyl, where the phenyl ring is optionally substituted with 1, 2, 3, 4 or 5 identical or different substituents R¹⁰,
   and a moiety Q-Het^{#} where Het^{#} represents a 3-, 4-, 5-, 6- or 7-membered saturated, partly saturated or unsaturated aromatic heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized;
R⁵ is selected from the group consisting of hydrogen CN, C₁-C₆-alkyl, C₃-C₈-cycloalkyl, wherein each of the two last mentioned radicals are unsubstituted, partly or completely halogenated, or may carry any combination of 1, 2 or 3 radicals R⁷,
   S(O)ₙNR^{9a}R^{9b}, O—NR^{9a}R^{9b}, NR^{9a}R^{9b}, NR^{9a}NR^{9a}R^{9b}, NR^{9a}C(═O)R^{7a}, NR^{9a}C(═S)R^{7a}, NR^{9a}C(═O)NR^{9a}R^{9b}, NR^{9a}C(═S)NR^{9a}R^{9b}, C(═O)OR⁸, C(═O)NR^{9a}R^{9b}, C(═S)NR^{9a}R^{9b}, C(═O)R^{7a}, C(═S)R^{7a},
   a moiety Q-phenyl, where the phenyl ring is optionally substituted with 1, 2, 3, 4 or 5 identical or different substituents R¹⁰,
   and a moiety Q-Het^{#} where Het^{#} represents a 3-, 4-, 5-, 6- or 7-membered saturated, partly saturated or unsaturated aromatic heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized, or
R⁴ and R⁵ together form a moiety selected from Alk, S(O)ₙ-Alk, NR^{9c}-Alk, S(O)ₙ-Alk′-S(O)ₙ, O-Alk′-O, O-Alk′-NR^{9d}, S(O)ₙ-Alk′-NR^{9d}, S(O)ₙ-Alk′-S and NR^{9c}-Alk′-NR^{9d}, where
   Alk represents a saturated or unsaturated 2-, 3-, 4- or 5-membered carbon chain group, which is unsubstituted or carries 1, 2, 3 or 4 radicals R⁷ or a fused phenylene ring, where the fused phenyl ring is unsubstituted or substituted with 1, 2, 3 or 4 radicals R¹⁰;
   Alk′ is CH₂, where one or both hydrogen atoms may optionally be replaced by R⁷ or represents a saturated or unsaturated 2-, 3- or 4-membered carbon chain group, which is unsubstituted or carries 1, 2, 3 or 4 radicals R⁷ or a fused phenylene ring, where the fused phenyl ring is unsubstituted or substituted with 1, 2, 3 or 4 radicals R¹⁰;
      provided that R⁵ is different from hydrogen and C₁-C₆-alkyl, if R³ is hydrogen;
R⁷ independently of its occurrence, is selected from the group consisting of cyano, azido, nitro, —SCN, SF₅, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, Si(R¹¹)₂R¹², OR⁸, OSO₂R⁸, S(O)ₙR⁸, S(O)ₙNR^{17a}R^{17b}, NR^{17a}R^{17b}, C(═O)NR^{17a}R^{17b}, C(═S)NR^{17a}R^{17b}, C(═O)OR⁸, C(═O)R¹⁵, C(═S)R¹⁵, C(═NR¹⁷)R¹⁵,
   phenyl, phenyl-C₁-C₄-alkyl, where the phenyl ring in the last two groups is optionally substituted with 1, 2, 3, 4 or 5 identical or different substituents R¹⁰, and a 3-, 4-, 5-, 6- or 7-membered saturated, partly saturated or unsaturated aromatic heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized, or two R⁷ present on one carbon atom may together form ═O, ═CR¹³R¹⁴, ═S, ═NR¹⁷, ═NOR¹⁶, ═NNR^{9a}R^{9b},
   or two R⁷ may form a 3-, 4-, 5-, 6-, 7- or 8-membered saturated or partly unsaturated carbocyclic or heterocyclic ring together with the carbon atoms to which the two R⁷ are bonded, where the heterocyclic ring comprises 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰;
R^{7a} independently of its occurrence, is selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl,
   phenyl, optionally substituted with 1, 2, 3, 4 or 5 identical or different substituents R¹⁰, and a 3-, 4-, 5-, 6- or 7-membered saturated, partly saturated or unsaturated aromatic heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized;
R⁸ independently of its occurrence, is selected from the group consisting of hydrogen, C₁-C₆-alkyl, C₁-C₆-haloalkyl, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆ haloalkynyl, C(═O)R¹⁵, C(═O)NR^{17a}R^{17b}, C(═S)NR^{17a}R^{17b}, C(═O)OR¹⁶,
   phenyl, phenyl-C₁-C-₄-alkyl, where the phenyl ring in the last two mentioned radicals is unsubstituted or substituted with 1, 2, 3, 4 or 5 identical or different substituents R¹⁰, and a 3-, 4-, 5-, 6- or 7-membered saturated, partly saturated or unsaturated aromatic heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized;
R⁹ independently of its occurrence, is selected from the group consisting of hydrogen, C₁-C₆-alkyl, C₁-C₆-haloalkyl, C₁-C₆-alkoxy, C₁-C₆-haloalkoxy, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆ haloalkynyl,
   phenyl, optionally substituted with 1, 2, 3, 4 or 5 identical or different substituents R¹⁰; a 3-, 4-, 5-, 6- or 7-membered saturated, partly saturated or unsaturated aromatic C-bound heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized,
R¹⁰ independently of its occurrence, is selected from the group consisting of halogen, cyano, azido, nitro, SCN, SF₅, C₁-C₁₀-alkyl, C₃-C₈-cycloalkyl, C₂-C₁₀-alkenyl, C₂-C₁₀-alkynyl, wherein the carbon atoms of the aforementioned aliphatic and cycloaliphatic radicals may optionally be substituted with 1, 2, 3, 4 or 5 identical or different radicals R⁷,
   Si(R¹¹)₂R¹², OR¹⁶, OS(O)ₙR¹⁶, S(O)ₙNR^{17a}R^{17b}, NR^{17a}R^{17b}, C(═O)R¹⁵, C(═S)R¹⁵,
   C(═O)OR¹⁶, C(═NR¹⁷)R¹⁵, C(═O)NR^{17a}R^{17b}, C(═S)NR^{17a}R^{17b},
   phenyl, optionally substituted with 1, 2, 3, 4 or 5 identical or different radicals selected from OH, halogen, cyano, nitro, C₁-C₆-alkyl, C₁-C₆-haloalkyl, C₁-C₆-alkoxy and C₁-C₆-haloalkoxy, and
   a 3-, 4-, 5-, 6- or 7-membered saturated, partly saturated or unsaturated aromatic heterocyclic ring comprising 1, 2 or 3 heteroatoms as ring members, which are identical or different and selected from oxygen, nitrogen and sulfur, where the heterocyclic ring is unsubstituted or may be substituted with 1, 2, 3, 4 or 5 substituents selected independently from one another from halogen, cyano, NO₂, C₁-C₆-alkyl, C₁-C₆-haloalkyl, C₁-C₆-alkoxy and C₁-C₆-haloalkoxy, and wherein the nitrogen and/or the sulfur atom(s) of the heterocyclic ring may optionally be oxidized;
   or
   two R¹⁰ present together on one carbon ring atom of a saturated or partly unsaturated heterocyclic radical may form ═O, ═CR¹³R¹⁴, ═S, ═NR¹⁷, ═NOR¹⁶, ═NNR^{17a}R^{17b};
   or,
   two R¹⁰ on adjacent carbon ring atoms may also be a bivalent radical selected from CH₂CH₂CH₂CH₂, CH═CH—CH═CH, N═CH—CH═CH, CH═N—CH═CH, N═CH—N═CH, OCH₂CH₂CH₂, OCH═CHCH₂, CH₂OCH₂CH₂, OCH₂CH₂O, OCH₂OCH₂, CH₂CH₂CH₂, CH═CHCH₂, CH₂CH₂O, CH═CHO, CH₂OCH₂, CH₂C(═O)O, C(═O)OCH₂, O(CH₂)O, SCH₂CH₂CH₂, SCH═CHCH₂, CH₂SCH₂CH₂, SCH₂CH₂S, SCH₂SCH₂, CH₂CH₂S, CH═CHS, CH₂SCH₂, CH₂C(═S)S, C(═S)SCH₂, S(CH₂)S, CH₂CH₂NR¹⁷, CH₂CH═N, CH═CH—NR¹⁷, OCH═N, SCH═N and form together with the carbon atoms to which the two R¹⁰ are bonded to a 5-membered or 6-membered partly saturated or unsaturated, aromatic carbocyclic or heterocyclic ring, wherein the ring may optionally be substituted with one or two substituents selected from ═O, OH, CH₃, OCH₃, halogen, cyano, halomethyl and halomethoxy;
R¹⁵ independently of its occurrence, is selected from the group consisting of hydrogen, C₁-C₆-alkyl, C₂-C₆-alkenyl, C₂-C₆-alkynyl, C₃-C₈-cycloalkyl, wherein the four last mentioned aliphatic and cycloaliphatic radicals may be unsubstituted, partially or fully halogenated and/or oxygenated and/or may carry 1 or 2 radicals selected from C₁-C₄ alkoxy; phenyl, benzyl and pyridyl, wherein the last three radicals may be unsubstituted, partially or fully halogenated and/or to carry 1, 2 or 3 substituents selected from C₁-C₆-alkyl, C₁-C₆-haloalkyl, C₁-C₆-alkoxy, C₁-C₆ haloalkoxy, (C₁-C₆-alkoxy)carbonyl, (C₁-C₆-alkyl)amino or di-(C₁-C₆-alkyl)amino;
R₁₆ independently of its occurrence, is selected from the group consisting of hydrogen, C₁-C₆-alkyl, C₂-C₆-alkenyl, C₂-C₆-alkynyl, C₃-C₈-cycloalkyl, C₃-C₈-cycloalkyl-C₁-C₄-alkyl, wherein the five last mentioned aliphatic and cycloaliphatic radicals may be unsubstituted, partially or fully halogenated and/or oxygenated and/or may carry 1 or 2 radicals selected from C₁-C₄ alkoxy,
   phenyl, benzyl and pyridyl, wherein the last three radicals may be unsubstituted, partially or fully halogenated and/or to carry 1, 2 or 3 substituents selected from C₁-C₆-alkyl, C₁-C₆-haloalkyl, C₁-C₆-alkoxy, C₁-C₆ haloalkoxy, (C₁-C₆-alkoxy)carbonyl, (C₁-C₆-alkyl)amino or di-(C₁-C₆-alkyl)amino;
R¹⁷ independently of its occurrence, is selected from the group consisting of hydrogen, C₁-C₆-alkoxy, C₁-C₆-haloalkoxy, C₁-C₆-alkylthio, trimethylsilyl, triethylsilyl, tertbutyldimethylsilyl, C₁-C₆-alkyl, C₂-C₆-alkenyl, C₂-C₆-alkynyl, C₃-C₈-cycloalkyl, wherein the four last mentioned aliphatic and cycloaliphatic radicals may be unsubstituted, partially or fully halogenated and/or oxygenated and/or may carry 1 or 2 radicals selected from C₁-C₄-alkoxy, phenyl, benzyl, pyridyl, phenoxy, wherein the four last mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy and (C₁-C₆-alkoxy)carbonyl,
R^{17c} independently of its occurrence, is selected from the group consisting of hydrogen, CN, C₁-C₆-alkyl, C₂-C₆-alkenyl, C₂-C₆-alkynyl, C₃-C₈-cycloalkyl, C₃-C₈-cycloalkyl-C₁-C₄-alkyl, wherein the five last mentioned aliphatic and cycloaliphatic radicals may be unsubstituted, partially or fully halogenated and/or oxygenated and/or may carry 1 or 2 radicals selected from C₁-C₄-alkoxy,
   phenyl, benzyl and pyridyl, wherein the last three radicals may be unsubstituted, partially or fully halogenated and/or may carry 1, 2 or 3 substituents selected from C₁-C₆-alkyl, C₆-haloalkyl, C₁-C₆-alkoxy, C₁-C₆-haloalkoxy, (C₁-C₆-alkoxy)carbonyl, (C₁-C₆-alkyl)amino or di-(C₁-C₆-alkyl)amino;
      where n, R^{9a}, R^{9b}, R^{9c}, R^{9d}, R¹⁰, R¹¹, R¹², R¹³, R¹⁴, R^{17a}, R^{17b}, Q, Het^{#} have one of the meanings given above.

The present invention relates in particular to compounds of the formula (I), their stereoisomers, their tautomers and their salts, where one or more of the following provisos (1) to (3) are met:
(1) at least one of the radicals R³, R⁴ and R⁵ is different from C₁-C₄-alkyl, hydrogen or halogen;
(2) at least one of the radicals R³, R⁴ and R⁵ is
   a moiety Q-phenyl, where the phenyl ring is optionally substituted with 1, 2, 3, 4 or 5 identical or different substituents R¹⁰,
   or
   a moiety Q-Het^{#};
(3) the total number of carbon atoms in the radicals R³, R⁴ and R⁵ is more than 4, in particular from 5 to 20.

Preferred are compounds of formula (I), wherein Het is selected from the group consisting of radicals of formulae Het-1 to Het-24, with preference given to compounds of the formula (I), their stereoisomers, there tautomers and their salts, where Het is selected from the radicals of the formulae Het-1, Het-10, Het-11, Het-23 and Het-24, in particular Het-1, Het-11 and Het-24.

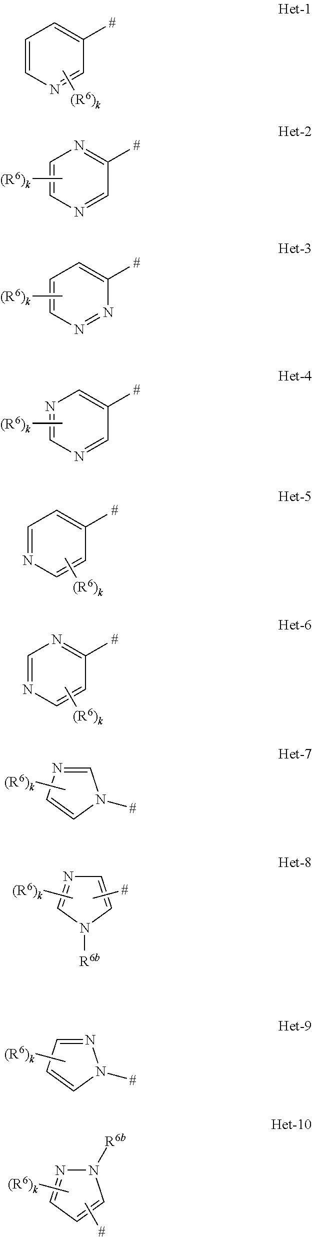

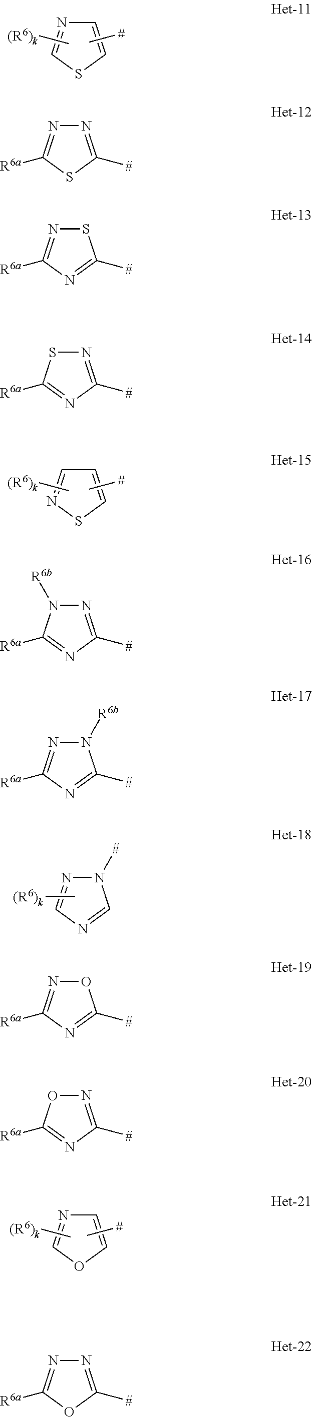

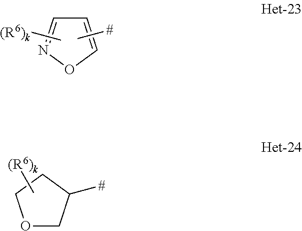

wherein # denotes the bond to the remainder of the molecule in formula (I), and wherein R⁶ and k are as defined above and where R^{6a} is hydrogen or has one of the meanings given for R⁶ and where R^{6b} is hydrogen or a C-bound radical mentioned as R⁶ and where R^{6b} is in particular hydrogen, C₁-C₄-alkyl or C₁-C₄-haloalkyl. In particular k is 0, 1 or 2, especially 0 or 1. In formulae Het-1, Het-2, Het-3, Het-4, Het-7, Het-8, Het-9, Het-10, Het-11, Het-18 and Het-21, k is especially 1. In formula Het-23, k is especially 0 or 1. In particular R^{6a} in formulae Het-12, Het-13, Het-14, Het-16, Het-17, Het-19, Het 20 and Het-22 is different from hydrogen.

Irrespectively of its occurrence, R⁶ is preferably selected from the group consisting of halogen, cyano, C₁-C₆-alkyl, C₃-C₈-cycloalkyl, C₂-C₆-alkenyl, C₂-C₆-alkynyl, wherein the carbon atoms of the aforementioned aliphatic and cycloaliphatic radicals may optionally be partly or completely halogenated, in particular by fluorine or chlorine, or may further substituted independently from one another with one or more R⁷, or R⁶ may also be a radical selected from the group consisting of OR⁸, NR^{17a}R^{17b}, S(O)ₙR⁸, S(O)ₙNR^{17a}R^{17b}, C(═O)R^{7a}, C(═O)NR^{17a}R^{17b}, C(═O)OR⁸, C(═S)R^{7a}, C(═S)NR^{17a}R^{17b}, C(═NR¹⁷)R^{7a}, C(═NR¹⁷)NR^{17a}R^{17b}. Irrespectively of its occurrence, R⁶ is in particular selected from the group consisting of halogen, such as chlorine or fluorine, C₁-C₄-alkyl, such as methyl or ethyl, C₁-C₄-alkoxy, such as methoxy or ethoxy, C₁-C₄-haloalkoxy, such as difluoromethoxy or trifluormethoxy, and C₁-C₄-haloalkyl, such as difluoromethyl, trifluoromethyl, 2,2-difluoroethyl, 2,2,2-trifluoroethyl or pentafluoroethyl, more preferably from halogen, C₁-C₄-alkyl and C₁-C₄-haloalkyl, even more preferably from fluorine, chlorine, C₁-C₂-alkyl, such as methyl or ethyl and C₁-C₂-haloalkyl such as difluoromethyl, trifluoromethyl, 2,2-difluoroethyl, 2,2,2-trifluoroethyl or pentafluoroethyl.

Irrespectively of its occurrence, R^{6a} is preferably selected from the group consisting of hydrogen, halogen, cyano, C₁-C₆-alkyl, C₃-C₈-cycloalkyl, C₂-C₆-alkenyl and C₂-C₆-alkynyl, wherein the carbon atoms of the aforementioned aliphatic and cycloaliphatic radicals may optionally be partly or completely halogenated, in particular by fluorine or chlorine, or may further substituted independently from one another with one or more R⁷, or R^{6a} may also be a radical selected from the group consisting of OR⁸, NR^{17a}R^{17b}, S(O)ₙR⁸, S(O)ₙNR^{17a}R^{17b}, C(═O)R^{7a}, C(═O)NR^{17a}R^{17b}, C(═O)OR⁸, C(═S)R^{7a}, C(═S)NR^{17a}R^{17b}, C(═NR¹⁷)R^{7a}, C(═NR¹⁷)NR^{17a}R^{17b}. Irrespectively of its occurrence, R^{6a} is in particular selected from the group consisting of hydrogen, halogen, such as chlorine or fluorine, C₁-C₄-alkyl, such as methyl or ethyl, C₁-C₄-alkoxy, such as methoxy or ethoxy, C₁-C₄-haloalkoxy, such as difluoromethoxy or trifluormethoxy, and C₁-C₄-haloalkyl, such as difluoromethyl, trifluoromethyl, 2,2-difluoroethyl, 2,2,2-trifluoroethyl or pentafluoroethyl, more preferably from halogen, C₁-C₄-alkyl and C₁-C₄-haloalkyl, even more preferably from fluorine, chlorine, C₁-C₂-alkyl, such as methyl or ethyl and C₁-C₂-haloalkyl such as difluoromethyl, trifluoromethyl, 2,2-difluoroethyl, 2,2,2-trifluoroethyl or pentafluoroethyl.

Irrespectively of its occurrence, R^{6b} is in particular selected from the group consisting of hydrogen, C₁-C₄-alkyl, such as methyl or ethyl, and C₁-C₄-haloalkyl, such as difluoromethyl, trifluoromethyl, 2,2-difluoroethyl, 2,2,2-trifluoroethyl or pentafluoroethyl, more preferably C₁-C₂-alkyl, such as methyl or ethyl and C₁-C₂-haloalkyl such as difluoromethyl, trifluoromethyl, 2,2-difluoroethyl, 2,2,2-trifluoroethyl or pentafluoroethyl.

Particularly preferred are compounds of formula (I), wherein Het is selected from the group consisting of radicals of formulae Het-1, Het-10a, Het-11a, Het-23a and Het-24, in particular Het-1, Het-11a and Het-24,

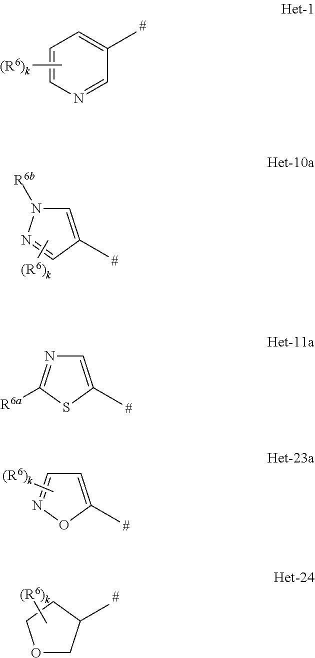

where
R⁶ is selected from the group consisting of halogen, such as chlorine or fluorine, C₁-C₄-alkyl, such as methyl or ethyl, C₁-C₄-alkoxy, such as methoxy or ethoxy, C₁-C₄-haloalkoxy, such as difluoromethoxy or trifluormethoxy, and C₁-C₄-haloalkyl, such as difluoromethyl, trifluoromethyl, 2,2-difluoroethyl, 2,2,2-trifluoroethyl or pentafluoroethyl, more preferably from halogen, C₁-C₄-alkyl and C₁-C₄-haloalkyl, even more preferably from fluorine, chlorine, C₁-C₂-alkyl, such as methyl or ethyl and C₁-C₂-haloalkyl such as difluoromethyl, trifluoromethyl, 2,2-difluoroethyl, 2,2,2-trifluoroethyl or pentafluoroethyl; and where
R^{6a} is selected from the group consisting of hydrogen, halogen, such as chlorine or fluorine, C₁-C₄-alkyl, such as methyl or ethyl, C₁-C₄-alkoxy, such as methoxy or ethoxy, C₁-C₄-haloalkoxy, such as difluoromethoxy or trifluormethoxy, and C₁-C₄-haloalkyl, such as difluoromethyl, trifluoromethyl, 2,2-difluoroethyl, 2,2,2-trifluoroethyl or pentafluoroethyl, more preferably from halogen, C₁-C₄-alkyl and C₁-C₄-haloalkyl, even more preferably from fluorine, chlorine, C₁-C₂-alkyl, such as methyl or ethyl and C₁-C₂-haloalkyl such as difluoromethyl, trifluoromethyl, 2,2-difluoroethyl, 2,2,2-trifluoroethyl or pentafluoroethyl;
R^{6b} is selected from C₁-C₄-alkyl, such as methyl or ethyl, C₁-C₄-alkoxy, such as methoxy or ethoxy, C₁-C₄-haloalkoxy, such as difluoromethoxy or trifluormethoxy, and C₁-C₄-haloalkyl, such as difluoromethyl, trifluoromethyl, 2,2-difluoroethyl, 2,2,2-trifluoroethyl or pentafluoroethyl, more preferably from C₁-C₄-alkyl and C₁-C₄-haloalkyl, even more preferably from C₁-C₂-alkyl, such as methyl or ethyl and C₁-C₂-haloalkyl such as difluoromethyl, trifluoromethyl, 2,2-difluoroethyl, 2,2,2-trifluoroethyl or pentafluoroethyl and
k is 0, 1 or 2.

A particularly preferred group of embodiments relates to compounds of formula (I) to the stereoisomers, the tautomers and to the salts thereof, wherein Het is a radical of formula Het-1, where k is 0, 1 or 2, in particular 1 or 2 and especially 1 and where R⁶ is as defined above and in particular selected from the group consisting of halogen, such as chlorine or fluorine, C₁-C₄-alkyl, such as methyl or ethyl, C₁-C₄-alkoxy, such as methoxy or ethoxy, C₁-C₄-haloalkoxy, such as difluoromethoxy or trifluormethoxy, and C₁-C₄-haloalkyl, such as difluoromethyl, trifluoromethyl, 2,2-difluoroethyl, 2,2,2-trifluoroethyl or pentafluoroethyl, more preferably from halogen, C₁-C₄-alkyl and C₁-C₄-haloalkyl, even more preferably from fluorine, chlorine, C₁-C₂-alkyl, such as methyl or ethyl and C₁-C₂-haloalkyl such as difluoromethyl, trifluoromethyl, 2,2-difluoroethyl, 2,2,2-trifluoroethyl or pentafluoroethyl. Amongst the compounds of this particular group of embodiments, a particular sub-group of embodiments relates to compounds of the formula (I), to the stereoisomers, the tautomers and to the salts thereof, wherein Het is a radical of formula Het-1a

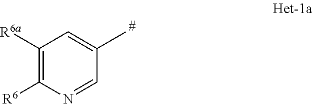

where
R⁶ is as defined above and in particular selected from the group consisting of halogen, such as chlorine or fluorine, C₁-C₄-alkyl, such as methyl or ethyl, and C₁-C₄-haloalkyl, such as difluoromethyl, trifluoromethyl, 2,2-difluoroethyl, 2,2,2-trifluoroethyl or pentafluoroethyl, and even more preferably from fluorine, chlorine, C₁-C₂-alkyl, such as methyl or ethyl and C₁-C₂-haloalkyl such as difluoromethyl, trifluoromethyl, 2,2-difluoroethyl, 2,2,2-trifluoroethyl or pentafluoroethyl;
R^{6a} is as defined above and in particular selected from the group consisting of hydrogen, halogen, such as chlorine or fluorine and C₁-C₄-alkyl, such as methyl or ethyl, more preferably is hydrogen.

A special embodiment of the radical Het-1a is 6-chloropyridin-3-yl, i.e. R^{6a} is hydrogen and R⁶ is chlorine. A further special embodiment of the radical Het-1a is 6-(trifluoromethyl)pyridin-3-yl, i.e. R^{6a} is hydrogen and R⁶ is trifluoromethyl.

Another particularly preferred group of embodiments relates to compounds of formula (I) to the stereoisomers, the tautomers and to the salts thereof, wherein Het is a radical of formula Het-10, where k is 0, 1 or 2, in particular 0 or 1, even more preferably 0, and where Het is in particular a radical of formula Het-10a,

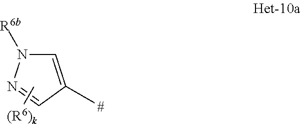

where R⁶ and R^{6b} are as defined above and wherein R^{6b} is in particular selected from the group consisting of C₁-C₄-alkyl, such as methyl or ethyl, C₁-C₄-alkoxy, such as methoxy or ethoxy, C₁-C₄-haloalkoxy, such as difluoromethoxy or trifluormethoxy, and C₁-C₄-haloalkyl, such as difluoromethyl, trifluoromethyl, 2,2-difluoroethyl, 2,2,2-trifluoroethyl or pentafluoroethyl, more preferably from C₁-C₄-alkyl and C₁-C₄-haloalkyl, even more preferably from C₁-C₂-alkyl, such as methyl or ethyl. A special embodiment of the radical Het-10a is 1-methylpyrazol-4-yl, i.e. R^{6b} is methyl and k is 0.

Another particularly preferred group of embodiments relates to compounds of formula (I) to the stereoisomers, the tautomers and to the salts thereof, wherein Het is a radical of formula Het-11, where k is 0, 1 or 2, in particular 0 or 1, and where Het is in particular a radical of formula Het-11a,

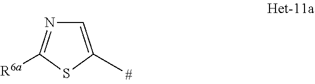

where R⁶ and R^{6a} are as defined above and wherein R^{6a} is in particular selected from the group consisting of hydrogen, halogen, such as chlorine or fluorine, C₁-C₄-alkyl, such as methyl or ethyl, C₁-C₄-alkoxy, such as methoxy or ethoxy, C₁-C₄-haloalkoxy, such as difluoromethoxy or trifluormethoxy, and C₁-C₄-haloalkyl, such as difluoromethyl, trifluoromethyl, 2,2-difluoroethyl, 2,2,2-trifluoroethyl or pentafluoroethyl, more preferably from halogen, C₁-C₄-alkyl and C₁-C₄-haloalkyl, even more preferably from fluorine, chlorine, C₁-C₂-alkyl, such as methyl or ethyl and C₁-C₂-haloalkyl such as difluoromethyl, trifluoromethyl, 2,2-difluoroethyl, 2,2,2-trifluoroethyl or pentafluoroethyl. A special embodiment of the radical Het-11a is 2-chlorothiazol-5-yl, i.e. R^{6a} is chlorine.

Another particularly preferred group of embodiments relates to compounds of formula (I) to the stereoisomers, the tautomers and to the salts thereof, wherein Het is a radical of formula Het-23, where k is 0, 1 or 2, in particular 0 or 1, and where Het is in particular a radical of formula Het-23a,

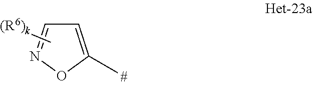

where R⁶ is as defined above and wherein R⁶ is in particular selected from the group consisting of C₁-C₄-alkyl, such as methyl or ethyl, C₁-C₄-alkoxy, such as methoxy or ethoxy, C₁-C₄-haloalkoxy, such as difluoromethoxy or trifluormethoxy, and C₁-C₄-haloalkyl, such as difluoromethyl, trifluoromethyl, 2,2-difluoroethyl, 2,2,2-trifluoroethyl or pentafluoroethyl, more preferably from C₁-C₄-alkyl and C₁-C₄-haloalkyl, even more preferably from C₁-C₂-alkyl, such as methyl or ethyl and C₁-C₂-haloalkyl such as difluoromethyl, trifluoromethyl, 2,2-difluoroethyl, 2,2,2-trifluoroethyl or pentafluoroethyl. A special embodiment of the radical Het-23a is 3-methylisoxazol-5-yl, i.e. k is 1 and R⁶ is methyl which is attached in the 3-position of the isoxazol ring.

Another particularly preferred group of embodiments relates to compounds of formula (I) to the stereoisomers, the tautomers and to the salts thereof, wherein Het is a radical of formula Het-24, where k is 0, 1 or 2, in particular 0 or 1, and where R⁶, if present, is as defined above and in particular selected from the group consisting of halogen, such as chlorine or fluorine, C₁-C₄-alkyl, such as methyl or ethyl, and C₁-C₄-haloalkyl, such as difluoromethyl, trifluoromethyl, 2,2-difluoroethyl, 2,2,2-trifluoroethyl or pentafluoroethyl, and even more preferably from fluorine, chlorine, C₁-C₂-alkyl, such as methyl or ethyl and C₁-C₂-haloalkyl such as difluoromethyl, trifluoromethyl, 2,2-difluoroethyl, 2,2,2-trifluoroethyl or pentafluoroethyl.

Preferred are compounds of formula (I), wherein R¹ and R² are independently from each other selected from the group consisting of hydrogen, halogen, such as fluorine or chlorine, CN, C₁-C₆-alkyl, in particular C₁-C₄-alkyl, such as methyl, ethyl, n-propyl or isopropyl, C₃-C₆-cycloalkyl, such as cyclopropyl or cyclobutyl, C₁-C₆-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, or C₃-C₆-halocycloalkyl such as 1-fluorocyclopropyl or 2,2-difluorocyclopropyl.

Preferred are also compounds of formula (I), wherein R¹ and R² may together be ═CR¹³R¹⁴.

Preferred are also compounds of formula (I), wherein R¹ and R² form, together with the carbon atom, which they attached to, a 3- to 5 membered saturated carbocyclic ring such as cyclopropyl, cyclobutyl or cyclopentyl.

Even more preferred are compounds of formula (I), wherein R¹ and R² are independently from each other selected from the group consisting of hydrogen, halogen, cyano, C₁-C₃-alkyl, such as methyl ethyl or isopropyl, or C₁-C₃-haloalkyl such as fluoromethyl, difluoromethyl, trifluoromethyl, 1,1-difluoroethyl, 2-fluoroethyl, 2,2-difluoroethyl or 2,2,2-trifluoroethyl.

Preferably at least one of the radicals R¹ and R² is hydrogen.

Especially more preferred are compounds of formula (I), wherein R¹ and R² are both hydrogen. Likewise, especially more preferred are compounds of formula (I), wherein one of R¹ and R² is hydrogen while the other is methyl.

Preferred are compounds of formula (I), wherein R³ is different from hydrogen.

Likewise preferred are compounds of formula (I), wherein R³ is selected from the group consisting of hydrogen, halogen, CN, C₁-C₆-alkyl, in particular C₁-C₃-alkyl, such as methyl, ethyl and isopropyl, and C₁-C₆-haloalkyl, in particular C₁-C₃-haloalkyl, such as fluoromethyl, difluoromethyl, trifluoromethyl, 1,1-difluoroethyl, 2-fluoroethyl, 2,2-difluoroethyl or 2,2,2-trifluoroethyl.

Particularly preferred are the compounds of formula (I), wherein R³ is fluorine or CN.

Preferred are compounds of formula (I), wherein R⁴ is selected from the group consisting of hydrogen, halogen, CN, NR^{9a}R^{9b}, C₁-C₆-alkyl, C₁-C₆-haloalkyl, or Q-phenyl, where Q is as defined above and Q is in particular a single bond, NR^{9a}, CH₂, or NR^{9a}CH₂ and where phenyl is unsubstituted or substituted with 1, 2, 3, 4 or 5, in particular 1, 2 or 3 identical or different substituents R¹⁰.

Even more preferred are compounds of formula (I), wherein R⁴ is selected from the group consisting of hydrogen, fluorine, chlorine, CN, C₁-C₄-alkyl, such as methyl, ethyl, n-propyl and isopropyl, and C₁-C₄-haloalkyl, such as fluoromethyl, difluoromethyl, trifluoromethyl, 1,1-difluoroethyl, 2-fluoroethyl, 2,2-difluoroethyl or 2,2,2-trifluoroethyl. Even most preferred are compounds of the formula (I), wherein R⁴ is selected from the group consisting of hydrogen and fluorine.

According to a first group of embodiments, compounds of formula (I) are preferred, wherein R⁵ is CN, C₁-C₄-cyanoalkyl, C₁-C₄-alkoxy-C₁-C₄-alkyl, C₁-C₄-alkthio-C₁-C₄-alkyl, NR^{9a}R^{9b}, C₁-C₆-alkyl, C₁-C₆-haloalkyl, C(═O)OR⁸, C(═O)NR^{9a}R^{9b}, C(═S)NR^{9a}R^{9b}, C(═O)R^{7a}, C(═S)R^{7a}, C₃-C₆-cycloalkyl-C₁-C₄-alkyl, C₃-C₈-cycloalkyl, where cycloalkyl in the last two radicals is unsubstituted or optionally substituted with 1, 2, 3 or 4 C₁-C₄-alkyl radicals, Q-phenyl, where phenyl is unsubstituted or substituted with 1, 2, 3, 4 or 5 identical or different substituents R¹⁰, or Q-Het^{#}, where Het^{#} is unsubstituted or substituted with 1, 2, 3, or 4 identical or different substituents R¹⁰, and where Q, irrespectively of its occurrence, is a single bond, NR^{9a}, CH₂, or NR^{9a}CH₂.

In this group of embodiments, preferred are compounds of formula (I), wherein R⁵ is selected from the group consisting of CN, NR^{9a}R^{9b}, C₁-C₆-alkyl, such as methyl, ethyl, n-propyl, isopropyl, n-butyl, 2-butyl or 2-methylpropyl, 1,1-dimethylethyl, n-pentyl, 1-methylbutyl, 1,2-dimethylpropyl, 1,2-dimethylbutyl, 1-ethylpentyl, 1,2,2-trimethylpropyl, C₁-C₆-haloalkyl such as dichloromethyl, C(═O)OR⁸, C(═O)NR^{9a}R^{9b}, C(═S)NR^{9a}R^{9b}, C(═O)R^{7a}, C(═S)R^{7a}, Q-phenyl, where phenyl is unsubstituted or substituted with 1, 2, 3, 4 or 5, in particular 1, 2 or 3, identical or different substituents R¹⁰, or Q-Het^{#}, where Het^{#} is unsubstituted or substituted with 1, 2, 3, or 4, in particular 1 or 2 identical or different substituents R¹⁰, and where Q is as defined above and preferably, irrespectively of its occurrence, selected from a single bond, NR^{9a}, CH₂, and NR^{9a}CH₂. Likewise preferred are compounds of formula (I), where Q is linear or branched C₂-C₄-alkylene such as CH₂CH₂, CH₂CH₂CH₂, CH(CH₃)CH₂ or C(CH₃)₂CH₂. In this group of embod-invents, likewise preference is given to compounds of formula (I), wherein R⁵ is C₃-C₈-cycloalkyl, which is unsubstituted or optionally substituted with 1, 2, 3 or 4 C₁-C₄-alkyl radicals, such as cyclopropyl, cyclobutyl, cyclopentyl, cyclohexyl, cycloheptyl, cyclooctyl, bicyclo[3.1.1]heptyl, 4-methylcyclohexyl, 4-ethylcyclohexyl, 6,6-dimethylnorpinan-2-yl. In this group of embodiments, preferred are also compounds of formula (I), wherein R⁵ is C₁-C₆-alkyl, which is substituted by 1 radical R⁷ selected from CN, C₁-C₄-alkoxy, C₁-C₄-alkylcarbonyloxy, C₁-C₄-alkoxy-C₁-C₄-alkoxy, C₁-C₄-alkylsulfanyl, C₃-C₈-cycloalkyl, C₃-C₈-cycloalkoxy, phenoxy and 5- or 6-membered saturated heterocyclyl having 1 heteroatom selected from O and S, such as tetrahydropyranyl, tetrahydrofuranyl, tetrahydrothiopyranyl, where C₃-C₈-cycloalkyl, C₃-C₈-cycloalkoxy and 5- or 6-membered saturated heterocyclyl may be unsubstituted or carry 1 or 2 radicals selected from C₁-C₄-alkyl and C₁-C₄-alkoxy, examples of substituted C₁-C₆-alkyl including cyanomethyl, 1-methoxyethyl, 2-methoxyethyl, 1-propoxyethyl, 2-propoxyethyl, 1-isopropoxyethyl, 2-isopropoxyethyl, 1-isobutoxyethyl, 2-isobutoxyethyl, 1-(cyclohexoxy)ethyl, 1-phenoxyethyl, 2-phenoxyethyl, 1-(2-methoxyethoxy)ethyl, (4-isopropylcyclohexyl)methyl, 1-acetoxyethyl, 2-acetoxyethyl, 3-acetoxy-1-methyl-propyl, 6-acetoxyhexyl, 2-methylsulfanylpropyl, 1-methyl-2-methylsulfanylethyl, (4-methylcyclohexyl)methyl or (4-ethylcyclohexyl)methyl, tetrahydropyran-4-ylmethyl and tetrahydrothiopyran-4-ylmethyl.

Particularly preferred are compounds of formula (I), wherein R⁵ is selected from the group consisting of cyanomethyl, NR^{9a}R^{9b}, C₁-C₆-alkyl, C₁-C₄-haloalkyl, C(═O)OR⁸, C(═O)NR^{9a}R^{9b}, C(═O)R^{7a}, Q-phenyl, where phenyl is unsubstituted or substituted with 1, 2, 3, 4 or 5 identical or different substituents R¹⁰, or Q-Het^{#}, where Het^{#} is unsubstituted or substituted with 1, 2, 3, or 4 identical or different substituents R¹⁰, and where Q, irrespectively of its occurrence, is a single bond, NH, N(C₁-C₄-alkyl), CH₂, NHCH₂ or N(C₁-C₄-alkyl)CH₂. Het^{#} is preferably a 3-, 4-, 5-, 6- or 7-membered saturated, partly saturated or unsaturated aromatic monocyclic heterocyclic ring comprising 1, 2 or 3 heteroatoms as ring members, which are identical or different and selected from oxygen, nitrogen and sulfur, such as tetrahydrofuryl, tetrahydropyranyl, tetrahydrothiopyranyl, pyrazolyl, pyrrolyl, isoxazolyl, thiazolyl, pyridyl or pyrimidinyl, where the heterocyclic ring is optionally substituted with 1, 2, 3 or 4 identical or different substituents R¹⁰. According to a further group of embodiments, Het^{#} is a 8-, 9- or 10-membered aromatic bicyclic ring comprising 1, 2 or 3 heteroatoms as ring members, which are identical or different and selected from oxygen, nitrogen and sulfur such as quinolinyl, isoquinolinyl, cinnolinyl, indolyl, indolizynyl, isoindolyl, indazolyl, 2-benzofuryl, 2-benzothienyl, 2-benzo[b]thiazolyl, 2-benzoxazolyl, 2-benzthiazolyl, 2-benzimidazol, imidazo[1,2-a]pyridine-2-yl, thieno[3,2-b]pyridine-5-yl, imidazo-[2,1-b]-thiazol-6-yl and 1,2,4-triazolo[1,5-a]pyridine-2-yl, where the heterocyclic ring is optionally substituted with 1, 2, 3 or 4 identical or different substituents R¹⁰.

In another preferred group of embodiments, the radicals R⁴ and R⁵ together form a moiety selected from Alk, S(O)ₙ-Alk, NR^{9c}-Alk, S(O)ₙ-Alk′-S(O)ₙ, (O)-Alk′-O and NR^{9c}-Alk′-NR^{9d}, where Alk represents a saturated or unsaturated 2-, 3- or 4-membered carbon chain group, such as CH₂CH₂, CH₂CH₂CH₂ or CH₂CH₂CH₂CH₂, which is unsubstituted or carries 1, 2, 3 or 4 radicals R⁷ or a fused phenylene ring, where the fused phenyl ring is unsubstituted or substituted with 1, 2, 3 or 4 radicals R¹⁰ and where Alk′ is CH₂ or has one of the meanings given for Alk. Particularly preferred are compounds of formula (I), wherein
R³ is selected from the group consisting of hydrogen, halogen, CN, C₁-C₆-alkyl, in particular C₁-C₃-alkyl, such as methyl, ethyl and isopropyl, and C₁-C₆-haloalkyl, in particular C₁-C₃-haloalkyl, such as fluoromethyl, difluoromethyl, trifluoromethyl, 1,1-difluoroethyl, 2-fluoroethyl, 2,2-difluoroethyl or 2,2,2-trifluoroethyl;
R⁴ is selected from the group consisting of hydrogen, halogen, CN, NR^{9a}R^{9b}, C₁-C₆-alkyl, C₆-haloalkyl, or Q-phenyl, where Q is as defined above and Q is in particular a single bond, NR^{9a}, CH₂, or NR^{9a}CH₂ and where phenyl is unsubstituted or substituted with 1, 2, 3, 4 or 5, in particular 1, 2 or 3 identical or different substituents R¹⁰; and
R⁵ is selected from the group consisting of CN, C₁-C₄-cyanoalkyl, C₁-C₄-alkoxy-C₁-C₄-alkyl, C₁-C₄-alkthio-C₁-C₄-alkyl, NR^{9a}R^{9b}, C₁-C₆-alkyl, such as methyl, ethyl, n-propyl, isopropyl, n-butyl, 2-butyl, 2-methylpropyl, 1,1-dimethylethyl, n-pentyl, 1-methylbutyl, 1,2-dimethylbutyl, 1-ethylpentyl or 1,2,2-trimethylpropyl, C₁-C₆-haloalkyl, C(═O)OR⁸, C(═O)NR^{9a}R^{9b}, C(═S)NR^{9a}R^{9b}, C(═O)R^{7a}, C(═S)R^{7a},
   C₃-C₆-cycloalkyl-C₁-C₄-alkyl, C₃-C₈-cycloalkyl, where cycloalkyl in the last two radicals is unsubstituted or optionally substituted with 1, 2, 3 or 4 C₁-C₄-alkyl radicals, Q-phenyl, where phenyl is unsubstituted or substituted with 1, 2, 3, 4 or 5, in particular 1, 2 or 3, identical or different substituents R¹⁰, or Q-Het^{#}, where Het^{#} is unsubstituted or substituted with 1, 2, 3, or 4, in particular 1 or 2 identical or different substituents R¹⁰, and where Q is as defined above and preferably, irrespectively of its occurrence, selected from a single bond, NR^{9a}, CH₂, and NR^{9a}CH₂.

In this group, particularly preferred are compounds of formula (I), wherein
R³ is selected from the group consisting of hydrogen, halogen, CN, C₁-C₆-alkyl, in particular C₁-C₃-alkyl, such as methyl, ethyl and isopropyl, and C₁-C₆-haloalkyl, in particular C₁-C₃-haloalkyl, such as fluoromethyl, difluoromethyl, trifluoromethyl, 1,1-difluoroethyl, 2-fluoroethyl, 2,2-difluoroethyl or 2,2,2-trifluoroethyl;
R⁴ is selected from the group consisting of hydrogen, halogen, CN, NR^{9a}R^{9b}, C₁-C₆-alkyl, C₁-C₆-haloalkyl, or Q-phenyl, where Q is as defined above and Q is in particular a single bond, NR^{9a}, CH₂, or NR^{9a}CH₂ and where phenyl is unsubstituted or substituted with 1, 2, 3, 4 or 5, in particular 1, 2 or 3 identical or different substituents R¹⁰; and
R⁵ is selected from the group consisting of CN, C₁-C₄-cyanoalkyl, NR^{9a}R^{9b}, C₁-C₆-alkyl, C₁-C₆-haloalkyl, C(═O)OR⁸, C(═O)NR^{9a}R^{9b}, C(═S)NR^{9a}R^{9b}, C(═O)R^{7a}, C(═S)R^{7a}, Q-phenyl, where phenyl is unsubstituted or substituted with 1, 2, 3, 4 or 5 identical or different substituents R¹⁰, or Q-Het^{#}, where Het^{#} is unsubstituted or substituted with 1, 2, 3, or 4 identical or different substituents R¹⁰,
   and where Q, irrespectively of its occurrence, is a single bond, NR^{9a}, CH₂, or NR^{9a}CH₂.

Likewise, particularly preferred are compounds of formula (I), wherein
R³ is selected from the group consisting of hydrogen, halogen, CN, C₁-C₆-alkyl, in particular C₁-C₃-alkyl, such as methyl, ethyl and isopropyl, and C₁-C₆-haloalkyl, in particular C₁-C₃-haloalkyl, such as fluoromethyl, difluoromethyl, trifluoromethyl, 1,1-difluoroethyl, 2-fluoroethyl, 2,2-difluoroethyl or 2,2,2-trifluoroethyl;
R⁴ is selected from the group consisting of hydrogen, halogen, CN, NR^{9a}R^{9b}, C₁-C₆-alkyl, C₁-C₆-haloalkyl, or Q-phenyl, where Q is as defined above and Q is in particular a single bond, NR^{9a}, CH₂, or NR^{9a}CH₂ and where phenyl is unsubstituted or substituted with 1, 2, 3, 4 or 5, in particular 1, 2 or 3 identical or different substituents R¹⁰; and
R⁵ is selected from the group consisting of C₁-C₄-alkoxy-C₁-C₄-alkyl, C₁-C₄-alkthio-C₁-C₄-alkyl, C₃-C₆-cycloalkyl-C₁-C₄-alkyl, C₃-C₈-cycloalkyl, where cycloalkyl in the last to radicals is unsubstituted or optionally substituted with 1, 2, 3 or 4 C₁-C₄-alkyl radicals.

Even more preferred are compounds of formula (I), wherein
R³ is fluorine or CN;
R⁴ is selected from the group consisting of hydrogen, fluorine, chlorine, CN, C₁-C₄-alkyl, such as methyl, ethyl, n-propyl and isopropyl, and C₁-C₄-haloalkyl, such as fluoromethyl, difluoromethyl, trifluoromethyl, 1,1-difluoroethyl, 2-fluoroethyl, 2,2-difluoroethyl or 2,2,2-trifluoroethyl; and
R⁵ is selected from the group consisting of CN, C₁-C₄-cyanoalkyl, C₁-C₄-alkoxy-C₁-C₄-alkyl, C₁-C₄-alkthio-C₁-C₄-alkyl, NR^{9a}R^{9b}, C₁-C₆-alkyl, such as methyl, ethyl, n-propyl, isopropyl, n-butyl, 2-butyl, 2-methylpropyl, 1,1-dimethylethyl, n-pentyl, 1-methylbutyl, 1,2-dimethylbutyl, 1-ethylpentyl or 1,2,2-trimethylpropyl, C₁-C₆-haloalkyl, C(═O)OR⁸, C(═O)NR^{9a}R^{9b}, C(═S)NR^{9a}R^{9b}, C(═O)R^{7a}, C(═S)R^{7a},
   C₃-C₆-cycloalkyl-C₁-C₄-alkyl and C₃-C₈-cycloalkyl, where cycloalkyl in the last two radicals is unsubstituted or optionally substituted with 1, 2, 3 or 4 C₁-C₄-alkyl radicals,
   Q-phenyl, where phenyl is unsubstituted or substituted with 1, 2, 3, 4 or 5, in particular 1, 2 or 3, identical or different substituents R¹⁰, or Q-Het^{#}, where Het^{#} is unsubstituted or substituted with 1, 2, 3, or 4, in particular 1 or 2 identical or different substituents R¹⁰, and
   where Q is as defined above and preferably, irrespectively of its occurrence, selected from a single bond, NR^{9a}, CH₂, and NR^{9a}CH₂.

In this group, even more preferred are compounds of formula (I), wherein
R³ fluorine or CN;
R⁴ is selected from the group consisting of hydrogen, fluorine, chlorine, CN, C₁-C₄-alkyl, such as methyl, ethyl, n-propyl and isopropyl, and C₁-C₄-haloalkyl, such as fluoromethyl, difluoromethyl, trifluoromethyl, 1,1-difluoroethyl, 2-fluoroethyl, 2,2-difluoroethyl or 2,2,2-trifluoroethyl; and
R⁵ is selected from the group consisting of CN, NR^{9a}R^{9b}, C₁-C₆-alkyl, such as methyl, ethyl, n-propyl, isopropyl, n-butyl, 2-butyl or 2-methylpropyl, C₁-C₆-haloalkyl, C(═O)OR⁸,
   C(═O)NR^{9a}R^{9b}, C(═S)NR^{9a}R^{9b}, C(═O)R^{7a}, C(═S)R^{7a}, Q-phenyl, where phenyl is unsubstituted or substituted with 1, 2, 3, 4 or 5, in particular 1, 2 or 3, identical or different substituents R¹⁰, or Q-Het^{#}, where Het^{#} is unsubstituted or substituted with 1, 2, 3, or 4, in particular 1 or 2 identical or different substituents R¹⁰, and where Q is as defined above and preferably, irrespectively of its occurrence, selected from a single bond, NR^{9a}, CH₂, and NR^{9a}CH₂.

Likewise, even more preferred are compounds of formula (I), wherein
R³ fluorine or CN;
R⁴ is selected from the group consisting of hydrogen, fluorine, chlorine, CN, C₁-C₄-alkyl, such as methyl, ethyl, n-propyl and isopropyl, and C₁-C₄-haloalkyl, such as fluoromethyl, difluoromethyl, trifluoromethyl, 1,1-difluoroethyl, 2-fluoroethyl, 2,2-difluoroethyl or 2,2,2-trifluoroethyl; and
R⁵ is selected from the group consisting of C₁-C₄-cyanoalkyl, C₁-C₄-alkoxy-C₁-C₄-alkyl, C₁-C₄-alkthio-C₁-C₄-alkyl, C₃-C₆-cycloalkyl-C₁-C₄-alkyl, C₃-C₈-cycloalkyl, where cycloalkyl in the last to radicals is unsubstituted or optionally substituted with 1, 2, 3 or 4 C₁-C₄-alkyl radicals.

Especially preferred are compounds of formula (I), wherein
R³ is fluorine or CN;
R⁴ is selected from the group consisting of hydrogen and fluorine;
R⁵ cyanomethyl, C₁-C₄-alkoxy-C₁-C₄-alkyl, C₁-C₄-alkthio-C₁-C₄-alkyl, NR^{9a}R^{9b}, C₁-C₆-alkyl, C₁-C₄-haloalkyl, C(═O)OR⁸, C(═O)NR^{9a}R^{9b}, C(═O)R^{7a},
   C₃-C₆-cycloalkyl-C₁-C₄-alkyl, C₃-C₈-cycloalkyl, where cycloalkyl in the last to radicals is unsubstituted or optionally substituted with 1, 2, 3 or 4 C₁-C₄-alkyl radicals,
   Q-phenyl, where phenyl is unsubstituted or substituted with 1, 2, 3, 4 or 5 identical or different substituents R¹⁰, or Q-Het^{#}, where Het^{#} is unsubstituted or substituted with 1, 2, 3, or 4 identical or different substituents R¹⁰,
   and where Q, irrespectively of its occurrence, is a single bond, NH, N(C₁-C₄-alkyl), CH₂, or N(C₁-C₄-alkyl)CH₂.

In this group, especially preferred are compounds of the formula (I), wherein
R³ fluorine or CN;
R⁴ is selected from the group consisting of hydrogen and fluorine; and
R⁵ is selected from the group consisting of cyanomethyl, NR^{9a}R^{9b}, C₁-C₆-alkyl, C₁-C₄-haloalkyl, C(═O)OR⁸, C(═O)NR^{9a}R^{9b}, C(═O)R^{7a}, Q-phenyl, where phenyl is unsubstituted or substituted with 1, 2, 3, 4 or 5 identical or different substituents R¹⁰, or Q-Het^{#}, where Het^{#} is unsubstituted or substituted with 1, 2, 3, or 4 identical or different substituents R¹⁰, and where Q, irrespectively of its occurrence, is a single bond, NH, N(C₁-C₄-alkyl), CH₂, NHCH₂ or N(C₁-C₄-alkyl)CH₂.

In this group, likewise especially preferred are compounds of the formula (I), wherein
R³ fluorine or CN;
R⁴ is selected from the group consisting of hydrogen and fluorine; and
R⁵ is selected from the group consisting of C₁-C₄-alkoxy-C₁-C₄-alkyl, C₁-C₄-alkthio-C₁-C₄-alkyl, C₃-C₆-cycloalkyl-C₁-C₄-alkyl and C₃-C₈-cycloalkyl, where cycloalkyl in the last two radicals is unsubstituted or optionally substituted with 1, 2, 3 or 4 C₁-C₄-alkyl radicals.

In context of R⁵, Het^{#} is preferably 5- or 6-membered hetaryl such as pyridyl, thienyl, furyl, pyrrolyl, thiazolyl, isothiazolyl, pyrazolyl, imidazolyl, oxazolyl or isoxazolyl, which is unsubstituted or substituted by 1, 2 or 3 radicals R¹⁰. In a further embodiment, Het^{#} is a 8-, 9- or 10-membered hetaryl such as imidazo[1,2-a]pyridyl. In a further alternative embodiment, Het^{#} is preferably a 5-, 6- or 7-membered saturated heterocyclic ring comprising 1 or 2 heteroatoms or heteroatom groups selected from N, O, S, NO, SO and SO₂, as ring members such as tetrahydrofuryl, tetrahydrothienyl, sulfur oxidized tetrahydrothienyl, tetrahydropyranyl, tetrahydrothiopyranyl or sulphur oxidized tetrahydrothiopyranyl, which is unsubstituted or substituted by 1, 2 or 3 radicals R¹⁰. In this context, R¹⁰ is preferably selected from the group consisting of halogen, such as bromine, chlorine or fluorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy.

In context of R⁵, the radical R^{7a} is preferably selected from the group consisting of hydrogen, C₁-C₄-alkyl, C₁-C₄-haloalkyl, C₃-C₆-cycloalkyl, C₃-C₆-cycloalkyl-C₁-C₄-alkyl, phenyl and benzyl, where the phenyl ring in the last two radicals is unsubstituted or substituted by 1, 2 or 3 identical or different radicals selected from the group consisting of halogen, such as chlorine or fluorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy.

In context of R⁵, the radical R⁸ is preferably selected from the group consisting of C₁-C₄-alkyl, C₁-C₄-haloalkyl, C₃-C₆-cycloalkyl, C₃-C₆-cycloalkyl-C₁-C₄-alkyl, phenyl and benzyl, where the phenyl ring in the last two radicals is unsubstituted or substituted by 1, 2 or 3 identical or different radicals selected from the group consisting of halogen, such as chlorine or fluorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy.

In context of R⁴ and R⁵, the radicals R^{9a} and R^{9b} are preferably selected from the group consisting of hydrogen, C₁-C₄-alkyl, such as methyl, ethyl, n-propyl, isopropyl, n-butyl, 2-butyl or isobutyl, and C₁-C₄-haloalkyl, such as fluoromethyl, difluoromethyl, trifluoromethyl, 1,1-difluoroethyl, 2-fluoroethyl, 2,2-difluoroethyl or 2,2,2-trifluoroethyl, or NR^{9a}R^{9b} may also be a saturated N-bound 3-, 4-, 5- or 6-membered heterocycle, which in addition to the nitrogen atom may have 1 further heteroatom as ring members, which is selected from 0 and N and where the N-bound 3-, 4-, 5- or 6-membered heterocycle may be unsubstituted or carry 1, 2, 3 or 4 radi-cats selected from C₁-C₄-alkyl and C₁-C₄-haloalkyl. Examples of such radicals NR^{9a}R^{9b} include, but are not limited to methylamino, ethylamino, n-propylamino, isopropylamino, n-butylamino, 2-butylamino, isobutylamino, dimethylamino, diethylamino, di-n-propylamino, di-n-butylamino, N-methyl-N-ethylamino, N-methyl-N-propylamino, N-methyl-N-n-propylamino, N-methyl-N-isopropylamino, N-methyl-N-n-butylamino, N-methyl-N-2-butylamino, N-methyl-N-isobutylamino, 1-pyrrolidinyl, 1-piperidinyl, 1-piperazinyl, 4-methyl-1-piperazinly and 4-morpholinyl.

In context of R⁴ and R⁵, the radical R¹⁰ is preferably selected from the group consisting of halogen, such as chlorine or fluorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy.

Preferred are compounds of formula (I), wherein W¹-W²-W³-W⁴ represents a carbon chain group connected to N and C═N, which is selected from the group consisting of CR^{w6}═CR^{w5}-CR^{w4}═CR^{w3}, CR^{w6}═CR^{w5}-CHR^{w4}-CHR^{w3}, CHR_{w6}-CHR_{w5}-CHR_{w4}-CHR_{w3}, CHR^{w6}-CHR^{w5}-CR^{w4}═CR^{w3}, and CHR^{w6}-CHR^{w5}-CHR^{w4}-CHR^{w3}, where in the five aforementioned radicals the carbon atom which carries R^{w6} is bound to the nitrogen atom and where R^{w3}, R^{w4}, R^{w5} and R^{w6}, independently of each other, have one of the meanings given for R^{w}. In this context, R^{w} is preferably selected from the group consisting of hydrogen, halogen, such as fluorine or chlorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy. Preferably, at most one of R^{w3}, R^{w4}, R^{w5} and R^{w6} is different from hydrogen.

In a particularly preferred group of embodiments R^{w3}, R^{w4} and R^{w6} are hydrogen while R^{w5} has one of the meanings given for R^{w}, and where R^{w5} is in particular selected from the group consisting of hydrogen, halogen, such as fluorine or chlorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy.

In another particularly preferred group of embodiments R^{w3}, R^{w4} and R^{w5} are hydrogen while R^{w6} has one of the meanings given for R^{w}, and where R^{w6} is in particular selected from the group consisting of hydrogen, halogen, such as fluorine or chlorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy.

Especially, all of R_{w3}, R_{w4}, R^{w5} and R^{w6} are hydrogen.

Preferred are compounds of formula (I), wherein the moiety of the formula (A)

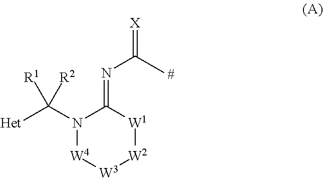

represents a radical selected from the group consisting of W.Het-1, W.Het-2, W.Het-3, W.Het-4, W.Het-5, W.Het-6, W.Het-7, W.Het-8, W.Het-9, W.Het-10, W.Het-11 and W.Het-12, in where the radical of formula (A) is in particular selected from the radicals W.Het-1, W.Het-2, W.Het-5, W.Het-6, W.Het-9 and W.Het-10.

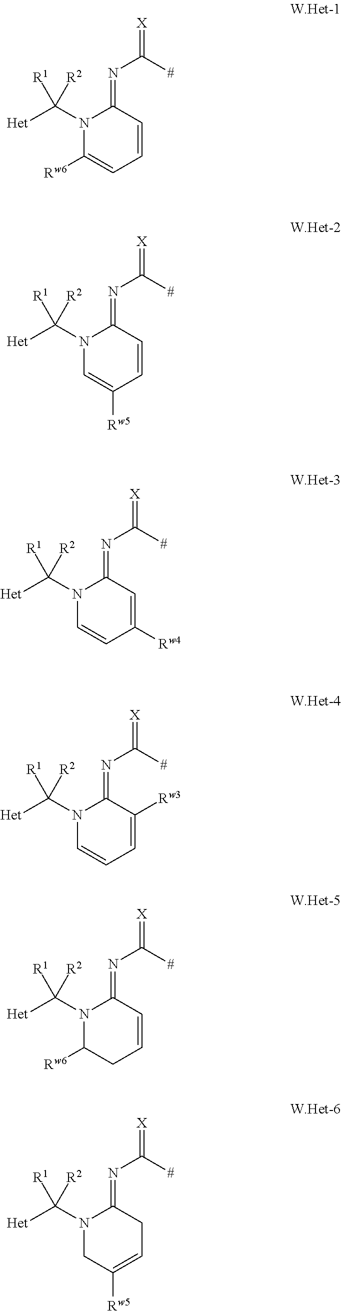

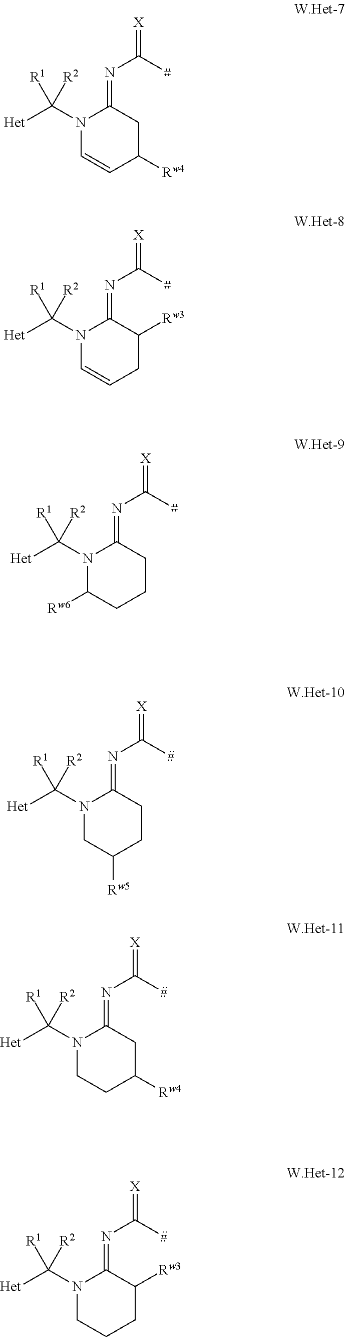

wherein # denotes the bond to the remainder of the molecule and where R¹, R² and Het are as defined herein and where R¹, R² and Het, individually or in combination have the meanings given as preferred meanings, and wherein R^{w3}, R^{w4}, R^{w5} and R^{w6} are as defined above and in particular selected from the group consisting of hydrogen, halogen, such as fluorine or chlorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy.

Particularly preferred are compounds of formula (I), wherein the moiety of the formula A is selected from the group consisting of W.Het-1, W.Het-5 and W.Het-9, wherein R^{w6} is as defined above and in particular selected from the group consisting of hydrogen, halogen, such as fluorine or chlorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy and where R^{w6} is especially hydrogen.

Likewise, particularly preferred are compounds of formula (I), wherein the moiety of the formula A is selected from the group consisting of W.Het-2, W.Het-6 and W.Het-10, wherein R^{w5} is as defined above and in particular selected from the group consisting of hydrogen, halogen, such as fluorine or chlorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy and where R^{w5} is especially hydrogen.

Likewise, particularly preferred are compounds of formula (I), wherein the moiety of the formula A is selected from the group consisting of W.Het-1, W.Het-2, W.Het-3 and W.Het-4, especially from the group consisting of W.Het-1 and W.Het-2, wherein R^{w3}, R^{w4}, R^{w5} and R^{w6}, independently of each other, are as defined above and in particular selected from the group consisting of hydrogen, halogen, such as fluorine or chlorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy and where at most one of R^{w3}, R^{w4}, R^{w5} and R^{w6} are especially hydrogen.

In the moieties W.Het-1, W.Het-2, W.Het-3, W.Het-4, W.Het-5, W.Het-6, W.Het-7, W.Het-8, W.Het-9, W.Het-10, W.Het-11 and W.Het-12, the heterocycle Het is in particular selected from the group consisting of the radicals of formulae Het-1 to Het-24, as defined above, and in particular selected from the group consisting of the radicals of the formulae Het-1 or Het-1a, Het-10 or Het-10a, Het-11a, Het-23 or Het-23a and Het-24, in particular the radicals of the formulae Het-1 or Het-1a, Het-11a and Het-24.

In the moieties W.Het-1, W.Het-2, W.Het-3, W.Het-4, W.Het-5, W.Het-6, W.Het-7, W.Het-8, W.Het-9, W.Het-10, W.Het-11 and W.Het-12, the radicals R¹ and R² are, independently from each other, in particular selected from the group consisting of hydrogen, halogen, such as fluorine or chlorine, CN, C₁-C₆-alkyl, in particular C₁-C₄-alkyl, such as methyl, ethyl, n-propyl or isopropyl, C₃-C₆-cycloalkyl, such as cyclopropyl or cyclobutyl, C₁-C₆-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, or C₃-C₆-halocycloalkyl such as 1-fluorocyclopropyl or 2,2-difluorocyclopropyl, or R¹ and R² may together be ═CR¹³R¹⁴ or R¹ and R² form, together with the carbon atom, which they attached to, a 3- to 5-membered saturated carbocyclic ring such as cyclopropyl, cyclobutyl or cyclopentyl.

In particular embodiments of moieties W.Het-1, W.Het-2, W.Het-3, W.Het-4, W.Het-5, W.Het-6, W.Het-7, W.Het-8, W.Het-9, W.Het-10, W.Het-11 and W.Het-12, the radicals R¹ and R² are, independently from each other, more particularly selected from the group consisting of hydrogen, halogen, cyano, C₁-C₃-alkyl, such as methyl ethyl or isopropyl, or C₁-C₃-haloalkyl such as fluoromethyl, difluoromethyl, trifluoromethyl, 1,1-difluoroethyl, 2-fluoroethyl, 2,2-difluoroethyl or 2,2,2-trifluoroethyl, where in particular at least one of the radicals R¹ and R² is hydrogen.

Especially, R¹ and R² in the moieties W.Het-1, W.Het-2, W.Het-3, W.Het-4, W.Het-5, W.Het-6, W.Het-7, W.Het-8, W.Het-9, W.Het-10, W.Het-11 and W.Het-12 are both hydrogen.

A particular group 1 of embodiments relates to compounds of the formula (I), to their stereoisomers, their tautomers and their salts, wherein the moiety of formula (A) represents a radical selected from the group consisting of W.Het-1, wherein Het is selected from the group consisting of radicals of the formulae Het-1 or Het-1a, Het-10 or Het-10a, Het-11a, Het-23 or Het-23a and Het-24, in particular radicals of formulae Het-1, Het-11a and Het-24.

A further particular group 2 of embodiments relates to compounds of the formula (I), to their stereoisomers, their tautomers and their salts, wherein the moiety of formula (A) represents a radical selected from the group consisting of W.Het-2, wherein Het is selected from the group consisting of radicals of the formulae Het-1 or Het-1a, Het-10 or Het-10a, Het-11a, Het-23 or Het-23a and Het-24, in particular radicals of formulae Het-1, Het-11a and Het-24.

A further particular group 3 of embodiments relates to compounds of the formula (I), to their stereoisomers, their tautomers and their salts, wherein the moiety of formula (A) represents a radical selected from the group consisting of W.Het-5, wherein Het is selected from the group consisting of radicals of the formulae Het-1 or Het-1a, Het-10 or Het-10a, Het-11a, Het-23 or Het-23a and Het-24, in particular radicals of formulae Het-1, Het-11a and Het-24.

A further particular group 4 of embodiments relates to compounds of the formula (I), to their stereoisomers, their tautomers and their salts, wherein the moiety of formula (A) represents a radical selected from the group consisting of W.Het-6, wherein Het is selected from the group consisting of radicals of the formulae Het-1 or Het-1a, Het-10 or Het-10a, Het-11a, Het-23 or Het-23a and Het-24, in particular radicals of formulae Het-1, Het-11a and Het-24.

A further particular group 5 of embodiments relates to compounds of the formula (I), to their stereoisomers, their tautomers and their salts, wherein the moiety of formula (A) represents a radical selected from the group consisting of W.Het-9, wherein Het is selected from the group consisting of radicals of the formulae Het-1 or Het-1a, Het-10 or Het-10a, Het-11a, Het-23 or Het-23a and Het-24, in particular radicals of formulae Het-1, Het-11a and Het-24.

A further particular group 6 of embodiments relates to compounds of the formula (I), to their stereoisomers, their tautomers and their salts, wherein the moiety of formula (A) represents a radical selected from the group consisting of W.Het-10, wherein Het is selected from the group consisting of radicals of the formulae Het-1 or Het-1a, Het-10 or Het-10a, Het-11a, Het-23 or Het-23a and Het-24, in particular radicals of formulae Het-1, Het-11a and Het-24.

A special group 1a of embodiments relates to compounds of the formula (I), to their stereoisomers, their tautomers and their salts, wherein the moiety of formula (A) represents a radical selected from the group consisting of W.Het-1, wherein Het is a radical of formulae Het-1a, Het-10a, Het-11a or Het-23a, in particular a radical of formula Het-1a.

A further special group 2a of embodiments relates to compounds of the formula (I), to their stereoisomers, their tautomers and their salts, wherein the moiety of formula (A) represents a radical selected from the group consisting of W.Het-2, wherein Het is a radical of formulae Het-1a, Het-10a, Het-11a or Het-23a, in particular a radical of formulae Het-1a.

A further special group 3a of embodiments relates to compounds of the formula (I), to their stereoisomers, their tautomers and their salts, wherein the moiety of formula (A) represents a radical selected from the group consisting of W.Het-5, wherein Het is a radical of formulae Het-1a, Het-10a, Het-11a or Het-23a, in particular a radical of formula Het-1a.

A further special group 4a of embodiments relates to compounds of the formula (I), to their stereoisomers, their tautomers and their salts, wherein the moiety of formula (A) represents a radical selected from the group consisting of W.Het-6, wherein Het is a radical of formulae Het-1a, Het-10a, Het-11a or Het-23a, in particular a radical of formula Het-1a.

A further special group 5a of embodiments relates to compounds of the formula (I), to their stereoisomers, their tautomers and their salts, wherein the moiety of formula (A) represents a radical selected from the group consisting of W.Het-9, wherein Het is a radical of formulae Het-1a, Het-10a, Het-11a or Het-23a, in particular a radical of formula Het-1a.

A further special group 6a of embodiments relates to compounds of the formula (I), to their stereoisomers, their tautomers and their salts, wherein the moiety of formula (A) represents a radical selected from the group consisting of W.Het-10, wherein Het is a radical of formulae Het-1a, Het-10a, Het-11a or Het-23a, in particular a radical of formula Het-1a.

In embodiments 1, 3, 5, 1a, 3a and 5a the radical R^{w6} is as defined above and in particular selected from the group consisting of hydrogen, halogen, such as fluorine or chlorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy. In embodiments 1, 3, 5, 1a, 3a and 5a the radical R^{w6} is especially hydrogen.

In embodiments 2, 4, 6, 2a, 4a and 6a the radical R^{w5} is as defined above and in particular selected from the group consisting of hydrogen, halogen, such as fluorine or chlorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy. In embodiments 2, 4, 6, 2a, 4a and 6a the radical R^{w5} is especially hydrogen.

In embodiments 1, 2, 3, 4, 5, 6, 1a, 2a, 3a, 4a, 5a and 6a the radicals R¹ and R² are, independently from each other, in particular selected from the group consisting of hydrogen, halogen, such as fluorine or chlorine, CN, C₁-C₆-alkyl, in particular C₁-C₄-alkyl, such as methyl, ethyl, n-propyl or isopropyl, C₃-C₆-cycloalkyl, such as cyclopropyl or cyclobutyl, C₁-C₆-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, or C₃-C₆-halocycloalkyl such and R² form, together with the carbon atom, which they attached to, a 3- to 5 membered saturated carbocyclic ring such as cyclopropyl, cyclobutyl or cyclopentyl.

In embodiments 1, 2, 3, 4, 5, 6, 1a, 2a, 3a, 4a, 5a and 6a the radicals R¹ and R² are, independently from each other, more particularly selected from the group consisting of hydrogen, halogen, cyano, C₁-C₃-alkyl, such as methyl ethyl or isopropyl, or C₁-C₃-haloalkyl such as fluoromethyl, difluoromethyl, trifluoromethyl, 1,1-difluoroethyl, 2-fluoroethyl, 2,2-difluoroethyl or 2,2,2-trifluoroethyl, where in particular at least one of the radicals R¹ and R² is hydrogen and where especially both R¹ and R² are hydrogen.

In the compounds of formula (I), where the moiety of formula (A) is selected from the moieties of formulae W.Het-1, W.Het-2, W.Het-3, W.Het-4, W.Het-5, W.Het-6, W.Het-7, W.Het-8, W.Het-9, W.Het-10, W.Het-11 and W.Het-12 and likewise in the embodiments 1, 2, 3, 4, 5, 6, 1a, 2a, 3a, 4a, 5a and 6a, the variables R¹, R², R³, R⁴ and R⁵ are as defined above and in particular have the preferred meanings.

In the compounds of formula (I), where the moiety of formula (A) is selected from the moieties of formulae W.Het-1, W.Het-2, W.Het-3, W.Het-4, W.Het-5, W.Het-6, W.Het-7, W.Het-8, W.Het-9, W.Het-10, W.Het-11 and W.Het-12 and likewise in the embodiments 1, 2, 3, 4, 5, 6, 1a, 2a, 3a, 4a, 5a and 6a, the variables R¹, R², R³, R⁴ and R⁵, independently of each other or in particular in combination, in particular have the following meanings:
R¹, R² are independently from each other selected from the group consisting of hydrogen, halogen, CN, C₁-C₆-alkyl, C₃-C₆-cycloalkyl, C₁-C₆-haloalkyl, C₃-C₆-halocycloalkyl;
   or R¹ and R² may together be ═CR¹³R¹⁴;
   or R¹ and R² form, together with the carbon atom, which they attached to, a 3- to 5-membered saturated carbocyclic ring.
R³ is selected from the group consisting of hydrogen, halogen, CN, C₁-C₆-alkyl and, C₁-C₆-haloalkyl.
R⁴ is hydrogen, halogen, CN, NR^{9a}R^{9b}, C₁-C₆-alkyl, C₁-C₆-haloalkyl, or Q-phenyl, where Q is as defined above and where phenyl is unsubstituted or substituted with 1, 2, 3, 4 or 5 identical or different substituents R¹⁰, and
R⁵ is —CN, C₁-C₄-cyanoalkyl, C₁-C₄-alkoxy-C₁-C₄-alkyl, C₁-C₄-alkthio-C₁-C₄-alkyl, NR^{9a}R^{9b}, C₁-C₆-alkyl, C₁-C₆-haloalkyl, C(═O)OR⁸, C(═O)NR^{9a}R^{9b}, C(═S)NR^{9a}R^{9b}, C(═O)R^{7a}, C(═S)R^{7a}, C₃-C₆-cycloalkyl-C₁-C₄-alkyl, C₃-C₈-cycloalkyl, where cycloalkyl in the last two radicals is unsubstituted or optionally substituted with 1, 2, 3 or 4 C₁-C₄-alkyl radicals,
   Q-phenyl, where phenyl is unsubstituted or substituted with 1, 2, 3, 4 or 5 identical or different substituents R¹⁰, or Q-Het^{#}, where Het^{#} is unsubstituted or substituted with 1, 2, 3, or 4 identical or different substituents R¹⁰, and where Q, irrespectively of its occurrence, is as defined above;
   or
R⁴ and R⁵ together form a moiety selected from Alk, S(O)ₙ-Alk, NR^{9c}-Alk,
   S(O)ₙ-Alk′-S(O)ₙ, (O)-Alk′-O and NR^{9c}-Alk′-NR^{9d}, where Alk represents a saturated or unsaturated 2-, 3- or 4-membered carbon chain group, which is unsubstituted or carries 1, 2, 3 or 4 radicals R⁷ or a fused phenylene ring, where the fused phenyl ring is unsubstituted or substituted with 1, 2, 3 or 4 radicals R¹⁰ and where Alk′ is CH₂ or has one of the meanings given for Alk.

Among these, in the compounds of formula (I), where the moiety of formula (A) is selected from the moieties of formulae W.Het-1, W.Het-2, W.Het-3, W.Het-4, W.Het-5, W.Het-6, W.Het-7, W.Het-8, W.Het-9, W.Het-10, W.Het-11 and W.Het-12 and likewise in the embodiments 1, 2, 3, 4, 5, 6, 1a, 2a, 3a, 4a, 5a and 6a, the variables R¹, R², R³, R⁴ and R⁵, independently of each other or in particular in combination, in particular have the following meanings:
R¹ and R² are, independently from each other, selected from the group consisting of hydrogen, halogen, such as fluorine or chlorine, CN, C₁-C₆-alkyl, in particular C₁-C₄-alkyl, such as methyl, ethyl, n-propyl or isopropyl, C₃-C₆-cycloalkyl, such as cyclopropyl or cyclobutyl, C₁-C₆-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, or C₃-C₆-halocycloalkyl such as 1-fluorocyclopropyl or 2,2-difluorocyclopropyl, or R¹ and R² may together be ═CR¹³R¹⁴ or R¹ and R² form, together with the carbon atom, which they attached to, a 3- to 5 membered saturated carbocyclic ring such as cyclopropyl, cyclobutyl or cyclopentyl;
R³ is selected from the group consisting of hydrogen, halogen, CN, C₁-C₆-alkyl, in particular C₁-C₃-alkyl, such as methyl, ethyl and isopropyl, and C₁-C₆-haloalkyl, in particular C₁-C₃-haloalkyl, such as fluoromethyl, difluoromethyl, trifluoromethyl, 1,1-difluoroethyl, 2-fluoroethyl, 2,2-difluoroethyl or 2,2,2-trifluoroethyl;
R⁴ is selected from the group consisting of hydrogen, halogen, CN, NR^{9a}R^{9b}, C₁-C₆-alkyl, C₆-haloalkyl, or Q-phenyl, where Q is as defined above and Q is in particular a single bond, NR^{9a}, CH₂, or NR^{9a}CH₂ and where phenyl is unsubstituted or substituted with 1, 2, 3, 4 or 5, in particular 1, 2 or 3 identical or different substituents R¹⁰; and
R⁵ is selected from the group consisting of CN, NR^{9a}R^{9b}, C₁-C₆-alkyl, such as methyl, ethyl, n-propyl, isopropyl, n-butyl, 2-butyl or 2-methylpropyl, C₁-C₆-haloalkyl, C(═O)OR⁸, C(═O)NR^{9a}R^{9b}, C(═S)NR^{9a}R^{9b}, C(═O)R^{7a}, C(═S)R^{7a}, Q-phenyl, where phenyl is unsubstituted or substituted with 1, 2, 3, 4 or 5, in particular 1, 2 or 3, identical or different substituents R¹⁰, or Q-Het^{#}, where Het^{#} is unsubstituted or substituted with 1, 2, 3, or 4, in particular 1 or 2 identical or different substituents R¹⁰, and where Q is as defined above and preferably, irrespectively of its occurrence, selected from a single bond, NR^{9a}, CH₂, and NR^{9a}CH₂;
   or
R⁴ and R⁵ together form a moiety selected from Alk, S(O)ₙ-Alk, NR^{9c}-Alk,
   S(O)ₙ-Alk′-S(O)ₙ, S(O)ₙ-Alk′-O and NR^{9c}-Alk′-NR^{9d}, where Alk represents a saturated or unsaturated 2-, 3- or 4-membered carbon chain group, which is unsubstituted or carries 1, 2, 3 or 4 radicals R⁷ or a fused phenylene ring, where the fused phenyl ring is unsubstituted or substituted with 1, 2, 3 or 4 radicals R¹⁰ and where Alk′ is CH₂ or has one of the meanings given for Alk.

In the compounds of formula (I), where the moiety of formula (A) is selected from the moieties of formulae W.Het-1, W.Het-2, W.Het-3, W.Het-4, W.Het-5, W.Het-6, W.Het-7, W.Het-8, W.Het-9, W.Het-10, W.Het-11 and W.Het-12 and likewise in the embodiments 1, 2, 3, 4, 5, 6, 1a, 2a, 3a, 4a, 5a and 6a, the variables R¹, R², R³, R⁴ and R⁵, independently of each other or in particular in combination, more particularly have the following meanings:
R¹ and R² are, independently from each other, selected from the group consisting of hydrogen, halogen, cyano, C₁-C₃-alkyl, such as methyl ethyl or isopropyl, or C₁-C₃-haloalkyl such as fluoromethyl, difluoromethyl, trifluoromethyl, 1,1-difluoroethyl, 2-fluoroethyl, 2,2-difluoroethyl or 2,2,2-trifluoroethyl, where in particular at least one of the radicals R¹ and R² is hydrogen and where especially both R¹ and R² are hydrogen;
R³ fluorine or CN;
R⁴ is selected from the group consisting of hydrogen, fluorine, chlorine, CN, C₁-C₄-alkyl, such as methyl, ethyl, n-propyl and isopropyl, and C₁-C₄-haloalkyl, such as fluoromethyl, difluoromethyl, trifluoromethyl, 1,1-difluoroethyl, 2-fluoroethyl, 2,2-difluoroethyl or 2,2,2-trifluoroethyl; and
R⁵ is selected from the group consisting of CN, NR^{9a}R^{9b}, C₁-C₆-alkyl, such as methyl, ethyl, n-propyl, isopropyl, n-butyl, 2-butyl or 2-methylpropyl, C₁-C₆-haloalkyl, C(═O)OR⁸, C(═O)NR^{9a}R^{9b}, C(═S)NR^{9a}R^{9b}, C(═O)R^{7a}, C(═S)R^{7a}, Q-phenyl, where phenyl is unsubstituted or substituted with 1, 2, 3, 4 or 5, in particular 1, 2 or 3, identical or different substituents R¹⁰, or Q-Het^{#}, where Het^{#} is unsubstituted or substituted with 1, 2, 3, or 4, in particular 1 or 2 identical or different substituents R¹⁰, and where Q is as defined above and preferably, irrespectively of its occurrence, selected from a single bond, NR^{9a}, CH₂, and NR^{9a}CH₂.

Likewise preference is given to compounds (I), where the moiety of formula (A) is selected from the moieties of formulae W.Het-1, W.Het-2, W.Het-3, W.Het-4, W.Het-5, W.Het-6, W.Het-7, W.Het-8, W.Het-9, W.Het-10, W.Het-11 and W.Het-12 and likewise in the embodiments 1, 2, 3, 4, 5, 6, 1a, 2a, 3a, 4a, 5a and 6a, the variables R¹, R², R³, R⁴ and R⁵, independently of each other or in particular in combination, in particular have the following meanings:
R¹ and R² are, independently from each other, selected from the group consisting of hydrogen, halogen, such as fluorine or chlorine, CN, C₁-C₆-alkyl, in particular C₁-C₄-alkyl, such as methyl, ethyl, n-propyl or isopropyl, C₃-C₆-cycloalkyl, such as cyclopropyl or cyclobutyl, C₁-C₆-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, or C₃-C₆-halocycloalkyl such as 1-fluorocyclopropyl or 2,2-difluorocyclopropyl, or R¹ and R² may together be ═CR¹³R¹⁴ or R¹ and R² form, together with the carbon atom, which they attached to, a 3- to 5-membered saturated carbocyclic ring such as cyclopropyl, cyclobutyl or cyclopentyl;
R³ is selected from the group consisting of hydrogen, halogen, CN, C₁-C₆-alkyl, in particular C₁-C₃-alkyl, such as methyl, ethyl and isopropyl, and C₁-C₆-haloalkyl, in particular C₁-C₃-haloalkyl, such as fluoromethyl, difluoromethyl, trifluoromethyl, 1,1-difluoroethyl, 2-fluoroethyl, 2,2-difluoroethyl or 2,2,2-trifluoroethyl;
R⁴ is selected from the group consisting of hydrogen, halogen, CN, NR^{9a}R^{9b}, C₁-C₆-alkyl, C₆-haloalkyl, or Q-phenyl, where Q is as defined above and Q is in particular a single bond, NR^{9a}, CH₂, or NR^{9a}CH₂ and where phenyl is unsubstituted or substituted with 1, 2, 3, 4 or 5, in particular 1, 2 or 3 identical or different substituents R¹⁰; and
R⁵ is selected from the group consisting of C₁-C₄-cyanoalkyl, C₁-C₄-alkoxy-C₁-C₄-alkyl such as 1-methoxyethyl, 1-ethoxyethyl, 1-isoproxyethyl, 2-isoproxyethyl, 1-isobutoxyethyl, 2-isobutoxyethyl, C₁-C₄-alkylthio-C₁-C₄-alkyl such as 2-methylsulfanylpropyl, C₃-C₆-cyclo-alkyl-C₁-C₄-alkyl and C₃-C₈-cycloalkyl, where cycloalkyl in the last two radicals is unsubstituted or optionally substituted with 1, 2, 3 or 4 C₁-C₄-alkyl radicals, such as cyclopropyl, cyclobutyl, cyclopentyl, cyclohexyl, cycloheptyl, cyclooctyl, bicyclo[3.1.1]heptyl, 4-methylcyclohexyl, 4-ethylcyclohexyl, 6,6-dimethylnorpinan-2-yl, (4-methylcyclohexyl)methyl, or (4-ethylcyclohexyl)methyl.

Likewise, preferred are compounds of formula (I), where the moiety of formula (A) is selected from the moieties of formulae W.Het-1, W.Het-2, W.Het-3, W.Het-4, W.Het-5, W.Het-6, W.Het-7, W.Het-8, W.Het-9, W.Het-10, W.Het-11 and W.Het-12 and likewise in the embodiments 1, 2, 3, 4, 5, 6, 1a, 2a, 3a, 4a, 5a and 6a, wherein the variables R¹, R², R³, R⁴ and R⁵, independently of each other or in particular in combination, more particularly have the following meanings:
R¹ and R² are, independently from each other, selected from the group consisting of hydrogen, halogen, cyano, C₁-C₃-alkyl, such as methyl ethyl or isopropyl, or C₁-C₃-haloalkyl such as fluoromethyl, difluoromethyl, trifluoromethyl, 1,1-difluoroethyl, 2-fluoroethyl, 2,2-difluoroethyl or 2,2,2-trifluoroethyl, where in particular at least one of the radicals R¹ and R² is hydrogen and where especially both R¹ and R² are hydrogen;
R³ fluorine or CN;
R⁴ is selected from the group consisting of hydrogen, fluorine, chlorine, CN, C₁-C₄-alkyl, such as methyl, ethyl, n-propyl and isopropyl, and C₁-C₄-haloalkyl, such as fluoromethyl, difluoromethyl, trifluoromethyl, 1,1-difluoroethyl, 2-fluoroethyl, 2,2-difluoroethyl or 2,2,2-trifluoroethyl; and
R⁵ is selected from the group consisting of C₁-C₄-cyanoalkyl, C₁-C₄-alkoxy-C₁-C₄-alkyl such as 1-methoxyethyl, 1-ethoxyethyl, 1-isoproxyethyl, 2-isoproxyethyl, 1-isobutoxyethyl, 2-isobutoxyethyl, C₁-C₄-alkylthio-C₁-C₄-alkyl such as 2-methylsulfanylpropyl, C₃-C₆-cycloalkyl-C₁-C₄-alkyl and C₃-C₈-cycloalkyl, where cycloalkyl in the last two radicals is unsubstituted or optionally substituted with 1, 2, 3 or 4 C₁-C₄-alkyl radicals, such as cyclopropyl, cyclobutyl, cyclopentyl, cyclohexyl, cycloheptyl, cyclooctyl, bicyclo[3.1.1]heptyl, 4-methylcyclohexyl, 4-ethylcyclohexyl, 6,6-dimethylnorpinan-2-yl, (4-methylcyclohexyl)-methyl, or (4-ethylcyclohexyl)methyl.

In the compounds of formula (I), where the moiety of formula (A) is selected from the moieties of formulae W.Het-1, W.Het-2, W.Het-3, W.Het-4, W.Het-5, W.Het-6, W.Het-7, W.Het-8, W.Het-9, W.Het-10, W.Het-11 and W.Het-12 and likewise in the embodiments 1, 2, 3, 4, 5, 6, 1a, 2a, 3a, 4a, 5a and 6a, the variables R¹, R², R³, R⁴ and R⁵, independently of each other or in particular in combination, especially have the following meanings:
both R¹ and R² are hydrogen;
R³ is fluorine or CN;
R⁴ is selected from the group consisting of hydrogen and fluorine;
R⁵ cyanomethyl, NR^{9a}R^{9b}, C₁-C₆-alkyl, C₁-C₄-haloalkyl, C₁-C₄-alkoxy-C₁-C₄-alkyl, C₁-C₄-alkthio-C₁-C₄-alkyl, C(═O)OR⁸, C(═O)NR^{9a}R^{9b}, C(═O)R^{7a},
   C₃-C₆-cycloalkyl-C₁-C₄-alkyl, C₃-C₈-cycloalkyl, where cycloalkyl in the last two radicals is unsubstituted or optionally substituted with 1, 2, 3 or 4 C₁-C₄-alkyl radicals,
   Q-phenyl, where phenyl is unsubstituted or substituted with 1, 2, 3, 4 or 5 identical or different substituents R¹⁰, or Q-Het^{#}, where Het^{#} is unsubstituted or substituted with 1, 2, 3, or 4 identical or different substituents R¹⁰,
   and where Q, irrespectively of its occurrence, is a single bond, NH, N(C₁-C₄-alkyl), CH₂, or N(C₁-C₄-alkyl)-CH₂.

Amongst these, in the compounds of formula (I), where the moiety of formula (A) is selected from the moieties of formulae W.Het-1, W.Het-2, W.Het-3, W.Het-4, W.Het-5, W.Het-6, W.Het-7, W.Het-8, W.Het-9, W.Het-10, W.Het-11 and W.Het-12 and likewise in the embodiments 1, 2, 3, 4, 5, 6, 1a, 2a, 3a, 4a, 5a and 6a, the variables R¹, R², R³, R⁴ and R⁵, independently of each other or in particular in combination, especially have the following meanings:
R¹ and R² are hydrogen;
R³ fluorine or CN;
R⁴ is selected from the group consisting of hydrogen and fluorine; and
R⁵ is selected from the group consisting of cyanomethyl, NR^{9a}R^{9b}, C₁-C₆-alkyl, C₁-C₄-haloalkyl, C(═O)OR⁸, C(═O)NR^{9a}R^{9b}, C(═O)R^{7a}, Q-phenyl, where phenyl is unsubstituted or substituted with 1, 2, 3, 4 or 5 identical or different substituents R¹⁰, or Q-Het^{#}, where Het^{#} is unsubstituted or substituted with 1, 2, 3, or 4 identical or different substituents R¹⁰, and where Q, irrespectively of its occurrence, is a single bond, NH, N(C₁-C₄-alkyl), CH₂, NHCH₂ or N(C₁-C₄-alkyl)CH₂.

Likewise, in the compounds of formula (I), where the moiety of formula (A) is selected from the moieties of formulae W.Het-1, W.Het-2, W.Het-3, W.Het-4, W.Het-5, W.Het-6, W.Het-7, W.Het-8, W.Het-9, W.Het-10, W.Het-11 and W.Het-12 and likewise in the embodiments 1, 2, 3, 4, 5, 6, 1a, 2a, 3a, 4a, 5a and 6a, the variables R¹, R², R³, R⁴ and R⁵, independently of each other or in particular in combination, especially have the following meanings:
R¹ and R² are hydrogen;
R³ fluorine or CN;
R⁴ is selected from the group consisting of hydrogen and fluorine
R⁵ is selected from the group consisting of C₁-C₄-alkoxy-C₁-C₄-alkyl such as 1-methoxyethyl, 1-ethoxyethyl, 1-isoproxyethyl, 2-isoproxyethyl, 1-isobutoxyethyl, 2-isobutoxyethyl, C₁-C₄-alkylthio-C₁-C₄-alkyl such as 2-methylsulfanylpropyl, C₃-C₆-cycloalkyl-C₁-C₄-alkyl and C₃-C₈-cycloalkyl, where cycloalkyl in the last two radicals is unsubstituted or optionally substituted with 1, 2, 3 or 4 C₁-C₄-alkyl radicals, such as cyclopropyl, cyclobutyl, cyclopentyl, cyclohexyl, cycloheptyl, cyclooctyl, bicyclo[3.1.1]heptyl, 4-methylcyclohexyl, 4-ethylcyclohexyl, 6,6-dimethylnorpinan-2-yl, (4-methylcyclohexyl)methyl, or (4-ethylcyclohexyl)methyl.

Especially, in the compounds of formula (I), where the moiety of formula (A) is selected from the moieties of formulae W.Het-1, W.Het-2, W.Het-3, W.Het-4, W.Het-5, W.Het-6, W.Het-7, W.Het-8, W.Het-9, W.Het-10, W.Het-11 and W.Het-12 and likewise in the embodiments 1, 2, 3, 4, 5, 6, 1a, 2a, 3a, 4a, 5a and 6a, 1b, 2b, 3b, 4b, 5b and 6b in an alternative embodiment, the variables R¹, R², R³, R⁴ and R⁵, independently of each other or in particular in combination, especially have the following meanings:
one of R¹ and R² is hydrogen while the other is methyl;
R³ fluorine or CN;
R⁴ is selected from the group consisting of hydrogen and fluorine; and
R⁵ is selected from the group consisting of cyanomethyl, C₁-C₄-alkoxy-C₁-C₄-alkyl, C₁-C₄-alkylthio-C₁-C₄-alkyl, NR^{9a}R^{9b}, C₁-C₆-alkyl, C₁-C₄-haloalkyl, C(═O)OR⁸, C(═O)NR^{9a}R^{9b}, C(═O)R^{7a},
   C₃-C₆-cycloalkyl-C₁-C₄-alkyl and C₃-C₈-cycloalkyl, where cycloalkyl in the last two radicals is unsubstituted or optionally substituted with 1, 2, 3 or 4 C₁-C₄-alkyl radicals Q-phenyl, where phenyl is unsubstituted or substituted with 1, 2, 3, 4 or 5 identical or different substituents R¹⁰, or Q-Het^{#}, where Het^{#} is unsubstituted or substituted with 1, 2, 3, or 4 identical or different substituents R¹⁰, and where Q, irrespectively of its occurrence, is a single bond, NH, N(C₁-C₄-alkyl), CH₂, NHCH₂ or N(C₁-C₄-alkyl)CH₂.

In the compounds of formula (I), in particular in those compounds of formula (I) where the moiety of formula (A) is selected from the moieties of formulae W.Het-1, W.Het-2, W.Het-3, W.Het-4, W.Het-5, W.Het-6, W.Het-7, W.Het-8, W.Het-9, W.Het-10, W.Het-11 and W.Het-12 and likewise in the embodiments 1, 2, 3, 4, 5, 6, 1a, 2a, 3a, 4a, 5a and 6a, the variable X is in particular O.

Apart from that, the variables Het^{#}, Q, R^{v}, R^{w}, R⁶, R⁷, R^{7a}, R⁸, R⁹, R^{9a}, R^{9b}, R^{9c}, R^{9d}, R¹⁰, R¹¹, R¹², R¹³, R¹⁴, R¹⁵, R¹⁶, R¹⁷, R^{17a}, R^{17b} and R^{17c}, irrespectively of their occurrence, in particular have the following meanings, if not stated otherwise:

Het^{#} irrespectively of its occurrence, is 5- or 6-membered hetaryl such as pyridyl, thienyl, furyl, pyrrolyl, thiazolyl, isothiazolyl, pyrazolyl, imidazolyl, oxazolyl or isoxazolyl, which is unsubstituted or substituted by 1, 2 or 3 radicals R¹⁰.

Q is, irrespectively of its occurrence, selected from a single bond, NR^{9a}, CH₂, and NR^{9a}CH₂, and it is in particular a single bond, NH, N(C₁-C₄-alkyl), CH₂, NHCH₂ or N(C₁-C₄-alkyl)CH₂.

R^{v} is hydrogen or together with an R^{v}, which is bound to an adjacent carbon atom, forms together with the existing bound a C═C-double bond.

R^{w} irrespectively of its occurrence, is selected from the group consisting of hydrogen, halogen, such as fluorine or chlorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy. R^{w} is more particularly hydrogen, chlorine, fluorine or methyl and especially hydrogen.

R⁶ irrespectively of its occurrence, is selected from the group consisting of halogen, such as chlorine or fluorine, C₁-C₄-alkyl, such as methyl or ethyl, C₁-C₄-alkoxy, such as methoxy or ethoxy, C₁-C₄-haloalkoxy, such as difluoromethoxy or trifluormethoxy, and C₁-C₄-haloalkyl, such as difluoromethyl, trifluoromethyl, 2,2-difluoroethyl, 2,2,2-trifluoroethyl or pentafluoroethyl, more preferably from halogen, C₁-C₄-alkyl and C₁-C₄-haloalkyl, even more preferably from fluorine, chlorine, C₁-C₂-alkyl, such as methyl or ethyl and C₁-C₂-haloalkyl such as difluoromethyl, trifluoromethyl, 2,2-difluoroethyl, 2,2,2-trifluoroethyl or pentafluoroethyl.

R⁷ irrespectively of its occurrence, is selected from the group consisting of CN, C₁-C₄-alkoxy, such as methoxy or ethoxy, C₁-C₄-haloalkoxy, such as difluoromethoxy or trifluormethoxy, such as difluoromethyl, trifluoromethyl, 2,2-difluoroethyl, 2,2,2-trifluoroethyl or pentafluoroethyl, more preferably from halogen, C₁-C₄-alkyl and C₁-C₄-haloalkyl, even more preferably from fluorine, chlorine, C₁-C₂-alkyl, such as methyl or ethyl and C₁-C₂-haloalkyl such as difluoromethyl, trifluoromethyl, 2,2-difluoroethyl, 2,2,2-trifluoroethyl or pentafluoroethyl, C₃-C₆-cycloalkyl such as cyclopropyl, cyclobutyl or cyclopropyl, and C₃-C₆-halocycloalkyl.

R^{7a} irrespectively of its occurrence, is selected from the group consisting of hydrogen, C₁-C₄-alkyl, C₁-C₄-haloalkyl, C₃-C₆-cycloalkyl, C₃-C₆-cycloalkyl-C₁-C₄-alkyl, phenyl and benzyl, where the phenyl ring in the last two radicals is unsubstituted or substituted by 1, 2 or 3 identical or different radicals selected from the group consisting of halogen, such as chlorine or fluorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy.

R⁸ irrespectively of its occurrence, is selected from the group consisting of C₁-C₄-alkyl, C₁-C₄-haloalkyl, C₃-C₆-cycloalkyl, C₃-C₆-cycloalkyl-C₁-C₄-alkyl, phenyl and benzyl, where the phenyl ring in the last two radicals is unsubstituted or substituted by 1, 2 or 3 identical or different radicals selected from the group consisting of halogen, such as chlorine or fluorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy. Likewise, R⁸ is selected from C₁-C₄-alkoxy-C₁-C₄-alkyl.

R⁹ irrespectively of its occurrence, is selected from the group consisting of C₁-C₄-alkyl, C₁-C₄-haloalkyl, C₃-C₆-cycloalkyl, C₃-C₆-cycloalkyl-C₁-C₄-alkyl, phenyl and benzyl, where the phenyl ring in the last two radicals is unsubstituted or substituted by 1, 2 or 3 identical or different radicals selected from the group consisting of halogen, such as chlorine or fluorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy.

R^{9a} and R^{9b} irrespectively of their occurrence, are preferably selected from the group consisting of hydrogen, C₁-C₄-alkyl, such as methyl, ethyl, n-propyl, isopropyl, n-butyl, 2-butyl or isobutyl, and C₁-C₄-haloalkyl, such as fluoromethyl, difluoromethyl, trifluoromethyl, 1,1-difluoroethyl, 2-fluoroethyl, 2,2-difluoroethyl or 2,2,2-trifluoroethyl, or NR^{9a}R^{9b} may also be a saturated N-bound 3-, 4-, 5- or 6-membered heterocycle, which in addition to the nitrogen atom may have 1 further heteroatom as ring members, which is selected from 0 and N and where the N-bound 3-, 4-, 5- or 6-membered heterocycle may be unsubstituted or carry 1, 2, 3 or 4 radicals selected from C₁-C₄-alkyl and C₁-C₄-haloalkyl. Examples of radicals NR^{9a}R^{9b} include, but are not limited to methylamino, ethylamino, n-propylamino, isopropylamino, n-butylamino, 2-butylamino, isobutylamino, dimethylamino, diethylamino, di-n-propylamino, di-n-butylamino, N-methyl-N-ethylamino, N-methyl-N-propylamino, N-methyl-N-n-propylamino, N-methyl-N-isopropylamino, N-methyl-N-n-butylamino, N-methyl-N-2-butylamino, N-methyl-N-isobutylamino, 1-pyrrolidinyl, 1-piperidinyl, 1-piperazinyl, 4-methyl-1-piperazinyl and 4-morpholinyl.

R^{9c} and R^{9d} irrespectively of their occurrence, are preferably selected from the group consisting of hydrogen, C₁-C₄-alkyl, such as methyl, ethyl, n-propyl, isopropyl, n-butyl, 2-butyl or isobutyl, and C₁-C₄-haloalkyl, such as fluoromethyl, difluoromethyl, trifluoromethyl, 1,1-difluoroethyl, 2-fluoroethyl, 2,2-difluoroethyl or 2,2,2-trifluoroethyl

R¹⁰ irrespectively of its occurrence, is selected from the group consisting of halogen, such as chlorine or fluorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy.

R¹¹, R¹² independently of their occurrence, are selected from the group consisting of C₁-C₆-alkyl, C₁-C₆-alkoxy, C₃-C₈-cycloalkyl, C₃-C₈-cycloalkyl-C₁-C₄-alkyl, phenyl and benzyl, where the phenyl ring in last two radicals are unsubstituted or substituted with 1, 2, or 3 identical or different radicals selected from fluorine, chlorine, C₁-C₃-alkyl, C₁-C₂-haloalkyl, C₁-C₂-alkoxy and C₁-C₂-haloalkoxy.

R¹³, R¹⁴ independently of their occurrence, are selected from the group consisting of hydrogen, fluorine, chlorine, CN, C₁-C₄-alkyl, such as methyl, ethyl, n-propyl or n-butyl, C₃-C₆-cycloalkyl, such as cyclopropyl, cyclobutyl or cyclopentyl, and phenyl.

R¹⁵ irrespectively of its occurrence, is selected from the group consisting of hydrogen, C₁-C₄-alkyl, C₁-C₄-haloalkyl, C₃-C₆-cycloalkyl, C₃-C₆-cycloalkyl-C₁-C₄-alkyl, phenyl and benzyl, where the phenyl ring in the last two radicals is unsubstituted or substituted by 1, 2 or 3 identical or different radicals selected from the group consisting of halogen, such as chlorine or fluorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy.

R₁₆ irrespectively of its occurrence, is selected from the group consisting of C₁-C₄-alkyl, C₁-C₄-haloalkyl, C₃-C₆-cycloalkyl, C₃-C₆-cycloalkyl-C₁-C₄-alkyl, phenyl and benzyl, where the phenyl ring in the last two radicals is unsubstituted or substituted by 1, 2 or 3 identical or different radicals selected from the group consisting of halogen, such as chlorine or fluorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy.

R¹⁷ irrespectively of its occurrence, is selected from the group consisting of hydrogen, C₁-C₆-alkyl, C₁-C₄-haloalkyl, C₁-C₆-alkoxy, C₁-C₄-haloalkoxy, C₃-C₆-alkenyl, C₃-C₆-cycloalkyl-C₁-C₄-alkyl, phenyl and benzyl, where the phenyl ring in the last two radicals is unsubstituted or substituted by 1, 2 or 3 identical or different radicals selected from the group consisting of halogen, such as chlorine or fluorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy.

R^{17a} and R^{17b} irrespectively of their occurrence, are preferably selected from the group consisting of hydrogen, C₁-C₄-alkyl, such as methyl, ethyl, n-propyl, isopropyl, n-butyl, 2-butyl or isobutyl, and C₁-C₄-haloalkyl, such as fluoromethyl, difluoromethyl, trifluoromethyl, 1,1-difluoroethyl, 2-fluoroethyl, 2,2-difluoroethyl or 2,2,2-trifluoroethyl.

R^{17c} irrespectively of its occurrence, is selected from the group consisting of hydrogen, C₁-C₄-alkyl, C₁-C₄-haloalkyl, C₃-C₆-cycloalkyl, C₃-C₆-cycloalkyl-C₁-C₄-alkyl, phenyl and benzyl, where the phenyl ring in the last two radicals is unsubstituted or substituted by 1, 2 or 3 identical or different radicals selected from the group consisting of halogen, such as chlorine or fluorine, CN, C₁-C₄-alkyl, such as methyl, ethyl, n-propyl and isopropyl, C₁-C₄-haloalkyl, in particular C₁-C₂-haloalkyl, such as fluoromethyl, difluoromethyl, trifluoromethyl, 1,1-difluoroethyl, 2-fluoroethyl, 2,2-difluoroethyl or 2,2,2-trifluoroethyl, C₁-C₄-alkoxy, such as methoxy, ethoxy, n-propoxy and isopropoxy, and C₁-C₄-haloalkoxy, in particular C₁-C₂-haloalkoxy, such as fluoromethoxy, difluoromethoxy, trifluoromethoxy, 1,1-difluoroethoxy, 2-fluoroethoxy, 2,2-difluoroethoxy or 2,2,2-trifluoroethoxy.

A special group of embodiments relates to the compounds of formula (I-A.1a), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.1a), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (I-A.2a), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.2a), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (I-A.1 b), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.1 b), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (I-A.2b), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.2b), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (I-A.1c), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.1c), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (I-A.2c), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.2c), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (-A.1dl), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.1 d), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A

A further special group of embodiments relates to the compounds of formula (I-A.2d), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.2d), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (I-A.3a), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.3a), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (I-A.4a), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.4a), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (I-A.3b), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.3b), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (I-A.4b), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.4b), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (I-A.3c), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.3c), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (I-A.4c), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.4c), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (I-A.3d), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.3d), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (I-A.4d), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.4d), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (I-A.5a), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.5a), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (I-A.6a), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.6a), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (I-A.5b), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.5b), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (I-A.6b), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.6b), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A

A further special group of embodiments relates to the compounds of formula (I-A.5c), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.5c), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (I-A.6c), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.6c), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (I-A.5d), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.5d), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A further special group of embodiments relates to the compounds of formula (I-A.6d), to their tautomers, to their stereoisomers and to their salts, where R¹ is hydrogen, R³, R⁴ and R⁵ are as defined above and where R³, R⁴ and R⁵ have in particular one of the meanings given in any of lines 1 to 693 of the following table A. A further special group of embodiments relates to the compounds of formula (I-A.6d), to their tautomers, to their stereoisomers and to their salts, where R¹ is methyl, R³, R⁴ and R⁵ are as defined above and where R³, R⁴ and R⁵ have in particular one of the meanings given in any of lines 1 to 693 of the following table A.

A skilled person will readily appreciate that the compounds of formulae (I-A.1a), (I-A.1 b), (I-A.1c), (I-A.1 d), (I-A.2a), (I-A.2b), (I-A.2c), (I-A.2d), (I-A.3a), (I-A.3b), (I-A.3c), (I-A.3d), (I-A.4a), (I-A.4b), (I-A.4c), (I-A.4d), (I-A.5a), (I-A.5b), (I-A.5c), (I-A.5d), (I-A.6a), (I-A.6b), (I-A.6c), (I-A.6d), where R³, R⁴ and R⁵ have one of the meanings given in any of lines 2 to 335, 339 to 670, 672 to 687 or 693 of the following table A may have E- or Z-configuration with regard to the carbon-carbon double between CR⁴R⁵ and CR³ (remainder of the molecule). The afore-mentioned embodiments include both the E-isomer and the Z-isomer or as well as a mixture of these geomet-ric isomers. A skilled person will also readily appreciate that the compounds of formulae (I-A.1a), (I-A.1b), (I-A.1c), (I-A.1 d), (I-A.2a), (I-A.2b), (I-A.2c), (I-A.2d), (I-A.3a), (I-A.3b), (I-A.3c), (I-A.3d), (I-A.4a), (I-A.4b), (I-A.4c), (I-A.4d), (I-A.5a), (I-A.5b), (I-A.5c), (I-A.5d), (I-A.6a), (I-A.6b), (I-A.6c), (I-A.6d), where R¹ is methyl, and R³, R⁴ and R⁵ have one of the meanings given in any of lines 1 to 693 of the following table A may have S- or R-configuration with regard to the carbon atom carrying the radical R¹. The aforementioned embodiments include both the S-enantiomers and the R-enantiomers as well as mixtures of these enantiomers, in particular ra-cemic mixtures.

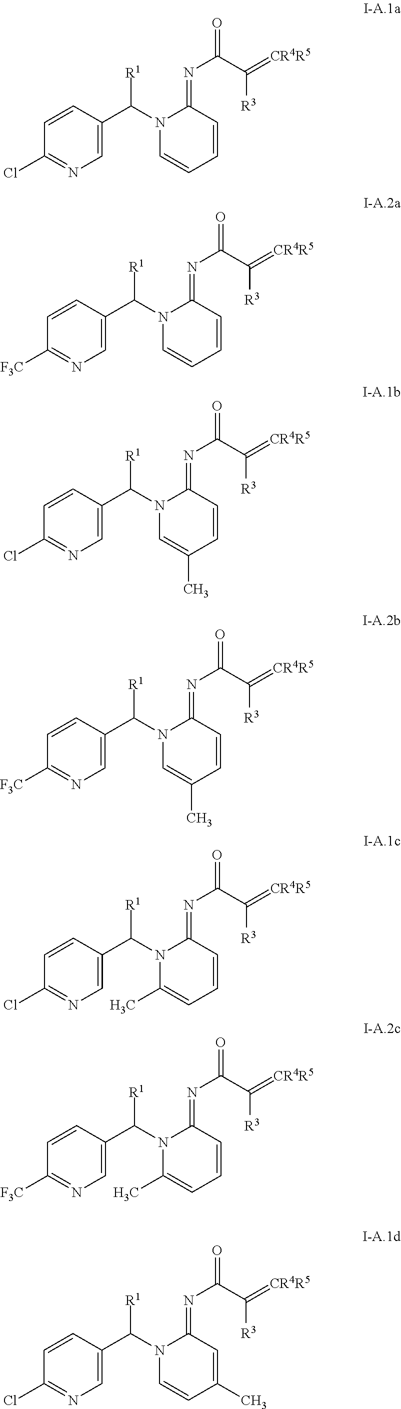

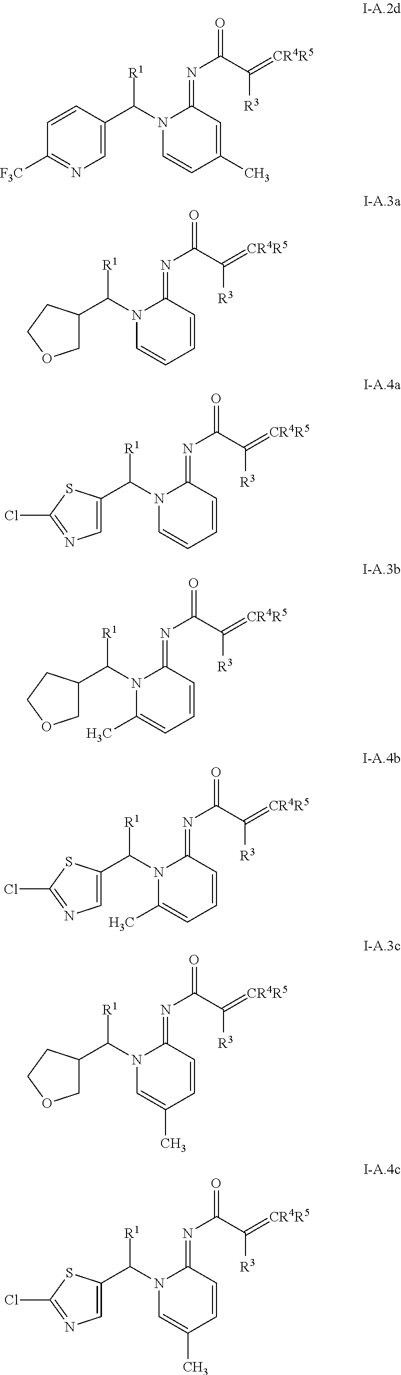

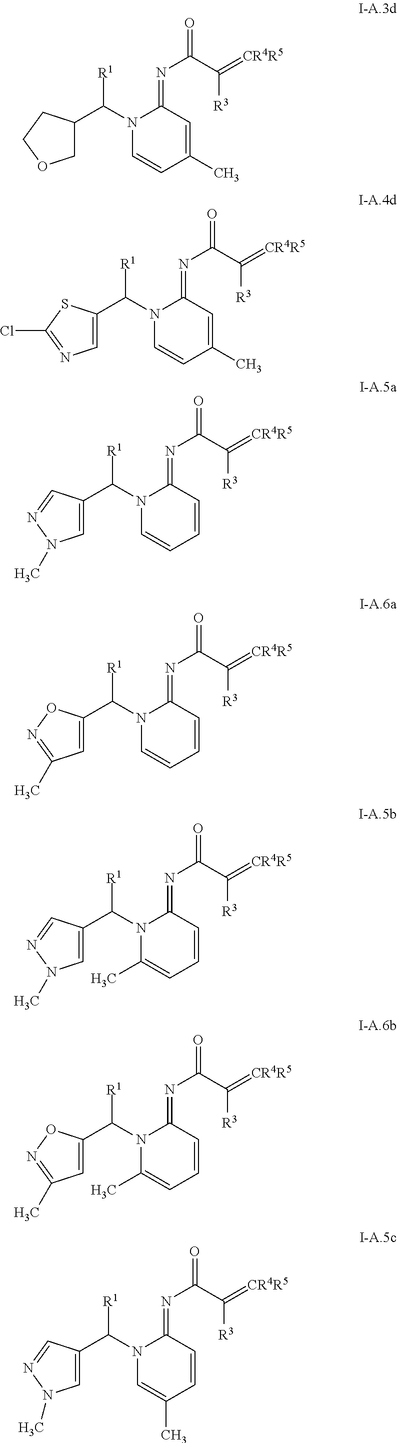

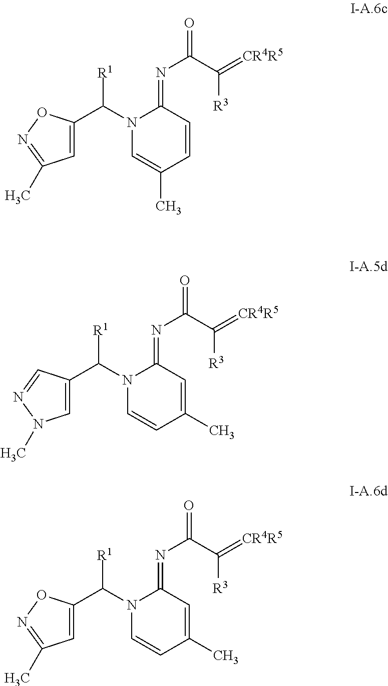

TABLE A

| # | R³ | R⁴ | R⁵ |
|---|---|---|---|
| 1 | F | H | H |
| 2 | F | H | CH₃ |
| 3 | F | H | CH₂CH₃ |
| 4 | F | H | CH₂CH₂CH₃ |
| 5 | F | H | CH(CH₃)₂ |
| 6 | F | H | CH(CH₃)CH(CH₃)₂ |
| 7 | F | H | CH₂CH₂CH₂CH₃ |
| 8 | F | H | C(CH₃)₃ |
| 9 | F | H | CH(CH₃)CH(CH₃)CH₂CH₃ |
| 10 | F | H | CH(CH₃)CH₂CH₃ |
| 11 | F | H | CH₂CH(CH₃)₂ |
| 12 | F | H | (CH₂)₄CH₃ |
| 13 | F | H | CH(CH₃)C(CH₃)₃ |
| 14 | F | H | CH₂C(CH₃)₃ |
| 15 | F | H | CH(CH₃)CH₂CH₂CH₃ |
| 16 | F | H | CH(C₂H₅)CH₂CH₂CH₂CH₃ |
| 17 | F | H | CHCl₂ |
| 18 | F | H | CH₂CN |
| 19 | F | H | 4-iP—c-C₆H₁₀—CH₂— |
| 20 | F | H | (CH₂)₂—C₆H₅ |
| 21 | F | H | CH₂CH₂C(H)═C(H)CH₂CH₂CH₂CH₂CH₃ |
| 22 | F | H | CH(CH₃)OCH₃ |
| 23 | F | H | CH(CH₃)OCH₂CH₂OCH₃ |
| 24 | F | H | CH(CH₃)OCH(CH₃)₂ |
| 25 | F | H | CH₂CH₂OCH₂CH(CH₃)₂ |
| 26 | F | H | CH(CH₃)O—c-C₆H₁₁ |
| 27 | F | H | CH(CH₃)OC₆H₅ |
| 28 | F | H | 3-Ac—O—1-Me-pr |
| 29 | F | H | 6-Ac—O—C₆H₁₂ |
| 30 | F | H | CH₂CH(CH₃)SCH₃ |
| 31 | F | H | CH(CH₃)CH₂SCH₃ |
| 32 | F | H | C(CH₃)₂CH₂—3-Py |
| 33 | F | H | c-C₃H₅ |
| 34 | F | H | c-C₄H₇ |
| 35 | F | H | c-C₅H₉ |
| 36 | F | H | c-C₆H₁₁ |
| 37 | F | H | c-C₇H₁₃ |
| 38 | F | H | c-C₈H₁₅ |
| 39 | F | H | 4-Me—c-C₆H₁₀ |
| 40 | F | H | 6,6-DMe-2-N |
| 41 | F | H | 2-EC—c-C₃H₅ |
| 42 | F | H | C₆H₅ |
| 43 | F | H | 4-Cl—C₆H₄ |
| 44 | F | H | 4-F—C₆H₄ |
| 45 | F | H | 4-Br—C₆H₄ |
| 46 | F | H | 4-OCH₃—C₆H₄ |
| 47 | F | H | 4-CF₃—C₆H₄ |
| 48 | F | H | 4-(C(CH₃)₃)—C₆H₄ |
| 49 | F | H | 3-Cl—C₆H₄ |
| 50 | F | H | 3-F—C₆H₄ |
| 51 | F | H | 3-Br—C₆H₄ |
| 52 | F | H | 3-OCH₃—C₆H₄ |
| 53 | F | H | 3-CF₃—C₆H₄ |
| 54 | F | H | 3-(C(CH₃)₃)—C₆H₄ |
| 55 | F | H | 2-Cl—C₆H₄ |
| 56 | F | H | 2-F—C₆H₄ |
| 57 | F | H | 2-Br—C₆H₄ |
| 58 | F | H | 2-OCH₃—C₆H₄ |
| 59 | F | H | 2-CF₃—C₆H₄ |
| 60 | F | H | 2-(C(CH₃)₃)—C₆H₄ |
| 61 | F | H | 3,4-(CH₃)₂—C₆H₃ |
| 62 | F | H | 3,4-Cl₂—C₆H₃ |
| 63 | F | H | 3,4-F₂—C₆H₃ |
| 64 | F | H | 3-Cl—4-F—C₆H₃ |
| 65 | F | H | 4-Cl—3-F—C₆H₃ |
| 66 | F | H | 3-Br—4-F—C₆H₃ |
| 67 | F | H | 4-Br—3-F—C₆H₃ |
| 68 | F | H | 3-Br—4-Cl—C₆H₃ |
| 69 | F | H | 4-Br—3-Cl—C₆H₃ |
| 70 | F | H | 4-Cl—3-CF₃—C₆H₃ |
| 71 | F | H | 3-Cl—4-CF₃—C₆H₃ |
| 72 | F | H | 3-OCH₃—4-CH₃—C₆H₃ |
| 73 | F | H | 4-OCH₃—3-CH₃—C₆H₃ |
| 74 | F | H | 3-Py |
| 75 | F | H | 4-Py |
| 76 | F | H | 5-Br—3-Py |
| 77 | F | H | 6-CF3—3-Py |
| 78 | F | H | 2-ImPy |
| 79 | F | H | 2-Pm |
| 80 | F | H | 5-CH₃—1-C₆H₅—4-Pz |
| 81 | F | H | 1H—3-Pyr |
| 82 | F | H | 1,2,5-(CH₃)₂—3-AcN—4-Pyr |
| 83 | F | H | 3-iPr—5-IsO |
| 84 | F | H | 2-Fu |
| 85 | F | H | 3-Th |
| 86 | F | H | 3-THF |
| 87 | F | H | 2-THP |
| 88 | F | H | 6-(CH₃O)—3-(CH₃)—2-THP |
| 89 | F | H | 3-THP |
| 90 | F | H | 4-THP |
| 91 | F | H | 3-THTP |
| 92 | F | H | 4-THTP |
| 93 | F | H | 3-THTP—CH₂ |
| 94 | F | H | 4-THTP—CH₂ |
| 95 | F | H | NH—CH₃ |
| 96 | F | H | NH—CH₂CH₃ |
| 97 | F | H | NH—CH₂CH₂CH₃ |
| 98 | F | H | NH—CH(CH₃)₂ |
| 99 | F | H | NH—CH₂CH₂CH₂CH₃ |
| 100 | F | H | NH—CH(CH₃)CH₂CH₃ |
| 101 | F | H | NH—CH₂CH(CH₃)₂ |
| 102 | F | H | N(CH₃)—CH₃ |
| 103 | F | H | N(CH₃)—CH₂CH₃ |
| 104 | F | H | N(CH₃)—CH₂CH₂CH₃ |
| 105 | F | H | N(CH₃)—CH(CH₃)₂ |
| 106 | F | H | N(CH₃)—CH₂CH₂CH₂CH₃ |
| 107 | F | H | N(CH₃)—CH(CH₃)CH₂CH₃ |
| 108 | F | H | N(CH₃)—CH₂CH(CH₃)₂ |
| 109 | F | H | NH—CH₂C₆H₅ |
| 110 | F | H | NH—CH₂(4-Cl—C₆H₄) |
| 111 | F | H | NH—CH₂(4-F—C₆H₄) |
| 112 | F | H | NH—CH₂(4-Br—C₆H₄) |
| 113 | F | H | NH—CH₂(3-Cl—C₆H₄) |
| 114 | F | H | NH—CH₂(3-F—C₆H₄) |
| 115 | F | H | NH—CH₂(3-Br—C₆H₄) |
| 116 | F | H | NH—CH₂(3,4-Cl₂—C₆H₃) |
| 117 | F | H | NH—CH₂(3,4-F₂—C₆H₃) |
| 118 | F | H | NH—CH₂(3-Cl-4-F—C₆H₃) |
| 119 | F | H | NH—CH₂(4-Cl—3-F—C₆H₃) |
| 120 | F | H | NH—CH₂(3-Br—4-F—C₆H₃) |
| 121 | F | H | NH—CH₂(4-Br—3-F—C₆H₃) |
| 122 | F | H | NH—CH₂(3-Br—4-Cl—C₆H₃) |
| 123 | F | H | NH—CH₂(4-Br—3-Cl—C₆H₃) |
| 124 | F | H | N(CH₃)—CH₂C₆H₅ |
| 125 | F | H | N(CH₃)—CH₂(4-Cl—C₆H₄) |
| 126 | F | H | N(CH₃)—CH₂(4-F—C₆H₄) |
| 127 | F | H | N(CH₃)—CH₂(4-Br—C₆H₄) |
| 128 | F | H | N(CH₃)—CH₂(3-Cl—C₆H₄) |
| 129 | F | H | N(CH₃)—CH₂(3-F—C₆H₄) |
| 130 | F | H | N(CH₃)—CH₂(3-Br—C₆H₄) |
| 131 | F | H | N(CH₃)—CH₂(3,4-Cl₂—C₆H₃) |
| 132 | F | H | N(CH₃)—CH₂(3,4-F₂—C₆H₃) |
| 133 | F | H | N(CH₃)—CH₂(3-Cl—4-F—C₆H₃) |
| 134 | F | H | N(CH₃)—CH₂(4-Cl—3-F—C₆H₃) |
| 135 | F | H | N(CH₃)—CH₂(3-Br—4-F—C₆H₃) |
| 136 | F | H | N(CH₃)—CH₂(4-Br—3-F—C₆H₃ |
| 137 | F | H | N(CH₃)—CH₂(3-Br—4-Cl—C₆H₃ |
| 138 | F | H | N(CH₃)—CH₂(4-Br—3-Cl—C₆H₃ |
| 139 | F | H | NH—C₆H₅ |
| 140 | F | H | NH—(4-Cl—C₆H₄) |
| 141 | F | H | NH—(4-F—C₆H₄) |
| 142 | F | H | NH—(4-Br—C₆H₄) |
| 143 | F | H | NH—(3-Cl—C₆H₄) |
| 144 | F | H | NH—(3-F—C₆H₄) |
| 145 | F | H | NH—(3-Br—C₆H₄) |
| 146 | F | H | NH—(3,4-Cl₂—C₆H₃) |
| 147 | F | H | NH—(3,4-F₂—C₆H₃) |
| 148 | F | H | NH—(3-Cl—4-F—C₆H₃) |
| 149 | F | H | NH—(4-Cl—3-F—C₆H₃) |
| 150 | F | H | NH—(3-Br—4-F—C₆H₃) |
| 151 | F | H | NH—(4-Br—3-F—C₆H₃) |
| 152 | F | H | NH—(3-Br—4-Cl—C₆H₃) |
| 153 | F | H | NH—(4-Br—3-Cl—C₆H₃) |
| 154 | F | H | N(CH₃)—C₆H₅ |
| 155 | F | H | N(CH₃)—(4-Cl—C₆H₄) |
| 156 | F | H | N(CH₃)—(4-F—C₆H₄) |
| 157 | F | H | N(CH₃)—(4-Br—C₆H₄) |
| 158 | F | H | N(CH₃)—(3-Cl—C₆H₄) |
| 159 | F | H | N(CH₃)—(3-F—C₆H₄) |
| 160 | F | H | N(CH₃)—(3-Br—C₆H₄) |
| 161 | F | H | N(CH₃)—(3,4-Cl₂—C₆H₃) |
| 162 | F | H | N(CH₃)—(3,4-F₂—C₆H₃) |
| 163 | F | H | N(CH₃)—(3-Cl—4-F—C₆H₃) |
| 164 | F | H | N(CH₃)—(4-Cl—3-F—C₆H₃) |
| 165 | F | H | N(CH₃)—(3-Br—4-F—C₆H₃) |
| 166 | F | H | N(CH₃)—(4-Br—3-F—C₆H₃) |
| 167 | F | H | N(CH₃)—(3-Br—4-Cl—C₆H₃) |
| 168 | F | H | N(CH₃)—(4-Br—3-Cl—C₆H₃) |
| 169 | F | F | CH₃ |
| 170 | F | F | CH₂CH₃ |
| 171 | F | F | CH₂CH₂CH₃ |
| 172 | F | F | CH(CH₃)₂ |
| 173 | F | F | CH(CH₃)CH(CH₃)₂ |
| 174 | F | F | CH₂CH₂CH₂CH₃ |
| 175 | F | F | C(CH₃)₃ |
| 176 | F | F | CH(CH₃)CH(CH₃)CH₂CH₃ |
| 177 | F | F | CH(CH₃)CH₂CH₃ |
| 178 | F | F | CH₂CH(CH₃)₂ |
| 179 | F | F | (CH₂)₄CH₃ |
| 180 | F | F | CH(CH₃)C(CH₃)₃ |
| 181 | F | F | CH₂C(CH₃)₃ |
| 182 | F | F | CH(CH₃)CH₂CH₂CH₃ |
| 183 | F | F | CH(C₂H₅)CH₂CH₂CH₂CH₃ |
| 184 | F | F | CHCl₂ |
| 185 | F | F | CH₂CN |
| 186 | F | F | 4-iP—c-C₆H₁₀—CH₂— |
| 187 | F | F | (CH₂)₂—C₆H₅ |
| 188 | F | F | CH₂CH₂C(H)═C(H)CH₂CH₂CH₂CH₂CH₃ |
| 189 | F | F | CH(CH₃)OCH₃ |
| 190 | F | F | CH(CH₃)OCH₂CH₂OCH₃ |
| 191 | F | F | CH(CH₃)OCH(CH₃)₂ |
| 192 | F | F | CH₂CH₂OCH₂CH(CH₃)₂ |
| 193 | F | F | CH(CH₃)O—c-C₆H₁₁ |
| 194 | F | F | CH(CH₃)OC₆H₅ |
| 195 | F | F | 3-Ac—O—1-Me-pr |
| 196 | F | F | 6-Ac—O—C₆H₁₂ |
| 197 | F | F | CH₂CH(CH₃)SCH₃ |
| 198 | F | F | CH(CH₃)CH₂SCH₃ |
| 199 | F | F | C(CH₃)₂CH₂—3-Py |
| 200 | F | F | c-C₃H₅ |
| 201 | F | F | c-C₄H₇ |
| 202 | F | F | c-C₅H₉ |
| 203 | F | F | c-C₆H₁₁ |
| 204 | F | F | c-C₇H₁₃ |
| 205 | F | F | c-C₈H₁₅ |
| 206 | F | F | 4-Me—c-C₆H₁₀ |
| 207 | F | F | 6,6-DMe-2-N |
| 208 | F | F | 2-EC—c-C₃H₅ |
| 209 | F | F | C₆H₅ |
| 210 | F | F | 4-Cl—C₆H₄ |
| 211 | F | F | 4-F—C₆H₄ |
| 212 | F | F | 4-Br—C₆H₄ |
| 213 | F | F | 4-OCH₃—C₆H₄ |
| 214 | F | F | 4-CF₃—C₆H₄ |
| 215 | F | F | 4-(C(CH₃)₃)—C₆H₃ |
| 216 | F | F | 3-Cl—C₆H₄ |
| 217 | F | F | 3-F—C₆H₄ |
| 218 | F | F | 3-Br—C₆H₄ |
| 219 | F | F | 3-OCH₃—C₆H₄ |
| 220 | F | F | 3-CF₃—C₆H₄ |
| 221 | F | F | 3-(C(CH₃)₃)—C₆H₃ |
| 222 | F | F | 2-Cl—C₆H₄ |
| 223 | F | F | 2-F—C₆H₄ |
| 224 | F | F | 2-Br—C₆H₄ |
| 225 | F | F | 2-OCH₃—C₆H₄ |
| 226 | F | F | 2-CF₃—C₆H₄ |
| 227 | F | F | 2-(C(CH₃)₃)—C₆H₃ |
| 228 | F | F | 3,4-(CH₃)₂—C₆H₃ |
| 229 | F | F | 3,4-Cl₂—C₆H₃ |
| 230 | F | F | 3,4-F₂—C₆H₃ |
| 231 | F | F | 3-Cl—4-F—C₆H₃ |
| 232 | F | F | 4-Cl—3-F—C₆H₃ |
| 233 | F | F | 4-Cl—3-CF₃—C₆H₃ |
| 234 | F | F | 3-Cl—4-CF₃—C₆H₃ |
| 235 | F | F | 3-Br—4-F—C₆H₃ |
| 236 | F | F | 4-Br—3-F—C₆H₃ |
| 237 | F | F | 3-OCH₃—4-CH₃—C₆H₃ |
| 238 | F | F | 4-OCH₃—3-CH₃—C₆H₃ |
| 239 | F | F | 3-Br—4-Cl—C₆H₃ |
| 240 | F | F | 4-Br—3-Cl—C₆H₃ |
| 241 | F | F | 3-Py |
| 242 | F | F | 4-Py |
| 243 | F | F | 5-Br—3-Py |
| 244 | F | F | 6-CF3—3-Py |
| 245 | F | F | 2-ImPy |
| 246 | F | F | 2-Pm |
| 247 | F | F | 5-CH₃—1-C₆H₅—4-Pz |
| 248 | F | F | 1H—3-Pyr |
| 249 | F | F | 1,2,5-(CH₃)₂—3-AcN—4-Pyr |
| 250 | F | F | 3-iPr—5-IsO |
| 251 | F | F | 2-Fu |
| 252 | F | F | 3-Th |
| 253 | F | F | 3-THF |
| 254 | F | F | 2-THP |
| 255 | F | F | 6-(CH₃O)—3-(CH₃)—2-THP |
| 256 | F | F | 3-THP |
| 257 | F | F | 4-THP |
| 258 | F | F | 3-THTP |
| 259 | F | F | 4-THTP |
| 260 | F | F | 3-THTP—CH₂ |
| 261 | F | F | 4-THTP—CH₂ |
| 262 | F | F | NH—CH₃ |
| 263 | F | F | NH—CH₂CH₃ |
| 264 | F | F | NH—CH₂CH₂CH₃ |
| 265 | F | F | NH—CH(CH₃)₂ |
| 266 | F | F | NH—CH₂CH₂CH₂CH₃ |
| 267 | F | F | NH—CH(CH₃)CH₂CH₃ |
| 268 | F | F | NH—CH₂CH(CH₃)₂ |
| 269 | F | F | N(CH₃)—CH₃ |
| 270 | F | F | N(CH₃)—CH₂CH₃ |
| 271 | F | F | N(CH₃)—CH₂CH₂CH₃ |
| 272 | F | F | N(CH₃)—CH(CH₃)₂ |
| 273 | F | F | N(CH₃)—CH₂CH₂CH₂CH₃ |
| 274 | F | F | N(CH₃)—CH(CH₃)CH₂CH₃ |
| 275 | F | F | N(CH₃)—CH₂CH(CH₃)₂ |
| 276 | F | F | NH—CH₂C₆H₅ |
| 277 | F | F | NH—CH₂(4-Cl—C₆H₄) |
| 278 | F | F | NH—CH₂(4-F—C₆H₄) |
| 279 | F | F | NH—CH₂(4-Br—C₆H₄) |
| 280 | F | F | NH—CH₂(3-Cl—C₆H₄) |
| 281 | F | F | NH—CH₂(3-F—C₆H₄) |
| 282 | F | F | NH—CH₂(3-Br—C₆H₄) |
| 283 | F | F | NH—CH₂(3,4-Cl₂—C₆H₃) |
| 284 | F | F | NH—CH₂(3,4-F₂—C₆H₃) |
| 285 | F | F | NH—CH₂(3-Cl—4-F—C₆H₃) |
| 286 | F | F | NH—CH₂(4-Cl—3-F—C₆H₃) |
| 287 | F | F | NH—CH₂(3-Br—4-F—C₆H₃) |
| 288 | F | F | NH—CH₂(4-Br—3-F—C₆H₃) |
| 289 | F | F | NH—CH₂(3-Br—4-Cl—C₆H₃) |
| 290 | F | F | NH—CH₂(4-Br—3-Cl—C₆H₃) |
| 291 | F | F | N(CH₃)—CH₂C₆H₅ |
| 292 | F | F | N(CH₃)—CH₂(4-Cl—C₆H₄) |
| 293 | F | F | N(CH₃)—CH₂(4-F—C₆H₄) |
| 294 | F | F | N(CH₃)—CH₂(4-Br—C₆H₄) |
| 295 | F | F | N(CH₃)—CH₂(3-Cl—C₆H₄) |
| 296 | F | F | N(CH₃)—CH₂(3-F—C₆H₄) |
| 297 | F | F | N(CH₃)—CH₂(3-Br—C₆H₄) |
| 298 | F | F | N(CH₃)—CH₂(3,4-Cl₂—C₆H₃) |
| 299 | F | F | N(CH₃)—CH₂(3,4-F₂—C₆H₃) |
| 300 | F | F | N(CH₃)—CH₂(3-Cl—4-F—C₆H₃) |
| 301 | F | F | N(CH₃)—CH₂(4-Cl—3-F—C₆H₃) |
| 302 | F | F | N(CH₃)—CH₂(3-Br—4-F—C₆H₃) |
| 303 | F | F | N(CH₃)—CH₂(4-Br—3-F—C₆H₃) |
| 304 | F | F | N(CH₃)—CH₂(3-Br—4-Cl—C₆H₃) |
| 305 | F | F | N(CH₃)—CH₂(4-Br—3-Cl—C₆H₃) |
| 306 | F | F | NH—C₆H₅ |
| 307 | F | F | NH—(4-Cl—C₆H₄) |
| 308 | F | F | NH—(4-F—C₆H₄) |
| 309 | F | F | NH—(4-Br—C₆H₄) |
| 310 | F | F | NH—(3-Cl—C₆H₄) |
| 311 | F | F | NH—(3-F—C₆H₄) |
| 312 | F | F | NH—(3-Br—C₆H₄) |
| 313 | F | F | NH—(3,4-Cl₂—C₆H₃) |
| 314 | F | F | NH—(3,4-F₂—C₆H₃) |
| 315 | F | F | NH—(3-Cl—4-F—C₆H₃) |
| 316 | F | F | NH—(4-Cl—3-F—C₆H₃) |
| 317 | F | F | NH—(3-Br—4-F—C₆H₃) |
| 318 | F | F | NH—(4-Br—3-F—C₆H₃) |
| 319 | F | F | NH—(3-Br—4-Cl—C₆H₃) |
| 320 | F | F | NH—(4-Br—3-Cl—C₆H₃) |
| 321 | F | F | N(CH₃)—C₆H₅ |
| 322 | F | F | N(CH₃)—(4-Cl—C₆H₄) |
| 323 | F | F | N(CH₃)—(4-F—C₆H₄) |
| 324 | F | F | N(CH₃)—(4-Br—C₆H₄) |
| 325 | F | F | N(CH₃)—(3-Cl—C₆H₄) |
| 326 | F | F | N(CH₃)—(3-F—C₆H₄) |
| 327 | F | F | N(CH₃)—(3-Br—C₆H₄) |
| 328 | F | F | N(CH₃)—(3,4-Cl₂—C₆H₃) |
| 329 | F | F | N(CH₃)—(3,4-F₂—C₆H₃) |
| 330 | F | F | N(CH₃)—(3-Cl—4-F—C₆H₃) |
| 331 | F | F | N(CH₃)—(4-Cl—3-F—C₆H₃) |
| 332 | F | F | N(CH₃)—(3-Br—4-F—C₆H₃) |
| 333 | F | F | N(CH₃)—(4-Br—3-F—C₆H₃ |
| 334 | F | F | N(CH₃)—(3-Br—4-Cl—C₆H₃ |
| 335 | F | F | N(CH₃)—(4-Br—3-Cl—C₆H₃ |
| 336 | Cl | H | H |
| 337 | Br | H | H |
| 338 | CH₃ | H | H |
| 339 | CN | H | CH₃ |
| 340 | CN | H | CH₂CH₃ |
| 341 | CN | H | CH₂CH₂CH₃ |
| 342 | CN | H | CH(CH₃)₂ |
| 343 | CN | H | CH(CH₃)CH(CH₃)₂ |
| 344 | CN | H | CH₂CH₂CH₂CH₃ |
| 345 | CN | H | C(CH₃)₃ |
| 346 | CN | H | CH(CH₃)CH(CH₃)CH₂CH₃ |
| 347 | CN | H | CH(CH₃)CH₂CH₃ |
| 348 | CN | H | CH₂CH(CH₃)₂ |
| 349 | CN | H | (CH₂)₄CH₃ |
| 350 | CN | H | CH(CH₃)C(CH₃)₃ |
| 351 | CN | H | CH₂C(CH₃)₃ |
| 352 | CN | H | CH(CH₃)CH₂CH₂CH₃ |
| 353 | CN | H | CH(C₂H₅)CH₂CH₂CH₂CH₃ |
| 354 | CN | H | CHCl₂ |
| 355 | CN | H | CH₂CN |
| 356 | CN | H | 4-iP—c-C₆H₁₀—CH₂— |
| 357 | CN | H | (CH₂)₂—C₆H₅ |
| 358 | CN | H | CH₂CH₂C(H)═C(H)CH₂CH₂CH₂CH₂CH₃ |
| 359 | CN | H | CH(CH₃)OCH₃ |
| 360 | CN | H | CH(CH₃)OCH₂CH₂OCH₃ |
| 361 | CN | H | CH(CH₃)OCH(CH₃)₂ |
| 362 | CN | H | CH₂CH₂OCH₂CH(CH₃)₂ |
| 363 | CN | H | CH(CH₃)O—c-C₆H₁₁ |
| 364 | CN | H | CH(CH₃)OC₆H₅ |
| 365 | CN | H | 3-Ac—O—1-Me-pr |
| 366 | CN | H | 6-Ac—O—C₆H₁₂ |
| 367 | CN | H | CH₂CH(CH₃)SCH₃ |
| 368 | CN | H | CH(CH₃)CH₂SCH₃ |
| 369 | CN | H | C(CH₃)₂CH₂—3-Py |
| 370 | CN | H | c-C₃H₅ |
| 371 | CN | H | c-C₄H₇ |
| 372 | CN | H | c-C₅H₉ |
| 373 | CN | H | c-C₆H₁₁ |
| 374 | CN | H | c-C₇H₁₃ |
| 375 | CN | H | c-C₈H₁₅ |
| 376 | CN | H | 4-Me—c-C₆H₁₀ |
| 377 | CN | H | 6,6-DMe-2-N |
| 378 | CN | H | 2-EC—c-C₃H₅ |
| 379 | CN | H | C₆H₅ |
| 380 | CN | H | 4-Cl—C₆H₄ |
| 381 | CN | H | 4-F—C₆H₄ |
| 382 | CN | H | 4-Br—C₆H₄ |
| 383 | CN | H | 4-OCH₃—C₆H₄ |
| 384 | CN | H | 4-CF₃—C₆H₄ |
| 385 | CN | H | 4-(C(CH₃)₃)—C₆H₄ |
| 386 | CN | H | 3-Cl—C₆H₄ |
| 387 | CN | H | 3-F—C₆H₄ |
| 388 | CN | H | 3-Br—C₆H₄ |
| 389 | CN | H | 3-OCH₃—C₆H₄ |
| 390 | CN | H | 3-CF₃—C₆H₄ |
| 391 | CN | H | 3-(C(CH₃)₃)—C₆H₄ |
| 392 | CN | H | 2-Cl—C₆H₄ |
| 393 | CN | H | 2-F—C₆H₄ |
| 394 | CN | H | 2-Br—C₆H₄ |
| 395 | CN | H | 2-OCH₃—C₆H₄ |
| 396 | CN | H | 2-CF₃—C₆H₄ |
| 397 | CN | H | 2-(C(CH₃)₃)—C₆H₄ |
| 398 | CN | H | 3,4-(CH₃)₂—C₆H₃ |
| 399 | CN | H | 3,4-Cl₂—C₆H₃ |
| 400 | CN | H | 3,4-F₂—C₆H₃ |
| 401 | CN | H | 3-Cl—4-F—C₆H₃ |
| 402 | CN | H | 4-Cl—3-F—C₆H₃ |
| 403 | CN | H | 3-Br—4-F—C₆H₃ |
| 404 | CN | H | 4-Br—3-F—C₆H₃ |
| 405 | CN | H | 3-Br—4-Cl—C₆H₃ |
| 406 | CN | H | 4-Br—3-Cl—C₆H₃ |
| 407 | CN | H | 4-Cl—3-CF₃—C₆H₃ |
| 408 | CN | H | 3-Cl—4-CF₃—C₆H₃ |
| 409 | CN | H | 3-OCH₃—4-CH₃—C₆H₃ |
| 410 | CN | H | 4-OCH₃—3-CH₃—C₆H₃ |
| 411 | CN | H | 3-Py |
| 412 | CN | H | 4-Py |
| 413 | CN | H | 5-Br—3-Py |
| 414 | CN | H | 6-CF₃—3-Py |
| 415 | CN | H | 2-ImPy |
| 416 | CN | H | 2-Pm |
| 417 | CN | H | 5-CH₃—1-C₆H₅—4-Pz |
| 418 | CN | H | 1H—3-Pyr |
| 419 | CN | H | 3-iPr—5-IsO |
| 420 | CN | H | 2-Fu |
| 421 | CN | H | 3-THF |
| 422 | CN | H | 2-THP |
| 423 | CN | H | 6-(CH₃O)—3-(CH₃)—2-THP |
| 424 | CN | H | 3-THP |
| 425 | CN | H | 4-THP |
| 426 | CN | H | 3-THTP |
| 427 | CN | H | 4-THTP |
| 428 | CN | H | 3-THTP—CH₂ |
| 429 | CN | H | 4-THTP—CH₂ |
| 430 | CN | H | NH—CH₃ |
| 431 | CN | H | NH—CH₂CH₃ |
| 432 | CN | H | NH—CH₂CH₂CH₃ |
| 433 | CN | H | NH—CH(CH₃)₂ |
| 434 | CN | H | NH—CH₂CH₂CH₂CH₃ |
| 435 | CN | H | NH—CH(CH₃)CH₂CH₃ |
| 436 | CN | H | NH—CH₂CH(CH₃)₂ |
| 437 | CN | H | N(CH₃)—CH₃ |
| 438 | CN | H | N(CH₃)—CH₂CH₃ |
| 439 | CN | H | N(CH₃)—CH₂CH₂CH₃ |
| 440 | CN | H | N(CH₃)—CH(CH₃)₂ |
| 441 | CN | H | N(CH₃)—CH₂CH₂CH₂CH₃ |
| 442 | CN | H | N(CH₃)—CH(CH₃)CH₂CH₃ |
| 443 | CN | H | N(CH₃)—CH₂CH(CH₃)₂ |
| 444 | CN | H | NH—CH₂C₆H₅ |
| 445 | CN | H | NH—CH₂(4-Cl—C₆H₄) |
| 446 | CN | H | NH—CH₂(4-F—C₆H₄) |
| 447 | CN | H | NH—CH₂(4-Br—C₆H₄) |
| 448 | CN | H | NH—CH₂(3-Cl—C₆H₄) |
| 449 | CN | H | NH—CH₂(3-F—C₆H₄) |
| 450 | CN | H | NH—CH₂(3-Br—C₆H₄) |
| 451 | CN | H | NH—CH₂(3,4-Cl₂—C₆H₃) |
| 452 | CN | H | NH—CH₂(3,4-F₂—C₆H₃) |
| 453 | CN | H | NH—CH₂(3-Cl—4-F—C₆H₃) |
| 454 | CN | H | NH—CH₂(4-Cl—3-F—C₆H₃) |
| 455 | CN | H | NH—CH₂(3-Br—4-F—C₆H₃) |
| 456 | CN | H | NH—CH₂(4-Br—3-F—C₆H₃) |
| 457 | CN | H | NH—CH₂(3-Br—4-Cl—C₆H₃) |
| 458 | CN | H | NH—CH₂(4-Br—3-Cl—C₆H₃) |
| 459 | CN | H | N(CH₃)—CH₂C₆H₅ |
| 460 | CN | H | N(CH₃)—CH₂(4-Cl—C₆H₄) |
| 461 | CN | H | N(CH₃)—CH₂(4-F—C₆H₄) |
| 462 | CN | H | N(CH₃)—CH₂(4-Br—C₆H₄) |
| 463 | CN | H | N(CH₃)—CH₂(3-Cl—C₆H₄) |
| 464 | CN | H | N(CH₃)—CH₂(3-F—C₆H₄) |
| 465 | CN | H | N(CH₃)—CH₂(3-Br—C₆H₄) |
| 466 | CN | H | N(CH₃)—CH₂(3,4-Cl₂—C₆H₃) |
| 467 | CN | H | N(CH₃)—CH₂(3,4-F₂—C₆H₃) |
| 468 | CN | H | N(CH₃)—CH₂(3-Cl—4-F—C₆H₃) |
| 469 | CN | H | N(CH₃)—CH₂(4-Cl—3-F—C₆H₃) |
| 470 | CN | H | N(CH₃)—CH₂(3-Br—4-F—C₆H₃) |
| 471 | CN | H | N(CH₃)—CH₂(4-Br—3-F—C₆H₃) |
| 472 | CN | H | N(CH₃)—CH₂(3-Br—4-Cl—C₆H₃) |
| 473 | CN | H | N(CH₃)—CH₂(4-Br—3-Cl—C₆H₃) |
| 474 | CN | H | NH—C₆H₅ |
| 475 | CN | H | NH—(4-Cl—C₆H₄) |
| 476 | CN | H | NH—(4-F—C₆H₄) |
| 477 | CN | H | NH—(4-Br—C₆H₄) |
| 478 | CN | H | NH—(3-Cl—C₆H₄) |
| 479 | CN | H | NH—(3-F—C₆H₄) |
| 480 | CN | H | NH—(3-Br—C₆H₄) |
| 481 | CN | H | NH—(3,4-Cl₂—C₆H₃) |
| 482 | CN | H | NH—(3,4-F₂—C₆H₃) |
| 483 | CN | H | NH—(3-Cl—4-F—C₆H₃) |
| 484 | CN | H | NH—(4-Cl—3-F—C₆H₃) |
| 485 | CN | H | NH—(3-Br—4-F—C₆H₃) |
| 486 | CN | H | NH—(4-Br—3-F—C₆H₃) |
| 487 | CN | H | NH—(3-Br—4-Cl—C₆H₃) |
| 488 | CN | H | NH—(4-Br—3-Cl—C₆H₃) |
| 489 | CN | H | N(CH₃)—C₆H₅ |
| 490 | CN | H | N(CH₃)—(4-Cl—C₆H₄) |
| 491 | CN | H | N(CH₃)—(4-F—C₆H₄) |
| 492 | CN | H | N(CH₃)—(4-Br—C₆H₄) |
| 493 | CN | H | N(CH₃)—(3-Cl—C₆H₄) |
| 494 | CN | H | N(CH₃)—(3-F—C₆H₄) |
| 495 | CN | H | N(CH₃)—(3-Br—C₆H₄) |
| 496 | CN | H | N(CH₃)—(3,4-Cl₂—C₆H₃) |
| 497 | CN | H | N(CH₃)—(3,4-F₂—C₆H₃) |
| 498 | CN | H | N(CH₃)—(3-Cl—4-F—C₆H₃) |
| 499 | CN | H | N(CH₃)—(4-Cl—3-F—C₆H₃) |
| 500 | CN | H | N(CH₃)—(3-Br—4-F—C₆H₃) |
| 501 | CN | H | N(CH₃)—(4-Br—3-F—C₆H₃) |
| 502 | CN | H | N(CH₃)—(3-Br—4-Cl—C₆H₃) |
| 503 | CN | H | N(CH₃)—(4-Br—3-Cl—C₆H₃) |
| 504 | CN | F | CH₃ |
| 505 | CN | F | CH₂CH₃ |
| 506 | CN | F | CH₂CH₂CH₃ |
| 507 | CN | F | CH(CH₃)₂ |
| 508 | CN | F | CH(CH₃)CH(CH₃)₂ |
| 509 | CN | F | CH₂CH₂CH₂CH₃ |
| 510 | CN | F | C(CH₃)₃ |
| 511 | CN | F | CH(CH₃)CH(CH₃)CH₂CH₃ |
| 512 | CN | F | CH(CH₃)CH₂CH₃ |
| 513 | CN | F | CH₂CH(CH₃)₂ |
| 514 | CN | F | (CH₂)₄CH₃ |
| 515 | CN | F | CH(CH₃)C(CH₃)₃ |
| 516 | CN | F | CH₂C(CH₃)₃ |
| 517 | CN | F | CH(CH₃)CH₂CH₂CH₃ |
| 518 | CN | F | CH(C₂H₅)CH₂CH₂CH₂CH₃ |
| 519 | CN | F | CHCl₂ |
| 520 | CN | F | CH₂CN |
| 521 | CN | F | 4-iP—c-C₆H₁₀—CH₂— |
| 522 | CN | F | (CH₂)₂—C₆H₅ |
| 523 | CN | F | CH₂CH₂C(H)═C(H)CH₂CH₂CH₂CH₂CH₃ |
| 524 | CN | F | CH(CH₃)OCH₃ |
| 525 | CN | F | CH(CH₃)OCH₂CH₂OCH₃ |
| 526 | CN | F | CH(CH₃)OCH(CH₃)₂ |
| 527 | CN | F | CH₂CH₂OCH₂CH(CH₃)₂ |
| 528 | CN | F | CH(CH₃)O—c-C₆H₁₁ |
| 529 | CN | F | CH(CH₃)OC₆H₅ |
| 530 | CN | F | 3-Ac—O—1-Me-pr |
| 531 | CN | F | 6-Ac—O—C₆H₁₂ |
| 532 | CN | F | CH₂CH(CH₃)SCH₃ |
| 533 | CN | F | CH(CH₃)CH₂SCH₃ |
| 534 | CN | F | C(CH₃)₂CH₂—3-Py |
| 535 | CN | F | c-C₃H₅ |
| 536 | CN | F | c-C₄H₇ |
| 537 | CN | F | c-C₅H₉ |
| 538 | CN | F | c-C₆H₁₁ |
| 539 | CN | F | c-C₇H₁₃ |
| 540 | CN | F | c-C₈H₁₅ |
| 541 | CN | F | 4-Me—c-C₆H₁₀ |
| 542 | CN | F | 6,6-DMe-2-N |
| 543 | CN | F | 2-EC—c-C₃H₅ |
| 544 | CN | F | C₆H₅ |
| 545 | CN | F | 4-Cl—C₆H₄ |
| 546 | CN | F | 4-F—C₆H₄ |
| 547 | CN | F | 4-Br—C₆H₄ |
| 548 | CN | F | 4-OCH₃—C₆H₄ |
| 549 | CN | F | 4-CF₃—C₆H₄ |
| 550 | CN | F | 4-(C(CH₃)₃)—C₆H₄ |
| 551 | CN | F | 3-Cl—C₆H₄ |
| 552 | CN | F | 3-F—C₆H₄ |
| 553 | CN | F | 3-Br—C₆H₄ |
| 554 | CN | F | 3-OCH₃—C₆H₄ |
| 555 | CN | F | 3-CF₃—C₆H₄ |
| 556 | CN | F | 3-(C(CH₃)₃)—C₆H₄ |
| 557 | CN | F | 2-Cl—C₆H₄ |
| 558 | CN | F | 2-F—C₆H₄ |
| 559 | CN | F | 2-Br—C₆H₄ |
| 560 | CN | F | 2-OCH₃—C₆H₄ |
| 561 | CN | F | 2-CF₃—C₆H₄ |
| 562 | CN | F | 2-(C(CH₃)₃)—C₆H₄ |
| 563 | CN | F | 3,4-(CH₃)₂—C₆H₃ |
| 564 | CN | F | 3,4-Cl₂—C₆H₃ |
| 565 | CN | F | 3,4-F₂—C₆H₃ |
| 566 | CN | F | 3-Cl—4-F—C₆H₃ |
| 567 | CN | F | 4-Cl—3-F—C₆H₃ |
| 568 | CN | F | 3-Br—4-F—C₆H₃ |
| 569 | CN | F | 4-Br—3-F—C₆H₃ |
| 570 | CN | F | 3-Br—4-Cl—C₆H₃ |
| 571 | CN | F | 4-Br—3-Cl—C₆H₃ |
| 572 | CN | F | 4-Cl—3-CF₃—C₆H₃ |
| 573 | CN | F | 3-Cl—4-CF₃—C₆H₃ |
| 574 | CN | F | 3-OCH₃—4-CH₃—C₆H₃ |
| 575 | CN | F | 4-OCH₃—3-CH₃—C₆H₃ |
| 576 | CN | F | 3-Py |
| 577 | CN | F | 4-Py |
| 578 | CN | F | 5-Br—3-Py |
| 579 | CN | F | 6-CF3—3-Py |
| 580 | CN | F | 2-ImPy |
| 581 | CN | F | 2-Pm |
| 582 | CN | F | 5-CH₃—1-C₆H₅—4-Pz |
| 583 | CN | F | 1H—3-Pyr |
| 584 | CN | F | 1,2,5-(CH₃)₂—3-AcN—4-Pyr |
| 585 | CN | F | 3-iPr—5-IsO |
| 586 | CN | F | 2-Fu |
| 587 | CN | F | 3-Th |
| 588 | CN | F | 3-THF |
| 589 | CN | F | 2-THP |
| 590 | CN | F | 6-(CH₃O)—3-(CH₃)—2-THP |
| 591 | CN | F | 3-THP |
| 592 | CN | F | 4-THP |
| 593 | CN | F | 3-THTP |
| 594 | CN | F | 4-THTP |
| 595 | CN | F | 3-THTP—CH₂ |
| 596 | CN | F | 4-THTP—CH₂ |
| 597 | CN | F | NH—CH₃ |
| 598 | CN | F | NH—CH₂CH₃ |
| 599 | CN | F | NH—CH₂CH₂CH₃ |
| 600 | CN | F | NH—CH(CH₃)₂ |
| 601 | CN | F | NH—CH₂CH₂CH₂CH₃ |
| 602 | CN | F | NH—CH(CH₃)CH₂CH₃ |
| 603 | CN | F | NH—CH₂CH(CH₃)₂ |
| 604 | CN | F | N(CH₃)—CH₃ |
| 605 | CN | F | N(CH₃)—CH₂CH₃ |
| 606 | CN | F | N(CH₃)—CH₂CH₂CH₃ |
| 607 | CN | F | N(CH₃)—CH(CH₃)₂ |
| 608 | CN | F | N(CH₃)—CH₂CH₂CH₂CH₃ |
| 609 | CN | F | N(CH₃)—CH(CH₃)CH₂CH₃ |
| 610 | CN | F | N(CH₃)—CH₂CH(CH₃)₂ |
| 611 | CN | F | NH—CH₂C₆H₅ |
| 612 | CN | F | NH—CH₂(4-Cl—C₆H₄) |
| 613 | CN | F | NH—CH₂(4-F—C₆H₄) |
| 614 | CN | F | NH—CH₂(4-Br—C₆H₄) |
| 615 | CN | F | NH—CH₂(3-Cl—C₆H₄) |
| 616 | CN | F | NH—CH₂(3-F—C₆H₄) |
| 617 | CN | F | NH—CH₂(3-Br—C₆H₄) |
| 618 | CN | F | NH—CH₂(3,4-Cl₂—C₆H₃) |
| 619 | CN | F | NH—CH₂(3,4-F₂—C₆H₃) |
| 620 | CN | F | NH—CH₂(3-Cl—4-F—C₆H₃) |
| 621 | CN | F | NH—CH₂(4-Cl—3-F—C₆H₃) |
| 622 | CN | F | NH—CH₂(3-Br—4-F—C₆H₃) |
| 623 | CN | F | NH—CH₂(4-Br—3-F—C₆H₃) |
| 624 | CN | F | NH—CH₂(3-Br—4-Cl—C₆H₃) |
| 625 | CN | F | NH—CH₂(4-Br—3-Cl—C₆H₃) |
| 626 | CN | F | N(CH₃)—CH₂C₆H₅ |
| 627 | CN | F | N(CH₃)—CH₂(4-Cl—C₆H₄) |
| 628 | CN | F | N(CH₃)—CH₂(4-F—C₆H₄) |
| 629 | CN | F | N(CH₃)—CH₂(4-Br—C₆H₄) |
| 630 | CN | F | N(CH₃)—CH₂(3-Cl—C₆H₄) |
| 631 | CN | F | N(CH₃)—CH₂(3-F—C₆H₄) |
| 632 | CN | F | N(CH₃)—CH₂(3-Br—C₆H₄) |
| 633 | CN | F | N(CH₃)—CH₂(3,4-Cl₂—C₆H₃) |
| 634 | CN | F | N(CH₃)—CH₂(3,4-F₂—C₆H₃) |
| 635 | CN | F | N(CH₃)—CH₂(3-Cl—4-F—C₆H₃) |
| 636 | CN | F | N(CH₃)—CH₂(4-Cl—3-F—C₆H₃) |
| 637 | CN | F | N(CH₃)—CH₂(3-Br—4-F—C₆H₃) |
| 638 | CN | F | N(CH₃)—CH₂(4-Br—3-F—C₆H₃) |
| 639 | CN | F | N(CH₃)—CH₂(3-Br—4-Cl—C₆H₃) |
| 640 | CN | F | N(CH₃)—CH₂(4-Br—3-Cl—C₆H₃) |
| 641 | CN | F | NH—C₆H₅ |
| 642 | CN | F | NH—(4-Cl—C₆H₄) |
| 643 | CN | F | NH—(4-F—C₆H₄) |
| 644 | CN | F | NH—(4-Br—C₆H₄) |
| 645 | CN | F | NH—(3-Cl—C₆H₄) |
| 646 | CN | F | NH—(3-F—C₆H₄) |
| 647 | CN | F | NH—(3-Br—C₆H₄) |
| 648 | CN | F | NH—(3,4-Cl₂—C₆H₃) |
| 649 | CN | F | NH—(3,4-F₂—C₆H₃) |
| 650 | CN | F | NH—(3-Cl—4-F—C₆H₃) |
| 651 | CN | F | NH—(4-Cl—3-F—C₆H₃) |
| 652 | CN | F | NH—(3-Br—4-F—C₆H₃) |
| 653 | CN | F | NH—(4-Br—3-F—C₆H₃ |
| 654 | CN | F | NH—(3-Br—4-Cl—C₆H₃) |
| 655 | CN | F | NH—(4-Br—3-Cl—C₆H₃) |
| 656 | CN | F | N(CH₃)—C₆H₅ |
| 657 | CN | F | N(CH₃)—(4-Cl—C₆H₄) |
| 658 | CN | F | N(CH₃)—(4-F—C₆H₄) |
| 659 | CN | F | N(CH₃)—(4-Br—C₆H₄) |
| 660 | CN | F | N(CH₃)—(3-Cl—C₆H₄) |
| 661 | CN | F | N(CH₃)—(3-F—C₆H₄) |
| 662 | CN | F | N(CH₃)—(3-Br—C₆H₄) |
| 663 | CN | F | N(CH₃)—(3,4-Cl₂—C₆H₃) |
| 664 | CN | F | N(CH₃)—(3,4-F₂—C₆H₃) |
| 665 | CN | F | N(CH₃)—(3-Cl—4-F—C₆H₃) |
| 666 | CN | F | N(CH₃)—(4-Cl—3-F—C₆H₃) |
| 667 | CN | F | N(CH₃)—(3-Br—4-F—C₆H₃) |
| 668 | CN | F | N(CH₃)—(4-Br—3-F—C₆H₃) |
| 669 | CN | F | N(CH₃)—(3-Br—4-Cl—C₆H₃) |
| 670 | CN | F | N(CH₃)—(4-Br—3-Cl—C₆H₃) |
| 671 | CN | CH₃ | CH₃ |
| 672 | CN | CH₃ | NH₂ |
| 673 | CN | Cl | CF₃ |
| 674 | CN | H | 1,2,5-(CH₃)₂—3-AcN—4-Pyr |
| 675 | CN | CH₃ | CH(OCH₃)₂ |
| 676 | CN | CH₃ | CH₂CH₃ |
| 677 | CN | H | NH—(3-OH—C₆H₄) |
| 678 | CN | CN | C(═O)OCH₃ |
| 679 | CN | NH₂ | CH₂C(═O)OCH₃ |
| 680 | CN | CH₂CH₃ | CH₂CH₃ |
| 681 | CN | H | NHNH₂ |
| 682 | CN | CH₃ | c-C₃H₅ |
| 683 | CN | H | 3,4,5-((OCH₃)₃C₆H₂) |
| 684 | CN | H | 3-iPr—5-IsO |
| 685 | CN | H | 3-Th |
| 686 | CN | H | 4-C₆H₅—C₆H₄ |
| 687 | CN | H | NH—(4-CH₃—C₆H₄) |
| 688 | CN | C₆H₅ | C₆H₅ |

| | | |
|---|---|---|
| 689 | CN | CH₂CH₂CH₂CH₂ |
| 690 | CN | S—CH₂—S |
| 691 | CN | S—CH₂CH₂—S |
| 692 | CN | S—CH═CH—S |
| 693 | CN | 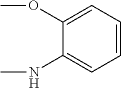 |

CH(CH₃)CH(CH₃)₂: 1,2-dimethylpropyl
CH(CH₃)CH(CH₃)CH₂CH₃: 1,2-dimethylbutyl
CH(C₂H₅)CH₂CH₂CH₂CH₃: 1-ethylpentyl
4-iP-c-C₆H₁₀—CH₂—: (4-isopropylcyclohexyl)methyl
(CH₂)₂—C₆H₅: 2-phenylethyl (phenethyl)
CH(CH₃)OCH₂CH₂OCH₃: 1-(2-methoxyethoxy)ethyl
CH(CH₃)OC₆H₅: 1-(phenoxy)ethyl
CH(CH₃)O-c-C₆H₁₁: 1-(cyclohexoxy)ethyl
3-Ac—O-1-Me-pr: 3-acetoxy-1-methyl-propyl
6-Ac—O—C₆H₁₂: 6-acetoxyhexyl
CH₂CH(CH₃)SCH₃: 2-methylsulfanylpropyl
CH(CH₃)CH₂SCH₃: 1-methyl-2-methylsulfanylethyl
C(CH₃)₂CH₂-3-Py: 1,1-dimethyl-2-(3-pyridyl)ethyl
c-C₃H₅: cyclopropyl
c-C₄H₇: cyclobutyl
c-C₅H₉: cyclopentyl
c-C₆H₁₁ cyclohexyl
c-C₇H₁₃: cyclohepyl
c-C₈H₁₅: cyclooctyl
4-Me-c-C₆H₁₀: 4-methylcyclohexyl
6,6-DMe-2-N 6,6-dimethyl-norpinan-2-yl
2-EC-c-C₃H₅: 2-ethoxycarbonylcyclopropyl
C₆H₅: phenyl4-Cl—C₆H₄: 4-chlorophenyl
4-F—C₆H₄: 4-fluorophenyl
4-Br—C₆H₄: 4-bromophenyl
4-OCH₃-C₆H₄: 4-methoxyphenyl
4-CF₃-C₆H₄: 4-trifluoromethylphenyl
4-(C(CH₃)₃)—C₆H₄: 4-tert-butylphenyl
3-Cl—C₆H₄: 3-chlorophenyl
3-F—C₆H₄: 3-fluorophenyl
3-Br—C₆H₄: 3-bromopheny
I3-OCH₃-C₆H₄: 3-methoxyphenyl
3-CF₃-C₆H₄: 3-trifluoromethylphenyl
3-(C(CH₃)₃)—C₆H₄: 3-tert-butylphenyl
2-OCH₃-C₆H₄: 2-methoxyphenyl
2-CF₃-C₆H₄: 2-trifluoromethylphenyl
2-Cl—C₆H₄: 2-chlorophenyl
2-F—C₆H₄: 2-fluorophenyl
2-Br—C₆H₄: 2-bromophenyl
2-(C(CH₃)₃)—C₆H₄: 2-tert-butylphenyl
3,4-(CH₃)₂—C₆H₃: 3,4-dimethylphenyl
3,4-Cl₂-C₆H₃: 3,4-dichlorophenyl
3,4-F₂-C₆H₃: 3,4-difluorophenyl
3-Cl-4-F—C₆H₃: 3-chloro-4-fluorophenyl
4-Cl-3-F—C₆H₃: 4-chloro-3-fluorophenyl
4-Cl-3-CF₃—C₆H₃: 4-chloro-3-trifluoromethylphenyl
3-Cl-4-CF₃-C₆H₃: 3-chloro-4-trifluoromethylphenyl
3-Br-4-F—C₆H₃: 3-bromo-4-fluorophenyl
4-Br-3-F—C₆H₃: 4-bromo-3-fluorophenyl
3-Br-4-Cl—C₆H₃: 3-bromo-4-chlorophenyl
4-Br-3-Cl—C₆H₃: 4-bromo-3-chlorophenyl
3-OH—C₆H₄: 3-hydroxyphenyl
4-CH₃-C₆H₄: 4-methylphenyl
3-OCH₃-4-CH₃-C₆H₃: 3-methoxy-4-methylphenyl
4-OCH₃-3-CH₃—C₆H₃: 4-methoxy-3-methylphenyl
3,4,5-((OCH₃)₃C₆H₂): 3,4,5-trimethoxyphenyl
4-C₆H₅-C₆H₄: 4-biphenyl
3-Py: 3-pyridyl
4-Py: 4-pyridyl
5-Br-3-Py: 5-bromo-3-pyridyl
6-CF₃-3-Py: 6-(trifluoromethyl)-3-pyridyl
2-ImPy: imidazo[1,2-a]pyridine-2-yl
2-Pm: pyrimidin-2-yl
5-CH₃-1-C₆H₅-4-Pz: 5-methyl-1-phenyl-pyrazol-4-yl

### 1H-3-Pyr: 1H-pyrrol-3-yl

1,2,5-(CH₃)₂-3-AcN-4-Pyr: 1,2,5-trimethyl-3-(acetylamino)-4-pyrrolyl
2-Fu: 2-furyl
3-Th: 3-thienyl
3-iPr-5-IsO: 3-isopropylisoxazol-5-yl
3-THF: tetrahydrofuran-3-yl
3-THP: tetrahydropyran-3-yl
2-THP: tetrahydropyran-2-yl
6-(CH₃O)-3-(CH₃)-2-THP: 6-methoxy-3-methyl-tetrahydropyran-2-yl
4-THP: tetrahydropyran-4-yl
4-THP-CH₂ tetrahydropyran-4-ylmethyl
3-THTP: tetrahydrothiopyran-3-yl
4-THTP: tetrahydrothiopyran-4-yl
3-THTP-CH₂: terahydrothiopyran-3-ylmethyl
4-THTP-CH₂: terahydrothiopyran-4-ylmethyl

Compounds of formula (I) according to the present invention can be prepared by standard methods of organic chemistry e.g. by the preparation methods and preparation schemes as described below. The definitions of Het, X, W¹, W², W³, W⁴, R¹, R², R³, R⁴, and R⁵ of the mo-lecular structures given in the schemes are as defined above. Room temperature means a temperature range between about 20 and 25° C.

The compounds of formula (I) according to the present invention can be prepared e.g. according to the preparation methods and preparation schemes as described below.

An example of a general method for the preparation of compounds of formula (I) is shown below in Scheme A. Thus, construction of the heterocyclic element 3 present in compounds of formula (I) can be achieved, for example, by alkylation of the appropriate 2-amino heterocycle precursor 1 with the appropriate reagent of formula 2. The transformation is preferably carried out in polar solvents such as acetonitrile, acetone, dichloromethane, 1,4-dioxane, tetrahydrofuran, N,N-dimethylformamide, N,N-dimethylacetamide, N-methylpyrolidinone or a C₁-C₆ alcohol ranging between room temperature and the reflux temperature of the solvent. Representative reaction conditions for the alkylation analogous to formula 1 are given in Tett. Lett. 2011, 52(23), 3033-3037. The synthesis of compounds of formula 5 can be achieved by acylation of the amine functionality in compounds of formula 3 using carboxylic acid derivatives 4 which are activated in situ. The transformation is preferably carried out in polar solvents such as acetonitrile, acetone, 1,4-dioxane, tetrahydrofuran, N,N-dimethylformamide, N,N-dimethylacetamide, N-methylpyrolidinone or in an inert solvent such as dichloromethane, 1,2-dichloroethane, or 1,2-dimethoxyethane at temperatures ranging between room temperature and the reflux temperature of the solvent. A representative procedure conditions for the acylation is given in Journal of Medicinal Chemistry, 1988, 31, 4, 807-814. Examples of suitable leaving groups (LG) in formula 2 include, but are not limited to, halogen, alkyl sulfonate, haloalkyl sulfonate, aryl sulfonate, alkyl phosphonate. Examples of suitable leaving groups (LG²) in formula 4 include, but are not limited to, halogen, alkyl sulfonate, haloalkyl sulfonate, aryl sulfonate, alkyl phosphonate, and various activated esters derived from the reaction of the free carboxylic acid with a peptide coupling reagent in the presence of an amine base (Chem. Rev., 2011, 111 (11), 6557-6602). A reversal of the order of these two steps would also result in an acceptable synthesis of the desired compounds.

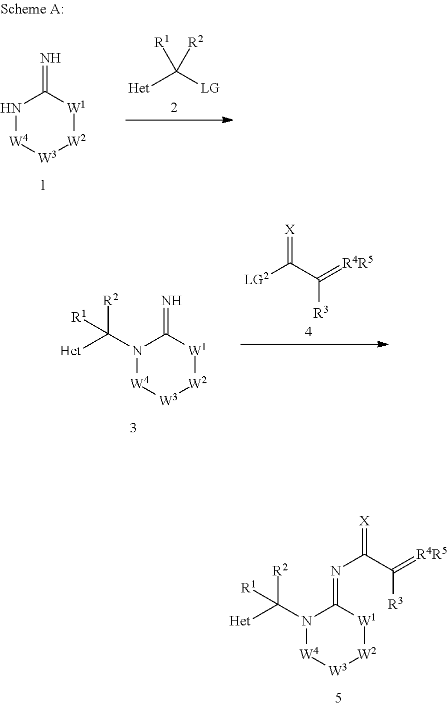

Compounds of formula (I) can also be prepared using an alternative strategy to install the olefin linkage as is shown below in Scheme B. Thus, construction intermediate 3 proceeds as described in Scheme A, and amine functionality present in intermediates of type 3 can then be acylated with a reagent of type 6. The transformation is preferably carried out in polar solvents such as acetonitrile, acetone, 1,4-dioxane, tetrahydrofuran, N,N-dimethylformamide, N,N-dimethylacetamide, N-methylpyrolidinone or in an inert solvent such as dichloromethane, 1,2-dichloroethane, or 1,2-dimethoxyethane at temperatures ranging between room temperature and the reflux temperature of the solvent. A representative procedure conditions for the acylation of is given in Journal of Medicinal Chemistry, 1988, 31, 4, 807-814. In reagent 6, suitable examples of substitutent Z are either a chlorine, bromine, iodine or hydrogen atom. Examples of suitable leaving groups (LG²) in formula 6 include, but are not limited to: halogen, alkyl sulfonate, haloalkyl sulfonate, aryl sulfonate, alkyl phosphonate, and various activated esters derived from the reaction of the free carbonic acid with a peptide coupling reagent in the presence of an amine base (Chem. Rev., 2011, 111 (11), 6557-6602). Examples of suitable leaving groups (LG³) in formula 6 include, but are not limited to: halogen, substituted alkoxide, substituted sulfide, SeCH₃, CN, acetate, aryl sulfonate, alkyl sulfonate, haloalkyl sulfonate, and alkyl phosphonate. In the next step LG³ is eliminated through the action of base (base) or single electron transfer agent (Met) to afford the desired compound of structure 5. When a base is used the reaction is best carried out in an inert solvents such as dichloromethane, tetrahydrofuran, N,N-dimethylformamide, 1,4-dioxane, N,N-dimethylacetamide, N-methylpyrolidinone, benzene, toluene, mesitylene, cymenes, or xylenes ranging between room temperature and the reflux temperature of the solvent. Examples of suitable bases to be used are: DBU, DBN, Et₃N, DIEA, LDA, LHMDS, NHMDS, KHMDS, KOt-Bu, NaOt-Bu, LiOt-Bu, NaH, KH. A representative procedure conditions for the acylation of 2-substituted pyridines are given in Journal of the American Chemical Society, 2013, 135, 17, 6677-6693. In cases where Z is a halogen atom this elimination can be affected through the use of a single electron transfer reagent in an inert or protic solvent such as dichloromethane, tetrahydrofuran, N,N-dimethylformamide, 1,4-dioxane, NMP, DMA, benzene, toluene, mesitylene, cymenes, xylenes, or a C₁-C₆ alcohol ranging between room temperature and the reflux temperature of the solvent. Examples of suitable single electron transfer agents are Sml₂, zinc, CrCl₂, indium(I) salts, or manganese (II) or (III) salts. A representative procedure conditions for the acylation of 3 is given in Angewandte Chemie International Edition, 2000, 39, 15, 2773-2775. A reversal of the order of these steps would also result in an acceptable synthesis of the desired compounds of formula (I).

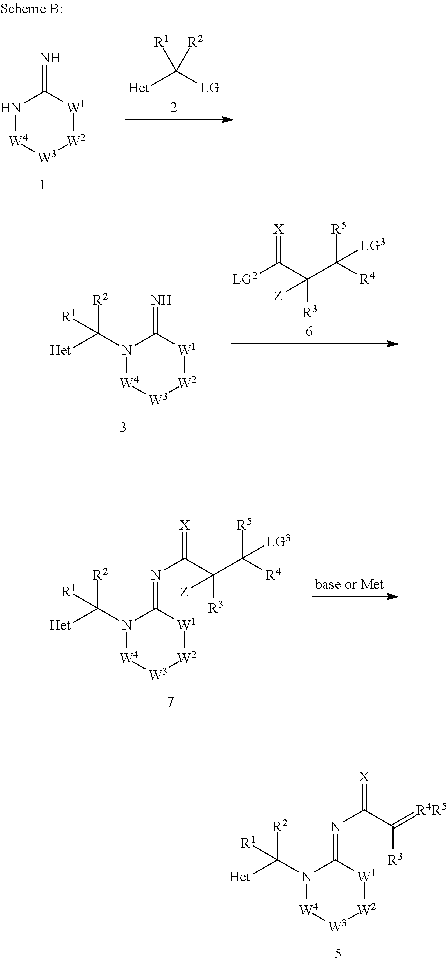

A further example of a general method for the preparation of compounds of formula (I), where R³ is cyano is shown below in Scheme C. The synthesis of compounds of formula 9 can be achieved by acylation of the amine functionality in compounds of formula 3 using carboxylic acid derivatives 8 which are activated in situ. The transformation is preferably carried out in polar solvents such as acetonitrile, acetone, 1,4-dioxane, tetrahydrofuran, N,N-dimethylformamide, N,N-dimethylacetamide, N-methylpyrolidinone or in an inert solvent such as dichloromethane, 1,2-dichloroethane, or 1,2-dimethoxyethane at temperatures ranging between room temperature and the reflux temperature of the solvent. A representative procedure condition for the acylation is given in Journal of Medicinal Chemistry, 2012, 55, 7378-7391. Examples of suitable leaving groups (LG²) in formula 8 include, but are not limited to: halogen, alkyl sulfonate, haloalkyl sulfonate, aryl sulfonate, alkyl phosphonate and various activated esters derived from the reaction of a free carboxylic acid with a peptide coupling reagent in the presence of an amine base (Chem. Rev., 2011, 111(11), 6557-6602). The compounds of formula 9 are subject matter of U.S. 61/879,691. In the next step, the compound of formula 9 is then reacted with a carbonyl compound 10 to afford compound 11. The transformation is usually carried out in an organic protic acid such as a C₁-C₆-alkanecarboxylic acid, e.g. acetic acid and in the presence of a base, preferably an amine such as piperidine.

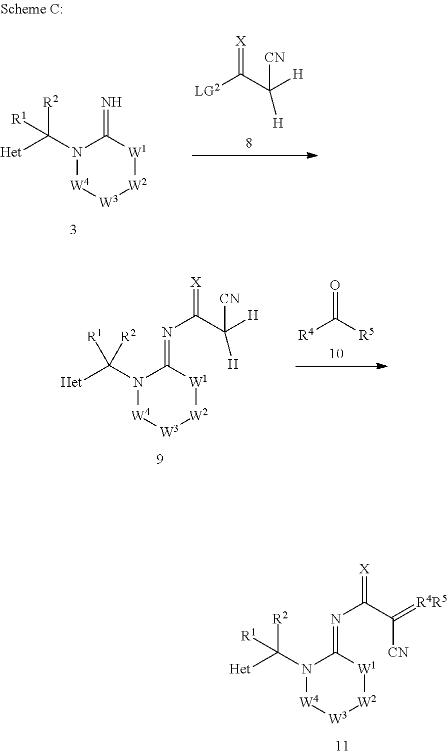

In cases where X is a sulfur atom, the sulfur atom is best installed in a subsequent step from the compound where X is an oxygen atom as detailed in scheme D.

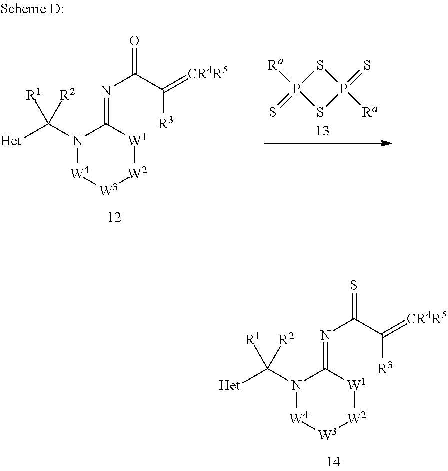

Here, the acrylate compound 12 is transformed into its thio analogue 14. The transformation is preferably achieved by using a reagent of formula 13. The reaction is preferably carried out in polar solvents such as acetonitrile, acetone, tetrahydrofuran, N,N-dimethylformamide, N,N-dimethylacetamide, N-methylpyrolidinone or in an inert solvent such as dichloromethane, 1,2-dichloroethane, or 1,2-dimethoxyethane at temperatures ranging between room temperature and the reflux temperature of the solvent. Suitable R^{a} groups for 13 are: thio, alkyl, aryl or substituted aryl. Representative reaction conditions for thionation analogous substrates are given in Tetrahedron. 1997, 53, 9, 3223-3230.

The compounds of the formula (I), and their salts are in particular suitable for efficiently controlling arthropodal pests such as arachnids, myriapedes and insects as well as nematodes.

The compounds of the formula (I) are especially suitable for efficiently combating insects, in particular the following pests:

Insects from the order of the lepidopterans (Lepidoptera), for example Acronicta major, Adoxophyes orana, Aedia leucomelas, Agrotis spp. such as Agrotis fucosa, Agrotis segetum, Agrotis ypsllon; Alabama argillacea, Anticarsia gemmatalis, Anticarsia spp., Argyresthia conjugella, Autographa gamma, Barathra brassicae, Bucculatrix thurberiella, Bupalus piniarius, Cacoecia murinana, Cacoecia podana, Capua reticulana, Carpocapsa pomonella, Cheimatobia brumata, Chilo spp. such as Chilo suppressalis; Choristoneura fumiferana, Choristoneura occidentalis, CirphiS unipuncta, Clysia ambiguella, Cnaphalocerus spp., Cydia pomonella, Dendrolimus pini, Diaphania nitidalis, Diatraea grandiosella, Earias insulana, Elasmopalpus lignosellus, Ephestia cautella, Ephestia kuehniella, Eupoecilia ambiguella, Euproctis chrysorrhoea, Euxoa spp., Evetria bouliana, Feltia spp. such as Feltia subterranean; Galleria mellonella, Grapholitha funebrana, Grapholitha molesta, Helicoverpa spp. such as Helicoverpa armigera, Helicoverpa zea; Heliothis spp. such as Heliothis armigera, Heliothis virescens, Heliothis zea; Hellula undalis, Hibernia defoliaria, Hofmannophlla pseudospretella, Homona magnanima, Hyphantria cunea, Hyponomeuta padella, Hyponomeuta malinellus, Keiferia lycopersicella, Lambdina fiscellaria, Laphygma spp. such as Laphygma exigua; Leucoptera coffeella, Leucoptera scitella, Lithocolletis blancardella, Lithophane antennata, Lobesia botrana, L oxagrotis albicosta, Loxostege sticticalis, Lymantria spp. such as Lymantria dispar, Lymantria monacha; Lyonetia clerkella, Malacosoma neustria, Mamestra spp. such as Mamestra brassicae; Mocis repanda, Mythimna separata, Orgyia pseudotsugata, Oria spp., Ostrinia spp. such as Ostrinia nubilalis; Oulema oryzae, Panolls flammea, Pectinophora spp. such as Pectinophora gossypiella; Peridroma saucia, Phalera bucephala, Phthorimaea spp. such as Phthorimaea operculella; Phyllocnistis citrella, Pieris spp. such as Pieris brassicae, Pieris rapae; Plathypena scabra, Plutella maculipennis, Plutella xylostella, Prodenia spp., Pseudaletia spp., Pseudoplusia includens, Pyrausta nubhalis, Rhyacionia frustrana, Scrobipalpula absoluta, Sitotroga cerealella, Sparganothis pilleriana, Spodoptera spp. such as Spodoptera frugiperda, Spodoptera littoralis, Spodoptera litura; Thaumatopoea pityocampa, Thermesia gemmatalis, Tinea pellionella, Tineola bisselliella, Tortrix viridana, Trichoplusia spp. such as Trichoplusia ni; Tuta absoluta, and Zeiraphera canadensis;

Beetles (Coleoptera), for example Acanthoscehdes obtectus, Adoretus spp., Agelastica alni, Agrilus sinuatus, Agriotes spp. such as Agriotes fuscicollis, Agriotes lineatus, Agriotes obscurus; Amphimallus solstitialis, Anisandrus dispar, Anobium punctatum, Anomala rufocuprea, Anoplophora spp. such as Anoplophora glabripennis; Anthonomus spp. such as Anthonomus grandis, Anthonomus pomorum; Anthrenus spp., Aphthona euphoridae, Apogonia spp., Athous haemorrhoidalis, Atomaria spp. such as Atomaria linearis; Attagenus spp., Aulacophora femoralis, Blastophagus piniperda, Blitophaga undata, Bruchidius obtectus, Bruchus spp. such as Bruchus lentis, Bruchus pisorum, Bruchus rufimanus; Byctiscus betulae, Callosobruchus chinensis, Cassida nebulosa, Cerotoma trifurcata, Cetonia aurata, Ceuthorhynchus spp. such as Ceuthorrhynchus assimilis, Ceuthorrhynchus napi; Chaetocnema tibialis, Cleonus mendicus, Conoderus spp. such as Conoderus vespertinus; Cosmopolites spp., Costelytra zealandica, Crioceris asparagi, Cryptorhynchus lapathi, Ctenicera ssp. such as Ctenicera destructor; Curculio spp., Dectes texanus, Dermestes spp., Diabrotica spp. such as Diabrotica 12-punctata Diabrotica speciosa, Diabrotica longicornis, Diabrotica semipunctata, Diabrotica virgifera; Epilachna spp. such as Epilachna varivestis, Epilachna vigintioctomaculata; Epitrix spp. such as Epitrix hirtipennis; Eutinobothrus brashiensis, Faustinus cubae, Gibbium psylloides, Heteronychus arator, Hylamorpha elegans, Hylobius abietis, Hylotrupes bajulus, Hypera brunneipennis, Hypera postica, Hypothenemus spp., Ips typographus, Lachnosterna consanguinea, Lema bilineata, Lema melanopus, Leptinotarsa spp. such as Leptinotarsa decemlineata; Limonius californicus, Lissorhoptrus orzophilus, Lissorhoptrus oryzophilus, Lixus spp., Lyctus spp. such as Lyctus bruneus; Melanotus communis, Meligethes spp. such as Meligethes aeneus; Melolontha hippocastani, Melolontha melolontha, Migdolus spp., Monochamus spp. such as Monochamus alternatus; Naupactus xanthographus, Niptus hololeucus, Oryctes rhinoceros, Oryzaephllus surinamensis, Otiorrhynchus sulcatus, Otiorrhynchus ovatus, Otiorrhynchus sulcatus, Oulema oryzae, Oxycetonia jucunda, Phaedon cochieariae, Phyllobius pyri, Phyllopertha horticola, Phyllophaga spp., Phyllotreta spp. such as Phyllotreta chrysocephala, Phyllotreta nemorum, Phyllotreta striolata; Phyllophaga spp., Phyllopertha horticola, Popillia japonica, Premnotrypes spp., Psylliodes chrysocephala, Ptinus spp., Rhizobius ventralis, Rhizopertha dominica, Sitona lineatus, Sitophilus spp. such as Sitophilus granaria, Sitophilus zeamais; Sphenophorus spp. such as Sphenophorus levis; Sternechus spp. such as Sternechus subsignatus; Symphyletes spp., Tenebrio molitor, Tribolium spp. such as Tribolium castaneum; Trogoderma spp., Tychius spp., Xylotrechus spp., and Zabrus spp. such as Zabrus tenebrioides;

Flies, mosquitoes (Diptera), e.g. Aedes spp. such as Aedes aegypti, Aedes albopictus, Aedes vexans; Anastrepha ludens, Anopheles spp. such as Anopheles albimanus, Anopheles crucians, Anopheles freeborni, Anopheles gambiae, Anopheles leucosphyrus, Anopheles maculipennis, Anopheles minimus, Anopheles quadrimaculatus, Anopheles sinensis; Bibio hortulanus, Calliphora erythrocephala, Calliphora vicina, Cerafitis capitata, Ceratitis capitata, Chrysomyia spp. such as Chrysomya bezziana, Chrysomya hominivorax, Chrysomya macellaria; Chrysops atlanticus, Chrysops discalis, Chrysops silacea, Cochliomyia spp. such as Cochliomyia hominivorax; Contarinia spp. such as Contarinia sorghicola; Cordylobia anthropophaga, Culex spp. such as Culex nigripalpus, Culex pipiens, Culex quinquefasciatus, Culex tarsalis, Culex tritaeniorhynchus; Culicoides furens, Culiseta inornata, Culiseta melanura, Cuterebra spp., Dacus cucurbitae, Dacus oleae, Dasineura brassicae, Delia spp. such as Delia antique, Delia coarctata, Delia platura, Delia radicum; Dermatobia hominis, Drosophila spp., Fannia spp. such as Fannia canicularis; Gastraphilus spp. such as Gasterophilus intestinalis; Geomyza Tripunctata, Glossina fuscipes, Glossina morsitans, Glossina palpalis, Glossina tachinoides, Haematobia irritans, HaplodiplosiS equestris, Hippelates spp., Hylemyia spp. such as Hylemyia platura; Hypoderma spp. such as Hypoderma lineata; Hyppobosca spp., Leptoconops torrens, Liriomyza spp. such as Liriomyza sativae, Liriomyza trifolii; Lucilia spp. such as Lucilia caprina, Lucilia cuprina, Lucilia sericata; Lycoria pectoralis, Mansonia titillanus, Mayetiola spp. such as Mayetiola destructor; Musca spp. such as Musca autumnalis, Musca domestica; Muscina stabulans, Oestrus spp. such as Oestrus ovis; Opomyza florum, Oscinella spp. such as Oscinella frit, Pegomya hysocyami, Phlebotomus argentipes, Phorbia spp. such as Phorbia antiqua, Phorbia brassicae, Phorbia coarctata; Prosimulium mixtum, Psila rosae, Psorophora columbiae, Psorophora discolor, Rhagoletis cerasi, Rhagoletis pomonella, Sarcophaga spp. such as Sarcophaga haemorrhoidalis; Simulium vittatum, Stomoxys spp. such as Stomoxys calcitrans; Tabanus spp. such as Tabanus atratus, Tabanus bovinus, Tabanus lineola, Tabanus similis; Tannia spp., Tipula oleracea, Tipula paludosa, and Wohlfahrtia spp.;

Thrips (Thysanoptera), e.g. Baliothrips biformis, Dichromothrips corbettl, Dichromothrips ssp., Enneothrips flavens, Frankliniella spp. such as Frankliniella fusca, Frankliniella occidentalis, Frankliniella tritici; Heliothrips spp., Hercinothrips femoralis, Kakothrips spp., Rhipiphorothrips cruentatus, Scirtothrips spp. such as Scirtothrips citri; Taeniothrips cardamoni, Thrips spp. such as Thrips oryzae, Thrips palmi, Thrips tabaci;

Termites (Isoptera), e.g. Calotermes flavicollis, Coptotermes formosanus, Heterotermes aureus, Heterotermes longiceps, Heterotermes tenuis, Leucotermes flavipes, Odontotermes spp., Reticulitermes spp. such as Reticulitermes speratus, Reticulitermes flavipes, Reticulitermes grassei, Reticulitermes lucifugus, Reticulitermes santonensis, Reticulitermes virginicus; Termes natalensis;

Cockroaches (Blattaria—Blattodea), e.g. Acheta domesticus, Blatta orientalis, Blattella asahinae, Blattella germanica, Gryllotalpa spp., Leucophaea maderae, Locusta spp., Melanoplus spp., Periplaneta americana, Periplaneta australasiae, Periplaneta brunnea, Periplaneta fuligginosa, Periplaneta japonica;

Bugs, aphids, leafhoppers, whiteflies, scale insects, cicadas (Hemiptera), e.g. Acrosternum spp. such as Acrosternum hilare; Acyrthosipon spp. such as Acyrthosiphon onothychis, Acyrthosiphon pisum; Adelges laricis, Aeneolamia spp., Agonoscena spp., Aleurodes spp., Aleurolobus barodensis, Aleurothnixus spp., Amrasca spp., Anasa tristis, Antestiopsis spp., Anuraphis cardui, Aonidiella spp., Aphanostigma piri, Aphidula nasturtii, Aphis spp. such as Aphis fabae, Aphis forbesi, Aphis gossypil, Aphis grossulariae, Aphis pomi, Aphis sambuci, Aphis schneideri, Aphis spiraecola; Arboridia apicalis, Arilus critatus, Aspidiella spp., Aspidiotus spp., Atanus spp., Aulacorthum solani, Bemisia spp. such as Bemisia argentifolii, Bemisia tabaci; Blissus spp. such as Blissus leucopterus; Brachycaudus cardui, Brachycaudus helichrysi, Brachycaudus persicae, Brachycaudus prunicola, Brachycolus spp., Brevicoryne brassicae, Calligypona marginata, Calocoris spp., Campylomma livida, Capitophorus horni, Carneocephala fulgida, Cavelerius spp., Ceraplastes spp., Ceratovacuna lanigera, Cercopidae, Cerosipha gossypil, Chaetosiphon fragaefolii, Chionaspis tegalensis, Chlorita onukii, Chromaphlis juglandicola, Chrysomphalus ficus, Cicadulina mblla, Cinnex spp. such as Cimex hemipterus, Cimex lectularius; Coccomytilus halli, Coccus spp., Creontiades Cryptomyzus ribis, Cryptomyzus ribis, Cyrtopeltis notatus, Dalbulus spp., Dasynus piperis, Dialeurades spp., Diaphorina spp., Diaspis spp., Dichelops furcatus, Diconocoris hewetti, Doralis spp., Dreyfusia nordmannianae, Dreyfusia piceae, Drosicha spp., Dysaphis spp. such as Dysaphis plantaginea, Dysaphis pyri, Dysaplis radicola; Dysaulacorthum pseudosolani, Dysdercus spp. such as Dysdercus cingulatus, Dysdercus intermedius; Dysmicoccus spp., Empoasca spp. such as Empoasca fabae, Empoasca solana; Eriosoma spp., Erythroneura spp., Eurygaster spp. such as Eurygaster integriceps; Euscells bilobatus, Euschistus spp. such as Euschistuos heros, Euschistus impictiventris, Euschistus servus; Geococcus coffeae, Halyomorpha spp. such as Halyomorpha halys; Heliopeltis spp., Homalodisca coagulata, Horcias nobilellus, Hyalopterus pruni, Hyperomyzus lactucae, Icerya spp., Idiocerus spp., Idioscopus spp., Laodelphax striatellus, Lecanium spp., Lepidosaphes spp., Leptocorisa spp., Leptoglossus phyllopus, Lipaphlis erysimi, Lygus spp. such as Lygus hesperus, Lygus lineolaris, Lygus pratensis; Macropes excavatus, Macrosiphum spp. such as Macrosiphum rosae, Macrosiphum avenae, Macrosiphum euphorbiae; Mahanarva fimbriolata, Megacopta cribraria, Megoura viciae, Melanaphis pyrarius, Melanaphis sacchari, Metcafiella spp., Metopolophium dirhodum, Miridae spp., Monellia costalis, Monelliopsis pecanis, Myzus spp. such as Myzus ascalonicus, Myzus cerasi, Myzus persicae, Myzus varians; Nasonovia ribis-nigri, Nephotettix spp. such as Nephotettix malayanus, Nephotettix nigropictus, Nephotettix parvus, Nephotettix virescens; Nezara spp. such as Nezara viridula; Nilaparvata lugens, Oebalus spp., Oncometopia spp., Orthezia praelonga, Parabemisia myricae, Paratrioza spp., Parlatoria spp., Pemphigus spp. such as Pemphigus bursarius; Pentomidae, Peregrinus maidis, Perkinsiella saccharicida, Phenacoccus spp., Phloeomyzus passerinii, Phorodon humuli, Phylloxera spp., Piesma quadrata, Piezodorus spp. such as Piezodorus guildinil, Pinnaspis aspidistrae, Planococcus spp., Protopulvinaria pyriformis, Psallus seriatus, Pseudacysta persea, Pseudaulacaspis pentagona, Pseudococcus spp. such as Pseudococcus comstocki; Psylla spp. such as Psylla mali, Psylla piri; Pteromalus spp., Pyrilla spp., Quadraspidiotus spp., Quesada gigas, Rastrococcus spp., Reduvius senilis, Rhodnius spp., Rhopalomyzus ascalonicus, Rhopalosiphum spp. such as Rhopalosiphum pseudobrassicas, Rhopalosiphum insertum, Rhopalosiphum maidis, Rhopalosiphum padi; Sagatodes spp., Sahlbergella singularis, Saissetia spp., Sappaphis mala, Sappaphis mali, Scaphoides titanus, Schizaphis graminum, Schizoneura lanuginosa, Scotinophora spp., Selenaspidus articulatus, Sitobion avenae, Sogata spp., Sogatella furcifera, Solubea insularis, Stephanitis nashi, Stictocephala festina, Tenalaphara malayensis, Thyanta spp. such as Thyanta perditor; Tibraca spp., Tinocallis caryaefoliae, Tomaspis spp., Toxoptera spp. such as Toxoptera aurantii; Trialeurodes spp. such as Trialeurodes vaporariorum; Triatoma spp., Trioza spp., Typhlocyba spp., Unaspis spp. such as Unaspis yanonensis; and Viteus vitifolii;

Ants, bees, wasps, sawflies (Hymenoptera), e.g. Athalia rosae, Atta capiguara, Atta cephalotes, Atta cephalotes, Atta laevigata, Atta robusta, Atta sexdens, Atta texana, Bombus spp., Camponotus floridanus, Crematogaster spp., Dasymutilla occidentalis, Diprion spp., Dolichovespula maculata, Hoplocampa spp. such as Hoplocampa minuta, Hoplocampa testudinea; Lasius spp. such as Lasius niger, Linepithema humile, Monomorium pharaonis, Paravespula germanica, Paravespula pennylvanica, Paravespula vulgaris, Pheidole megacephala, Pogonomyrmex barbatus, Pogonomyrmex californicus, Pollistes rubiginosa, Solenopsis geminata, Solenopsis invicta, Solenopsis richteri, Solenopsis xyloni, Vespa spp. such as Vespa crabro, and Vespula squamosal;

Crickets, grasshoppers, locusts (Orthoptera), e.g. Acheta domestica, Calliptamus italicus, Chortoicetes terminifera, Dociostaurus maroccanus, Gryllotalpa africana, Gryllotalpa gryllotalpa, Hieroglyphus daganensis, Kraussaria angulifera, Locusta migratoria, Locustana pardalina, Melanoplus bivittatus, Melanoplus femurrubrum, Melanoplus mexicanus, Melanoplus sanguinipes, Melanoplus spretus, Nomadacris septemfasciata, Oedaleus senegalensis, Schistocerca americava, Schistocerca gregaria, Tachycines asynamorus, and Zonozerus variegatus;

Earwigs (Dermaptera), e.g. forticula auricularia,

Lice (Phthiraptera), e.g. Damalinia spp., Pediculus spp. such as Pediculus humanus captis, Pediculus humanus corporis; Pthirus pubis, Haematopinus spp. such as Haematopinus eurysternus, Haematopinus suis, Linognathus spp. such as Linognathus vituli; Bovicola bovis, Menopon gallinae, Menacanthus stramineus and Solenopotes capillatus, Trichodectes spp.;

Fleas (Siphonaptera), e.g. Ceratophyllus spp., Ctenocephalides felis, Ctenocephalides canis, Xenopsylla cheopis, Pulex irritans, Tunga penetrans, and Nosopsyllus fasciatus.

The compounds of the formula (I) are also suitable for efficiently combating arthropd pests different from insects such as, in particular the following pests:

arachnids (Arachnida), such as acari, e.g. of the families Argasidae, Ixodidae and Sarcoptidae, such as Amblyomma spp. (e.g. Amblyomma americanum, Amblyomma variegatum, Amblyomma maculatum), Argas spp. (e.g. Argas persicus), Boophilus spp. (e.g. Boophilus annulatus, Boophilus decoloratus, Boophilus microplus), Dermacentor silvarum, Dermacentor andersoni, Dermacentor variabilis, Hyalomma spp. (e.g. Hyalomma truncatum), Ixodes spp. (e.g. Ixodes ricinus, Ixodes rubicundus, Ixodes scapularis, Ixodes holocyclus, Ixodes pacificus), Ornithodorus spp. (e.g. Ornithodorus moubata, Ornithodorus hermsi, Ornithodorus turicata), Ornithonyssus bacoti, Otobius megnini, Dermanyssus gallinae, Psoroptes spp. (e.g. Psoroptes ovis), Rhipicephalus spp. (e.g. Rhipicephalus sanguineus, Rhipicephalus appendiculatus, Rhipicephalus evertsi), Rhizoglyphus spp., Sarcoptes spp. (e.g. Sarcoptes scabiei), and Eriophyidae spp. such as Acaria sheldoni, Aculops spp. (e.g. Aculops pelekassi) Aculus spp. (e.g. Aculus schlechtendali), Epitrimerus pyri, Phyllocoptruta oleivora and Eriophyes spp. (e.g. Eriophyes sheldoni); Tarsonemidae spp. such as Hemitarsonemus spp., Phytonemus pallidus and Polyphagotarsonemus latus, Stenotarsonemus spp.; Tenuipalpidae spp. such as Brevipalpus spp. (e.g. Brevipalpus phoenicis); Tetranychidae spp. such as Eotetranychus spp., Eutetranychus spp., Oligonychus spp., Tetranychus cinnabarinus, Tetranychus kanzawai, Tetranychus pacificus, Tetranychus telarius and Tetranychus urticae; Bryobia praetiosa, Panonychus spp. (e.g. Panonychus ulmi, Panonychus citri), Metatetranychus spp. and Oligonychus spp. (e.g. Oligonychus pratensis), Vasates lycopersici; Araneida, e.g. Latrodectus mactans, and Loxosceles reclusa. And Acarus siro, Chorioptes spp., Scorpio maurus;

Silverfish, firebrat (Thysanura), e.g. Lepisma saccharina and Thermobia domestica;

Centipedes (Chilopoda), e.g. Geophilus spp., Scutigera spp. such as Scutigera coleoptrata;

Millipedes (Diplopoda), e.g. Blaniulus guttulatus, Narceus spp.,

Springtails (Collembola), e.g. Onychiurus ssp. such as Onychiurus armatus,

They are also suitable for controlling nematodes: plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species such as Aphelenchoides besseyi; Sting nematodes, Belonolaimus longicaudatus and other Belonolaimus species; Pine nematodes, Bursaphelenchus lignicolus Mamiya et Kiyohara, Bursaphelenchus xylophllus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Lesion nematodes, Pratylenchus brachyurus, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species such as Tylenchulus semipenetrans; Dagger nematodes, Xiphinema species; and other plant parasitic nematode species.

Examples of further pest species which may be controlled by compounds of formula (I) include: from the class of the Bivalva, for example, Dreissena spp.; from the class of the Gastropoda, for example, Arion spp., Biomphalaria spp., Bulinus spp., Deroceras spp., Galba spp., Lymnaea spp., Oncomelania spp., Succinea spp.; from the class of the helminths, for example, Ancylostoma duodenale, Ancylostoma ceylanicum, Acylostoma braziliensis, Ancylostoma spp., Ascaris lumbricoides, Ascaris spp., Brugia malayi, Brugia timori, Bunostomum spp., Chabertia spp., Clonorchis spp., Cooperia spp., Dicrocoelium spp., Dictyocaulus filaria, Diphyllobothrium latum, Dracunculus medinensis, Echinococcus granulosus, Echinococcus multllocularis, Enterobius vermicularis, Faciola spp., Haemonchus spp. such as Haemonchus contortus; Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercora lis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichiura, Wuchereria bancrofti; from the order of the Isopoda, for example, Armadillidium vulgare, Oniscus asellus, Porcellio scaber; from the order of the Symphyla, for example, Scutigerella immaculata.

Further examples of pest species which may be controlled by compounds of formula (I) include: Anisoplia austriaca, Apamea spp., Austroasca viridigrisea, Baliothrips biformis, Caenorhabditis elegans, Cephus spp., Ceutorhynchus napi, Chaetocnema aridula, Chilo auricilius, Chilo indicus, Chilo polychrysus, Chortiocetes terminifera, Cnaphalocroci medinalis, Cnaphalocrosis spp., Collas eurytheme, Collops spp., Cornitermes cumulans, Creontiades spp., Cyclocephala spp., Dalbulus maidis, Deraceras reticulatum, Diatrea saccharalis, Dichelops furcatus, Dicladispa armigera, Diloboderus spp. such as Diloboderus abderus; Edessa spp., Epinotia spp., Formicidae, Geocoris spp., Globitermes sulfureus, Gryllotalpidae, Halotydeus destructor, Hipnodes bicolor, Hydrellia phllippina, Julus spp., Laodelphax spp., Leptocorsia acuta, Leptocorsia oratorius, Liogenys fuscus, Lucilia spp., Lyogenys fuscus, Mahanarva spp., Maladera matrida, Marasmia spp., Mastotermes spp., Mealybugs, Megascelis ssp, Metamasius hemipterus, Microtheca spp., Mocis latipes, Murgantia spp., Mythemina separata, Neocapritermes opacus, Neocapritermes parvus, Neomegalotomus spp., Neotermes spp., Nymphula depunctalis, Oebalus pugnax, Orseolia spp. such as Orseolia oryzae; Oxycaraenus hyalinipennis, Plusia spp., Pomacea canaliculata, Procornitermes ssp, Procornitermes triacifer, Psylloides spp., Rachiplusia spp., Rhodopholus spp., Scaptocoris castanea, Scaptocoris spp., Scirpophaga spp. such as Scirpophaga incertulas, Scirpophaga innotata; Scotinophara spp. such as Scotinophara coarctata; Sesamia spp. such as Sesamia inferens, Sogaella frucifera, Solenapsis geminata, Spissistilus spp., Stalk borer, Stenchaetothrips biformis, Steneotarsonemus spinki, Sylepta derogata, Telehin licus, Trichostrongylus spp.

Compounds of the formula (I) are particularly useful for controlling insects of the orders Hemiptera and Thysanoptera.

For use in a method according to the present invention, the compounds of the formula (I) can be converted into the customary formulations, e.g. solutions, emulsions, suspensions, dusts, powders, pastes, granules and directly sprayable solutions. The use form depends on the particular purpose and application method. Formulations and application methods are chosen to ensure in each case a fine and uniform distribution of the compound of the formula (I) according to the present invention.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publish-ers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, antifoaming agents, anti-freezing agents, for seed treatment formulation also optionally colorants and/or binders and/or gelling agents.

Solvents/carriers, which are suitable, are e.g.:
solvents such as water, aromatic solvents (for example Solvesso products, xylene and the like), paraffins (for example mineral fractions), alcohols (for example methanol, butanol, pentanol, benzyl alcohol), ketones (for example cyclohexanone, gamma-butyrolactone), pyrrolidones (N-methyl-pyrrolidone (NMP), N-octylpyrrolidone NOP), acetates (glycol diac-etate), alkyl lactates, lactones such as g-butyrolactone, glycols, fatty acid dimethylamides, fatty acids and fatty acid esters, triglycerides, oils of vegetable or animal origin and modified oils such as alkylated plant oils. In principle, solvent mixtures may also be used.
carriers such as ground natural minerals and ground synthetic minerals, such as silica gels, finely divided silicic acid, silicates, talc, kaolin, attaclay, limestone, lime, chalk, bole, loess, clay, dolomite, diatomaceous earth, calcium sulfate and magnesium sulfate, magnesium oxide, ground synthetic materials, fertilizers, such as, for example, ammonium sulfate, ammonium phosphate, ammonium nitrate, ureas and products of vegetable origin, such as cereal meal, tree bark meal, wood meal and nutshell meal, cellulose powders and other solid carriers.

Suitable emulsifiers are nonionic and anionic emulsifiers, for example polyoxyethylene fatty alcohol ethers, alkylsulfonates and arylsulfonates.

Examples of dispersants are lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters,

Also anti-freezing agents such as glycerin, ethylene glycol, propylene glycol and bacteri-cides such as can be added to the formulation.

Suitable antifoaming agents are for example antifoaming agents based on silicon or magnesium stearate.

Suitable preservatives are for example dichlorophen and benzyl alcohol hemiformal

Suitable thickeners are compounds which confer a pseudoplastic flow behavior to the formulation, i.e. high viscosity at rest and low viscosity in the agitated stage. Mention may be made, in this context, for example, of commercial thickeners based on polysaccharides, such as Xanthan Gum® (Kelzan® from Kelco), Rhodopol®23 (Rhone Poulenc) or Veegum® (from R.T. Vanderbilt), or organic phyllosilicates, such as Attaclay® (from Engelhardt). Antifoam agents suitable for the dispersions according to the invention are, for example, silicone emulsions (such as, for example, Silikon® SRE, Wacker or Rhodorsil® from Rhodia), long-chain alcohols, fatty acids, organofluorine compounds and mixtures thereof. Biocides can be added to stabilize the compositions according to the invention against attack by microorganisms. Suitable biocides are, for example, based on isothiazolones such as the compounds marketed under the trade-marks Proxel® from Avecia (or Arch) or Acticide® RS from Thor Chemie and Kathon® MK from Rohm & Haas. Suitable antifreeze agents are organic polyols, for example ethylene glycol, propylene glycol or glycerol. These are usually employed in amounts of not more than 10% by weight, based on the total weight of the active compound composition. If appropriate, the active compound compositions according to the invention may comprise 1 to 5% by weight of buffer, based on the total amount of the formulation prepared, to regulate the pH, the amount and type of the buffer used depending on the chemical properties of the active compound or the active compounds. Examples of buffers are alkali metal salts of weak inorganic or organic acids, such as, for example, phosphoric acid, boronic acid, acetic acid, propionic acid, citric acid, fumaric acid, tartaric acid, oxalic acid and succinic acid.

Substances which are suitable for the preparation of directly sprayable solutions, ennui-sions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dusts can be prepared by mixing or concomitantly grinding the active substances with a solid carrier.

Granules, for example coated granules, impregnated granules and homogeneous granules, can be prepared by binding the active ingredients to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active ingredient. The active ingredients are employed in a purity of from 90% to 100%, preferably 95% to 100% (according to NMR spectrum).

For seed treatment purposes, respective formulations can be diluted 2-10 fold leading to concentrations in the ready to use preparations of 0.01 to 60% by weight active compound by weight, preferably 0.1 to 40% by weight.

The compound of formula (I) can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compounds according to the invention.

The following are examples of formulations:
1. Products for dilution with water. For seed treatment purposes, such products may be applied to the seed diluted or undiluted.

A) Water-soluble concentrates (SL, LS)

10 parts by weight of the active compound is dissolved in 90 parts by weight of water or a water-soluble solvent. As an alternative, wetters or other auxiliaries are added. The active compound dissolves upon dilution with water, whereby a formulation with 10% (w/w) of active conn-pound is obtained.

B) Dispersible concentrates (DC)

20 parts by weight of the active compound is dissolved in 70 parts by weight of cyclohexanone with addition of 10 parts by weight of a dispersant, for example polyvinylpyrrolidone. Dilution with water gives a dispersion, whereby a formulation with 20% (w/w) of active compounds is obtained.

C) Emulsifiable concentrates (EC)

15 parts by weight of the active compounds is dissolved in 7 parts by weight of xylene with addition of calcium dodecylbenzenesulfonate and castor oil ethoxylate (in each case 5 parts by weight). Dilution with water gives an emulsion, whereby a formulation with 15% (w/w) of active compounds is obtained.

D) Emulsions (EW, EO, ES)

25 parts by weight of the active compound is dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of active compound is obtained.

E) Suspensions (SC, OD, FS)

In an agitated ball mill, 20 parts by weight of the active compound is comminuted with addition of 10 parts by weight of dispersants, wetters and 70 parts by weight of water or of an organic solvent to give a fine active compound suspension. Dilution with water gives a stable suspension of the active compound, whereby a formulation with 20% (w/w) of active compound is obtained.

F) Water-dispersible granules and water-soluble granules (WG, SG)

50 parts by weight of the active compound is ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the active compound, whereby a formulation with 50% (w/w) of active compound is obtained.

G) Water-dispersible powders and water-soluble powders (WP, SP, SS, WS)

75 parts by weight of the active compound are ground in a rotor-stator mill with addition of 25 parts by weight of dispersants, wetters and silica gel. Dilution with water gives a stable dispersion or solution of the active compound, whereby a formulation with 75% (w/w) of active compound is obtained.

H) Gel-Formulation (GF)

In an agitated ball mill, 20 parts by weight of the active compound is comminuted with addition of 10 parts by weight of dispersants, 1 part by weight of a gelling agent wetters and 70 parts by weight of water or of an organic solvent to give a fine active compound suspension. Dilution with water gives a stable suspension of the active compound, whereby a formulation with 20% (w/w) of active compound is obtained.
2. Products to be applied undiluted for foliar applications. For seed treatment purposes, such products may be applied to the seed diluted or undiluted.

I) Dustable powders (DP, DS)

5 parts by weight of the active compound are ground finely and mixed intimately with 95 parts by weight of finely divided kaolin. This gives a dustable product having 5% (w/w) of active compound.

J) Granules (GR, FG, GG, MG)

0.5 part by weight of the active compound is ground finely and associated with 95.5 parts by weight of carriers, whereby a formulation with 0.5% (w/w) of active compound is obtained. Current methods are extrusion, spray-drying or the fluidized bed. This gives granules to be applied undiluted for foliar use.

K) ULV solutions (UL)

10 parts by weight of the active compound is dissolved in 90 parts by weight of an organic solvent, for example xylene. This gives a product having 10% (w/w) of active compound, which is applied undiluted for foliar use.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use products can be varied within rel-atively wide ranges. In general, they are from 0.0001 to 10%, preferably from 0.01 to 1%.

The active ingredients may also be used successfully in the ultra-low-volume process (ULV), it being possible to apply formulations comprising over 95% by weight of active ingredient, or even to apply the active ingredient without additives.

In the method of this invention compounds of formula (I) may be applied with other active ingredients, for example with other pesticides, insecticides, herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

M.1 Acetylcholine esterase (AChE) inhibitors from the class of

M.1A carbamates, for example aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb and triazamate; or from the class of

M.1B organophosphates, for example acephate, azamethiphos, azinphos-ethyl, azinphosmethyl, cadusafos, chlorethoxyfos, chlorfenvinphos, chlormephos, chlorpyrifos, chlorpyri-fos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxy-aminothio-phosphoryl) salicylate, isoxathion, malathion, mecarbam, methamidophos, methida-thion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupi-rimfos, temephos, terbufos, tetrachlorvinphos, thiometon, triazophos, trichlorfon and vamidothi-on;

M.2. GABA-gated chloride channel antagonists such as:

M.2A cyclodiene organochlorine compounds, as for example endosulfan or chlordane; or

M.2B fiproles (phenylpyrazoles), as for example ethiprole, fipronil, flufiprole, pyrafluprole and pyriprole;

M.3 Sodium channel modulators from the class of

M.3A pyrethroids, for example acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, del-tamethrin, empenthrin, esfenvalerate, etofenprox, fenpropathrin, fenvalerate, flucythrinate, flumethrin, tau-fluvalinate, halfenprox, imiprothrin, meperfluthrin, metofluthrin, momfluorothrin, permethrin, phenothrin, prallethrin, profluthrin, pyrethrin (pyrethrum), resmethrin, silafluofen, tefluthrin, tetramethylfluthrin, tetramethrin, tralomethrin and transfluthrin; or

M.3B sodium channel modulators such as DDT or methoxychlor;

M.4 Nicotinic acetylcholine receptor agonists (nAChR) from the class of

M.4A neonicotinoids, for example acteamiprid, chlothianidin, dinotefuran, imidacloprid, ni-tenpyram, thiacloprid and thiamethoxam; or the compounds

M.4A.1: 1-[(6-chloro-3-pyridinyl)methyl]-2,3,5,6,7,8-hexahydro-9-nitro-(5S,8R)-5,8-Epoxy-1H-imidazo[1,2-a]azepine; or

M.4A.2: 1-[(6-chloro-3-pyridyl)methyl]-2-nitro-1-[(E)-pentylideneamino]guanidine; or

M4.A.3: 1-[(6-chloro-3-pyridyl)methyl]-7-methyl-8-nitro-5-propoxy-3,5,6,7-tetrahydro-2H-imidazo[1,2-a]pyridine;

or M.4B nicotine.

M.5 Nicotinic acetylcholine receptor allosteric activators from the class of spinosyns, for example spinosad or spinetoram;

M.6 Chloride channel activators from the class of avermectins and milbemycins, for example abamectin, emamectin benzoate, ivermectin, lepimectin or milbemectin;

M.7 Juvenile hormone mimics, such as

M.7A juvenile hormone analogues as hydroprene, kinoprene and methoprene; or others as M.7B fenoxycarb or M.7C pyriproxyfen;

M.8 miscellaneous non-specific (multi-site) inhibitors, for example

M.8A alkyl halides as methyl bromide and other alkyl halides, or

M.8B chloropicrin, or M.8C sulfuryl fluoride, or M.8D borax, or M.8E tartar emetic;

M.9 Selective homopteran feeding blockers, for example

M.9B pymetrozine, or M.9C flonicamid;

M.10 Mite growth inhibitors, for example

M.10A clofentezine, hexythiazox and diflovidazin, or M.10B etoxazole;

M.11 Microbial disruptors of insect midgut membranes, for example bacillus thuringiensis or bacillus sphaericus and the insecticdal proteins they produce such as bacillus thuringiensis subsp. israelensis, bacillus sphaericus, bacillus thuringiensis subsp. aizawai, bacillus thuringiensis subsp. kurstaki and bacillus thuringiensis subsp. tenebrionis, or the Bt crop proteins: Cry1Ab, Cry1Ac, Cry1Fa, Cry2Ab, mCry3A, Cry3Ab, Cry3Bb and Cry34/35Ab1;

M.12 Inhibitors of mitochondrial ATP synthase, for example

M.12A diafenthiuron, or

M.12B organotin miticides such as azocyclotin, cyhexatin or fenbutatin oxide, or M.12C propargite, or M.12D tetradifon;

M.13 Uncouplers of oxidative phosphorylation via disruption of the proton gradient, for example chlorfenapyr, DNOC or sulfluramid;

M.14 Nicotinic acetylcholine receptor (nAChR) channel blockers, for example nereistoxin analogues as bensultap, cartap hydrochloride, thiocyclam or thiosultap sodium;

M.15 Inhibitors of the chitin biosynthesis type 0, such as benzoylureas as for example bis-trifluron, chlorfluazuron, diflubenzuron, flucycloxuron, flufenoxuron, hexaflumuron, lufenuron, novaluron, noviflumuron, teflubenzuron or triflumuron;

M.16 Inhibitors of the chitin biosynthesis type 1, as for example buprofezin;

M.17 Moulting disruptors, Dipteran, as for example cyromazine;

M.18 Ecdyson receptor agonists such as diacylhydrazines, for example methoxyfenozide, tebufenozide, halofenozide, fufenozide or chromafenozide;

M.19 Octopamin receptor agonists, as for example amitraz;

M.20 Mitochondrial complex III electron transport inhibitors, for example

M.20A hydramethylnon, or M.20B acequinocyl, or M.20C fluacrypyrim;

M.21 Mitochondrial complex I electron transport inhibitors, for example

M.21A METI acaricides and insecticides such as fenazaquin, fenpyroximate, pyrimidifen, pyridaben, tebufenpyrad or tolfenpyrad, or M.21B rotenone;

M.22 Voltage-dependent sodium channel blockers, for example

M.22A indoxacarb, or M.22B metaflumizone, or M.22C 1-[(E)-[2-(4-cyanophenyl)-1-[3-(trifluoromethyl)phenyl]ethylidene]amino]-3-[4-(difluoromethoxy)phenyl]urea;

M.23 Inhibitors of the of acetyl CoA carboxylase, such as Tetronic and Tetramic acid derivatives, for example spirodiclofen, spiromesifen or spirotetramat;

M.24 Mitochondrial complex IV electron transport inhibitors, for example

M.24A phosphine such as aluminium phosphide, calcium phosphide, phosphine or zinc phosphide, or M.24B cyanide.

M.25 Mitochondrial complex II electron transport inhibitors, such as beta-ketonitrile derivatives, for example cyenopyrafen or cyflumetofen;

M.28 Ryanodine receptor-modulators from the class of diamides, as for example flubendi-amide, chlorantraniliprole (Rynaxypyr®), cyantraniliprole (Cyazypyr®), or the phthalamide compounds

M.28.1: (R)-3-Chlor-N1-{2-methyl-4-[1,2,2,2-tetrafluor-1-(trifluormethyl)ethyl]phenyl}-N2-(1-methyl-2-methylsulfonylethyl)phthalamid and

M.28.2: (S)-3-Chlor-N1-{2-methyl-4-[1,2,2,2-tetrafluor-1-(trifluormethyl)ethyl]phenyl}-N2-(1-methyl-2-methylsulfonylethyl)phthalamid, or the compound

M.28.3: 3-bromo-N-{2-bromo-4-chloro-6-[(1-cyclopropylethyl)carbamoyl]phenyl}-1-(3-chlorpyridin-2-yl)-1H-pyrazole-5-carboxamide (proposed ISO name: cyclaniliprole), or the compound

M.28.4: methyl-2-[3,5-dibromo-2-({[3-bromo-1-(3-chlorpyridin-2-yl)-1H-pyrazol-5-yl]carbonyl}amino)benzoyl]-1,2-dimethylhydrazinecarboxylate; or a compound selected from M.28.5a) to M.28.51):

M.28.5a) N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide;

M.28.5b) N-[4-chloro-2-[(diethyl-lambda-4-sulfanylidene)carbamoyl]-6-methyl-phenyl]-2-(3-chloro-2-pyridyl)-5-(trifluoromethyl)pyrazole-3-carboxamide;

M.28.5c) N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide;

M.28.5d) N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide;

M.28.5e) N-[4,6-dichloro-2-[(diethyl-lambda-4-sulfanylidene)carbamoyl]-phenyl]-2-(3-chloro-2-pyridyl)-5-(difluoromethyl)pyrazole-3-carboxamide;

M.28.5f) N-[4,6-dibromo-2-[(di-2-propyl-lambda-4-sulfanylidene)carbamoyl]-phenyl]-2-(3-chloro-2-pyridyl)-5-(trifluoromethyl)pyrazole-3-carboxamide;

M.28.5g) N-[4-chloro-2-[(di-2-propyl-lambda-4-sulfanylidene)carbamoyl]-6-cyano-phenyl]-2-(3-chloro-2-pyridyl)-5-(trifluoromethyl)pyrazole-3-carboxamide;

M.28.5h) N-[4,6-dibromo-2-[(diethyl-lambda-4-sulfanylidene)carbamoyl]-phenyl]-2-(3-chloro-2-pyridyl)-5-(trifluoromethyl)pyrazole-3-carboxamide;

M.28.5i) N-[2-(5-amino-1,3,4-thiadiazol-2-yl)-4-chloro-6-methyl-phenyl]-5-bromo-2-(3-chloro-2-pyridyl)pyrazole-3-carboxamide;

M.28.5j) 5-chloro-2-(3-chloro-2-pyridyl)-N-[2,4-dichloro-6-[(1-cyano-1-methyl-ethyl)carbamoyl]phenyl]pyrazole-3-carboxamide;

M.28.5k) 5-bromo-N-[2,4-dichloro-6-(methylcarbamoyl)phenyl]-2-(3,5-dichloro-2-pyridyl)pyrazole-3-carboxamide;

M.28.5l) N-[2-(tert-butylcarbamoyl)-4-chloro-6-methyl-phenyl]-2-(3-chloro-2-pyridyl)-5-(fluoromethoxy)pyrazole-3-carboxamide; or a compound selected from

M.28.6 N2-(1-cyano-1-methyl-ethyl)-N1-(2,4-dimethylphenyl)-3-iodo-phthalamide; or

M.28.7 3-chloro-N2-(1-cyano-1-methyl-ethyl)-N1-(2,4-dimethylphenyl)phthalamide;

M.UN.X insecticidal active compounds of unknown or uncertain mode of action, as for example afidopyropen, azadirachtin, amidoflumet, benzoximate, bifenazate, bromopropylate, chinomethionat, cryolite, dicofol, flufenerim, flometoquin, fluensulfone, flupyradifurone, piperonyl butoxide, pyridalyl, pyrifluquinazon, sulfoxaflor, pyflubumide or the compounds

M.UN.X.1: 4-[5-(3,5-Dichloro-phenyl)-5-trifluoromethyl-4,5-dihydro-isoxazol-3-yl]-2-methyl-N-[(2,2,2-trifluoro-ethylcarbamoyl)-methyl]-benzamide, or the compound

M.UN.X.2: 4-[5-[3-chloro-5-(trifluoromethyl)phenyl]-5-(trifluoromethyl)-4H-isoxazol-3-yl]-N-[2-oxo-2-(2,2,2-trifluoroethylamino)ethyl]naphthalene-1-carboxamide, or the compound

M.UN.X.3: 11-(4-chloro-2,6-dimethylphenyl)-12-hydroxy-1,4-dioxa-9-azadispiro[4.2.4.2]-tetradec-11-en-10-one, or the compound

M.UN.X.4: 3-(4′-fluoro-2,4-dimethylbiphenyl-3-yl)-4-hydroxy-8-oxa-1-azaspiro[4.5]dec-3-en-2-one, or the compound

M.UN.X.5: 1-[2-fluoro-4-methyl-5-[(2,2,2-trifluoroethyl)sulfinyl]phenyl]-3-(trifluoromethyl)-1H-1,2,4-triazole-5-amine, or actives on basis of bacillus firmus (Votivo, 1-1582); or

M.UN.X.6; a compound selected from the group of

M.UN.X.6a) (E/Z)—N-[1-[(6-chloro-3-pyridyl)methyl]-2-pyridylidene]-2,2,2-trifluoro-acetamide;

M.UN.X.6b) (E/Z)—N-[1-[(6-chloro-5-fluoro-3-pyridyl)methyl]-2-pyridylidene]-2,2,2-trifluoro-acetamide;

M.UN.X.6c) (E/Z)-2,2,2-trifluoro-N-[1-[(6-fluoro-3-pyridyl)methyl]-2-pyridylidene]acetamide;

M.UN.X.6d) (E/Z)—N-[1-[(6-bromo-3-pyridyl)methyl]-2-pyridylidene]-2,2,2-trifluoro-acetamide;

M.UN.X.6e) (E/Z)—N-[1-[1-(6-chloro-3-pyridyl)ethyl]-2-pyridylidene]-2,2,2-trifluoro-acetamide;

M.UN.X.6f) (E/Z)—N-[1-[(6-chloro-3-pyridyl)methyl]-2-pyridylidene]-2,2-difluoro-acetamide;

M.UN.X.6g) (E/Z)-2-chloro-N-[1-[(6-chloro-3-pyridyl)methyl]-2-pyridylidene]-2,2-difluoro-acetamide;

M.UN.X.6h) (E/Z)—N-[1-[(2-chloropyrimidin-5-yl)methyl]-2-pyridylidene]-2,2,2-trifluoro-acetamide and

M.UN.X.6i) (E/Z)—N-[1-[(6-chloro-3-pyridyl)methyl]-2-pyridylidene]-2,2,3,3,3-pentafluoro-propanamide.); or of the compounds

M.UN.X.7: 3-[3-chloro-5-(trifluoromethyl)phenyl]-4-oxo-1-(pyrimidin-5-ylmethyl)pyrido[1,2-a]pyrimidin-1-ium-2-olate; or

M.UN.X.8: 8-chloro-N-[2-chloro-5-methoxyphenyl)sulfonyl]-6-trifluoromethyl)-imidazo[1,2-a]pyridine-2-carboxamide; or

M.UN.X.9: 4-[5-(3,5-dichlorophenyl)-5-(trifluoromethyl)-4H-isoxazol-3-yl]-2-methyl-N-(1-oxothietan-3-yl)benzamide; or

M.UN.X.10: 5-[3-[2,6-dichloro-4-(3,3-dichloroallyloxy)phenoxy]propoxy]-1H-pyrazole.

The commercially available compounds of the group M listed above may be found in The Pesticide Manual, 15th Edition, C. D. S. Tomlin, British Crop Protection Council (2011) among other publications.

The quinoline derivative flometoquin is shown in WO 2006/013896. The aminofuranone compounds flupyradifurone is known from WO 2007/115644. The sulfoximine compound sulfoxaflor is known from WO 2007/149134. The pyrethroid momfluorothrin is known from U.S. Pat. No. 6,908,945. The pyrazole acaricide pyflubumide is known from WO 2007/020986. The isoxazoline compounds have been described likewise M.UN.X.1 in WO 2005/085216, M.UN.X2. in WO 2009/002809 and in WO 2011/149749 and the isoxazoline M.UN.X.9 in WO 2013/050317. The pyripyropene derivative afidopyropen has been described in WO 2006/129714. The spiro-ketal-substituted cyclic ketoenol derivative M.UN.X.3 is known from WO 2006/089633 and the biphenyl-substituted spirocyclic ketoenol derivative M.UN.X.4 from WO 2008/067911. Finally triazoylphenylsulfide like M.UN.X.5 have been described in WO 2006/043635 and biological control agents on basis of bacillus firmus in WO 2009/124707. The neonicotionids 4A.1 is known from WO 20120/069266 and WO 2011/06946, the M.4.A.2 from WO 2013/003977, the M4.A.3.from WO 2010/069266.

The Metaflumizone analogue M.22C is described in CN 10171577. The phthalimides M.28.1 and M.28.2 are both known from WO 2007/101540. The anthranilamide M.28.3 has been described in WO 2005/077934. The hydrazide compound M.28.4 has been described in WO 2007/043677. The anthranilamides M.28.5a) to M.28.5h) can be prepared as described in WO 2007/006670, WO 2013/024009 and WO 2013/024010, the anthranilamide M.28.5i) is described in WO 2011/085575, the M.28.5j) in WO 2008/134969, the M.28.5k) in US2011/046186 and the M.28.51) in WO 2012/034403. The diamide compounds M.28.6 and M.28.7 can be found in CN 102613183.

The compounds M.UN.X.6a) to M.UN.X.6i) listed in M.UN.X.6 have been described in WO 2012/029672. The mesoionic antagonist compound M.UN.X.7 was described in WO 2012/092115, the nematicide M.UN.X.8 in WO 2013/055584 and the Pyridalyl-type analogue M.UN.X.10 in WO 2010/060379.

In another embodiment of the invention, the compounds of formula (I), or their stereoisomers, salts, tautomers and N-oxides, may also be applied with fungicides as compound II.

The following list F of active substances, in conjunction with which the compounds according to the invention can be used, is intended to illustrate the possible combinations but does not limit them:

F.I) Respiration Inhibitors

F.I-1) Inhibitors of complex III at Qo site:

strobilurins: azoxystrobin, coumethoxystrobin, coumoxystrobin, dimoxystrobin, enestro-burin, fluoxastrobin, kresoxim-methyl, metominostrobin, orysastrobin, picoxystrobin, pyra-clostrobin, pyrametostrobin, pyraoxystrobin, pyribencarb, triclopyricarb/chlorodincarb, tri-floxystrobin, 2-[2-(2,5-dimethyl-phenoxymethyl)-phenyl]-3-methoxy-acrylic acid methyl ester and 2 (2-(3-(2,6-dichlorophenyl)-1-methyl-allylideneaminooxymethyl)-phenyl)-2-methoxyimino-N methyl-acetamide;

oxazolidinediones and imidazolinones: famoxadone, fenamidone;

F.I-2) Inhibitors of complex II (e.g. carboxamides):

carboxanilides: benodanil, benzovindiflupyr, bixafen, boscalid, carboxin, fenfuram, fenhexamid, fluopyram, flutolanil, furametpyr, isopyrazam, isotianil, mepronil, oxycarboxin, pen-flufen, penthiopyrad, sedaxane, tecloftalam, thifluzamide, tiadinil, 2-amino-4 methyl-thiazole-5-carboxanilide, N-(3′,4′,5′ trifluorobiphenyl-2 yl)-3-difluoromethyl-1-methyl-1H-pyrazole-4 carboxamide (fluxapyroxad), N-(4′-trifluoromethylthiobiphenyl-2-yl)-3 difluoromethyl-1-methyl-1H pyrazole-4-carboxamide, N-(2-(1,3,3-trimethyl-butyl)-phenyl)-1,3-dimethyl-5 fluoro-1H-pyrazole-4 carboxamide, 3-(difluoromethyl)-1-methyl-N-(1,1,3-trimethylindan-4-yl)pyrazole-4-carboxamide, 3-(trifluoromethyl)-1-methyl-N-(1,1,3-trimethylindan-4-yl)pyrazole-4-carboxamide, 1,3-dimethyl-N-(1,1,3-trimethylindan-4-yl)pyrazole-4-carboxamide, 3-(trifluoromethyl)-1,5-dimethyl-N-(1,1,3-trimethylindan-4-yl)pyrazole-4-carboxamide, 3-(difluoromethyl)-1,5-dimethyl-N-(1,1,3-trimethylindan-4-yl)pyrazole-4-carboxamide, 1,3,5-trimethyl-N-(1,1,3-trimethylindan-4-yl)pyrazole-4-carboxamide, 3-(difluoromethyl)-1-methyl-N-(1,1,3-trimethylindan-4-yl)pyrazole-4-carboxamide, 3-(trifluoromethyl)-1-methyl-N-(1,1,3-trimethylindan-4-yl)pyrazole-4-carboxamide, 1,3-dimethyl-N-(1,1,3-trimethylindan-4-yl)pyrazole-4-carboxamide, 3-(trifluoromethyl)-1,5-dimethyl-N-(1,1,3-trimethylindan-4-yl)pyrazole-4-carboxamide, 3-(difluoromethyl)-1,5-dimethyl-N-(1,1,3-trimethylindan-4-yl)pyrazole-4-carboxamide, 1,3,5-trimethyl-N-(1,1,3-trimethylindan-4-yl)pyrazole-4-carboxamide;

F.I-3) Inhibitors of complex III at Qi site: cyazofamid, amisulbrom, [(3S,6S,7R,8R)-8-benzyl-3-[(3-acetoxy-4-methoxy-pyridine-2-carbonyl)amino]-6-methyl-4,9-dioxo-1,5-dioxonan-7-yl] 2-methylpropanoate, [(3S,6S,7R,8R)-8-benzyl-3-[[3-(acetoxymethoxy)-4-methoxy-pyridine-2-carbonyl]amino]-6-methyl-4,9-dioxo-1,5-dioxonan-7-yl] 2-methylpropanoate, [(3S,6S,7R,8R)-8-benzyl-3-[(3-isobutoxycarbonyloxy-4-methoxy-pyridine-2-carbonyl)amino]-6-methyl-4,9-dioxo-1,5-dioxonan-7-yl] 2-methylpropanoate, [(3S,6S,7R,8R)-8-benzyl-3-[[3-(1,3-benzodioxol-5-ylmethoxy)-4-methoxy-pyridine-2-carbonyl]amino]-6-methyl-4,9-dioxo-1,5-dioxonan-7-yl] 2-methylpropanoate, 3S,6S,7R,8A)-3-[[(3-hydroxy-4-methoxy-2-pyridinyl)carbonyl]amino]-6-methyl-4,9-dioxo-8-(phenylmethyl)-1,5-dioxonan-7-yl 2-methylpropanoate;

F.I-4) Other respiration inhibitors (complex I, uncouplers) diflumetorim; (5,8-difluoro-quinazolin-4-yl)-{2-[2-fluoro-4-(4-trifluoromethylpyridin-2-yloxy)-phenyl]-ethyl}-amine; tec-nazen; ametoctradin; silthiofam; nitrophenyl derivates: binapacryl, dinobuton, dinocap, fluazi-nam, ferimzone, nitrthal-isopropyl,

and including organometal compounds: fentin salts, such as fentin-acetate, fentin chloride or fentin hydroxide;

F.I1) Sterol biosynthesis inhibitors (SBI fungicides)

F.II-1) C14 demethylase inhibitors (DMI fungicides, e.g. triazoles, imidazoles)

triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, paclobutrazole, penconazole, propiconazole, prothioconazole, simeconazole, tebuconazole, tetraconazole, triadimefon, triadimenol, triticonazole, uniconazole, 1-[rel-(2S;3R)-3-(2-chlorophenyl)-2-(2,4-difluorophenyl)-oxiranylmethyl]-5-thiocyanato-1H-[1,2,4]triazole, 2-[rel-(2 S;3R)-3-(2-chlorophenyl)-2-(2,4-difluorophenyl)-oxiranylmethyl]-2H-[1,2,4]triazole-3-thiol;

imidazoles: imazalil, pefurazoate, oxpoconazole, prochloraz, triflumizole;

pyrimidines, pyridines and piperazines: fenarimol, nuarimol, pyrifenox, triforine, 1-[rel-(2S;3R)-3-(2-chlorophenyl)-2-(2,4-difluorophenyl)-oxiranylmethyl]-5-thiocyanato-1H-[1,2,4]triazole, 2-[rel-(2S;3R)-3-(2-chlorophenyl)-2-(2,4-difluorophenyl)-oxiranylmethyl]-2H-[1,2,4]triazole-3-thiol;

F.II-2) Delta14-reductase inhibitors (Amines, e.g. morpholines, piperidines) morpholines: aldimorph, dodemorph, dodemorph-acetate, fenpropimorph, tridemorph; piperidines: fenpropidin, piperalin; spiroketalamines: spiroxamine;

F.II-3) Inhibitors of 3-keto reductase: hydroxyanilides: fenhexamid;

F.III) Nucleic acid synthesis inhibitors

F.III-1) RNA, DNA synthesis

phenylamides or acyl amino acid fungicides: benalaxyl, benalaxyl-M, kiralaxyl, metalaxyl, metalaxyl-M (mefenoxam), ofurace, oxadixyl;

isoxazoles and iosothiazolones: hymexazole, octhilinone;

F.III-2) DNA topisomerase inhibitors: oxolinic acid;

F.III-3) Nucleotide metabolism (e.g. adenosin-deaminase), hydroxy (2-amino)-pyrimidines: bupirimate;

F.IV) Inhibitors of cell division and or cytoskeleton

F.IV-1) Tubulin inhibitors: benzimidazoles and thiophanates: benomyl, carbendazim, fuberidazole, thiabendazole, thiophanate-methyl;

triazolopyrimidines: 5-chloro-7 (4-methylpiperidin-1-yl)-6-(2,4,6-trifluorophenyl)-[1,2,4]triazolo[1,5 a]pyrimidine;

F.IV-2) Other cell division inhibitors

benzamides and phenyl acetamides: diethofencarb, ethaboxam, pencycuron, fluopicolide, zoxamide;

F.IV-3) Actin inhibitors: benzophenones: metrafenone; pyriofenone;

F.V) Inhibitors of amino acid and protein synthesis

F.V-1) Methionine synthesis inhibitors (anilino-pyrimidines)

anilino-pyrimidines: cyprodinil, mepanipyrim, nitrapyrin, pyrimethanil;

F.V-2) Protein synthesis inhibitors (anilino-pyrimidines)

antibiotics: blasticidin-S, kasugamycin, kasugamycin hydrochloride-hydrate, mildiomycin, streptomycin, oxytetracyclin, polyoxine, validamycin A;

F.VI) Signal transduction inhibitors

F.VI-1) MAP/Histidine kinase inhibitors (e.g. anilino-pyrimidines)

dicarboximides: fluoroimid, iprodione, procymidone, vinclozolin;

phenylpyrroles: fenpiclonil, fludioxonil;

F.VI-2) G protein inhibitors: quinolines: quinoxyfen;

F.VII) Lipid and membrane synthesis inhibitors

F.VII-1) Phospholipid biosynthesis inhibitors

organophosphorus compounds: edifenphos, iprobenfos, pyrazophos;

dithiolanes: isoprothiolane;

F.VII-2) Lipid peroxidation: aromatic hydrocarbons: dicloran, quintozene, tecnazene, tolclofos-methyl, biphenyl, chloroneb, etridiazole;

F.VII-3) Carboxyl acid amides (CAA fungicides)

cinnamic or mandelic acid amides: dimethomorph, flumorph, mandiproamid, pyrimorph;

valinamide carbamates: benthiavalicarb, iprovalicarb, pyribencarb, valifenalate and N-(1-(1-(4-cyano-phenyl)ethanesulfonyl)-but-2-yl) carbamic acid-(4-fluorophenyl) ester;

F.VII-4) Compounds affecting cell membrane permeability and fatty acids:

1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone, carbamates: propamocarb, propamocarb-hydrochlorid,

F.VII-5) fatty acid amide hydrolase inhibitors: 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone;

F.VIII) Inhibitors with Multi Site Action

F.VIII-1) Inorganic active substances: Bordeaux mixture, copper acetate, copper hydroxide, copper oxychloride, basic copper sulfate, sulfur;

F.VIII-2) Thio- and dithiocarbamates: ferbam, mancozeb, maneb, metam, methasulpho-carb, metiram, propineb, thiram, zineb, ziram;

F.VIII-3) Organochlorine compounds (e.g. phthalimides, sulfamides, chloronitriles):

anilazine, chlorothalonil, captafol, captan, folpet, dichlofluanid, dichlorophen, flusulfamide, hexachlorobenzene, pentachlorphenole and its salts, phthalide, tolylfluanid, N-(4-chloro-2-nitro-phenyl)-N-ethyl-4-methyl-benzenesulfonamide;

F.VIII-4) Guanidines and other: guanidine, dodine, dodine free base, guazatine, guazatine-acetate, iminoctadine, iminoctadine-triacetate, iminoctadine-tris(albesilate), 2,6-dimethyl-1H,5H-[1,4]dithiino[2,3-c:5,6-c′]dipyrrole-1,3,5,7(2H,6H)-tetraone;

F.VIII-5) Ahtraquinones: dithianon;

F.IX) Cell wall synthesis inhibitors

F.IX-1) Inhibitors of glucan synthesis: validamycin, polyoxin B;

F.IX-2) Melanin synthesis inhibitors: pyroquilon, tricyclazole, carpropamide, dicyclomet, fenoxanil;

F.X) Plant defence inducers

F.X-1) Salicylic acid pathway: acibenzolar-S-methyl;

F.X-2) Others: probenazole, isotianil, tiadinil, prohexadione-calcium;

phosphonates: fosetyl, fosetyl-aluminum, phosphorous acid and its salts;

F.XI) Unknown mode of action:bronopol, chinomethionat, cyflufenamid, cymoxanil, daz-omet, debacarb, diclomezine, difenzoquat, difenzoquat-methylsulfate, diphenylamin, fenpyra-zamine, flumetover, flusulfamide, flutianil, methasulfocarb, nitrapyrin, nitrothal-isopropyl, oxa-thiapiprolin, oxin-copper, proquinazid, tebufloquin, tecloftalam, triazoxide, 2-butoxy-6-iodo-3-propylchromen-4-one, N-(cyclopropylmethoxyimino-(6-difluoro-methoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N methyl formamidine, N′ (4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl formamidine, N′-(5-difluoromethyl-2 methyl-4-(3-trimethylsilanyl-propoxy)-phenyl)-N-ethyl-N-methyl formamidine, 2-{1-[2-(5-methyl-3-trifluoromethyl-pyrazole-1-yl)-acetyl]-piperidin-4-yl}-thiazole-4-carboxylic acid methyl-(1,2,3,4-tetrahydro-naphthalen-1-yl)-amide, 2-{1-[2-(5-methyl-3-trifluoromethyl-pyrazole-1-yl)-acetyl]-piperidin-4-yl}-thiazole-4-carboxylic acid methyl-(A)-1,2,3,4-tetrahydro-naphthalen-1-yl-amide, methoxy-acetic acid 6-tert-butyl-8-fluoro-2,3-dimethyl-quinolin-4-yl ester and N-Methyl-2-{1-[(5-methyl-3-trifluoromethyl-1H-pyrazol-1-yl)-acetyl]-piperidin-4-yl}-N-[(1R)-1,2,3,4-tetrahydronaphthalen-1-yl]-4-thiazolecarboxamide, 3-[5-(4-chloro-phenyl)-2,3-dimethyl-isoxazolidin-3 yl]-pyridine, pyrisoxa-zole, 5-amino-2-isopropyl-3-oxo-4-ortho-tolyl-2,3-dihydro-pyrazole-1 carbothioic acid S-allyl ester, N-(6-methoxy-pyridin-3-yl) cyclopropanecarboxylic acid amide, 5-chloro-1 (4,6-dimethoxy-pyrimidin-2-yl)-2-methyl-1H-benzoimidazole, 2-(4-chloro-phenyl)-N-[4-(3,4-dimethoxy-phenyl)-isoxazol-5-yl]-2-prop-2-ynyloxyacetamide;

F.XI) Growth regulators: abscisic acid, amidochlor, ancymidol, 6-benzylaminopurine, brassinolide, butralin, chlormequat (chlormequat chloride), choline chloride, cyclanilide, damino-zide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N 6-benzyladenine, paclobutrazol, prohexadione (prohexadione-calcium), prohydrojasmon, thidiazuron, triapenthenol, tributyl phos-phorotrithioate, 2,3,5 tri iodobenzoic acid, trinexapac-ethyl and uniconazole;

F.XII) Biological control agents

Ampelomyces quisqualis (e.g. AQ 10® from Intrachem Bio GmbH & Co. KG, Germany), Aspergillus flavus (e.g. AFLAGUARD® from Syngenta, CH), Aureobasidium pullulans (e.g. BO-TECTOR® from bio-ferm GmbH, Germany), Bacillus pumilus (e.g. NRRL Accession No. B-30087 in SONATA® and BALLAD® Plus from AgraQuest Inc., USA), Bacillus subtilis (e.g. isolate NRRL-Nr. B-21661 in RHAPSODY®, SERENADE® MAX and SERENADE® ASO from AgraQuest Inc., USA), Bacillus subtilis var. amyloliquefaciens FZB24 (e.g. TAEGRO® from Novozyme Biologicals, Inc., USA), Candida oleophlla I-82 (e.g. ASPIRE® from Ecogen Inc., USA), Candida saitoana (e.g. BIOCURE® (in mixture with lysozyme) and BIOCOAT® from Micro Flo Company, USA (BASF SE) and Arysta), Chitosan (e.g. ARMOUR-ZEN from BotriZen Ltd., NZ), Clonostachys rosea f. catenulata, also named Gliocladium catenulatum (e.g. isolate J1446: PRESTOP® from Verdera, Finland), Coniothyrium minitans (e.g. CONTANS® from Prophyta, Germany), Cryphonectria parasitica (e.g. Endothia parasitica from CNICM, France), Cryptococcus albidus (e.g. YIELD PLUS® from Anchor Bio-Technologies, South Africa), Fusarium oxysporum (e.g. BIOFOX® from S.I.A.P.A., Italy, FUSACLEAN® from Natural Plant Protection, France), Metschnikowia fructicola (e.g. SHEMER® from Agrogreen, Israel), Microdochium dimerum (e.g. ANTIBOT® from Agrauxine, France), Phlebiopsis gigantea (e.g. ROTSOP® from Verdera, Finland), Pseudozyma flocculosa (e.g. SPORODEX® from Plant Products Co. Ltd., Canada), Pythium oligandrum DV74 (e.g. POLYVERSUM® from Remeslo SSRO, Biopreparaty, Czech Rep.), Reynoutria sachlinensis (e.g. REGALIA® from Marrone Biolnnovations, USA), Talaromyces flavus V117b (e.g. PROTUS® from Prophyta, Germany), Trichoderma asperellum SKT-1 (e.g. ECO-HOPE® from Kumiai Chemical Industry Co., Ltd., Japan), T. atrovinde LC52 (e.g. SENTINEL® from Agrimm Technologies Ltd, NZ), T. harzianum T-22 (e.g. PLANTSHIELD® der Firma BioWorks Inc., USA), T. harzianum TH 35 (e.g. ROOT PRO® from Mycontrol Ltd., Israel), T. harzianum T-39 (e.g. TRICHODEX® and TRICHODERMA 2000® from Mycontrol Ltd., Israel and Makhteshim Ltd., Israel), T. harzianum and T. viride (e.g. TRICHOPEL from Agrimm Technologies Ltd, NZ), T. harzianum ICC012 and T. viride ICC080 (e.g. REMEDIER® WP from Isagro Ricerca, Italy), T. polysporum and T. harzianum (e.g. BINAB® from BINAB Bio-Innovation AB, Sweden), T. stromaticum (e.g. TRICOVAB® from C.E.P.L.A.C., Brazil), T. virens GL-21 (e.g. SOILGARD® from Certis LLC, USA), T. viride (e.g. TRIECO® from Ecosense Labs. (India) Pvt. Ltd., Indien, BIO-CURE® F from T. Stanes & Co. Ltd., Indien), T. viride TV1 (e.g. T. viride TV1 from Agribiotec srl, Italy), Ulocladium oudemansii HRU3 (e.g. BOTRY-ZEN® from Botry-Zen Ltd, NZ).

The commercially available compounds II of the group F listed above may be found in The Pesticide Manual, 15th Edition, C. D. S. Tomlin, British Crop Protection Council (2011) among other publications. Their preparation and their activity against harmful fungi is known (cf.: http://www.alanwood.net/pesticides/); these substances are commercially available. The compounds described by IUPAC nomenclature, their preparation and their fungicidal activity are also known (cf. Can. J. Plant Sci. 48(6), 587-94, 1968; EPA 141 317; EP-A 152 031; EP-A 226 917; EP A 243 970; EP A 256 503; EP-A 428 941; EP-A 532 022; EP-A 1 028 125; EP-A 1 035 122; EP A 1 201 648; EP A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 11/028657).

The invertebrate pest, e.g. the insects, arachnids and nematodes, the plant, soil or water in which the plant is growing can be contacted with the present compounds of formula (I), including their stereoisomers and tautomers, as well the salts thereof, or composition(s) containing them by any application method known in the art. As such, “contacting” includes both direct contact (applying the compound/compositions directly on the animal pest or plant—typically to the foliage, stem or roots of the plant) and indirect contact (applying the compounds/compositions to the locus of the animal pest or plant).

The compounds of formula (I), including their stereoisomers and tautomers, as well the salts thereof, or the pesticidal compositions comprising them may be used to protect growing plants and crops from attack or infestation by animal pests, especially insects, acaridae or arachnids by contacting the plant/crop with a pesticidally effective amount of compounds of formula (I). The term “crop” refers both to growing and harvested crops.

The compounds of the present invention and the compositions comprising them are particularly important in the control of a multitude of insects on various cultivated plants, such as cereal, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

The compounds of the present invention are employed as such or in form of compositions by treating the insects or the plants, plant propagation materials, such as seeds, soil, surfaces, materials or rooms to be protected from insecticidal attack with a insecticidally effective amount of the active compounds. The application can be carried out both before and after the infection of the plants, plant propagation materials, such as seeds, soil, surfaces, materials or rooms by the insects.

The present invention also includes a method of combating animal pests which comprises contacting the animal pests, their habit, breeding ground, food supply, cultivated plants, seed, soil, area, material or environment in which the animal pests are growing or may grow, or the materials, plants, seeds, soils, surfaces or spaces to be protected from animal attack or infestation with a pesticidally effective amount of at least one active compound of the formula (I), a stereoisomers, a tautomere or a salt thereof.

Moreover, animal pests may be controlled by contacting the target pest, its food supply, habitat, breeding ground or its locus with a pesticidally effective amount of compounds of formula (I), a stereoisomer, a tautomere or a salt thereof. As such, the application may be carried out before or after the infection of the locus, growing crops, or harvested crops by the pest.

The compounds of the invention can also be applied preventively to places at which occurrence of the pests is expected.

The compounds of formula (I), including their stereoisomers and their tautomers, as well as their salts may be also used to protect growing plants from attack or infestation by pests. The use includes contacting the plant with a pesticidally effective amount of compounds of formula (I), a stereoisomer, a tautomere or a salt thereof. As such, “contacting” includes both direct contact, i.e. applying the compound/compositions directly on the pest and/or plant—typically to the foliage, stem or roots of the plant, and indirect contact, i.e. applying the compounds/compositions to the locus of the pest and/or plant.

“Locus” means a habitat, breeding ground, plant, seed, soil, area, material or environment in which a pest or parasite is growing or may grow.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “cultivated plants” is to be understood as including plants which have been modified by breeding, mutagenesis or genetic engineering. Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-transitional modification of protein(s) (oligo- or polypeptides) poly for example by glycosylation or polymer additions such as prenyl-ated, acetylated or farnesylated moieties or PEG moieties (e.g. as disclosed in Biotechnol Prog. 2001 July-August; 17(4):720-8., Protein Eng Des Set. 2004 January; 17(1):57-66, Nat Protoc. 2007; 2(5):1225-35., Curr Opin Chem Biol. 2006 October; 10(5):487-91. Epub 2006 Aug. 28., Bio-materials. 2001 March; 22(5):405-17, Bioconjug Chem. 2005 January-February; 16(1):113-21).

The term “cultivated plants” is to be understood also including plants that have been rendered tolerant to applications of specific classes of herbicides, such as hydroxy-phenylpyruvate dioxygenase (HPPD) inhibitors; acetolactate synthase (ALS) inhibitors, such as sulfonyl ureas (see e. g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073) or imidazolinones (see e. g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073); enolpyruvylshikimate-3-phosphate synthase (EPSPS) inhibitors, such as glyphosate (see e. g. WO 92/00377); glutamine synthetase (GS) inhibitors, such as glufosinate (see e. g. EP-A-0242236, EP-A-242246) or oxynil herbicides (see e. g. U.S. Pat. No. 5,559,024) as a result of conventional methods of breeding or genetic engineering. Several cultivated plants have been rendered tolerant to herbicides by conventional methods of breeding (mutagenesis), for example Clearfield® summer rape (Canola) being tolerant to imidazolinones, e. g. imazamox. Genetic engineering methods have been used to render cultivated plants, such as soybean, cotton, corn, beets and rape, tolerant to herbicides, such as glyphosate and glufosinate, some of which are commercially available under the trade names Round-upReady® (glyphosate) and LibertyLink® (glufosinate).

The term “cultivated plants” is to be understood also including plants that are by the use of recombinant DNA techniques capable to synthesize one or more insecticidal proteins, especially those known from the bacterial genus Bacillus, particularly from Bacillus thuringiensis, such as ä-endotoxins, e. g. CryIA(b), CryIA(c), CryIF, CryIF(a2), CryIIA(b), CryIIIA, CryIIIB(b1) or Cry9c; vegetative insecticidal proteins (VIP), e. g. VIP1, VIP2, VIP3 or VIP3A; insecticidal proteins of bacteria colonizing nematodes, for example Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, for example WO 02/015701). Further examples of such toxins or genetically-modified plants capable of synthesizing such toxins are dis-closed, for example, in EP-A 374 753, WO 93/007278, WO 95/34656, EP-A 427 529, EP-A 451 878, WO 03/018810 and WO 03/052073. The methods for producing such genetically modified plants are generally known to the person skilled in the art and are described, for example, in the publications mentioned above. These insecticidal proteins contained in the genetically modified plants impart to the plants producing these proteins protection from harmful pests from certain taxonomic groups of arthropods, particularly to beetles (Coleoptera), flies (Diptera), and butter-flies and moths (Lepidoptera) and to plant parasitic nematodes (Nematoda).

The term “cultivated plants” is to be understood also including plants that are, e.g. by the use of recombinant DNA techniques, capable of synthesizing one or more proteins in order to increase the resistance or tolerance of those plants to bacterial, viral or fungal pathogens. Examples of such proteins are the so-called “pathogenesis-related proteins”, also termed PR proteins—see, for example EP-A 0 392 225-, or plant disease resistance genes—for example potato cultivars, which express resistance genes acting against Phytophthora infestans derived from the mexican wild potato Solanum bulbocastanum—or T4-lysozym—e. g. potato cultivars capable of synthesizing these proteins with increased resistance against bacteria such as Erwinia amylvora. The methods for producing such genetically modified plants are generally known to the person skilled in the art and are described, for example, in the publications mentioned above.

The term “cultivated plants” is to be understood also including plants that are, e.g. by the use of recombinant DNA techniques, capable of synthesizing one or more proteins to increase the productivity, e. g. bio mass production, grain yield, starch content, oil content or protein content, or to improve tolerance to drought, salinity or other growth-limiting environmental factors or tolerance to pests and fungal, bacterial or viral pathogens of those plants.

The term “cultivated plants” is to be understood also including plants that contain by the use of recombinant DNA techniques a modified amount of substances of content or new substances of content, specifically to improve human or animal nutrition, for example oil crops that produce health-promoting long-chain omega-3 fatty acids or unsaturated omega-9 fatty acids (e. g. Nexera® rape).

The term “cultivated plants” is to be understood also including plants that contain by the use of recombinant DNA techniques a modified amount of substances of content or new substances of content, specifically to improve raw material production, for example potatoes that produce increased amounts of amylopectin (e. g. Amflora® potato).

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

In the case of soil treatment or of application to the pests dwelling place or nest, the quan-tity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in treating crop plants, the rate of application of the active ingredients of this invention may be in the range of 0.1 g to 4000 g per hectare, desirably from 25 g to 600 g per hectare, more desirably from 50 g to 500 g per hectare.

The compounds of formula (I), including the tautomers and stereoisomers, as well as their salts, are effective through both contact, e.g. via soil, glass, wall, bed net, carpet, plant parts or animal parts, and ingestion, e.g. via ingestion of bait or plant part.

The compounds of the invention may also be applied against non-crop insect pests, such as ants, termites, wasps, flies, mosquitoes, crickets, or cockroaches. For use against said non-crop pests, compounds of formula (I), including the tautomers and stereoisomers, as well as their salts, are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickiness, moisture retention or aging characteristics.

The bait employed in the composition is a product, which is sufficiently attractive to incite insects such as ants, termites, wasps, flies, mosquitoes, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

For use in bait compositions, the typical content of active ingredient is from 0.001 weight % to 15 weight %, desirably from 0.001 weight % to 5% weight % of active compound.

Formulations of compounds of formula (I), including the tautomers and stereoisomers, as well as their salts, as aerosols, e.g in spray cans, oil sprays or pump sprays are highly suitable for the non-professional user for controlling pests such as flies, fleas, ticks, mosquitoes or cockroaches. Aerosol recipes are preferably composed of the active compound, solvents such as lower alcohols, e.g. methanol, ethanol, propanol or butanol, ketones, e.g. acetone, methyl ethyl ketone, paraffin hydrocarbons, e.g. kerosenes or mineral oils, having boiling ranges of approximately 50 to 250° C., dimethylformamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

For use in spray compositions, the content of active ingredient is from 0.001 to 80 weights %, preferably from 0.01 to 50 weight % and most preferably from 0.01 to 15 weight %.

The compounds of formula (I), including the tautomers and stereoisomers, as well as their salts, and their respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects, such as malaria, dengue and yellow fever, lymphatic filariasis, and leishmaniasis, with compounds of formula (I) or the stereoisomers, tautomers or salts thereof, and with their respective compositions also comprise treating surfaces of huts and houses, air spraying and impregnation of curtains, tents, clothing items, bed nets, tsetse-fly trap or the like. Insecticidal compositions for application to fibers, fab-ric, knitgoods, nonwovens, netting material or foils and tarpaulins preferably comprise a mixture including the insecticide, optionally a repellent and at least one binder. Suitable repellents for example are N,N-Diethyl-meta-toluamide (DEET), N,N-diethylphenylacetamide (DEPA), 1-(3-cyclohexan-1-yl-carbonyl)-2-methylpiperine, (2-hydroxymethylcyclohexyl) acetic acid lactone, 2-ethyl-1,3-hexandiol, indalone, Methylneodecanamide (MNDA), a pyrethroid not used for insect control such as {(+/−)-3-allyl-2-methyl-4-oxocyclopent-2-(+)-enyl-(+)-trans-chrysantennate (Esbio-thrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and di-ethylenically unsaturated hydrocarbons, such as styrene, and aliphatic diens, such as butadiene.

The impregnation of curtains and bednets is done in general by dipping the textile material into emulsions or dispersions of the insecticide or spraying them onto the nets.

The compounds of formula (I), including the tautomers and stereoisomers, as well as their salts, and their compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being, e.g. when the pests invade into houses and public facilities. The compounds of formula (I), their stereoisomers, their tautomers or their salts are applied not only to the surrounding soil surface or into the under-floor soil in order to protect wooden materials but it can also be applied to lum-bered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

The compounds of formula (I), including the tautomers and stereoisomers, as well as their salts, are also suitable for the treatment of seeds in order to protect the seed from insect pest, in particular from soil-living insect pests and the resulting plant's roots and shoots against soil pests and foliar insects.

The compounds of formula (I), including the tautomers and stereoisomers, as well as their salts, are particularly useful for the protection of the seed from soil pests and the resulting plants roots and shoots against soil pests and foliar insects. The protection of the resulting plants roots and shoots is preferred. More preferred is the protection of resulting plants shoots from piercing and sucking insects, wherein the protection from aphids is most preferred.

The present invention therefore comprises a method for the protection of seeds from insects, in particular from soil insects and of the seedling's roots and shoots from insects, in particular from soil and foliar insects, said method comprising contacting the seeds before sowing and/or after pregermination with a compound of the general formula (I), a tautomer, a stereosi-omer or a salt thereof. Particularly preferred is a method, wherein the plants roots and shoots are protected, more preferably a method, wherein the plants shoots are protected form piercing and sucking insects, most preferably a method, wherein the plants shoots are protected from aphids.

The term seed includes seeds and plant propagules of all kinds including but not limited to true seeds, seed pieces, suckers, corms, bulbs, fruit, tubers, grains, cuttings, cut shoots and the like and means in a preferred embodiment true seeds.

The term seed treatment includes all suitable seed treatment techniques known in the art, such as seed dressing, seed coating, seed dusting, seed soaking and seed pelleting.

The present invention also relates to seeds coated with or containing the active compound of the present invention, i.e. containing a compound of formula (I), a stereoisomer, a tautomer or a salt thereof.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the compounds of formula (I), including the tautomers and stereoisomers, as well as their salts, may also be used for the treatment seeds from plants, which tolerate the action of herbicides or fungicides or insecticides owing to breeding, including genetic engineering methods.

For example, the compounds of formula (I), including the tautomers and stereoisomers, as well as their salts, can be employed in treatment of seeds from plants, which are resistant to herbicides from the group consisting of the sulfonylureas, imidazolinones, glufosinate-ammonium or glyphosate-isopropylammonium and analogous active substances (see for example, EP-A-0242236, EP-A-242246) (WO 92/00377) (EP-A-0257993, U.S. Pat. No. 5,013,659) or in transgenic crop plants, for example cotton, with the capability of producing Bacillus thuringiensis toxins (Bt toxins) which make the plants resistant to certain pests (EP-A-0142924, EP-A-0193259),

Furthermore, the compounds of formula (I), including the tautomers and stereoisomers, as well as their salts, can be used also for the treatment of seeds from plants, which have modified characteristics in comparison with existing plants consist, which can be generated for example by traditional breeding methods and/or the generation of mutants, or by recombinant procedures). For example, a number of cases have been described of recombinant modifications of crop plants for the purpose of modifying the starch synthesized in the plants (e.g. WO 92/11376, WO 92/14827, WO 91/19806) or of transgenic crop plants having a modified fatty acid composition (WO 91/13972).

The seed treatment application of the active compound is carried out by spraying or by dusting the seeds before sowing of the plants and before emergence of the plants.

Compositions which are especially useful for seed treatment are e.g.:

A Soluble concentrates (SL, LS)

### D Emulsions (EW, EO, ES)

### E Suspensions (SC, OD, FS)

F Water-dispersible granules and water-soluble granules (WG, SG)
G Water-dispersible powders and water-soluble powders (WP, SP, WS)

### H Gel-Formulations (GF)

I Dustable powders (DP, DS)

Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter

In a preferred embodiment a FS formulation is used for seed treatment. Typically, a FS formulation may comprise 1-800 g/l of active ingredient, 1-200 g/l Surfactant, 0 to 200 g/l anti-freezing agent, 0 to 400 g/l of binder, 0 to 200 g/l of a pigment and up to 1 liter of a solvent, preferably water.

Especially preferred FS formulations of a compound of formula (I), a stereoisomer, a tautomer or a salt, for seed treatment usually comprise from 0.1 to 80% by weight (1 to 800 g/l) of the compound of formula (I), including its tautomers and stereoisomers, or a salt thereof, from 0.1 to 20% by weight (1 to 200 g/l) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxi-dant or the like, e.g. in an amount from 0.01 to 1% by weight and a filler/vehicle up to 100% by weight.

Seed Treatment formulations may additionally also comprise binders and optionally colorants.

Binders can be added to improve the adhesion of the active materials on the seeds after treatment. Suitable binders are homo- and copolymers from alkylene oxides like ethylene oxide or propylene oxide, polyvinylacetate, polyvinylalcohols, polyvinylpyrrolidones, and copolymers thereof, ethylene-vinyl acetate copolymers, acrylic homo- and copolymers, polyethyleneamines, polyethyleneamides and polyethyleneimines, polysaccharides like celluloses, tylose and starch, polyolefin homo- and copolymers like olefin/maleic anhydride copolymers, polyurethanes, poly-esters, polystyrene homo and copolymers

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of a gelling agent is carrageen (Satiagel®)

In the treatment of seed, the application rates of the compounds of formula (I) are generally from 0.1 g to 10 kg per 100 kg of seed, preferably from 1 g to 5 kg per 100 kg of seed, more preferably from 1 g to 1000 g per 100 kg of seed and in particular from 1 g to 200 g per 100 kg of seed.

The invention therefore also relates to seed comprising a compound of the formula (I), a tautomer, a stereoisomer or an agriculturally useful salt thereof, as defined herein. The amount of the compound of the formula (I) or the agriculturally useful salt thereof will in general vary from 0.1 g to 10 kg per 100 kg of seed, preferably from 1 g to 5 kg per 100 kg of seed, in particular from 1 g to 1000 g per 100 kg of seed. For specific crops such as lettuce the rate can be higher.

The compounds of formula (I), including their stereoisomers and their tautomers, and the veterinarily acceptable salts thereof are in particular also suitable for being used for combating parasites in and on animals.

An object of the present invention is therefore also to provide new methods to control parasites in and on animals. Another object of the invention is to provide safer pesticides for animals. Another object of the invention is further to provide pesticides for animals that may be used in lower doses than existing pesticides. And another object of the invention is to provide pesticides for animals, which provide a long residual control of the parasites.

The invention also relates to compositions containing a parasiticidally effective amount of a compound of formula (I) or a stereoisomer or a tautomer or a veterinarily acceptable salt thereof and an acceptable carrier, for combating parasites in and on animals.

The present invention also provides a method for treating, controlling, preventing and protecting animals against infestation and infection by parasites, which comprises orally, topically or parenterally administering or applying to the animals a parasiticidally effective amount of a compound of formula (I) or a stereoisomer or a tautomer or a veterinarily acceptable salt thereof or a composition comprising it.

The invention also provides a process for the preparation of a composition for treating, controlling, preventing or protecting animals against infestation or infection by parasites which comprises a parasiticidally effective amount of a compound of formula (I) or a stereoisomer or a tautomer or a veterinarily acceptable salt thereof or a composition comprising it.

Activity of compounds against agricultural pests does not suggest their suitability for control of endo- and ectoparasites in and on animals which requires, for example, low, non-emetic dosages in the case of oral application, metabolic compatibility with the animal, low toxicity, and a safe handling.

Surprisingly it has now been found that compounds of formula (I), including their stereoisomers and tautomers, and the salts thereof, are suitable for combating endo- and ectoparasites in and on animals.

Compounds of formula (I), including their stereoisomers and their tautomers, and the veterinarily acceptable salts thereof, and compositions comprising them are preferably used for controlling and preventing infestations and infections animals including warm-blooded animals, including humans, and fish. They are for example suitable for controlling and preventing infestations and infections in mammals such as cattle, sheep, swine, camels, deer, horses, pigs, poul-try, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Compounds of formula (I), including their stereoisomers and their tautomers, and the veterinarily acceptable salts thereof and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of formula (I), including their stereoisomers and their tautomers, and the veterinarily acceptable salts thereof and compositions comprising them are suitable for systemic and/or non-systemic control of ecto- and/or endoparasites. They are active against all or some stages of development.

The compounds of formula (I) including their stereoisomers and their tautomers, and the veterinarily acceptable salts thereof are especially useful for combating ectoparasites.

The compounds of formula (I), including their stereoisomers and their tautomers, and the veterinarily acceptable salts thereof are especially useful for combating parasites of the following orders and species, respectively:

fleas (Siphonaptera), e.g. Ctenocephalides fells, Ctenocephalides canis, Xenopsylla cheopis, Pulex irritans, Tunga penetrans, and Nosopsyllus fasciatus;

cockroaches (Blattaria—Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis;

flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pipiens, Culex nignpalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprin, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus simllis;

lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus;

ticks and parasitic mites (Parasitiformes): ticks (Ixodida), e.g. Ixodes scapularis, Ixodes holocyclus, Ixodes pacificus, Rhiphicephalus sanguineus, Dermacentor andersom, Dermacentor variabilis, Amblyomma americanum, Ambryomma maculatum, Ornithodorus hermsi, Ornithodorus turicata and parasitic mites (Mesostigmata), e.g. Ornithonyssus bacoti and Dermanyssus gallinae;

Actinedida (Prostigmata) und Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp;

Bugs (Heteropterida): Cimex lectularius, Cimex hemipterus, Reduvius senilis, Triatoma spp., Rhodnius ssp., Panstrongylus ssp. and Arilus critatus;

Anoplurida, e.g. Haematopinus spp., Linognathus spp., Pediculus spp., Phtirus spp., and Solenopotes spp.;

Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp;

Roundworms Nematoda:

Wipeworms and Trichinosis (Trichosyringida), e.g. Trichinellidae (Trichinella spp.), (Tri-churidae) Trichuris spp., Capillaria spp.;

Rhabditida, e.g. Rhabditis spp, Strongyloides spp., Helicephalobus spp;

Strongylida, e.g. Strongylus spp., Ancylostoma spp., Necator americanus, Bunostomum spp. (Hookworm), Trichostrongylus spp., Haemonchus contortus., Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma renale;

Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi;

Camallanida, e.g. Dracunculus medinensis (guinea worm);

Spirurida, e.g. Thelazia spp. Wuchereria spp., Brugia spp., Onchocerca spp., Dirofilari spp. a, Dipetalonema spp., Setaria spp., Elaeophora spp., Spirocerca lupi, and Habronema spp.;

Thorny headed worms (Acanthocephala), e.g. Acanthocephalus spp., Macracanthorhynchus hirudinaceus and Oncicola spp;

Planarians (Plathelminthes):

Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp., and Nanocyetes spp;

Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The compounds of formula (I), including their stereoisomers and their tautomers, and the salts thereof and compositions containing them are particularly useful for the control of pests from the orders Diptera, Siphonaptera and Ixodida.

Moreover, the use of the compounds of formula (I), including their stereoisomers and their tautomers, and the salts thereof and compositions containing them for combating mosquitoes is especially preferred.

The use of the compounds of formula (I), including their stereoisomers and their tautomers, and the salts thereof and compositions containing them for combating flies is a further preferred embodiment of the present invention.

Furthermore, the use of the compounds of formula (I), including their stereoisomers and their tautomers, and the salts thereof and compositions containing them for combating fleas is especially preferred.

The use of the compounds of formula (I), including their stereoisomers and their tautomers, and the salts thereof and compositions containing them for combating ticks is a further preferred embodiment of the present invention.

The compounds of formula (I), including their stereoisomers and their tautomers, and the salts thereof also are especially useful for combating endoparasites (roundworms nematoda, thorny headed worms and planarians).

Administration can be carried out both prophylactically and therapeutically.

Administration of the active compounds is carried out directly or in the form of suitable preparations, orally, topically/dermally or parenterally.

For oral administration to warm-blooded animals, the compounds of the present invention may be formulated as animal feeds, animal feed premixes, animal feed concentrates, pills, solutions, pastes, suspensions, drenches, gels, tablets, boluses and capsules. In addition, the compounds of the present invention may be administered to the animals in their drinking water. For oral administration, the dosage form chosen should provide the animal with 0.01 mg/kg to 100 mg/kg of animal body weight per day of the formula (I) compound, preferably with 0.5 mg/kg to 100 mg/kg of animal body weight per day.

Alternatively, the compounds of the present invention may be administered to animals parenterally, for example, by intraruminal, intramuscular, intravenous or subcutaneous injection. The compounds of the present invention may be dispersed or dissolved in a physiologically acceptable carrier for subcutaneous injection. Alternatively, the compounds of the present invention may be formulated into an implant for subcutaneous administration. In addition the compounds of the present invention may be transdermally administered to animals. For parenteral administration, the dosage form chosen should provide the animal with 0.01 mg/kg to 100 mg/kg of animal body weight per day of a compound of the present invention.

The compounds of the present invention may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the compounds of the present invention. In addition, the compounds of the present invention may be formulated as ear tags for animals, particularly quadrupeds such as cattle and sheep.

Suitable preparations are:
Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-in-water or water-in-oil emulsion base;
Solid preparations such as powders, premixes or concentrates, granules, pellets, tablets, boluses, capsules; aerosols and inhalants, and active compound-containing shaped articles.

Compositions suitable for injection are prepared by dissolving the active ingredient in a suitable solvent and optionally adding further ingredients such as acids, bases, buffer salts, preservatives, and solubilizers. The solutions are filtered and filled sterile.

Suitable solvents are physiologically tolerable solvents such as water, alkanols such as ethanol, butanol, benzyl alcohol, glycerol, propylene glycol, polyethylene glycols, N-methyl-pyrrolidone, 2-pyrrolidone, and mixtures thereof.

The compounds of the present invention can optionally be dissolved in physiologically tolerable vegetable or synthetic oils which are suitable for injection.

Suitable solubilizers are solvents which promote the dissolution of the active compound in the main solvent or prevent its precipitation. Examples are polyvinylpyrrolidone, polyvinyl alcohol, polyoxyethylated castor oil, and polyoxyethylated sorbitan ester.

Suitable preservatives are benzyl alcohol, trichlorobutanol, p-hydroxybenzoic acid esters, and n-butanol.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on.

Solutions for use on the skin are prepared according to the state of the art and according to what is described above for injection solutions, sterile procedures not being necessary.

Further suitable solvents are polypropylene glycol, phenyl ethanol, phenoxy ethanol, ester such as ethyl or butyl acetate, benzyl benzoate, ethers such as alkyleneglycol alkylether, e.g. dipropylenglycol monomethylether, ketons such as acetone, methylethylketone, aromatic hydrocarbons, vegetable and synthetic oils, dimethylformamide, dimethylacetamide, transcutol, solketal, propylencarbonate, and mixtures thereof.

It may be advantageous to add thickeners during preparation. Suitable thickeners are inorganic thickeners such as bentonites, colloidal silicic acid, aluminium monostearate, organic thickeners such as cellulose derivatives, polyvinyl alcohols and their copolymers, acrylates and methacrylates.

Gels are applied to or spread on the skin or introduced into body cavities. Gels are prepared by treating solutions which have been prepared as described in the case of the injection solutions with sufficient thickener that a clear material having an ointment-like consistency re-sults. The thickeners employed are the thickeners given above.

Pour-on formulations are poured or sprayed onto limited areas of the skin, the active compound penetrating the skin and acting systemically.

Pour-on formulations are prepared by dissolving, suspending or emulsifying the active compound in suitable skin-compatible solvents or solvent mixtures. If appropriate, other auxiliaries such as colorants, bioabsorption-promoting substances, antioxidants, light stabilizers, adhesives are added.

Suitable solvents are, for example, water, alkanols, glycols, polyethylene glycols, polypropylene glycols, glycerol, aromatic alcohols such as benzyl alcohol, phenylethanol, phenoxy-ethanol, esters such as ethyl acetate, butyl acetate, benzyl benzoate, ethers such as alkylene glycol alkyl ethers such as dipropylene glycol monomethyl ether, diethylene glycol mono-butyl ether, ketones such as acetone, methyl ethyl ketone, cyclic carbonates such as propylene carbonate, ethylene carbonate, aromatic and/or aliphatic hydrocarbons, vegetable or synthetic oils, DMF, dimethylacetamide, n-alkylpyrrolidones such as methylpyrrolidone, n-butylpyrrolidone or n-octylpyrrolidone, N-methylpyrrolidone, 2-pyrrolidone, 2,2-dimethyl-4-oxy-methylene-1,3-diox-olane and glycerol formal.

Suitable colorants are all colorants permitted for use on animals and which can be dissolved or suspended.

Suitable absorption-promoting substances are, for example, DMSO, spreading oils such as isopropyl myristate, dipropylene glycol pelargonate, silicone oils and copolymers thereof with polyethers, fatty acid esters, triglycerides, fatty alcohols.

Suitable antioxidants are, for example, sulfites or metabisulfites such as potassium meta-bisulfite, ascorbic acid, butylhydroxytoluene, butylhydroxyanisole, tocopherol.

Suitable light stabilizers are, for example, novantisolic acid.

Suitable adhesives are, for example, cellulose derivatives, starch derivatives, polyacrylates, natural polymers such as alginates, gelatin.

Emulsions can be administered orally, dermally or as injections.

Emulsions are either of the water-in-oil type or of the oil-in-water type.

They are prepared by dissolving the active compound either in the hydrophobic or in the hydrophilic phase and homogenizing this with the solvent of the other phase with the aid of suitable emulsifiers and, if appropriate, other auxiliaries such as colorants, absorption-promoting substances, preservatives, antioxidants, light stabilizers, viscosity-enhancing substances.

Suitable hydrophobic phases (oils) are, for example: liquid paraffins, silicone oils, natural vegetable oils such as sesame oil, almond oil, castor oil, synthetic triglycerides such as caprylic/capric biglyceride, triglyceride mixture with vegetable fatty acids of the chain length C₈-C₁₂ or other specially selected natural fatty acids, partial glyceride mixtures of saturated or unsaturated fatty acids possibly also containing hydroxyl groups, mono- and diglycerides of the C₈-C₁₀ fatty acids, fatty acid esters such as ethyl stearate, di-n-butyryl adipate, hexyl laurate, dipropylene glycol perlargonate, esters of a branched fatty acid of medium chain length with saturated fatty alcohols of chain length C₁₆-C₁₈, isopropyl myristate, isopropyl palmitate, caprylic/capric acid esters of saturated fatty alcohols of chain length C₁₂-C₁₈, isopropyl stearate, oleyl oleate, decyl oleate, ethyl oleate, ethyl lactate, waxy fatty acid esters such as synthetic duck coccygeal gland fat, dibutyl phthalate, diisopropyl adipate, and ester mixtures related to the latter, fatty alcohols such as isotridecyl alcohol, 2-octyldodecanol, cetylstearyl alcohol, oleyl alcohol, and fatty acids such as oleic acid and mixtures thereof.

Suitable hydrophilic phases are, for example, water, alcohols such as propylene glycol, glycerol, sorbitol and mixtures thereof.

Suitable emulsifiers are, for example,
non-ionic surfactants, e.g. polyethoxylated castor oil, polyethoxylated sorbitan monooleate, sorbitan monostearate, glycerol monostearate, polyoxyethyl stearate, alkylphenol polyglycol ether;
ampholytic surfactants such as di-sodium N-lauryl-p-iminodipropionate or lecithin;
anionic surfactants, such as sodium lauryl sulfate, fatty alcohol ether sulfates, mono/dialkyl polyglycol ether orthophosphoric acid ester monoethanolamine salt;
cation-active surfactants, such as cetyltrimethylammonium chloride.

Suitable further auxiliaries are substances which enhance the viscosity and stabilize the emulsion, such as carboxymethylcellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silicic acid or mixtures of the substances mentioned.

Suspensions can be administered orally or topically/dermally. They are prepared by suspending the active compound in a suspending agent, if appropriate with addition of other auxiliaries such as wetting agents, colorants, bioabsorption-promoting substances, preservatives, antioxidants, light stabilizers.

Liquid suspending agents are all homogeneous solvents and solvent mixtures.

Suitable wetting agents (dispersants) are the emulsifiers given above.

Other auxiliaries, which may be mentioned, are those given above.

Semi-solid preparations can be administered orally or topically/dermally. They differ from the suspensions and emulsions described above only by their higher viscosity.

For the production of solid preparations, the active compound is mixed with suitable excipients, if appropriate with addition of auxiliaries, and brought into the desired form.

Suitable excipients are all physiologically tolerable solid inert substances. Those used are inorganic and organic substances. Inorganic substances are, for example, sodium chloride, carbonates such as calcium carbonate, hydrogencarbonates, aluminium oxides, titanium oxide, silicic acids, argillaceous earths, precipitated or colloidal silica, or phosphates. Organic substances are, for example, sugar, cellulose, foodstuffs and feeds such as milk powder, animal meal, grain meals and shreds, starches.

Suitable auxiliaries are preservatives, antioxidants, and/or colorants which have been mentioned above.

Other suitable auxiliaries are lubricants and glidants such as magnesium stearate, stearic acid, talc, bentonites, disintegration-promoting substances such as starch or crosslinked polyvinylpyrrolidone, binders such as starch, gelatin or linear polyvinylpyrrolidone, and dry binders such as microcrystalline cellulose.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

The compositions which can be used in the invention can comprise generally from about 0.001 to 95% of a compound of formula (I), a stereoisomer, a tautomer or a salt thereof.

Generally it is favorable to apply the compounds of the present invention in total amounts of 0.5 mg/kg to 100 mg/kg per day, preferably 1 mg/kg to 50 mg/kg per day.

Ready-to-use preparations contain the compounds acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight.

Preparations which are diluted before use contain the compounds acting against ectoparasites in concentrations of 0.5 to 90 percent by weight, preferably of 1 to 50 percent by weight.

Furthermore, the preparations for controlling endoparasites comprise a compound of the present invention usually in concentrations of 10 ppm to 2 percent by weight, preferably of 0.05 to 0.9 percent by weight, very particularly preferably of 0.005 to 0.25 percent by weight.

In a preferred embodiment of the present invention, the compositions comprising the a compound of the present invention are applied dermally/topically.

In a further preferred embodiment, the topical application is conducted in the form of conn-pound-containing shaped articles such as collars, medallions, ear tags, bands for fixing at body parts, and adhesive strips and foils.

Generally it is favorable to apply solid formulations which release compounds of the present invention in total amounts of 10 mg/kg to 300 mg/kg, preferably 20 mg/kg to 200 mg/kg, most preferably 25 mg/kg to 160 mg/kg body weight of the treated animal in the course of three weeks.

For the preparation of the shaped articles, thermoplastic and flexible plastics as well as elastomers and thermoplastic elastomers are used. Suitable plastics and elastomers are polyvinyl resins, polyurethane, polyacrylate, epoxy resins, cellulose, cellulose derivatives, polyamides and polyester which are sufficiently compatible with the compounds of the present invention. A detailed list of plastics and elastomers as well as preparation procedures for the shaped articles is given e.g. in WO 03/086075.

The present invention is now illustrated in further details by the following examples, without imposing any limitation thereto.

The following abbreviations are used:

DCE: dichloroethane

DCM: dichloromethane

TFA: trifluoroacetic acid

EtOAc: ethyl acetate

HPLC: High Performance Liquid Chromatography

MS: Mass spectrometry

MeOH: Methanol

t_{R} or r.t.: retention time

The Compound examples can be characterized by their physic-chemical data*), e.g. by coupled High Performance Liquid Chromatography, by mass spectrometry (HPLC/MS) or by their melting point.

*)Analytical UPLC column: Phenomenex Kinetex 1.7 μm XB-C18 100A; 50×2.1 mm; mobile phase: A: water+0.1% trifluoroacetic acid (TFA); B: acetonitrile+0.1% TFA; gradient: 5-100% B in 1.50 minutes; 100% B 0.20 min; flow: 0.8-1.0 mL/min in 1.50 minutes at 60° C.

MS-method: ESI positive

A. Preparation Examples

### Synthesis of Amine Salt E1.1

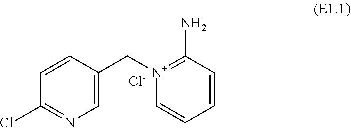 [SMILES: Nc1cccc[n+]1Cc1ccc(Cl)nc1 ; [Cl-]]

A solution 2-chloro-5-chloromethyl (16.20 g, 100 mmol) and 2-amino-pyridine (9.60 g, 102 mmol) in ethanol (100 mL) was refluxed for 24 hours. The reaction was then cooled to room temperature, and concentrated in vacuo. Then 100 mL of toluene was added to the residue, and the mixture was concentrated in vacuo. The 75 mL of CH₂Cl₂ was added to the residue and the mixture was stirred rapidly for 15 minutes, during which time a precipitate forms. The precipitate was then filtered, and washed with CH₂Cl₂ (50 mL), diethyl ether (50 mL), and dried under vacuum to afford the product as a pale yellow solid (14.0 g, 55% yield).

LC-MS: mass calculated for C₁₁H₁₁ClN3 [M]⁺220.1. found 220.1; t_{R}=0.529 min.

Synthesis of Amine Salt E1.2

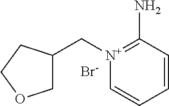 [SMILES: Nc1cccc[n+]1CC1CCOC1 ; [Br-]]

A solution of 3-(bromomethyl)tetrahydrofuran (8.00 g, 48.48 mmol) and 2-aminopyridine (9.12 g, 97.02 mmol) in sulfolane (250 mL) was heated to 80° C. for 70 hours. The reaction was then cooled to room temperature and then diluted with 5 L of CH₂Cl₂, and twice extracted with 500 mL of water. The aqueous phase was then concentrated in vacuo. To the residue was then added CH₂Cl₂ (100 mL) and a thick orange oil precipitated. The CH₂Cl₂ was decanted and the oil dried in vacuo to afford the desired salt as a wax contaminated with a trace amount of sulfolane (1.1 g, 9% yield).

LC-MS: mass calculated for C₁₀H₁₅N₂O [M]⁺179.1. found 179.3; t_{R}=0.326 min.

### Example 1

### Compound E1.3

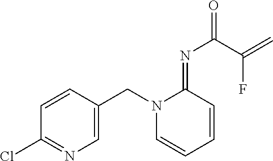 [SMILES: C=C(F)C(=O)/N=c1\ccccn1Cc1ccc(Cl)nc1]

To a suspension of amine salt E1.1 (1.00 g, 3.90 mmol), 2-fluoroacrylic acid (0.530 g, 5.86 mmol), and triethylamine (1.19 g, 11.71 mmol) in dichloroethane (10 mL) at room temperature was added a solution of propyl phosphoric anhydride (50% wt) (1.86 g, 5.86 mmol, a 50% wt solution in EtOAc was used). The reaction was then heated in the microwave at 100 C for 3 hrs. The reaction was then diluted with ethyl acetate (100 mL), washed with saturated aqueous NaHCO₃ (100 mL) and water (100 mL), layers separated, and the organic layer dried over Na₂SO₄ and concentrated in vacuo to afford a residue which was purified using column chromatography over silica gel (0450% MeOH/EtOAc) to afford the desired product as a beige solid (0.110 g, 10% yield).

LC-MS: mass calculated for C₁₄H₁₂ClFN₃O [M+H]⁺ 291.7. found 292.2; t_{R}=0.678 min.

### Example 2

### Compound E1.4

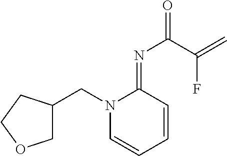 [SMILES: C=C(F)C(=O)/N=c1\ccccn1CC1CCOC1]

To a suspension of amine salt E1.2 (0.500 g, 1.93 mmol) in DCE (6 mL) at room temperature was sequentially added pyrazine-2-carboxylic acid (0.311 g, 2.50 mmol), triethylamine (0.527 g, 5.21 mmol), and propylphosphonic anhydride (0.318 g, 3.08 mmol) as a 50% solution in ethyl acetate. The reaction was then heated in a microwave at 90 C for 3 hours. The reaction was then diluted with EtOAc (100 mL), washed with saturated aqueous NaHCO₃, dried over Na₂SO₄ and concentrated in vacuo to afford a residue. The residue was purified using silica gel chromatography eluting with 5-20% MeOH/EtOAc to afford the desired compound as an off-white solid (0.134 g, 24% yield).

LC-MS: mass calculated for C₁₅H₁₇N₄O₂ [M+H]⁺ 285.1. found 285.4; t_{R}=0.521 min.

Synthesis of Intermediate I1.1

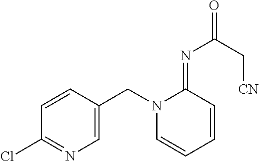 [SMILES: N#CCC(=O)/N=c1\ccccn1Cc1ccc(Cl)nc1]

To a suspension of amine salt E1.1 (1.50 g, 5.86 mmol) in DCE (30 mL) at room temperature was sequentially added 2-cyanoacetic acid (0.60 g, 7.03 mmol), triethylamine (1.48 g, 14.6 mmol), and propylphosphonic anhydride (4.47 g, 7.03 mmol) as a 50% solution in ethyl acetate. The reaction was then heated in a microwave at 90° C. for 3 hours. The reaction was then diluted with dichloromethane (100 mL), washed with saturated aqueous NaHCO₃, dried over Na₂SO₄ and concentrated in vacuo to afford a residue. The residue was purified using silica gel chromatography eluting with 5-20% MeOH/EtOAc to afford the desired compound as an off-white solid (0.52 g, 31% yield).

Synthesis of Intermediate I1.2

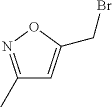

To a stirred solution of acetaldehyde oxime (10 g, 169.49 mmol) in DCM (200 mL) was added propargyl bromide (13 mL, 169.49 mmol) followed by triethylamine (10 mL) at 0° C., then 4% NaOCl (1.08 mL) was added drop wise to the reaction mixture at 0° C. After that the reaction mixture was stirred at room temperature for 3 h. Reaction mixture was extracted with DCM (3×200 mL) dried over Na₂SO₄, concentrated under reduced pressure to afford the crude product. The crude product was purified by flash column chromatography (Silica gel: 100-200 mesh) and eluted with 0-10% EtOAC in hexane to afford 3 g of I1.2 as pale yellow liquid. ¹H-NMR (CDCl₃, 400 MHz) (ppm): 6.18 (s, 1H), 4.42 (s, 2H), 2.31 (s, 3H).

Synthesis of Intermediate I1.3

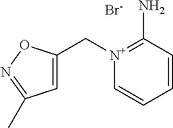

To a stirred solution of pyridine-2-amine (1.61 g, 17.14 mmol) in ethanol (40 ml) was added I1.2 (3 g, 17.14 mmol) at room temperature, then the reaction mixture was stirred at reflux condition for 20 h. The reaction mixture was concentrated under reduced pressure and triturated with DCM. The solid was filtered off and dried to afford the desired product as pale yellow solid. ¹H-NMR (CDCl₃, 400 MHz) (ppm): 8.67 (s, 2H), 8.18 (m, 1H), 7.94 (m, 1H), 7.12 (m, 1H), 6.98 (m, 1H), 6.43 (m, 1H), 5.62 (s, 2H), 2.23 (s, 3H).

Synthesis of Intermediate I1.4

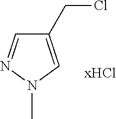

To a stirred solution of (1-methylpyrazol-4-yl)methanol (9 g, 80.36 mmol) in DCM (100 mL) was added SOCl₂ (26 mL, 361.60 mmol) dropwise at 0° C., followed by stirring at room temperature for 3 h. The reaction mixture was concentrated under reduced pressure to afford the crude product (12 g) as brown solid. The product was used in the next step without purification. ¹H-NMR (CDCl₃, 400 MHz) (ppm): 7.86 (m, 1H), 7.51 (m, 1H), 4.76 (m, 2H), 3.81 (m, 3H).

Synthesis of Intermediate I1.5

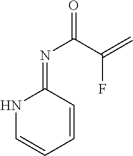

To a stirred solution of pyridine-2-amine (5 g, 53.19 mmol) and methyl 2-fluoroprop-2-enoate in toluene (20 mL) was added trimethylamine (159.57 mmol) dropwise at 0° C. The reaction was heated to 110° C. and stirred at this temperature for 3 h until completion of reaction was monitored by TLC. The reaction mixture was quenched with ice cold water and filtered through celite (rinsed with 200 mL EtOAc). Then, the mixture was extracted with ethyl acetate (3×100 mL), the organic phases were dried over Na₂SO₄ and concentrated to afford 4.6 g of crude I1.4. The product was used in the next step without purification.

¹H-NMR (CDCl₃, 400 MHz) (ppm): 8.58 (s, 1H), 8.38 (m, 1H), 8.28 (m, 1H), 7.78 (m, 1H), 7.12 (m, 1H), 5.82 (m, 1H), 5.21 (m, 1H).

### Example 14

### Compound E1.16

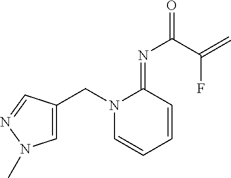 [SMILES: C=C(F)C(=O)/N=c1\ccccn1Cc1cnn(C)c1]

To a stirred solution of I1.5 (2 g, 12.04 mmol) in acetonitrile (30 mL) was added I1.4 (2.6 g, 15.66 mmol) followed by K₂CO₃ (5.8 g, 42.17 mmol) at room temperature and the reaction mixture was stirred at reflux temperature for 16 h. The reaction mixture was filtered and the precipitate was rinsed with EtOAc (3×60 mL). The combined organic layers were concentrated under reduced pressure to afford the crude product (1.5 g). The crude product was purified by flash column chromatography (silica gel: 100-200 mesh) and eluted with 30% EtOAC in hexane to afford 624 mg of the title compound as off white solid (750 mg).

LC-MS: mass calculated for C₁₃H₁₄FN₄O [M+H]⁺ 261.3. found 261.1; t_{R}=0.526 min.

### Example 26

### Compound E1.28

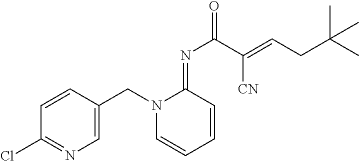 [SMILES: CC(C)(C)C/C=C(\C#N)C(=O)/N=c1\ccccn1Cc1ccc(Cl)nc1]

To a solution of I1.1 (0.50 g, 1.74 mmol) in acetic acid (4 mL) and piperidine (0.1 mL) was added 3,3-dimethylbutanal (210 mg, 2.09 mmol). After stirring the reaction at room temperature for 16 h, water (10 mL) was added. The mixture was extracted two times with ethyl acetate, dried over Na₂SO₄ and concentrated in vacuo to afford a residue. The residue was purified using silica gel chromatography eluting with 5-20% MeOH/EtOAc to afford the desired compound as an off-white solid (0.29 g, 41% yield).

The compounds of the formula (I-A) summarized in tables 1 and 2 were prepared by anal-ogy to the methods described in examples 1, 2, 14 and 26:

TABLE 1
Compounds of the Formula (I-A)
(I-A)

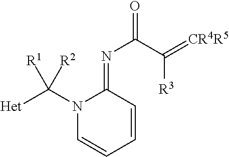

| Ex | No. | Het | R¹ | R² | R³ | R⁴ | R⁵ | |
|---|---|---|---|---|---|---|---|---|
| 3 | E1.5 | 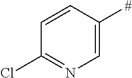 | H | H | F | H | CH₃ | r.t. = 0.705 m/z [M + H]⁺ = 306.5 |
| 4 | E1.6 | 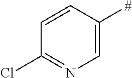 | H | H | F | H | i-Pr | r.t. = 0.829 m/z [M + H]⁺ = 334.5 |
| 5 | E1.7 | 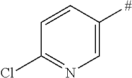 | H | H | F | H | n-Pr | r.t. = 0.823 m/z [M + H]⁺ = 334.5 |
| 6 | E1.8 | 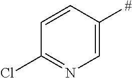 | H | H | H | H | CO₂C₂H₅ | r.t. = 0.750 m/z [M + H]⁺ = 346.5 |
| 7 | E1.9 | 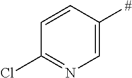 | H | H | Cl | H | H | r.t. = 0.735 m/z [M + H]⁺ = 308.4 |
| 8 | E1.10 | 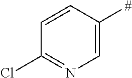 | H | H | Br | H | H | r.t. = 0.766 m/z [M + H]⁺ = 354.4 |

If not indicated otherwise, the compounds of table 1 are mixtures of E/Z-isomers.

TABLE 2
(I-A)

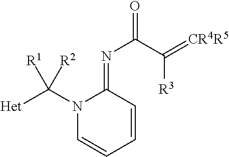

| Ex. | No. | Het^{*)} | R¹ | R² | R³ | R⁴ | R^{5 #)} | r.t. [min] | m/z [M + H]⁺ |
|---|---|---|---|---|---|---|---|---|---|
| 9 | E1.11 | Het-A | H | H | CH₃ | H | H | 0.641 | 288.1 |
| 10 | E1.12 | Het-A | H | H | CN | H | 2-chloro-3-thienyl | 1.158 | 415.2 |
| 11 | E1.13 | Het-C | H | H | F | H | H | 0.823 | 297.8 |
| 12 | E1.14 | Het-A | H | H | CN | H | 4-Cl—C₆H₄ | 1.163 | 411.2 |
| 13 | E1.15 | Het-D | H | H | F | H | H | 0.643 | 261.9 |
| 14 | E1.16 | Het-B | H | H | F | H | H | 0.526 | 261.0 |
| 15 | E1.17 | Het-A | H | H | F | F | 4-OCH₃—C₆H₄ | 0.997 | 416.6 |
| 16 | E1.18 | Het-A | H | H | F | F | 4-Cl-3-CF₃—C₆H₃ | 1.231 | 490.5 |
| 17 | E1.19 | Het-A | H | H | F | F | 4-CF₃—C₆H₄ | 1.172 | 454.6 |
| 18 | E1.20 | Het-A | H | H | F | F | 3-CF₃—C₆H₄ | 1.126 | 453.8 |
| 19 | E1.21 | Het-A | H | H | F | F | 4-CF₃—C₆H₄ | 1.132 | 453.8 |
| 20 | E1.22 | Het-A | H | H | F | F | 3-CF₃—C₆H₄ | 1.130 | 453.8 |
| 21 | E1.23 | Het-A | H | H | CN | H | 3-thienyl | 1.086 | 380.7 |
| 22 | E1.24¹⁾ | Het-A | H | H | F | F | 4-Cl—C₆H₄ | 1.110 | 420.6 |
| 23 | E1.25²⁾ | Het-A | H | H | F | 4-Cl—C₆H₄ | F | 1.093 | 420.5 |
| 24 | E1.26 | Het-A | H | H | CN | CH₃ | CH₃ | 0.850 | 326.8 |
| 24 | E1.27 | Het-A | H | H | CN | H | N(CH₃)₂ | 0.656 | 341.4 |
| 26 | E1.28 | Het-A | H | H | CN | H | CH₂C(CH₃)₃ | 1.113 | 368.8 |
| 27 | E1.29 | Het-A | H | H | CN | H | CHCl₂ | 1.088 | 383.1 |
| 28 | E1.30 | Het-A | H | H | CN | H | CH₂CH(CH₃)₂ | 1.095 | 355.3 |
| 29 | E1.31 | Het-A | H | H | CN | H | 1-methylbutyl | 1.160 | 369.0 |
| 30 | E1.32 | Het-A | H | H | CN | H | CH₂CH₂CH₂CH₃ | 1.089 | 354.9 |
| 31 | E1.33 | Het-A | H | H | CN | H | CH₂CH₂CH₃ | 1.016 | 340.8 |
| 32 | E1.34 | Het-A | H | H | CN | H | c-C₃H₅ | 0.932 | 338.8 |
| 33 | E1.35 | Het-A | H | H | CN | H | CH₂CH₃ | 0.947 | 326.8 |
| 34 | E1.36 | Het-A | H | H | CN | H | c-C₅H₉ | 1.094 | 367.4 |
| 35 | E1.37 | Het-A | H | H | CN | H | 2-methylsulfanylpropyl | 1.050 | 386.8 |
| 36 | E1.38 | Het-A | H | H | CN | H | 2-furyl | 0.966 | 365.3 |
| 37 | E1.39 | Het-A | H | H | CN | H | 1H-pyrrol-3-yl | 0.902 | 363.8 |
| 38 | E1.40 | Het-A | H | H | CN | H | C₆H₅ | 1.072 | 374.9 |
| 39 | E1.41 | Het-A | H | H | CN | H | 3-methoxy-4-methyl-phenyl | 1.136 | 418.9 |
| 40 | E1.42 | Het-A | H | H | CN | H | 4-pyridyl | 0.969 | 364.8 |
| 41 | E1.43 | Het-A | H | H | CN | H | 4-methoxyphenyl | 1.065 | 404.9 |
| 42 | E1.44 | Het-A | H | H | CN | H | 3-chloro-2-thienyl | 1.110 | 414.8 |
| 43 | E1.45 | Het-A | H | H | CN | H | 3-(trifluoromethyl)phenyl | 1.200 | 443.3 |
| 44 | E1.46 | Het-A | H | H | CN | H | 3-pyridyl | 0.758 | 376.3 |
| 45 | E1.47 | Het-A | H | H | CN | H | (CH₂)₂—C₆H₅ | 1.139 | 403.4 |
| 46 | E1.48 | Het-A | H | H | CN | H | 1,2-dimethylbutyl | 1.204 | 383.4 |
| 47 | E1.49 | Het-A | H | H | CN | H | tetrahydropyran-4-yl | 0.907 | 383.4 |
| 48 | E1.50 | Het-A | H | H | CN | H | (CH₂)₄CH₃ | 1.177 | 369.4 |
| 49 | E1.51 | Het-A | H | H | CN | H | 6-(trifluoromethyl)-3-pyridyl | 1.125 | 444.3 |
| 50 | E1.52 | Het-A | H | H | CN | H | imidazo[1,2-a]pyridin-2-yl | 0.771 | 415.4 |
| 51 | E1.53 | Het-A | H | H | CN | H | c-C₇H₁₃ | 1.232 | 395.5 |
| 52 | E1.54 | Het-A | H | H | CN | H | 3-nonenyl | 1.376 | 423.5 |
| 53 | E1.55 | Het-A | H | H | CN | H | 1-ethylpentyl | 1.273 | 397.5 |
| 54 | E1.56 | Het-A | H | H | CN | H | 1,1-dimethyl-2-(3-pyridyl)ethyl | 0.780 | 432.4 |
| 55 | E1.57 | Het-A | H | H | CN | H | 2-F—C₆H₄ | 1.108 | 393.3 |
| 56 | E1.58 | Het-A | H | H | CN | H | C(CH₃)₃ | 1.077 | 355.4 |
| 57 | E1.59 | Het-A | H | H | CN | H | c-C₆H₁₁ | 1.183 | 381.4 |
| 58 | E1.60 | Het-A | H | H | CN | H | 6,6-dimethylnorpinan-2-yl | 1.305 | 421.5 |
| 59 | E1.61 | Het-A | H | H | CN | H | tetrahydrothiopyran-3-yl | 1.070 | 399.4 |
| 60 | E1.62 | Het-A | H | H | CN | H | pyrimidin-2-yl | 0.853 | 377.3 |
| 61 | E1.63 | Het-A | H | H | CN | H | 2-furyl | 0.974 | 365.3 |
| 62 | E1.64 | Het-A | H | H | CN | H | C₆H₅ | 1.092 | 375.3 |
| 63 | E1.65 | Het-A | H | H | CN | H | CH(CH₃)C(CH₃)₃ | 1.184 | 383.5 |
| 64 | E1.66 | Het-A | H | H | CN | H | 1-isopropoxyethyl | 1.060 | 385.4 |
| 65 | E1.67 | Het-A | H | H | CN | H | 1-methyl-2-methylsulfanyl-ethyl | 1.047 | 387.4 |
| 66 | E1.68 | Het-A | H | H | CN | H | 4-methylcyclohexyl | 1.244 | 395.5 |
| 67 | E1.69 | Het-A | H | H | CN | H | tetrahydropyran-4-ylmethyl | 0.944 | 397.4 |
| 68 | E1.70 | Het-A | H | H | CN | H | 2-isobutoxyethyl | 1.134 | 399.4 |
| 69 | E1.71 | Het-A | H | H | CN | H | 1-(2-methoxyethoxy)ethyl | 0.938 | 401.4 |
| 70 | E1.72 | Het-A | H | H | CN | H | c-C₈H₁₅ | 1.283 | 409.4 |
| 71 | E1.73 | Het-A | H | H | CN | H | 2-ethoxycarbonylcyclopropyl | 1.019 | 411.3 |
| 72 | E1.74 | Het-A | H | H | CN | H | 2-(trifluoromethyl)phenyl | 1.191 | 443.3 |
| 73 | E1.75 | Het-A | H | H | CN | H | 5-bromo-3-pyridyl | 1.075 | 456.2 |
| 74 | E1.76 | Het-A | H | H | CN | H | 1-methoxyethyl | 0.932 | 357.4 |
| 75 | E1.77 | Het-A | H | H | CN | H | 1,2-dimethylpropyl | 1.138 | 369.4 |
| 76 | E1.78 | Het-A | H | H | CN | H | tetrahydropyran-3-yl | 0.943 | 383.4 |
| 77 | E1.79 | Het-A | H | H | CN | | tetrahydrothiopyran-3-yl | 1.069 | 399.3 |
| 78 | E1.80 | Het-A | H | H | CN | H | 4-methoxyphenyl | 1.081 | 405.4 |
| 79 | E1.81 | Het-A | H | H | CN | H | 3-thienyl | 1.054 | 381.3 |
| 80 | E1.82 | Het-A | H | H | CN | H | 3-acetoxy-1-methyl-propyl | 0.985 | 412.9 |
| 81 | E1.83 | Het-A | H | H | CN | H | tetrahydrothiopyran-4-ylmethyl | 1.074 | 412.8 |
| 82 | E1.84 | Het-A | H | H | CN | H | 1-phenoxyethyl | 1.130 | 418.9 |
| 83 | E1.85 | Het-A | H | H | CN | H | 1-(cyclohexoxy)ethyl | 1.190 | 424.9 |
| 84 | E1.86 | Het-A | H | H | CN | H | 6-methoxy-3-methyl-tetrahydropyran-2-yl | 1.065 | 426.9 |
| 85 | E1.87 | Het-A | H | H | CN | H | (4-isopropylcyclohexyl)methyl | 1.389 | 437.0 |
| 86 | E1.88 | Het-A | H | H | CN | H | 6-acetoxyhexyl | 1.086 | 440.9 |
| 87 | E1.89 | Het-A | H | H | CN | H | 5-methyl-1-phenyl-pyrazol-4-yl | 1.069 | 454.9 |
| 88 | E1.90 | Het-A | H | H | CN | H | 4-tert-butylphenyl | 1.314 | 431.5 |
| 89 | E1.91 | Het-A | H | H | CN | H | 3,4-dimethylphenyl | 1.197 | 403.4 |
| 90 | E1.92 | Het-A | H | H | CN | H | tetrahydrofuran-3-yl | 0.874 | 369.4 |
| 91 | E1.93 | Het-A | H | H | CN | H | 4-Cl—C₆H₄ | 1.185 | 411.1 |
| 92 | E1.94 | Het-A | H | H | CN | H | 4-(trifluoromethyl)phenyl | 1.219 | 443.3 |

If not indicated otherwise and except for compounds of formula (I-A), where R⁴ and R⁵ have the same meaning, the compounds of table 2 are mixtures of E/Z-isomers.
¹⁾isomer 1
²⁾isomer 2
^{*)}Het-A: 6-chloro-3-pyridyl
Het-B: 1-methylpyrazol-4-yl
Het-C: 2-chlorothiazol-5-yl
Het-D: 3-methylisoxazol-5-yl
^{#)} the abbreviations used have the same meaning as in table A.

B. Biological Examples

The biological activity of the compounds of formula (I) of the present invention may be evaluated in biological tests as described in the following.

General conditions: If not otherwise specified, most test solutions are to be prepared as follows: The active compound is to be dissolved at the desired concentration in a mixture of 1:1 (vol:vol) distilled water:acteon. Further, the test solutions are to be prepared at the day of use (and, if not otherwise specified, in general at concentrations wt/vol).

B.1 Green Peach Aphid (Myzus persicae)

For evaluating control of green peach aphid (Myzus persicae) through systemic means, the test unit consists of 96-well-microtiter plates containing liquid artificial diet under an artificial membrane.

The compounds are formulated using a solution containing 75% v/v water and 25% v/v DMSO. Different concentrations of formulated compounds are pipetted into the aphid diet, using a custom built pipetter, at two replications.

After application, 5-8 adult aphids are placed on the artificial membrane inside the microtiter plate wells. The aphids are then allowed to suck on the treated aphid diet and incubated at about 23+1° C. and about 50+5% relative humidity for 3 days. Aphid mortality and fecundity is then visually assessed.

In this test, compound E 1.3, 1.7, 1.8, 1.9, E1.10, E1.12, E1.13, E1.14, E1.16, E1.17, E1.23, E1.26, E1.27, E1.28, E1.29, E1.30, E1.31, E1.32, E1.33, E1.34, E1.35, E1.36, E1.37, E1.38, E1.39, E1.40, E1.41, E1.42, E1.43, E1.44, E1.45, E1.46, E1.47, E1.48, E1.49, E1.50, E1.51, E1.52, E1.53, E1.55, E1.56, E1.57, E1.59, E1.60, E1.61, E1.62, E1.63, E1.64, E1.65, E1.66, E1.67, E1.68, E1.69, E1.71, E1.72, E1.73, E1.74, E1.75, E1.76, E1.77, E1.78, E1.79, E1.80, E1.81, E1.82, E1.83, E1.84, E1.85, E1.86, E1.88, E1.89, E1.90, E1.91, E1.92, E1.93, and E1.94 at 2500 ppm showed at least 75% mortality in comparison with untreated controls.

B.2 Vetch Aphid (Megoura viciae)

For evaluating control of vetch aphid (Megoura viciae) through contact or systemic means the test unit consisted of 24-well-microtiter plates containing broad bean leaf disks.

The compounds were formulated using a solution containing 75% v/v water and 25% v/v DMSO. Different concentrations of formulated compounds were sprayed onto the leaf disks at 2.5 μl, using a custom built micro atomizer, at two replications.

After application, the leaf disks were air-dried and 5-8 adult aphids were placed on the leaf disks inside the microtiter plate wells. The aphids were then allowed to suck on the treated leaf disks and were incubated at about 23±1° C. and about 50±5% relative humidity for 5 days. Aphid mortality and fecundity was then visually assessed.

In this test, compound E1.3, E1.7, E1.8, E1.12, E1.13, E1.14, E1.15, E1.16, E1.26, E1.27, E1.28, E1.29, E1.30, E1.31, E1.32, E1.33, E1.34, E1.35, E1.36, E1.37, E1.38, E1.39, E1.40, E1.41, E1.43, E1.44, E1.45, E1.46, E1.50, E1.69, E1.71, E1.72, E1.73, E1.74, E1.75, E1.76, E1.77, E1.78, E1.79, E1.82, E1.83, E1.84, E1.85, E1.87, E1.88, E1.90, E1.91, E1.92, E1.93, and E1.94 at 2500 ppm showed at least 75% mortality in comparison with untreated controls.

B.3 Cotton Aphid (Aphis gossypii)

The active compound is dissolved at the desired concentration in a mixture of 1:1 (vol:vol) distilled water: acetone. Surfactant (Alkamuls® EL 620) is added at a rate of 0.1% (vol/vol). The test solution is prepared at the day of use.

Potted cowpea plants are colonized with approximately 50-100 aphids of various stages by manually transferring a leaf tissue cut from infested plant 24 hours before application. Plants are sprayed after the pest population has been recorded. Treated plants are maintained on light carts at about 28° C. Percent mortality is assessed after 72 hours.

In this test, compound E1.3, E.1.26, E.1.27, and E.1.33 at 500 ppm showed at least 75% mortality in comparison with untreated controls.

B.4 Cowpea Aphid (Aphis craccivora)

The active compound is dissolved at the desired concentration in a mixture of 1:1 (vol:vol) distilled water: acetone. Surfactant (Alkamuls® EL 620) is added at a rate of 0.1% (vol/vol). The test solution is prepared at the day of use.

Potted cowpea plants are colonized with approximately 50-100 aphids of various stages by manually transferring a leaf tissue cut from infested plant 24 hours before application. Plants are sprayed after the pest population has been recorded. Treated plants are maintained on light carts at about 28° C. Percent mortality is assessed after 72 hours.

In this test, compounds E1.3, E1.11, E1.25, E1.26, E1.27, E1.28, E1.30, E1.31, E1.32, E1.33, E1.34, and E1.35 at 500 ppm showed at least 75% mortality in comparison with untreated controls.

B.5 Orchid Thrips (Dichromothrips Corbetti)

Dichromothrips corbetti adults used for bioassay are obtained from a colony maintained continuously under laboratory conditions. For testing purposes, the test compound is diluted in a 1:1 mixture of acetone:water (vol:vol), plus 0.01% vol/vol Alkamuls® EL 620 surfactant.

Thrips potency of each compound is evaluated by using a floral-immersion technique. Plastic petri dishes are used as test arenas. All petals of individual, intact orchid flowers are dipped into treatment solution and allowed to dry. Treated flowers are placed into individual petri dishes along with about 20 adult thrips. The petri dishes are then covered with lids. All test arenas are held under continuous light and a temperature of about 28° C. for duration of the assay. After 3 days, the numbers of live thrips are counted on each flower, and along inner walls of each petri dish. The percent mortality is recorded 72 hours after treatment.

In this test, compounds E1.3, E1.11, E1.13, E1.24, E1.26, E1.34, E1.35, and E1.36 at 500 ppm showed at least 75% mortality in comparison with untreated controls.

B.6 Rice Green Leafhopper (Nephotettix virescens)

Rice seedlings are cleaned and washed 24 hours before spraying. The active compounds are formulated in 50:50 acetone:water (vol:vol), and 0.1% vol/vol surfactant (EL 620) is added. Potted rice seedlings are sprayed with 5 ml test solution, air dried, placed in cages and inoculated with 10 adults. Treated rice plants are kept at about 28-29° C. and relative humidity of about 50-60%. Percent mortality is recorded after 72 hours.

In this test, compounds E1.3, E1.11, E1.16, E1.26, E1.27, E1.29, E1.30, E1.32, E1.33, E1.34, E1.35, and E1.38 at 500 ppm showed at least 75% mortality in comparison with untreated controls.

B.7 Rice Brown Plant Hopper (Nilaparvata Lugens)

Rice seedlings are cleaned and washed 24 hours before spraying. The active compounds is formulated in 50:50 acetone:water (vol:vol) and 0.1% vol/vol surfactant (EL 620) was added. Potted rice seedlings are sprayed with 5 ml test solution, air dried, placed in cages and inoculated with 10 adults. Treated rice plants are kept at about 28-29° C. and relative humidity of about 50-60%. Percent mortality is recorded after 72 hours.

In this test, compound E1.3, E1.26, E1.30, and E1.32 at 500 ppm showed at least 75% mortality in comparison with untreated controls.

B.8 Boll Weevil (Anthonomus grandis)

For evaluating control of boll weevil (Anthonomus grandis) the test unit consisted of 96-well-microtiter plates containing an insect diet and 5-10 A. grandis eggs.

The compounds were formulated using a solution containing 75% v/v water and 25% v/v DMSO. Different concentrations of formulated compounds were sprayed onto the insect diet at 5 μl, using a custom built micro atomizer, at two replications.

After application, microtiter plates were incubated at about 25±1° C. and about 75±5% relative humidity for 5 days. Egg and larval mortality was then visually assessed.

In this test, compounds E1.13, E1.15, E1.23, E1.26, E1.28, and E1.85 at 2500 ppm showed at least 75% mortality in comparison with untreated controls.

B.9 Mediterranean Fruitfly (Ceratitis capitata)

For evaluating control of Mediterranean fruitfly (Ceratitis capitata) the test unit consisted of microtiter plates containing an insect diet and 50-80 C. capitata eggs.

The compounds were formulated using a solution containing 75% v/v water and 25% v/v DMSO. Different concentrations of formulated compounds were sprayed onto the insect diet at 5 μl, using a custom built micro atomizer, at two replications.

After application, microtiter plates were incubated at about 28±1° C. and about 80±5% relative humidity for 5 days. Egg and larval mortality was then visually assessed.

In this test, compounds E1.13, E1.23, E1.48, E1.51, E1.62, and E1.92 at 2500 ppm showed at least 75% mortality in comparison with untreated controls.

B.10 Tobacco Budworm (Heliothis virescens)

For evaluating control of tobacco budworm (Heliothis virescens) the test unit consisted of 96-well-microtiter plates containing an insect diet and 15-25 H. virescens eggs.

The compounds were formulated using a solution containing 75% v/v water and 25% v/v DMSO. Different concentrations of formulated compounds were sprayed onto the insect diet at 10 μl, using a custom built micro atomizer, at two replications.

After application, microtiter plates were incubated at about 28±1° C. and about 80±5% relative humidity for 5 days. Egg and larval mortality was then visually assessed.

In this test, compounds E1.13, E1.15, E1.23, E1.26, E1.45, E1.93, and E1.94 at 2500 ppm showed at least 75% mortality in comparison with untreated controls.

B.11 Diamond Back Moth (Plutella xylostella)

The active compound is dissolved at the desired concentration in a mixture of 1:1 (vol:vol) distilled water: acetone. Surfactant (Kinetic HV) is added at a rate of 0.01% (vol/vol). The test solution is prepared at the day of use.

Leaves of cabbage were dipped in test solution and air-dried. Treated leaves were placed in petri dishes lined with moist filter paper and inoculated with ten 3^{rd} instar larvae. Mortality was recorded 72 hours after treatment. Feeding damages were also recorded using a scale of 0-100%.

In this test, compounds E1.15, E1.16, E1.18, E1.19, and E1.26 at 500 ppm showed at least 75% mortality in comparison with untreated controls.

1-27. (canceled)

28. An N-acrylimino compound of formula (I): 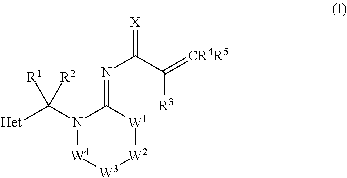
wherein
X is O or S;
Het is a 5- or 6-membered carbon-bound or nitrogen-bound heterocyclic ring, comprising 1, 2, 3, 4 or 5 carbon atoms and 1, 2 or 3 heteroatoms as ring members, which are independently selected from sulfur, oxygen or nitrogen, wherein the carbon, sulfur and nitrogen ring members can independently be partly or fully oxidized, and wherein each ring is optionally substituted by k identical or different substituents R⁶, wherein k is an integer selected from 0, 1, 2, 3 or 4;
wherein the moiety of the formula 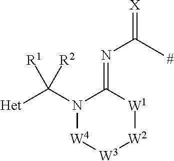
represents a radical A selected from the group consisting of W.Het-1, W.Het-2, W.Het-3, W.Het-4: 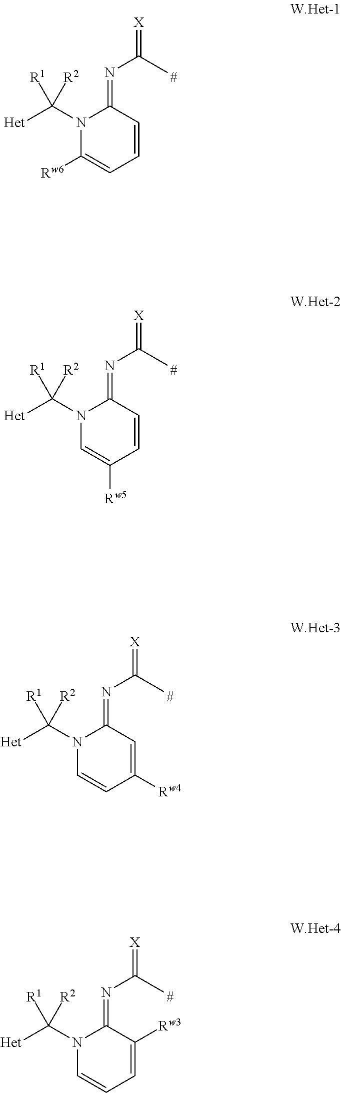
wherein # denotes the bond to the remainder of the molecule,
wherein
each R^{w3}, R^{w4}, R^{w5}, R^{w6} independently from each other, is selected from the group consisting hydrogen, halogen, cyano, azido, nitro, SCN, SF₅, C₁-C₁₀-alkyl, C₃-C₈-cycloalkyl, C₂-C₁₀-alkenyl, C₂-C₁₀-alkynyl, and wherein the carbon atoms of the aforementioned aliphatic and cycloaliphatic radicals may be unsubstituted or may be partly or fully halogenated and/or may optionally be substituted with 1, 2 or 3 identical or different radicals R⁷,
OR⁸, NR^{9a}R^{9b}, S(O)ₙR⁸, S(O)ₙNR^{9a}R^{9b}, C(═O)R^{7a}, C(═O)NR^{9a}R^{9b}, C(═O)OR⁸, C(═S)R^{7a}, C(═S)NR^{9a}R^{9b}, C(═S)OR⁸, C(═S)SR⁸, C(═NR¹⁷)R^{7a}, C(═NR¹⁷)NR^{9a}R^{9b} and Si(R¹¹)₂R¹²;
R¹, R² are independently from each other selected from the group consisting of hydrogen, halogen, CN, SCN, nitro, C₁-C₆-alkyl, C₃-C₆-cycloalkyl, wherein each of the two aforementioned radicals are unsubstituted, partly or completely halogenated or may carry any combination of 1, 2 or 3 radicals R⁷,
Si(R¹¹)₂R¹², OR⁸, OSO₂R⁸, S(O)ₙR⁸, S(O)ₙNR^{9a}R^{9b}, NR^{9a}R^{9b}, C(═O)NR^{9a}R^{9b}, C(═S)NR^{9a}R^{9b}, C(═O)OR⁸, C(═O)R^{7a}, C(═S)R^{7a}, phenyl, benzyl, where the phenyl ring in the last two radicals is unsubstituted or optionally substituted with 1, 2, 3, 4 or 5 identical or different substituents R¹⁰,
a 3-, 4-, 5-, 6- or 7-membered saturated, partly saturated or unsaturated aromatic heterocyclic ring comprising 1, 2 or 3 identical or different heteroatoms as ring members, which are selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized,
or
R¹ and R² form, together with the carbon atom, which they attached to, a 3-, 4-, 5- or 6-membered saturated or partly unsaturated carbocyclic or heterocyclic ring, wherein each of the carbon atoms of said cycle are unsubstituted or may carry any combination of 1 or 2 identical or different radicals R⁷,
or
R¹ and R² may together be ═O, ═CR¹³R¹⁴, ═S, ═NR¹⁷, ═NOR¹⁶ or ═NNR^{9a}R^{9b};
R³ is selected from the group consisting of hydrogen, halogen, CN, C₁-C₆-alkyl, C₃-C₈-cycloalkyl, C₃-C₈-cycloalkyl-C₁-C₄-alkyl, wherein each of the three last mentioned radicals are unsubstituted, partly or completely halogenated, or may carry any combination of 1, 2 or 3 radicals R⁷, OR⁸, S(O)ₙR⁸, S(O)ₙNR^{9a}R^{9b}, NR^{9a}R^{9b}, C(═O)OR⁸, C(═O)NR^{9a}R^{9b}, C(═S)NR^{9a}R^{9b}, C(═O)R^{7a}, C(═S)R^{7a},
a moiety Q-phenyl, where the phenyl ring is optionally substituted with 1, 2, 3, 4 or 5 identical or different substituents R¹⁰,
and a moiety Q-Het^{#} where Het^{#} represents a 3- to 10-membered saturated, partly saturated or unsaturated aromatic heterocyclic ring comprising 1, 2, 3 or 4 heteroatoms as ring members, which are identical or different and selected from oxygen, nitrogen and sulfur, where the heterocyclic ring is optionally substituted with 1, 2, 3, 4, 5 or 6 identical or different substituents R¹⁰, and wherein the nitrogen and/or the sulfur atom(s) of the heterocyclic ring may optionally be oxidized,
R⁴ is selected from the group consisting of hydrogen, halogen, CN, C₁-C₁₀-alkyl, C₂-C₁₀-alkenyl, C₃-C₈-cycloalkyl, wherein each of the three last mentioned radicals are unsubstituted, partly or completely halogenated, or may carry any combination of 1, 2 or 3 radicals R⁷,
S(O)ₙNR^{9a}R^{9b}, NR^{9a}R^{9b}, C(═O)OR⁸, C(═O)NR^{9a}R^{9b}, C(═S)NR^{9a}R^{9b}, C(═O)R^{7a}, C(═S)R^{7a},
a moiety Q-phenyl, where the phenyl ring is optionally substituted with 1, 2, 3, 4 or 5 identical or different substituents R¹⁰,
and a moiety Q-Het^{#} where Het^{#} represents a 3- to 10-membered saturated, partly saturated or unsaturated aromatic heterocyclic ring comprising 1, 2, 3 or 4 heteroatoms as ring members, which are identical or different and selected from oxygen, nitrogen and sulfur, where the heterocyclic ring is optionally substituted with 1, 2, 3, 4, 5 or 6 identical or different substituents R¹⁰, and wherein the nitrogen and/or the sulfur atom(s) of the heterocyclic ring may optionally be oxidized;
R⁵ is selected from the group consisting of hydrogen, CN, C₁-C₁₀-alkyl, C₂-C₁₀-alkenyl, C₃-C₈-cycloalkyl, wherein each of the three last mentioned radicals are unsubstituted, partly or completely halogenated, or may carry any combination of 1, 2 or 3 radicals R⁷,
S(O)ₙNR^{9a}R^{9b}, O—NR^{9a}R^{9b}, NR^{9a}R^{9b}, NR^{9a}NR^{9a}R^{9b}, NR^{9a}C(═O)R^{7a}, NR^{9a}C(═S)R^{7a}, NR^{9a}C(═O)NR^{9a}R^{9b}, NR^{9a}C(═S)NR^{9a}R^{9b}, C(═O)OR⁸, C(═O)NR^{9a}R^{9b}, C(═S)NR^{9a}R^{9b}, C(═O)R^{7a}, C(═S)R^{7a},
a moiety Q-phenyl, where the phenyl ring is optionally substituted with 1, 2, 3, 4 or 5 identical or different substituents R¹⁰,
and a moiety Q-Het^{#} where Het^{#} represents a 3- to 10-membered saturated, partly saturated or unsaturated aromatic heterocyclic ring comprising 1, 2, 3 or 4 heteroatoms as ring members, which are identical or different and selected from oxygen, nitrogen and sulfur, where the heterocyclic ring is optionally substituted with 1, 2, 3, 4, 5 or 6 identical or different substituents R¹⁰, and wherein the nitrogen and/or the sulfur atom(s) of the heterocyclic ring may optionally be oxidized, or
R⁴ and R⁵ together form a moiety selected from Alk, S(O)ₙ-Alk, S(O)ₙ-Alk, NR^{9c}-Alk, S(O)ₙ-Alk′-S(O)ₙ, (O)-Alk′-O, O-Alk′-NR^{9d}, S(O)ₙ-Alk′-NR^{9d}, O-Alk′-S and NR^{9c}-Alk′-NR^{9d}, where
Alk represents a saturated or unsaturated 2-, 3-, 4- or 5-membered carbon chain group, which is unsubstituted or carries 1, 2, 3 or 4 radicals R⁷ or a fused phenylene ring, where the fused phenyl ring is unsubstituted or substituted with 1, 2, 3 or 4 radicals R¹⁰;
Alk′ is CH₂, where one or both hydrogen atoms may optionally be replaced by R⁷ or represents a saturated or unsaturated 2-, 3- or 4-membered carbon chain group, which is unsubstituted or carries 1, 2, 3 or 4 radicals R⁷ or a fused phenylene ring, where the fused phenyl ring is unsubstituted or substituted with 1, 2, 3 or 4 radicals R¹⁰;
provided that R⁵ is different from hydrogen and C₁-C₆-alkyl, if R³ is hydrogen;
where, independently of their occurrence,
n is 0, 1 or 2;
Q is a single bond, C₁-C₄-alkylene, a moiety NR^{9a}, or NR^{9a}—C₁-C₄-alkylene, where the heteroatom in the last 2 moieties is bound to the carbon atom of CR⁴R⁵;
R⁶ is selected from the group consisting of halogen, cyano, azido, nitro, SCN, SF₅, C₁-C₁₀-alkyl, C₃-C₈-cycloalkyl, C₂-C₁₀-alkenyl, C₂-C₁₀-alkynyl, and wherein the carbon atoms of the aforementioned aliphatic and cyclo-aliphatic radicals may optionally be further substituted independently from one another with 1, 2 or 3 radicals R⁷,
OR⁸, NR^{17a}R^{17b}, S(O)ₙR⁸, S(O)ₙNR^{17a}R^{17b}, C(═O)R^{7a}, C(═O)NR^{17a}R^{17b}, C(═O)OR⁸, C(═S)R^{7a}, C(═S)NR^{17a}R^{17b}, C(═S)OR⁸, C(═S)SR⁸, C(═NR¹⁷)R^{7a}, C(═NR¹⁷)NR^{17a}R^{17b}, Si(R¹¹)₂R¹²;
phenyl, optionally substituted with 1, 2, 3, 4 or 5 identical or different substituents R¹⁰,
a 3-, 4-, 5-, 6- or 7-membered saturated, partly saturated or unsaturated aromatic heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized,
or two of R⁶ present on one ring carbon may together form ═O, ═CR¹³R¹⁴, ═S, ═NR¹⁷, ═NOR¹⁶, ═NNR^{9a}R^{9b},
or two R⁶, which are bound to adjacent ring atoms, together may form a linear C₂-C₇ alkylene chain, thus forming, together with the ring atoms to which they are bound, a 4-, 5-, 6-, 7-, 8- or 9-membered ring, where 1 or 2 CH₂ moieties of the alkylene chain may be replaced by 1 or 2 heteroatom moieties selected from O, S and NR^{17c} and/or 1 or 2 of the CH₂ groups of the alkylene chain may be replaced by a group C═O, C═S and/or C═NR¹⁷; and where the alkylene chain is unsubstituted or may be substituted with 1, 2, 3, 4, 5 or 6 radicals selected from the group consisting of halogen, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted with 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted with 1, 2, 3, 4 or 5 radicals R¹⁰;
R⁷ independently of its occurrence, is selected from the group consisting of cyano, azido, nitro, —SCN, SF₅, C₁-C₆-alkyl, C₁-C₆-haloalkyl, C₃-C₈-cycloalkyl, where the C₃-C₈-cycloalkyl ring in the last group is optionally substituted with 1, 2, 3 or 4 C₁-C₄-radicals, C₃-C₈-halocycloalkyl, C₂-C₆-alkenyl, C₂-C₆-haloalkenyl, C₂-C₆-alkynyl, C₂-C₆-haloalkynyl, Si(R¹¹)₂R¹², OR⁸, OSO₂R⁸, S(O)ₙR⁸, S(O)ₙNR^{17a}R^{17b}, NR^{17a}R^{17b}, C(═O)NR^{17a}R^{17b}, C(═S)NR^{17a}R^{17b}, C(═O)OR⁸, C(═O)R¹⁵, C(═S)R¹⁵, C(═NR¹⁷)R¹⁵,
phenyl, phenyl-C₁-C₄-alkyl, where the phenyl ring in the last two groups is optionally substituted with 1, 2, 3, 4 or 5 identical or different substituents R¹⁰, and
a 3-, 4-, 5-, 6- or 7-membered saturated, partly saturated or unsaturated aromatic heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized,
or two R⁷ present on one carbon atom may together form ═O, ═CR¹³R¹⁴, ═S, ═NR¹⁷, ═NOR¹⁶, ═NNR^{9a}R^{9b},
or two R⁷ may form a 3-, 4-, 5-, 6-, 7- or 8-membered saturated or partly unsaturated carbocyclic or heterocyclic ring together with the carbon atoms to which the two R⁷ are bonded, where the heterocyclic ring comprises 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰;
R^{7a} independently of its occurrence, is selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl,
phenyl, optionally substituted with 1, 2, 3, 4 or 5 identical or different substituents R¹⁰,
benzyl, where the phenyl ring in the last radical is unsubstituted or substituted by 1, 2, or 3 identical or different substituents selected from the group consisting of halogen, CN, C₁-C₄-alkyl, C₁-C₄-haloalkyl, C₁-C₄-alkoxy and C₁-C₄-haloalkoxy and
a 3-, 4-, 5-, 6- or 7-membered saturated, partly saturated or unsaturated aromatic heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized;
R⁸ independently of its occurrence, is selected from the group consisting of hydrogen, C₁-C₆-alkyl, C₁-C₄-alkoxy-C₁-C₄-alkyl, C₁-C₆-haloalkyl, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl, C(═O)R¹⁵, C(═O)NR^{17a}R^{17b}, C(═S)NR^{17a}R^{17b}, C(═O)OR¹⁶,
phenyl, phenyl-C₁-C-₄-alkyl, where the phenyl ring in the last two mentioned radicals is unsubstituted or substituted with 1, 2, 3, 4 or 5 identical or different substituents R¹⁰, and
a 3-, 4-, 5-, 6- or 7-membered saturated, partly saturated or unsaturated aromatic heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized,
R⁹ independently of its occurrence, is selected from the group consisting of hydrogen, C₁-C₆-alkyl, C₁-C₆-haloalkyl, C₁-C₆-alkoxy, C₁-C₆-haloalkoxy, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl,
phenyl, optionally substituted with 1, 2, 3, 4 or 5 identical or different substituents R¹⁰;
benzyl, where the phenyl ring in the last radical is unsubstituted or substituted by 1, 2, or 3 identical or different substituents selected from the group consisting of halogen, CN, C₁-C₄-alkyl, C₁-C₄-haloalkyl, C₁-C₄-alkoxy and C₁-C₄-haloalkoxy, a 3-, 4-, 5-, 6- or 7-membered saturated, partly saturated or unsaturated aromatic C-bound heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized,
R^{9a}, R^{9b} are each independently from one another selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₃-C₈-cycloalkyl-C₁-C₄-alkyl, C₂-C₆-alkenyl, C₂-C₆-haloalkenyl, C₂-C₆-alkynyl, C₂-C₆ haloalkynyl,
S(O)ₙR¹⁶, —S(O)ₙNR^{17a}R^{17b}, C(═O)R¹⁵, C(═O)OR¹⁶, C(═O)NR^{17a}R^{17b}, C(═S)R¹⁵, C(═S)SR¹⁶, C(S)NR^{17a}R^{17b}, C(═NR¹⁷)R¹⁵;
phenyl, benzyl, 1-phenethyl or 2-phenethyl, where the phenyl ring in the last four mentioned radicals is unsubstituted or may be substituted with 1, 2, 3, 4 or 5 identical or different substituents R¹⁰; and
a 3-, 4-, 5-, 6- or 7-membered saturated, partly saturated or unsaturated aromatic C-bound heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized,
or,
R^{9a} and R^{9b} are together a C₂-C₇-alkylene chain and form a 3-, 4-, 5-, 6-, 7- or 8-membered saturated, partly saturated or unsaturated aromatic ring together with the nitrogen atom they are bonded to, wherein the alkylene chain may contain one or two heteroatoms, which are, independently of each other, selected from oxygen, sulfur and nitrogen, and where the alkylene chain may optionally be substituted with 1, 2, 3 or 4 radicals selected from halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl, optionally substituted with 1, 2, 3, 4 or 5 identical or different substituents R¹⁰, and a 3-, 4-, 5-, 6- or 7-membered saturated, partly saturated or unsaturated aromatic C-bound heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized,
or
R^{9a} and R^{9b} together may form ═CR¹³R¹⁴, ═NR¹⁷═NOR¹⁶, ═NNR^{17a}R^{17b} moiety;
R^{9c}, R^{9d} are each independently from one another selected from the group consisting of hydrogen, C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl,
S(O)ₙR¹⁶, —S(O)ₙNR^{17a}R^{17b}, C(═O)R¹⁵, C(═O)OR¹⁶, C(═O)NR^{17a}R^{17b}, C(═S)R¹⁵, C(═S)SR¹⁶, C(═S)NR^{17a}R^{17b}, C(═NR¹⁷)R¹⁵;
phenyl, benzyl, where the phenyl ring in the last two mentioned radicals is unsubstituted or may be substituted with 1, 2, 3, 4 or 5 identical or different substituents R¹⁰; and
a 3-, 4-, 5-, 6- or 7-membered saturated, partly saturated or unsaturated aromatic C-bound heterocyclic ring comprising 1, 2 or 3 heteroatoms as ring members, which are identical or different and selected from oxygen, nitrogen and sulfur, where the heterocyclic ring is optionally substituted with 1, 2, 3 or 4 identical or different substituents R¹⁰, and wherein the nitrogen and/or the sulfur atom(s) of the heterocyclic ring may optionally be oxidized;
R¹⁰ independently of its occurrence, is selected from the group consisting of halogen, cyano, azido, nitro, SCN, SF₅, C₁-C₁₀-alkyl, C₃-C₈-cycloalkyl, C₂-C₁₀-alkenyl, C₂-C₁₀-alkynyl, wherein the carbon atoms of the aforementioned aliphatic and cycloaliphatic radicals may optionally be substituted with 1, 2, 3, 4 or 5 identical or different radicals R^{10a},
Si(R¹¹)₂R¹², OR¹⁶, OS(O)ₙR¹⁶, —S(O)ₙR¹⁶, S(O)ₙNR^{17a}R^{17b}, NR^{17a}R^{17b}, C(═O)R¹⁵, C(═S)R¹⁵, C(═O)OR¹⁶, —C(═NR¹⁷)R¹⁵, C(═O)NR^{17a}R^{17b}, C(═S)NR^{17a}R^{17b},
phenyl, optionally substituted with 1, 2, 3, 4 or 5 identical or different radicals selected from OH, halogen, cyano, nitro, C₁-C₆-alkyl, C₁-C₆-haloalkyl, C₁-C₆-alkoxy and C₁-C₆-haloalkoxy, and
a 3-, 4-, 5-, 6- or 7-membered saturated, partly saturated or unsaturated aromatic heterocyclic ring comprising 1, 2 or 3 heteroatoms as ring members, which are identical or different and selected from oxygen, nitrogen and sulfur, where the heterocyclic ring is unsubstituted or may be substituted with 1, 2, 3, 4 or 5 substituents selected independently from one another from halogen, cyano, NO₂, C₁-C₆-alkyl, C₁-C₆-haloalkyl, C₁-C₆-alkoxy and C₁-C₆-haloalkoxy, and wherein the nitrogen and/or the sulfur atom(s) of the heterocyclic ring may optionally be oxidized;
or
two R¹⁰ present together on one carbon ring atom of a saturated or partly unsaturated heterocyclic radical may form ═O, ═CR¹³R¹⁴, ═S, ═NR¹⁷, ═NOR¹⁶, ═NNR^{17a}R^{17b};
or,
two R¹⁰ on adjacent carbon ring atoms may also be a bivalent radical selected from CH₂CH₂CH₂CH₂, CH═CH—CH═CH, N═CH—CH═CH, CH═N—CH═CH, N═CH—N═CH, OCH₂CH₂CH₂, OCH═CHCH₂, CH₂OCH₂CH₂, OCH₂CH₂O, OCH₂OCH₂, CH₂CH₂CH₂, CH═CHCH₂, CH₂CH₂O, CH═CHO, CH₂OCH₂, CH₂C(═O)O, C(═O)OCH₂, O(CH₂)O, SCH₂CH₂CH₂, SCH═CHCH₂, CH₂SCH₂CH₂, SCH₂CH₂S, SCH₂SCH₂, CH₂CH₂S, CH═CHS, CH₂SCH₂, CH₂C(═S)S, C(═S)SCH₂, S(CH₂)S, CH₂CH₂NR¹⁷, CH₂CH═N, CH═CH—NR¹⁷, OCH═N, SCH═N and form together with the carbon atoms to which the two R¹⁰ are bonded to a 5-membered or 6-membered partly saturated or unsaturated, aromatic carbocyclic or heterocyclic ring, wherein the ring may optionally be substituted with one or two substituents selected from ═O, OH, CH₃, OCH₃, halogen, cyano, halomethyl and halomethoxy;
R^{10a} independently of its occurrence, is halogen or has one of the meanings given for R⁷;
R¹¹, R¹² independently of their occurrence, are selected from the group consisting of C₁-C₆-alkyl, C₁-C₆-haloalkyl, C₁-C₆-alkoxy, C₁-C₆-alkoxy-C₁-C₄-alkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₃-C₈-cycloalkyl-C₁-C₄-alkyl, C₃-C₈-halocycloalkyl-C₁-C₄-alkyl, C₁-C₆-haloalkoxy-C₁-C₄-alkyl, phenyl and benzyl, where the phenyl ring in last two radicals are unsubstituted or substituted with 1, 2, 3, 4 or 5 identical or different radicals selected from halogen, OH, cyano, NO₂, C₁-C₆-alkyl, C₁-C₆-haloalkyl, C₁-C₆-alkoxy and C₁-C₆-haloalkoxy;
R¹³, R¹⁴ independently of their occurrence, are selected from the group consisting of hydrogen, halogen, CN, C₁-C₆-alkyl, C₃-C₆-cycloalkyl, C₁-C₄-alkoxy-C₁-C₄-alkyl, phenyl and benzyl;
R¹⁵ independently of its occurrence, is selected from the group consisting of hydrogen, C₁-C₆-alkyl, C₂-C₆-alkenyl, C₂-C₆-alkynyl, C₃-C₈-cycloalkyl, wherein the four last mentioned aliphatic and cycloaliphatic radicals may be unsubstituted, partially or fully halogenated and/or oxygenated and/or may carry 1 or 2 radicals selected from C₁-C₄-alkoxy;
C₃-C₈-cycloalkyl-C₁-C₄-alkyl;
phenyl, benzyl and pyridyl, wherein the last three radicals may be unsubstituted, partially or fully halogenated and/or may carry 1, 2 or 3 substituents selected from C₁-C₆-alkyl, C₁-C₆-haloalkyl, C₁-C₆-alkoxy, C₁-C₆-haloalkoxy, (C₁-C₆-alkoxy)carbonyl, (C₁-C₆-alkyl)amino and di-(C₁-C₆-alkyl)amino;
R¹⁶ independently of its occurrence, is selected from the group consisting of hydrogen, C₁-C₆-alkyl, C₂-C₆-alkenyl, C₂-C₆-alkynyl, C₃-C₈-cycloalkyl, C₃-C₈-cycloalkyl-C₁-C₄-alkyl, wherein the five last mentioned aliphatic and cycloaliphatic radicals may be unsubstituted, partially or fully halogenated and/or oxygenated and/or may carry 1 or 2 radicals selected from C₁-C₄-alkoxy, phenyl, benzyl and pyridyl, wherein the last three radicals may be unsubstituted, partially or fully halogenated and/or may carry 1, 2 or 3 substituents selected from CN, C₁-C₆-alkyl, C₁-C₆-haloalkyl, C₁-C₆-alkoxy, C₁-C₆-haloalkoxy, (C₁-C₆-alkoxy)carbonyl, (C₁-C₆-alkyl)amino or di-(C₁-C₆-alkyl)amino;
R¹⁷ independently of its occurrence, is selected from the group consisting of hydrogen, C₁-C₆-alkoxy, C₁-C₆-haloalkoxy, C₁-C₆-alkylthio, trimethylsilyl, triethylsilyl, tertbutyldimethylsilyl,
C₁-C₆-alkyl, C₂-C₆-alkenyl, C₂-C₆-alkynyl, C₃-C₈-cycloalkyl, wherein the four last mentioned aliphatic and cycloaliphatic radicals may be unsubstituted, partially or fully halogenated and/or oxygenated and/or may carry 1 or 2 radicals selected from C₁-C₄-alkoxy;
C₃-C₈-cycloalkyl-C₁-C₄-alkyl;
phenyl, benzyl, pyridyl, phenoxy, wherein the four last mentioned radicals may be unsubstituted, partially or fully halogenated and/or carry 1, 2 or 3 substituents selected from CN, C₁-C₆-alkyl, C₁-C₆-haloalkyl, C₁-C₆-alkoxy, C₁-C₆-haloalkoxy and (C₁-C₆-alkoxy)carbonyl,
R^{17a}, R^{17b} are each independently from one another selected from the group consisting of hydrogen, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₂-C₆-alkynyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄-alkoxy,
phenyl, benzyl, pyridyl, phenoxy, wherein the four last mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy and (C₁-C₆-alkoxy)carbonyl,
or,
R^{17a} and R^{17b} may together be a C₂-C₆-alkylene chain forming a 3- to 7-membered saturated, partly saturated or unsaturated ring together with the nitrogen atom R^{17a} and R^{17b} are bonded to, wherein the alkylene chain may contain 1 or 2 heteroatoms selected, independently of each other, from oxygen, sulfur and nitrogen, and may optionally be substituted with halogen, C₁-C₄-haloalkyl, C₁-C₄-alkoxy or C₁-C₄-haloalkoxy, and wherein the nitrogen and/or the sulfur atom(s) of the heterocyclic ring may optionally be oxidized;
or
R^{17a} and R^{17b} together may form ═CR¹³R¹⁴, ═NR¹⁷ or ═NOR¹⁶ moiety;
R^{17c} independently of its occurrence, is selected from the group consisting of hydrogen, CN, C₁-C₆-alkyl, C₂-C₆-alkenyl, C₂-C₆-alkynyl, C₃-C₈-cycloalkyl, C₃-C₈-cycloalkyl-C₁-C₄-alkyl, wherein the five last mentioned aliphatic and cycloaliphatic radicals may be unsubstituted, partially or fully halogenated and/or oxygenated and/or may carry 1 or 2 radicals selected from C₁-C₄-alkoxy, phenyl, benzyl and pyridyl, wherein the last three radicals may be unsubstituted, partially or fully halogenated and/or may carry 1, 2 or 3 substituents selected from CN, C₁-C₆-alkyl, C₁-C₆-haloalkyl, C₁-C₆-alkoxy, C₁-C₆-haloalkoxy, (C₁-C₆-alkoxy)carbonyl, (C₁-C₆-alkyl)amino or di-(C₁-C₆-alkyl)amino;
the stereoisomers, tautomers and the salts thereof.

29. The compound of claim 28, wherein
Het is selected from the group consisting of radicals of the following formula Het-1 to Het-24: 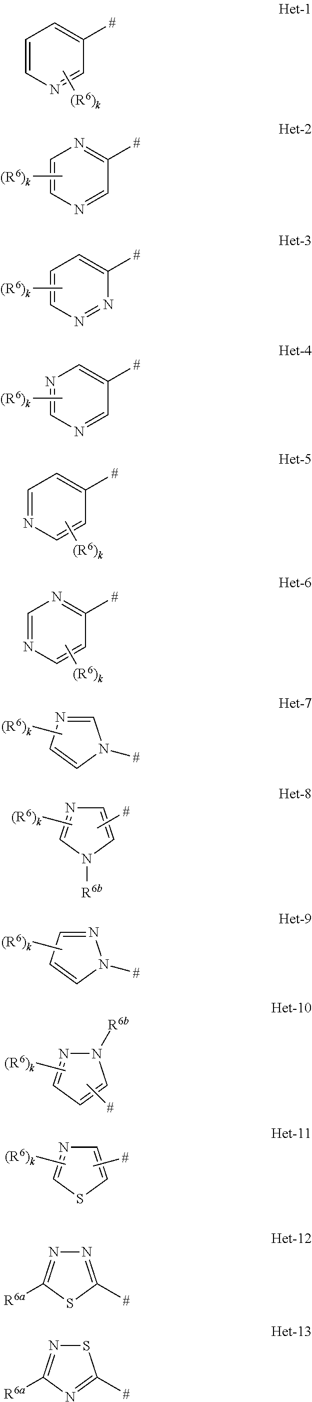 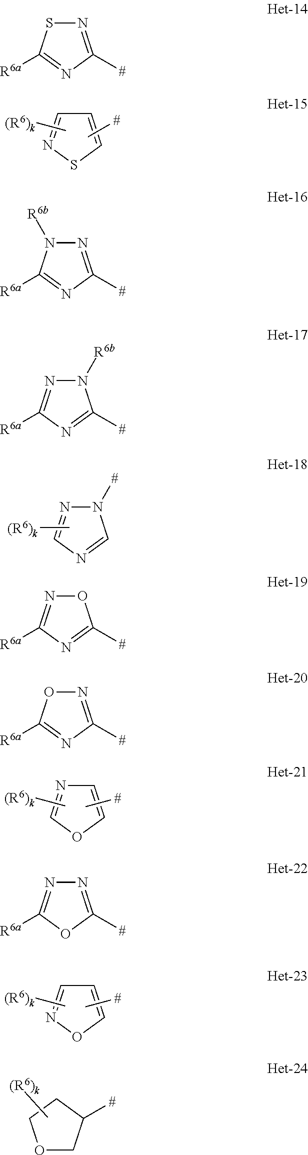
wherein # denotes the bond in formula (I), and wherein
k is 0, 1 or 2; and
R^{6a} is hydrogen or has one of the meanings given for R⁶ and
R^{6b} is hydrogen, C₁-C₄-alkyl or C₁-C₄-haloalkyl.

30. The compound of claim 28, wherein
Het is selected from the group consisting of radicals of formulae Het-1, Het-10a, Het-11a, Het-23a and Het-24, 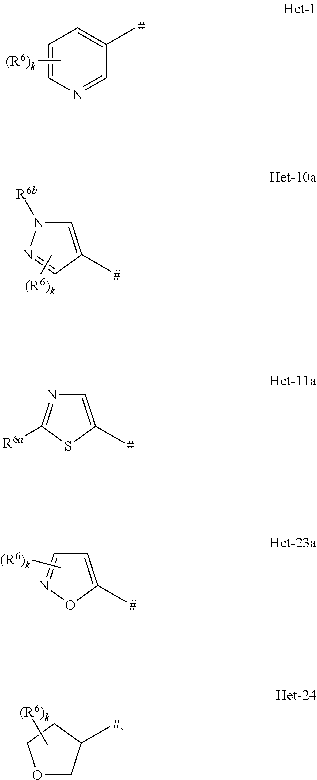
wherein # denotes the bond in formula (I), and wherein
R⁶ is selected from halogen, C₁-C₄-alkyl, C₁-C₄-alkoxy, C₁-C₄-haloalkoxy and C₁-C₄-haloalkyl;
R^{6a} is selected from hydrogen, halogen, C₁-C₄-alkyl, C₁-C₄-alkoxy, C₁-C₄-haloalkoxy and C₁-C₄-haloalkyl;
R^{6b} is selected from C₁-C₄-alkyl, C₁-C₄-alkoxy, C₁-C₄-haloalkoxy and C₁-C₄-haloalkyl; and
k is 0, 1 or 2.

31. The compound of claim 30, wherein Het is Het-1a 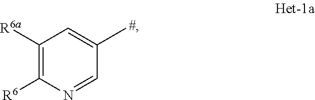
wherein # denotes the bond in formula (I),
R⁶ is selected from halogen, C₁-C₄-alkyl and C₁-C₄-haloalkyl and
R^{6a} is hydrogen or halogen, in particular hydrogen.

32. The compound of claim 28, wherein
R¹, R² are independently from each other selected from the group consisting of hydrogen, halogen, CN, C₁-C₆-alkyl, C₃-C₆-cycloalkyl, C₁-C₆-haloalkyl, C₃-C₆-halocycloalkyl;
or
R¹ and R² may together be ═CR¹³R¹⁴;
or
R¹ and R² form, together with the carbon atom, which they attached to, a 3- to 5-membered saturated carbocyclic ring.

33. The compound of claim 32, wherein both R¹ and R² are hydrogen or one of R¹ and R² is hydrogen while the other is methyl.

34. The compound of claim 28, wherein
R³ is selected from the group consisting of hydrogen, halogen, CN, C₁-C₆-alkyl and, C₁-C₆-haloalkyl.

35. The compound of claim 34, wherein
R³ is fluorine or CN.

36. The compound of claim 34, wherein
R³ is hydrogen.

37. The compound of claim 28, wherein
R⁴ is hydrogen, halogen, CN, NR^{9a}R^{9b}, C₁-C₆-alkyl, C₁-C₆-haloalkyl, or Q-phenyl, where Q is a single bond, NR^{9a}, CH₂, or NR^{9a}CH₂ and where phenyl is unsubstituted or substituted with 1, 2, 3, 4 or 5 identical or different substituents R¹⁰.

38. The compound of claim 37, wherein
R⁴ is selected from the group consisting of hydrogen and fluorine.

39. The compound of claim 28, wherein
R⁵ is —CN, C₁-C₄-cyanoalkyl, C₁-C₄-alkoxy-C₁-C₄-alkyl, C₁-C₄-alkthio-C₁-C₄-alkyl, NR^{9a}R^{9b}, C₁-C₆-alkyl, C₁-C₆-haloalkyl, C(═O)OR⁸, C(═O)NR^{9a}R^{9b}, C(═S)NR^{9a}R^{9b}, C(═O)R^{7a}, C(═S)R^{7a},
C₃-C₆-cycloalkyl-C₁-C₄-alkyl, C₃-C₈-cycloalkyl, where cycloalkyl in the last two radicals is unsubstituted or optionally substituted with 1, 2, 3 or 4 C₁-C₄-alkyl radicals,
Q-phenyl, where phenyl is unsubstituted or substituted with 1, 2, 3, 4 or 5 identical or different substituents R¹⁰, or Q-Het^{#}, where Het^{#} is unsubstituted or substituted with 1, 2, 3, or 4 identical or different substituents R¹⁰,
and where Q, irrespectively of its occurrence, is a single bond, NR^{9a}, CH₂, or NR^{9a}CH₂.

40. The compound of claim 28, wherein
R³ is fluorine or CN;
R⁴ is selected from the group consisting of hydrogen and fluorine;
R⁵ cyanomethyl, C₁-C₄-alkoxy-C₁-C₄-alkyl, C₁-C₄-alkthio-C₁-C₄-alkyl, NR^{9a}R^{9b}, C₁-C₆-alkyl, C₁-C₄-haloalkyl, C(═O)OR⁸, C(═O)NR^{9a}R^{9b}, C(═O)R^{7a},
C₃-C₆-cycloalkyl-C₁-C₄-alkyl, C₃-C₈-cycloalkyl, where cycloalkyl in the last to radicals is unsubstituted or optionally substituted with 1, 2, 3 or 4 C₁-C₄-alkyl radicals,
Q-phenyl, where phenyl is unsubstituted or substituted with 1, 2, 3, 4 or 5 identical or different substituents R¹⁰, or Q-Het^{#}, where Het^{#} is unsubstituted or substituted with 1, 2, 3, or 4 identical or different substituents R¹⁰,
and where Q, irrespectively of its occurrence, is a single bond, NH, N(C₁-C₄-alkyl), CH₂, or N(C₁-C₄-alkyl) CH₂.

41. The compounds of claim 28,
wherein
R^{w3}, R^{w4}, R^{w5} and R^{w6} are, independently of each other, selected from hydrogen, halogen, C₁-C₄-alkoxy, C₁-C₄-haloalkoxy, C₁-C₄-alkyl and C₁-C₄-haloalkyl.

42. The compound of claim 41, wherein the radical A is W.Het-2.

43. The compound of claim 42, wherein R^{w5} is hydrogen.

44. The compound of claim 42, wherein the radical A is W.Het-2 and wherein Het is selected from the group consisting of radicals of formulae Het-1, Het-11a and Het-24, 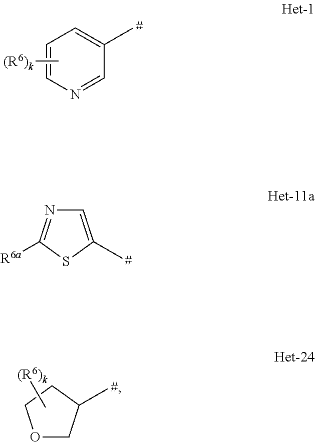
wherein # denotes the bond in formula (I), and wherein
R⁶ is selected from halogen, C₁-C₄-alkyl, C₁-C₄-alkoxy, C₁-C₄-haloalkoxy and C₁-C₄-haloalkyl;
R^{6a} is selected from hydrogen, halogen, C₁-C₄-alkyl, C₁-C₄-alkoxy, C₁-C₄-haloalkoxy and C₁-C₄-haloalkyl; and
k is 0, 1 or 2.

45. The compound of claim 41, wherein
R¹, R² are independently from each other selected from the group consisting of hydrogen, halogen, CN, C₁-C₆-alkyl, C₃-C₆-cycloalkyl, C₁-C₆-haloalkyl, C₃-C₆-halocycloalkyl;
or
R¹ and R² may together be ═CR¹³R¹⁴;
or
R¹ and R² form, together with the carbon atom, which they attached to, a 3- to 5-membered saturated carbocyclic ring.
R³ is selected from the group consisting of hydrogen, halogen, CN, C₁-C₆-alkyl and, C₁-C₆-haloalkyl.
R⁴ is hydrogen, halogen, CN, NR^{9a}R^{9b}, C₁-C₆-alkyl, C₁-C₆-haloalkyl, or Q-phenyl, where Q is a single bond, NR^{9a}, CH₂, or NR^{9a}CH₂ and where phenyl is unsubstituted or substituted with 1, 2, 3, 4 or 5 identical or different substituents R¹⁰, and
R⁵ is —CN, C₁-C₄-cyanoalkyl, C₁-C₄-alkoxy-C₁-C₄-alkyl, C₁-C₄-alkthio-C₁-C₄-alkyl, NR^{9a}R^{9b}, C₁-C₆-alkyl, C₁-C₆-haloalkyl, C(═O)OR⁸, C(═O)NR^{9a}R^{9b}, C(═S)NR^{9a}R^{9b}, C(═O)R^{7a}, C(═S)R^{7a},
C₃-C₆-cycloalkyl-C₁-C₄-alkyl, C₃-C₈-cycloalkyl, where cycloalkyl in the last two radicals is unsubstituted or optionally substituted with 1, 2, 3 or 4 C₁-C₄-alkyl radicals,
Q-phenyl, where phenyl is unsubstituted or substituted with 1, 2, 3, 4 or 5 identical or different substituents R¹⁰, or Q-Het^{#}, where Het^{#} is unsubstituted or substituted with 1, 2, 3, or 4 identical or different substituents R¹⁰,
and where Q, irrespectively of its occurrence, is a single bond, NR^{9a}, CH₂, or NR^{9a}CH₂;
or
R⁴ and R⁵ together form a moiety selected from Alk, S(O)ₙ-Alk, (O)-Alk, NR^{9c}-Alk, S(O)ₙ-Alk′-S(O)ₙ, (O)-Alk′-O and NR^{9c}-Alk′-NR^{9d}, where Alk represents a saturated or unsaturated 2-, 3- or 4-membered carbon chain group, which is unsubstituted or carries 1, 2, 3 or 4 radicals R⁷ or a fused phenylene ring, where the fused phenyl ring is unsubstituted or substituted with 1, 2, 3 or 4 radicals R¹⁰ and where Alk′ is CH₂ or has one of the meanings given for Alk.

46. The compound of claim 41, wherein
both R¹ and R² are hydrogen;
R³ is fluorine or CN;
R⁴ is selected from the group consisting of hydrogen and fluorine;
R⁵ cyanomethyl, NR^{9a}R^{9b}, C₁-C₆-alkyl, C₁-C₄-haloalkyl, C₁-C₄-alkoxy-C₁-C₄-alkyl, C₁-C₄-alkthio-C₁-C₄-alkyl, C(═O)OR⁸, C(═O)NR^{9a}R^{9b}, C(═O)R^{7a}, C₃-C₆-cycloalkyl-C₁-C₄-alkyl, C₃-C₈-cycloalkyl, where cycloalkyl in the last two radicals is unsubstituted or optionally substituted with 1, 2, 3 or 4 C₁-C₄-alkyl radicals,
Q-phenyl, where phenyl is unsubstituted or substituted with 1, 2, 3, 4 or 5 identical or different substituents R¹⁰, or Q-Het^{#}, where Het^{#} is unsubstituted or substituted with 1, 2, 3, or 4 identical or different substituents R¹⁰,
and where Q, irrespectively of its occurrence, is a single bond, NH, N(C₁-C₄-alkyl), CH₂, or N(C₁-C₄-alkyl)-CH₂.

47. The compound of claim 28, wherein
X is O.

48. An agricultural or veterinary composition for combating animal pests comprising at least one compound as defined in claim 28 and at least one inert liquid and/or solid acceptable carrier and optionally, if desired, at least one surfactant.

49. The use of a compound as defined in claim 28 for combating or controlling invertebrate pests, for protecting growing plants from attack or infestation by invertebrate pests, for protecting plant propagation material, especially seeds, from soil insects, or for protect the seedlings roots and shoots of plants from soil and foliar insects.

50. A method for combating or controlling invertebrate pests, which method comprises contacting said pest or its food supply, habitat or breeding grounds with a pesticidally effective amount of at least one compound as defined in claim 28.

51. A method for protecting growing plants from attack or infestation by invertebrate pests, which method comprises contacting a plant, or soil or water in which the plant is growing, with a pesticidally effective amount of at least one compound as defined in claim 28.

52. A method for the protection of plant propagation material, especially seeds, from soil insects and of the seedlings roots and shoots from soil and foliar insects comprising contacting the plant propagation material before sowing and/or after pregermination with at least one compound as defined in claim 28.

53. A method for treating animals infested or infected by parasites or preventing animals of getting infected or infested by parasites or protecting animals against infestation or infection by parasites which comprises administering or applying to the animals a parasiticidally effective amount of a compound as defined in claim 28.

54. The compound as defined in claim 28 for the use in the treatment of animals infested or infected by parasites, for preventing animals of getting infected or infested by parasites or for protecting animals against infestation or infection by parasites.
